SEQUENCE LISTING (74 sequences)

SEQ ID NO: 1-20 - skipped (20 entries)

SEQ ID NO: 21 - DNA, 29 bp, synthetic construct
  source 1..29: mol_type = other DNA
acaaaggcagccacgcactccttccctgt

SEQ ID NO: 22 - DNA, 33 bp, synthetic construct
  source 1..33: mol_type = other DNA
acactctttccctacacgacgctcttccgatct

SEQ ID NO: 23 - DNA, 29 bp, synthetic construct
  source 1..29: mol_type = other DNA
ctccagcgagatgaccctcaccaaccact

SEQ ID NO: 24 - DNA, 34 bp, synthetic construct
  source 1..34: mol_type = other DNA
gtgactggagttcagacgtgtgctcttccgatct

SEQ ID NO: 25 - DNA, 19 bp, synthetic construct
  source 1..19: mol_type = other DNA
aatgatacggcgaccaccg

SEQ ID NO: 26 - DNA, 24 bp, synthetic construct
  source 1..24: mol_type = other DNA
caagcagaagacggcatacgagat

SEQ ID NO: 27 - DNA, 32 bp, synthetic construct
  source 1..32: mol_type = other DNA
aacgccaaacctacggctttacttcctgtggc

SEQ ID NO: 28 - DNA, 32 bp, synthetic construct
  source 1..32: mol_type = other DNA
tcttgagtcattcgcagggcatgtgccagacc

SEQ ID NO: 29 - DNA, 32 bp, synthetic construct
  source 1..32: mol_type = other DNA
tcggcgttgtctgctatcgttcttggcactcc

SEQ ID NO: 30 - DNA, 32 bp, synthetic construct
  source 1..32: mol_type = other DNA
ggagcaataaccataaggccgttgacaagccc

SEQ ID NO: 31 - DNA, 32 bp, synthetic construct
  source 1..32: mol_type = other DNA
ggcgtattgccttggttctggcagcctcattg

SEQ ID NO: 32 - DNA, 32 bp, synthetic construct
  source 1..32: mol_type = other DNA
cagcagagggaacgatttcaacttcctgtggc

SEQ ID NO: 33 - DNA, 32 bp, synthetic construct
  source 1..32: mol_type = other DNA
ctactgcaagggtgtctagaatgtgccagacc

SEQ ID NO: 34 - DNA, 32 bp, synthetic construct
  source 1..32: mol_type = other DNA
gaccgactcgtgaaacgtaatcttggcactcc

SEQ ID NO: 35 - DNA, 32 bp, synthetic construct
  source 1..32: mol_type = other DNA
acacattctttgcgcccagagttgacaagccc

SEQ ID NO: 36 - DNA, 32 bp, synthetic construct
  source 1..32: mol_type = other DNA
atttcattcgacacccggtcgcagcctcattg

SEQ ID NO: 37 - DNA, 32 bp, synthetic construct
  source 1..32: mol_type = other DNA
agccacaggaagtaaagccgtaggtttggcgt

SEQ ID NO: 38 - DNA, 33 bp, synthetic construct
  source 1..33: mol_type = other DNA
aggtctggcacatgccctgcgaatgactcaaga

SEQ ID NO: 39 - DNA, 33 bp, synthetic construct
  source 1..33: mol_type = other DNA
aggagtgccaagaacgatagcagacaacgccga

SEQ ID NO: 40 - DNA, 33 bp, synthetic construct
  source 1..33: mol_type = other DNA
agggcttgtcaacggccttatggttattgctcc

SEQ ID NO: 41 - DNA, 33 bp, synthetic construct
  source 1..33: mol_type = other DNA
acaatgaggctgccagaaccaaggcaatacgcc

SEQ ID NO: 42 - DNA, 33 bp, synthetic construct
  source 1..33: mol_type = other DNA
agccacaggaagttgaaatcgttccctctgctg

SEQ ID NO: 43 - DNA, 33 bp, synthetic construct
  source 1..33: mol_type = other DNA
aggtctggcacattctagacacccttgcagtag

SEQ ID NO: 44 - DNA, 33 bp, synthetic construct
  source 1..33: mol_type = other DNA
aggagtgccaagattacgtttcacgagtcggtc

SEQ ID NO: 45 - DNA, 33 bp, synthetic construct
  source 1..33: mol_type = other DNA
agggcttgtcaactctgggcgcaaagaatgtgt

SEQ ID NO: 46 - DNA, 33 bp, synthetic construct
  source 1..33: mol_type = other DNA
acaatgaggctgcgaccgggtgtcgaatgaaat

SEQ ID NO: 47 - DNA, 32 bp, synthetic construct
  source 1..32: mol_type = other DNA
tgttgcatctccacccggattgagccttcagc

SEQ ID NO: 48 - DNA, 32 bp, synthetic construct
  source 1..32: mol_type = other DNA
cacaacgggagctgtggaattggttcacctgg

SEQ ID NO: 49 - DNA, 32 bp, synthetic construct
  source 1..32: mol_type = other DNA
tggactaagactcgtcctccagcggacctaag

SEQ ID NO: 50 - DNA, 32 bp, synthetic construct
  source 1..32: mol_type = other DNA
gtatgatggtgttgcggcttctcgcttaacgc

SEQ ID NO: 51 - DNA, 32 bp, synthetic construct
  source 1..32: mol_type = other DNA
tctgagtgccagtgacttcacgcattcgcttg

SEQ ID NO: 52 - DNA, 32 bp, synthetic construct
  source 1..32: mol_type = other DNA
tacgacacactcgggctctatgggcttcatgg

SEQ ID NO: 53 - DNA, 32 bp, synthetic construct
  source 1..32: mol_type = other DNA
gtttgagtgaaggcggtccaacccttagtgcg

SEQ ID NO: 54 - DNA, 32 bp, synthetic construct
  source 1..32: mol_type = other DNA
ctataagtttgtcgtgcccgtgagccttcagc

SEQ ID NO: 55 - DNA, 32 bp, synthetic construct
  source 1..32: mol_type = other DNA
ggagtgacactgactacgtttggttcacctgg

SEQ ID NO: 56 - DNA, 32 bp, synthetic construct
  source 1..32: mol_type = other DNA
gtcaacgccctagcagacatagcggacctaag

SEQ ID NO: 57 - DNA, 32 bp, synthetic construct
  source 1..32: mol_type = other DNA
ccagaacctattgagcctgactcgcttaacgc

SEQ ID NO: 58 - DNA, 32 bp, synthetic construct
  source 1..32: mol_type = other DNA
aggtgttcgtacaatgaggccgcattcgcttg

SEQ ID NO: 59 - DNA, 32 bp, synthetic construct
  source 1..32: mol_type = other DNA
tggtcaagggcaactaatcctgggcttcatgg

SEQ ID NO: 60 - DNA, 32 bp, synthetic construct
  source 1..32: mol_type = other DNA
acaattacccgtttaccggcacccttagtgcg

SEQ ID NO: 61 - DNA, 33 bp, synthetic construct
  source 1..33: mol_type = other DNA
agctgaaggctcaatccgggtggagatgcaaca

SEQ ID NO: 62 - DNA, 33 bp, synthetic construct
  source 1..33: mol_type = other DNA
accaggtgaaccaattccacagctcccgttgtg

SEQ ID NO: 63 - DNA, 33 bp, synthetic construct
  source 1..33: mol_type = other DNA
acttaggtccgctggaggacgagtcttagtcca

SEQ ID NO: 64 - DNA, 33 bp, synthetic construct
  source 1..33: mol_type = other DNA
agcgttaagcgagaagccgcaacaccatcatac

SEQ ID NO: 65 - DNA, 33 bp, synthetic construct
  source 1..33: mol_type = other DNA
acaagcgaatgcgtgaagtcactggcactcaga

SEQ ID NO: 66 - DNA, 33 bp, synthetic construct
  source 1..33: mol_type = other DNA
accatgaagcccatagagcccgagtgtgtcgta

SEQ ID NO: 67 - DNA, 33 bp, synthetic construct
  source 1..33: mol_type = other DNA
acgcactaagggttggaccgccttcactcaaac

SEQ ID NO: 68 - DNA, 33 bp, synthetic construct
  source 1..33: mol_type = other DNA
agctgaaggctcacgggcacgacaaacttatag

SEQ ID NO: 69 - DNA, 33 bp, synthetic construct
  source 1..33: mol_type = other DNA
accaggtgaaccaaacgtagtcagtgtcactcc

SEQ ID NO: 70 - DNA, 33 bp, synthetic construct
  source 1..33: mol_type = other DNA
acttaggtccgctatgtctgctagggcgttgac

SEQ ID NO: 71 - DNA, 33 bp, synthetic construct
  source 1..33: mol_type = other DNA
agcgttaagcgagtcaggctcaataggttctgg

SEQ ID NO: 72 - DNA, 33 bp, synthetic construct
  source 1..33: mol_type = other DNA
acaagcgaatgcggcctcattgtacgaacacct

SEQ ID NO: 73 - DNA, 33 bp, synthetic construct
  source 1..33: mol_type = other DNA
accatgaagcccaggattagttgcccttgacca

SEQ ID NO: 74 - DNA, 33 bp, synthetic construct
  source 1..33: mol_type = other DNA
acgcactaagggtgccggtaaacgggtaattgt

(19) United States
(12) Patent Application Publication
(10) Pub. No.: US 2024/0229118 A1
(43) Pub. Date: Jul. 11, 2024

(54) CONTROLLED ROLLING CIRCLE AMPLIFICATION

(71) Applicant: Singular Genomics Systems, Inc., San Diego, CA (US)

(72) Inventors: Richard Que, San Diego, CA (US); Martin Maria Fabani, Encinitas, CA (US); Zhenmin Hong, San Diego, CA (US); Michael Lawson, Pasadena, CA (US)

(21) Appl. No.: 18/537,266

(22) Filed: Dec. 12, 2023

Related U.S. Application Data
(60) Provisional application No. 63/434,908, filed on Dec. 22, 2022.
(60) Provisional application No. 63/484,633, filed on Feb. 13, 2023.

(51) Int. Cl. C12Q 1/6841; C12Q 1/682

(52) U.S. Cl. CPC C12Q 1/6841; C12Q 1/682

(57) ABSTRACT

Disclosed herein, inter alia, are methods and compositions for improved circular polynucleotide amplification and detection.

### CROSS-REFERENCES TO RELATED APPLICATIONS

This application claims the benefit of U.S. Provisional Application No. 63/434,908, filed Dec. 22, 2022 and U.S. Provisional Application No. 63/484,633, filed Feb. 13, 2023, each of which are incorporated herein by reference in their entirety and for all purposes.

### SEQUENCE LISTING

The instant application contains a Sequence Listing, which has been submitted electronically in XML format and is hereby incorporated by reference in its entirety. Said Sequence Listing, created on Dec. 11, 2023, is named 051385-591001US_ST26.xml and is 53,184 bytes in size.

### BACKGROUND

Genetic analysis is taking on increasing importance in modern society as a diagnostic, prognostic, and forensic tool. DNA sequencing is a fundamental tool in biological and medical research; it is an essential technology for the paradigm of personalized precision medicine. Additionally, single-cell technologies have emerged to enable profiling the composition of the genome, epigenome, transcriptome, or proteome of a single cell. Uncovering the distribution, heterogeneity, spatial gene and protein co-expression patterns within cells and tissues is vital for understanding how cell co-localization influences tissue development and the spread of diseases such as cancer, which could lead to important new discoveries and therapeutics. Beyond quantifying gene and protein expression, obtaining precise sequencing information enables identification, monitoring, and possible treatment at the molecular level.

Current sequencing platforms require clonal amplification of the initial template library molecules to create clusters (i.e., polonies), each containing 100s to 10,000s of forward and reverse copies of an initial template library molecule. Cluster generation is useful for increasing the signal-to-noise ratio because typical systems are not sensitive enough to detect the extension of one base at the individual DNA template molecule level. Amplification methods employed in commercial sequencing devices typically amplify a template molecule using surface immobilized primers to produce a plurality of double-stranded nucleic acid molecules, wherein at least one strand of each double-stranded nucleic acid molecule is attached to the solid support at its 5′ ends. Typical isothermal nucleic acid amplification procedures may lead to non-specific amplification artifacts or may produce amplification products that are sterically unfavorable for detection, making the amplicons harder to reliably use in next-generation sequencing applications. Disclosed herein, inter alia, are solutions to these and other problems in the art.

### BRIEF SUMMARY

In an aspect is provided a method of forming single-stranded polynucleotides in situ, the method including: (a) within a cell or tissue, extending a first primer hybridized to a circular polynucleotide with a strand-displacing polymerase to generate a first extension product including one or more complements of the circular polynucleotide; (b) contacting the first extension product with a second immobilized primer and extending the second immobilized primer with a polymerase to generate a second immobilized extension product, wherein the second primer is immobilized to a cellular component or a matrix within the cell or tissue; and (c) nicking the first extension product with an endonuclease, thereby generating one or more polynucleotide fragments, and removing the polynucleotide fragments, thereby forming single-stranded polynucleotides in situ.

In an aspect is provided a method of forming single-stranded polynucleotides on a solid support, the method including: (a) extending a first primer hybridized to a circular polynucleotide with a strand-displacing polymerase to generate a first extension product including one or more complements of the circular polynucleotide; (b) contacting the first extension product with a second immobilized primer and extending the second immobilized primer with a polymerase to generate a second immobilized extension product, wherein the second primer is immobilized to a solid support; and (c) nicking the first extension product with an endonuclease, thereby generating one or more polynucleotide fragments, and removing the polynucleotide fragments, thereby forming single-stranded polynucleotides on a solid support.

In an aspect is provided a method of sequencing a circular polynucleotide, the method including: i) amplifying the circular polynucleotide in a cell or tissue by extending a first primer hybridized to the circular polynucleotide with a strand-displacing polymerase to generate a first extension product including one or more complements of the circular polynucleotide; ii) contacting the first extension product with a second primer and extending the second primer with a polymerase to generate a second immobilized extension product, wherein the second primer is immobilized to a cellular component or a matrix within the cell or tissue; iii) nicking the first extension product with an endonuclease, thereby generating one or more polynucleotide fragments, and removing the polynucleotide fragments, thereby forming single-stranded polynucleotides on the solid support; and iv) hybridizing a sequencing primer to the single-stranded polynucleotides, and extending the sequencing primer to generate a first sequencing read, wherein the sequencing primer is immobilized to a cellular component or a matrix within the cell or tissue.

In an aspect is provided a kit including a circularizable probe, a ligase, and an endonuclease, wherein the circularizable probe includes a first hybridization sequence capable of hybridizing to a first sequence of a target polynucleotide, a second hybridization sequence capable of hybridizing to a second sequence of the target polynucleotide, and a sequence recognized by the endonuclease.

### BRIEF DESCRIPTION OF THE DRAWINGS

FIGS. 1A-1E. A cartoon depiction of a cell that is attached to a surface, and is also fixed (e.g., using a fixing agent) and permeabilized according to known methods is illustrated in FIG. 1A. The cell may have been cultured on the surface, or the cell may have been initially cultured in suspension and then fixed to the surface. The RNA molecules present in the cell (depicted as a wavy lines) may be subjected to an amplification technique where a targeted padlock probe which contains an oligonucleotide barcode (e.g., 10-15 nucleotides) hybridizes on the RNA. Following padlock probe ligation, the excess content is washed away (e.g., unhybridized padlock probes). As shown in FIG. 1B, the padlock probe is primed across the ligation site (i.e., an amplification primer hybridizes to both the 3′ and 5′ ends of the padlock probe) and amplified to produce an amplicon. Alternatively, as shown in FIG. 1C, the padlock probe is primed at a region within the padlock probe (i.e., an amplification primer hybridizes a region of the padlock probe) and amplified to produce an amplicon. Here, the ligation site is depicted as a dashed line. The amplicon may be primed with a sequencing primer and subjected to a sequencing process, whereby the identity of the oligonucleotide barcode, and thus the identity of the RNA molecule is obtained, as illustrated in FIG. 1D. FIG. 1E shows the original cell wherein one resolved pixel (depicted by the dotted box) includes a plurality of amplicons.

FIG. 2. Resolving one pixel of the cell as depicted in FIGS. 1A-IE, which includes the detection of a plurality of sequencing signals (i.e., fluorescent signals in this example) arising from the barcoded RNA as described according to the methods disclosed herein. Crowding within cells leads to spatial overlap of fluorescence signals within the optically resolved volume when visualized simultaneously. A linear decomposition of the sequencing signal (i.e., the multiplexed signal) into the oligonucleotide barcode basis set allows for the detection and quantitative measurement of multiple RNA in each resolved volume.

FIG. 3. A cartoon depiction of a voxel with the primary coordinate system in Cartesian coordinates. The optically resolved volume has a lateral resolution corresponding to the xy plane, and an axial resolution, corresponding to the z axis as observed in FIG. 3. In embodiments, the dimensions (i.e., the x, y, and z dimensions) of the optically resolved volume are given as (x-dimension) x (y-dimension) x (z-dimension); for example 0.5 μm×0.5 μm×2 μm.

FIGS. 4A-4D. A cartoon depiction of a cell that is attached to a substrate surface (FIG. 4A) and fixed (e.g., using a fixing agent) and permeabilized according to known methods. The cell may have been cultured on the surface, or the cell may have been initially cultured in suspension and then fixed to the surface. The nucleic acid (e.g., mRNA, oncogene, or nucleic acid sequence of interest) present in the cell (depicted as a wavy line) is subjected to an amplification technique where a targeted oligonucleotide primer anneals to the nucleic acid of interest. The black wedges represent the first and second regions of the oligonucleotide primer that hybridize to the first and second complementary regions of the target nucleic acid. As shown in FIG. 4B, the oligonucleotide probe hybridizes to regions adjacent to (i.e., flanking) the target nucleic acid sequence, referred to as the first and the second complementary regions (depicted as white boxes). In the presence of a polymerase (e.g., a non-strand displacing polymerase), the complement to the target sequence is generated by extending from the first complementary region of the oligonucleotide primer, and is ligated (not shown) to the second complementary region to form a circularized oligonucleotide, as found in FIG. 4C. The resulting circularized oligonucleotide is primed with an amplification primer and extended with a strand-displacing polymerase to generate a concatemer containing multiple copies of the target nucleic acid sequence, as depicted in FIG. 4D.

FIGS. 5A-5E illustrate an embodiment of the invention described herein for amplifying (e.g., by exponential rolling circle amplification (eRCA)) a circular template polynucleotide (e.g., a circularized probe or circularized polynucleotide). FIG. 5A depicts annealing of the circular template polynucleotide to a first immobilized amplification oligonucleotide (e.g., an oligonucleotide or primer immobilized at a 5′ end of the primer to a solid support, or immobilized at a 5′ end of the primer to a cellular component or polymer matrix in situ), and subsequent extension (e.g., extension with a strand-displacing polymerase) of the first immobilized oligonucleotide to generate an immobilized amplicon (e.g., an immobilized concatemer including a plurality of complements of the circular template polynucleotide). For clarity, the solid support or cellular component is illustrated as a flat black line. While only a single circular template polynucleotide is illustrated, it will be apparent to one of skill in the art that a plurality of circular template polynucleotides may be annealed and amplified across a plurality of first immobilized oligonucleotides (e.g., a first plurality of immobilized primers) using the methods described herein. FIG. 5B depicts hybridization of a second immobilized amplification oligonucleotide (e.g., a second amplification primer) to the immobilized RCA product of FIG. 5A, followed by extension of the second amplification primer to generate an extension product complementary to a portion of the immobilized RCA product. Additional second amplification primers may anneal to the immobilized RCA product and be extended with a strand-displacing polymerase, as illustrated in FIG. 5C, generating a plurality of immobilized complements of the RCA product, wherein each immobilized complement includes the complement of a portion of the RCA product, and wherein generating each additional immobilized complement displaces the previously generated immobilized complement. While not shown for clarity, it is to be understood that as additional immobilized complements are generated and displaced, one or more of the plurality of first amplification primers are then able to hybridize to the displaced immobilized complements and be extended. A nicking reaction is then performed (e.g., nicking by a nicking endonuclease that nicks one strand of a double-stranded nucleic acid substrate) as illustrated in FIG. 5D, generating nicks across the immobilized RCA product strand, for example. After washing under denaturing conditions, for example, the nicked portions of the RCA product are removed, leaving behind a plurality of immobilized complements of the RCA product as illustrated in FIG. 5E. The immobilized complements may then be detected using, for example, labeled probes or subjected to a sequencing process as described herein. As illustrated, immobilized complements with longer portions of sequence may be bound by an increased number of labeled probes, resulting in greater signal intensity.

FIG. 6 illustrates a series of fluorescence microscopy images of U-138MG cells targeted with circularizable probes specific for a target gene and subjected to the amplification and nicking process described herein and illustrated in FIGS. 5A-5E. Amplification (e.g., eRCA) of the circularized probes was performed for 20, 25, 30, or 35 minutes, as indicated in each panel. Following the nicking reaction, labeled probes specific for the immobilized amplification products were hybridized, and imaging performed. Arrows indicate the detected amplicon-specific probes. A poly-T-specific probe was also used to detect total mRNA in the cell (detected as an outline of the cell body). The intensity and number of clusters increases throughout each time point. Quantification of the amplicon cluster intensities is provided in Table 1. The scattered puncta in the images are detectable focusing beads added to the sample prior to imaging.

FIGS. 7A-7E illustrate an additional embodiment of the invention described herein for amplifying (e.g., by exponential rolling circle amplification (eRCA)) a circular template polynucleotide (e.g., a circularized probe or circularized polynucleotide) followed by sequencing with immobilized sequencing primers. FIG. 7A depicts annealing of the circular template polynucleotide to a first immobilized amplification oligonucleotide (e.g., an oligonucleotide or primer immobilized at a 5′ end of the primer to a solid support, or immobilized at a 5′ end of the primer to a cellular component or matrix in situ), and subsequent extension (e.g., extension with a strand-displacing polymerase) of the first immobilized oligonucleotide to generate an immobilized amplicon (e.g., an immobilized concatemer including a plurality of complements of the circular template polynucleotide). While only a single circular template polynucleotide is illustrated, it will be apparent to one of skill in the art that a plurality of circular template polynucleotides may be annealed and amplified across a plurality of first immobilized oligonucleotides (e.g., a first plurality of immobilized primers) using the methods described herein. As highlighted in FIG. 7A, the immobilized amplicon includes a plurality of sequencing primer binding sequences that are complementary to an immobilized sequencing primer. The immobilized sequencing primer is blocked (denoted by the “X”) to prevent extension from the 3′ end until the blocking moiety (e.g., a reversible terminator moiety) is removed. FIG. 5B depicts hybridization of a second immobilized amplification oligonucleotide (e.g., a second amplification primer) to the immobilized RCA product of FIG. 7A, followed by extension of the second amplification primer to generate an extension product complementary to a portion of the immobilized RCA product. Additional second amplification primers may anneal to the immobilized RCA product and be extended with a strand-displacing polymerase, as illustrated in FIG. 7C, generating a plurality of immobilized complements of the RCA product, wherein each immobilized complement includes the complement of a portion of the RCA product, and wherein generating each additional immobilized complement displaces the previously generated immobilized complement. While not shown for clarity, it is to be understood that as additional immobilized complements are generated and displaced, one or more of the plurality of first amplification primers are then able to hybridize to the displaced immobilized complements and be extended. A nicking reaction is then performed (e.g., nicking by a nicking endonuclease that nicks one strand of a double-stranded nucleic acid substrate) as illustrated in FIG. 7D, generating nicks across the immobilized RCA product strand, for example. After washing under denaturing conditions, for example, the nicked portions of the RCA product are removed, leaving behind a plurality of immobilized complements of the RCA product. Subsequently, the immobilized complements may hybridize to the immobilized sequencing primers, as shown in FIG. 7E. For clarity, only a single immobilized amplicon is illustrated, but it will be understood that the plurality of immobilized complements of the RCA product, for example as depicted in FIG. 7D, are present and also able to hybridize to the immobilized sequencing primer. The blocking moiety is removed from the sequencing primers, and a sequencing reaction (using, for example, reversibly-terminated labeled nucleotide incorporation and detection) is performed to determine the sequence of the amplification products.

FIG. 8 illustrates a portion of a circularizable probe, wherein the circularizable probe includes a first target hybridization sequence (e.g., Arm1), a first primer binding sequence (PBS1), a nicking site complement, a second primer binding sequence (PBS2′), and a second target hybridization sequence (e.g., Arm2). Following circularization and rolling circle amplification (e.g., RCA or eRCA), an RCA amplicon strand and an RCA strand complement are generated, including nicking sites and complements thereof.

FIGS. 9A-9H. Cartoon illustration of an in situ detection method in according with some embodiments. FIG. 9A depicts a fixed cellular matrix 900 including a nucleus 910 and circular polynucleotides 915. FIG. 9B depicts annealing of a first primer (e.g., a first amplification primer) 920 to one of the circular polynucleotides 915 from FIG. 9A. FIG. 9C depicts extension of primer 920 from FIG. 4B to generate a first extension product 930. FIG. 9D depicts annealing of the first extension product 930 to immobilized second primers 925 attached to a cellular component (or a matrix) 920. FIG. 9E depicts a step of extending the immobilized second primers 925 hybridized to the first extension product 930 and generating an immobilized second extension product 940. A third immobilized primer (e.g., an immobilized first primer) 950 is then annealed to the immobilized second extension product and extended to generate an immobilized third extension product, as shown in FIG. 9F. FIG. 9G depicts cleaving the immobilized second extension products with a nicking endonuclease 960, and removing the nicked fragments. FIG. 9H illustrates detection of the first and third extension products, for example, with a labeled probe oligonucleotide 970. Note that cellular components and oligos are not drawn to scale and are spread apart for clarity of illustration.

### DETAILED DESCRIPTION

The aspects and embodiments described herein relate to improved amplification and sequencing methods for circular template polynucleotides. The methods provide significant advantages in terms of speed and detection efficiency of target polynucleotides, and may be performed on solid supports or in cells or tissue sections in situ.

### I. Definitions

All patents, patent applications, articles and publications mentioned herein, both supra and infra, are hereby expressly incorporated herein by reference in their entireties. The practice of the technology described herein will employ, unless indicated specifically to the contrary, conventional methods of chemistry, biochemistry, organic chemistry, molecular biology, bioinformatics, microbiology, recombinant DNA techniques, genetics, immunology, and cell biology that are within the skill of the art, many of which are described below for the purpose of illustration. Examples of such techniques are available in the literature. See, e.g., Singleton et al., DICTIONARY OF MICROBIOLOGY AND MOLECULAR BIOLOGY 2nd ed., J. Wiley & Sons (New York, NY 1994); and Sambrook and Green, Molecular Cloning: A Laboratory Manual, 4th Edition (2012). Methods, devices and materials similar or equivalent to those described herein can be used in the practice of this invention.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole. It is to be understood that this disclosure is not limited to the particular methodology, protocols, and reagents described, as these may vary, depending upon the context in which they are used by those of skill in the art. The following definitions are provided to facilitate understanding of certain terms used frequently herein and are not meant to limit the scope of the present disclosure.

As used herein, the singular terms “a”, “an”, and “the” include the plural reference unless the context clearly indicates otherwise. Reference throughout this specification to, for example, “one embodiment”, “an embodiment”, “another embodiment”, “a particular embodiment”, “a related embodiment”, “a certain embodiment”, “an additional embodiment”, or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

As used herein, the term “control” or “control experiment” is used in accordance with its plain and ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

As used herein, the term “complement” is used in accordance with its plain and ordinary meaning and refers to a nucleotide (e.g., RNA nucleotide or DNA nucleotide) or a sequence of nucleotides capable of base pairing with a complementary nucleotide or sequence of nucleotides (e.g., Watson-Crick base pairing). As described herein and commonly known in the art the complementary (matching) nucleotide of adenosine is thymidine and the complementary (matching) nucleotide of guanosine is cytosine. Thus, a complement may include a sequence of nucleotides that base paired with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence. Another example of complementary sequences are a template sequence and an amplicon sequence polymerized by a polymerase along the template sequence. “Duplex” means at least two oligonucleotides and/or polynucleotides that are fully or partially complementary undergo Watson-Crick type base pairing among all or most of their nucleotides so that a stable complex is formed. Complementary single stranded nucleic acids and/or substantially complementary single stranded nucleic acids can hybridize to each other under hybridization conditions, thereby forming a nucleic acid that is partially or fully double stranded. When referring to a double-stranded polynucleotide including a first strand hybridized to a second strand, it is understood that each of the first strand and the second strand are independently single-stranded polynucleotides. All or a portion of a nucleic acid sequence may be substantially complementary to another nucleic acid sequence, in some embodiments. As referred to herein, “substantially complementary” refers to nucleotide sequences that can hybridize with each other under suitable hybridization conditions. Hybridization conditions can be altered to tolerate varying amounts of sequence mismatch within complementary nucleic acids that are substantially complementary. Substantially complementary portions of nucleic acids that can hybridize to each other can be 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more complementary to each other. In some embodiments substantially complementary portions of nucleic acids that can hybridize to each other are 100% complementary. Nucleic acids, or portions thereof, that are configured to hybridize to each other often comprise nucleic acid sequences that are substantially complementary to each other.

As described herein, the complementarity of sequences may be partial, in which only some of the nucleic acids match according to base pairing, or complete, where all the nucleic acids match according to base pairing. Thus, two sequences that are complementary to each other, may have a specified percentage of nucleotides that complement one another (e.g., about 60%, preferably 65%, 70%, 75%, 80%, 85%, 90%, 91%, 92%, 93%, 94%, 95%, 96%, 97%, 98%, 99%, or higher complementarity over a specified region). In embodiments, two sequences are complementary when they are completely complementary, having 100% complementarity. In embodiments, sequences in a pair of complementary sequences form portions of a single polynucleotide with non-base-pairing nucleotides (e.g., as in a hairpin or loop structure, with or without an overhang) or portions of separate polynucleotides. In embodiments, one or both sequences in a pair of complementary sequences form portions of longer polynucleotides, which may or may not include additional regions of complementarity.

As used herein, the term “contacting” is used in accordance with its plain ordinary meaning and refers to the process of allowing at least two distinct species (e.g., chemical compounds including biomolecules, particles, solid supports, or cells) to become sufficiently proximal to react, interact or physically touch. It should be appreciated, however, that the resulting reaction product can be produced directly from a reaction between the added reagents or from an intermediate from one or more of the added reagents which can be produced in the reaction mixture. The term “contacting” may include allowing two species to react, interact, or physically touch, wherein the two species may be a compound as described herein and a protein or enzyme.

As may be used herein, the terms “nucleic acid,” “nucleic acid molecule,” “nucleic acid sequence,” “strand,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may include natural nucleic acid sequences and variants thereof, artificial nucleic acid sequences, or a combination of such sequences. As may be used herein, the terms “nucleic acid oligomer” and “oligonucleotide” are used interchangeably and are intended to include, but are not limited to, nucleic acids having a length of 200 nucleotides or less. In some embodiments, an oligonucleotide is a nucleic acid having a length of 2 to 200 nucleotides, 2 to 150 nucleotides, 5 to 150 nucleotides or 5 to 100 nucleotides. The terms “polynucleotide,” “oligonucleotide,” “oligo” or the like refer, in the usual and customary sense, to a linear sequence of nucleotides. Oligonucleotides are typically from about 5, 6, 7, 8, 9, 10, 12, 15, 25, 30, 40, 50 or more nucleotides in length, up to about 100 nucleotides in length. In some embodiments, an oligonucleotide is a primer configured for extension by a polymerase when the primer is annealed completely or partially to a complementary nucleic acid template. A primer is often a single stranded nucleic acid. In certain embodiments, a primer, or portion thereof, is substantially complementary to a portion of an adapter. In some embodiments, a primer has a length of 200 nucleotides or less. In certain embodiments, a primer has a length of 10 to 150 nucleotides, 15 to 150 nucleotides, 5 to 100 nucleotides, 5 to 50 nucleotides or 10 to 50 nucleotides. In some embodiments, an oligonucleotide may be immobilized to a solid support.

As used herein, the terms “polynucleotide primer” and “primer” refers to any polynucleotide molecule that may hybridize to a polynucleotide template, be bound by a polymerase, and be extended in a template-directed process for nucleic acid synthesis (e.g., amplification and/or sequencing). The primer may be a separate polynucleotide from the polynucleotide template, or both may be portions of the same polynucleotide (e.g., as in a hairpin structure having a 3′ end that is extended along another portion of the polynucleotide to extend a double-stranded portion of the hairpin). Primers (e.g., forward or reverse primers) may be attached to a solid support. A primer can be of any length depending on the particular technique it will be used for. For example, PCR primers are generally between 10 and 40 nucleotides in length. The length and complexity of the nucleic acid fixed onto the nucleic acid template may vary. In some embodiments, a primer has a length of 200 nucleotides or less. In certain embodiments, a primer has a length of 10 to 150 nucleotides, 15 to 150 nucleotides, 5 to 100 nucleotides, 5 to 50 nucleotides or 10 to 50 nucleotides. In certain embodiments, a primer has a length of 10 to 150 nucleotides, 15 to 150 nucleotides, 5 to 100 nucleotides, 5 to 50 nucleotides or 10 to 50 nucleotides. A primer typically has a length of 10 to 50 nucleotides. For example, a primer may have a length of 10 to 40, 10 to 30, 10 to 20, 25 to 50, 15 to 40, 15 to 30, 20 to 50, 20 to 40, or 20 to 30 nucleotides. In some embodiments, a primer has a length of 18 to 24 nucleotides. One of skill can adjust these factors to provide optimum hybridization and signal production for a given hybridization procedure. The primer permits the addition of a nucleotide residue thereto, or oligonucleotide or polynucleotide synthesis therefrom, under suitable conditions. In an embodiment the primer is a DNA primer, i.e., a primer consisting of, or largely consisting of, deoxyribonucleotide residues. The primers are designed to have a sequence that is the complement of a region of template/target DNA to which the primer hybridizes. The addition of a nucleotide residue to the 3′ end of a primer by formation of a phosphodiester bond results in a DNA extension product. The addition of a nucleotide residue to the 3′ end of the DNA extension product by formation of a phosphodiester bond results in a further DNA extension product. In another embodiment the primer is an RNA primer. In embodiments, a primer is hybridized to a target polynucleotide. A “primer” is complementary to a polynucleotide template, and complexes by hydrogen bonding or hybridization with the template to give a primer/template complex for initiation of synthesis by a polymerase, which is extended by the addition of covalently bonded bases linked at its 3′ end complementary to the template in the process of DNA synthesis.

As used herein, the term “primer binding sequence” refers to a polynucleotide sequence that is complementary to at least a portion of a primer (e.g., a sequencing primer or an amplification primer). Primer binding sequences can be of any suitable length. In embodiments, a primer binding sequence is about or at least about 10, 15, 20, 25, 30, or more nucleotides in length. In embodiments, a primer binding sequence is 10-50, 15-30, or 20-25 nucleotides in length. The primer binding sequence may be selected such that the primer (e.g., sequencing primer) has the preferred characteristics to minimize secondary structure formation or minimize non-specific amplification, for example having a length of about 20-30 nucleotides; approximately 50% GC content, and a Tm of about 55° C. to about 65° C.

Nucleic acids, including e.g., nucleic acids with a phosphorothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

As used herein, a platform primer is a primer oligonucleotide immobilized or otherwise bound to a solid support (i.e. an immobilized oligonucleotide). Examples of platform primers include P7 and P5 primers, or S1 and S2 sequences, or the reverse complements thereof. A “platform primer binding sequence” refers to a sequence or portion of an oligonucleotide that is capable of binding to a platform primer (e.g., the platform primer binding sequence is complementary to the platform primer). In embodiments, a platform primer binding sequence may form part of an adapter. In embodiments, a platform primer binding sequence is complementary to a platform primer sequence. In embodiments, a platform primer binding sequence is complementary to a primer.

The order of elements within a nucleic acid molecule is typically described herein from 5′ to 3′. In the case of a double-stranded molecule, the “top” strand is typically shown from 5′ to 3′, according to convention, and the order of elements is described herein with reference to the top strand.

The term “messenger RNA” or “mRNA” refers to an RNA that is without introns and is capable of being translated into a polypeptide. The term “RNA” refers to any ribonucleic acid, including but not limited to mRNA, tRNA (transfer RNA), rRNA (ribosomal RNA), and/or noncoding RNA (such as lncRNA (long noncoding RNA)). The term “cDNA” refers to a DNA that is complementary or identical to an RNA, in either single stranded or double stranded form.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

As used herein, the term “associated” or “associated with” can mean that two or more species are identifiable as being co-located at a point in time. An association can mean that two or more species are or were within a similar container. An association can be an informatics association, where for example digital information regarding two or more species is stored and can be used to determine that one or more of the species were co-located at a point in time. An association can also be a physical association. In some instances two or more associated species are “tethered”, “coated”, “attached”, or “immobilized” to one another or to a common solid or semisolid support (e.g. a receiving substrate). An association may refer to a relationship, or connection, between two entities. For example, a barcode sequence may be associated with a particular target by binding a probe including the barcode sequence to the target. In embodiments, detecting the associated barcode provides detection of the target. Associated may refer to the relationship between a sample and the DNA molecules, RNA molecules, or polynucleotides originating from or derived from that sample. These relationships may be encoded in oligonucleotide barcodes, as described herein. A polynucleotide is associated with a sample if it is an endogenous polynucleotide, i.e., it occurs in the sample at the time the sample is obtained, or is derived from an endogenous polynucleotide. For example, the RNAs endogenous to a cell are associated with that cell. cDNAs resulting from reverse transcription of these RNAs, and DNA amplicons resulting from PCR amplification of the cDNAs, contain the sequences of the RNAs and are also associated with the cell. The polynucleotides associated with a sample need not be located or synthesized in the sample, and are considered associated with the sample even after the sample has been destroyed (for example, after a cell has been lysed). Barcoding can be used to determine which polynucleotides in a mixture are associated with a particular sample. In embodiments, a proximity probe is associated with a particular barcode, such that identifying the barcode identifies the probe with which it is associated. Because the proximity probe specifically binds to a target, identifying the barcode thus identifies the target.

The term “adapter” as used herein refers to any oligonucleotide that can be ligated to a nucleic acid molecule, thereby generating nucleic acid products that can be sequenced on a sequencing platform (e.g., an Illumina or Singular Genomics G4™ sequencing platform). In embodiments, adapters include two reverse complementary oligonucleotides forming a double-stranded structure. In embodiments, an adapter includes two oligonucleotides that are complementary at one portion and mismatched at another portion, forming a Y-shaped or fork-shaped adapter that is double stranded at the complementary portion and has two overhangs at the mismatched portion. Since Y-shaped adapters have a complementary, double-stranded region, they can be considered a special form of double-stranded adapters. When this disclosure contrasts Y-shaped adapters and double stranded adapters, the term “double-stranded adapter” or “blunt-ended” is used to refer to an adapter having two strands that are fully complementary, substantially (e.g., more than 90% or 95%) complementary, or partially complementary. In embodiments, adapters include sequences that bind to sequencing primers. In embodiments, adapters include sequences that bind to immobilized oligonucleotides (e.g., P7 and P5 sequences) or reverse complements thereof. In embodiments, the adapter is substantially non-complementary to the 3′ end or the 5′ end of any target polynucleotide present in the sample. In embodiments, the adapter can include a sequence that is substantially identical, or substantially complementary, to at least a portion of a primer, for example a universal primer. In embodiments, the adapter can include an index sequence (also referred to as barcode or tag) to assist with downstream error correction, identification or sequencing.

As used herein, the term “hairpin adapter” refers to a polynucleotide including a double-stranded stem portion and a single-stranded hairpin loop portion. In some embodiments, an adapter is hairpin adapter (also referred to herein as a hairpin). In some embodiments, a hairpin adapter includes a single nucleic acid strand including a stem-loop structure. In some embodiments, a hairpin adapter includes a nucleic acid having a 5′-end, a 5′-portion, a loop, a 3′-portion and a 3′-end (e.g., arranged in a 5′ to 3′ orientation). In some embodiments, the 5′ portion of a hairpin adapter is annealed and/or hybridized to the 3′ portion of the hairpin adapter, thereby forming a stem portion of the hairpin adapter. In some embodiments, the 5′ portion of a hairpin adapter is substantially complementary to the 3′ portion of the hairpin adapter. In certain embodiments, a hairpin adapter includes a stem portion (i.e., stem) and a loop, wherein the stem portion is substantially double stranded thereby forming a duplex. In some embodiments, the loop of a hairpin adapter includes a nucleic acid strand that is not complementary (e.g., not substantially complementary) to itself or to any other portion of the hairpin adapter. In some embodiments, a method herein includes ligating a first adapter to a first end of a double stranded nucleic acid, and ligating a second adapter to a second end of a double stranded nucleic acid. In some embodiments, the first adapter and the second adapter are different. For example, in certain embodiments, the first adapter and the second adapter may include different nucleic acid sequences or different structures. In some embodiments, the first adapter is a Y-adapter and the second adapter is a hairpin adapter. In some embodiments, the first adapter is a hairpin adapter and a second adapter is a hairpin adapter. In certain embodiments, the first adapter and the second adapter may include different primer binding sites, different structures, and/or different capture sequences (e.g., a sequence complementary to a capture nucleic acid). In some embodiments, some, all or substantially all of the nucleic acid sequence of a first adapter and a second adapter are the same. In some embodiments, some, all or substantially all of the nucleic acid sequence of a first adapter and a second adapter are substantially different.

As used herein, the terms “analogue” and “analog”, in reference to a chemical compound, refers to compound having a structure similar to that of another one, but differing from it in respect of one or more different atoms, functional groups, or substructures that are replaced with one or more other atoms, functional groups, or substructures. In the context of a nucleotide, a nucleotide analog refers to a compound that, like the nucleotide of which it is an analog, can be incorporated into a nucleic acid molecule (e.g., an extension product) by a suitable polymerase, for example, a DNA polymerase in the context of a nucleotide analogue. The terms also encompass nucleic acids containing known nucleotide analogs or modified backbone residues or linkages, which are synthetic, naturally occurring, or non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphorothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see, e.g., see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA)), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Other analog nucleic acids include bis-locked nucleic acids (bisLNAs; e.g., including those described in Moreno PMD et al. Nucleic Acids Res. 2013; 41(5):3257-73), twisted intercalating nucleic acids (TINAs; e.g., including those described in Doluca O et al. Chembiochem. 2011; 12(15):2365-74), bridged nucleic acids (BNAs; e.g., including those described in Soler-Bistue A et al. Molecules. 2019; 24(12): 2297), 2′-O-methyl RNA:DNA chimeric nucleic acids (e.g., including those described in Wang S and Kool E T. Nucleic Acids Res. 1995; 23(7):1157-1164), minor groove binder (MGB) nucleic acids (e.g., including those described in Kutyavin I V et al. Nucleic Acids Res. 2000; 28(2):655-61), morpholino nucleic acids (e.g., including those described in Summerton J and Weller D. Antisense Nucleic Acid Drug Dev. 1997; 7(3):187-95), C5-modified pyrimidine nucleic acids (e.g., including those described in Kumar P et al. J. Org. Chem. 2014; 79(11): 5047-5061), peptide nucleic acids (PNAs; e.g., including those described in Gupta A et al. J. Biotechnol. 2017; 259: 148-59), and/or phosphorothioate nucleotides (e.g., including those described in Eckstein F. Nucleic Acid Ther. 2014; 24(6):374-87).

As used herein, a “native” nucleotide is used in accordance with its plain and ordinary meaning and refers to a naturally occurring nucleotide that does not include an exogenous label (e.g., a fluorescent dye, or other label) or chemical modification such as may characterize a nucleotide analog. Examples of native nucleotides useful for carrying out procedures described herein include: dATP (2′-deoxyadenosine-5′-triphosphate); dGTP (2′-deoxyguanosine-5′-triphosphate); dCTP (2′-deoxycytidine-5′-triphosphate); dTTP (2′-deoxythymidine-5′-triphosphate); and dUTP (2′-deoxyuridine-5′-triphosphate).

In embodiments, the nucleotides of the present disclosure use a cleavable linker to attach the label to the nucleotide. The use of a cleavable linker ensures that the label can, if required, be removed after detection, avoiding any interfering signal with any labelled nucleotide incorporated subsequently. The use of the term “cleavable linker” is not meant to imply that the whole linker is required to be removed from the nucleotide base. The cleavage site can be located at a position on the linker that ensures that part of the linker remains attached to the nucleotide base after cleavage. The linker can be attached at any position on the nucleotide base provided that Watson-Crick base pairing can still be carried out. In the context of purine bases, it is preferred if the linker is attached via the 7-position of the purine or the preferred deazapurine analogue, via an 8-modified purine, via an N-6 modified adenosine or an N-2 modified guanine. For pyrimidines, attachment is preferably via the 5-position on cytidine, thymidine or uracil and the N-4 position on cytosine.

The term “cleavable linker” or “cleavable moiety” as used herein refers to a divalent or monovalent, respectively, moiety which is capable of being separated (e.g., detached, split, disconnected, hydrolyzed, a stable bond within the moiety is broken) into distinct entities. A cleavable linker is cleavable (e.g., specifically cleavable) in response to external stimuli (e.g., enzymes, nucleophilic/basic reagents, reducing agents, photo-irradiation, electrophilic/acidic reagents, organometallic and metal reagents, or oxidizing reagents). A chemically cleavable linker refers to a linker which is capable of being split in response to the presence of a chemical (e.g., acid, base, oxidizing agent, reducing agent, Pd(0), tris-(2-carboxyethyl)phosphine, dilute nitrous acid, fluoride, tris(3-hydroxypropyl)phosphine), sodium dithionite (Na₂S₂O₄), or hydrazine (N₂H₄)). A chemically cleavable linker is non-enzymatically cleavable. In embodiments, the cleavable linker is cleaved by contacting the cleavable linker with a cleaving agent. In embodiments, the cleaving agent is a phosphine containing reagent (e.g., TCEP or THPP), sodium dithionite (Na₂S₂O₄), weak acid, hydrazine (N₂H₄), Pd(0), or light-irradiation (e.g., ultraviolet radiation). In embodiments, cleaving includes removing. A “cleavable site” or “scissile linkage” in the context of a polynucleotide is a site which allows controlled cleavage of the polynucleotide strand (e.g., the linker, the primer, or the polynucleotide) by chemical, enzymatic, or photochemical means known in the art and described herein. A scissile site may refer to the linkage of a nucleotide between two other nucleotides in a nucleotide strand (i.e., an internucleosidic linkage). In embodiments, the scissile linkage can be located at any position within the one or more nucleic acid molecules, including at or near a terminal end (e.g., the 3′ end of an oligonucleotide) or in an interior portion of the one or more nucleic acid molecules. In embodiments, conditions suitable for separating a scissile linkage include a modulating the pH and/or the temperature. In embodiments, a scissile site can include at least one acid-labile linkage. For example, an acid-labile linkage may include a phosphoramidate linkage. In embodiments, a phosphoramidate linkage can be hydrolysable under acidic conditions, including mild acidic conditions such as trifluoroacetic acid and a suitable temperature (e.g., 30° C.), or other conditions known in the art, for example Matthias Mag, et al Tetrahedron Letters, Volume 33, Issue 48, 1992, 7319-7322. In embodiments, the scissile site can include at least one photolabile internucleosidic linkage (e.g., o-nitrobenzyl linkages, as described in Walker et al, J. Am. Chem. Soc. 1988, 110, 21, 7170-7177), such as o-nitrobenzyloxymethyl or p-nitrobenzyloxymethyl group(s). In embodiments, the scissile site includes at least one uracil nucleobase. In embodiments, a uracil nucleobase can be cleaved with a uracil DNA glycosylase (UDG) or Formamidopyrimidine DNA Glycosylase Fpg. In embodiments, the scissile linkage site includes a sequence-specific nicking site having a nucleotide sequence that is recognized and nicked by a nicking endonuclease enzyme or a uracil DNA glycosylase. Cleavage agents used in methods described herein may be selected from nicking endonucleases, DNA glycosylases, or any single-stranded cleavage agents described in further detail elsewhere herein. Enzymes for cleavage of single-stranded DNA may be used for cleaving heteroduplexes in the vicinity of mismatched bases, D-loops, heteroduplexes formed between two strands of DNA which differ by a single base, an insertion or deletion. Mismatch recognition proteins that cleave one strand of the mismatched DNA in the vicinity of the mismatch site may be used as cleavage agents. Nonenzymatic cleaving may also be done through photodegredation of a linker introduced through a custom oligonucleotide used in a PCR reaction.

As used herein, the term “modified nucleotide” refers to nucleotide modified in some manner. Typically, a nucleotide contains a single 5-carbon sugar moiety, a single nitrogenous base moiety and 1 to three phosphate moieties. In embodiments, a nucleotide can include a blocking moiety and/or a label moiety. A blocking moiety on a nucleotide prevents formation of a covalent bond between the 3′ hydroxyl moiety of the nucleotide and the 5′ phosphate of another nucleotide. A blocking moiety on a nucleotide can be reversible, whereby the blocking moiety can be removed or modified to allow the 3′ hydroxyl to form a covalent bond with the 5′ phosphate of another nucleotide. A blocking moiety can be effectively irreversible under particular conditions used in a method set forth herein. In embodiments, the blocking moiety is attached to the 3′ oxygen of the nucleotide and is independently —NH₂, —CN, —CH₃, C₂-C₆ allyl (e.g., —CH₂—CH═CH₂), methoxyalkyl (e.g., —CH₂—O—CH₃), or —CH₂N₃. In embodiments, the blocking moiety is attached to the 3′ oxygen of the nucleotide and is independently

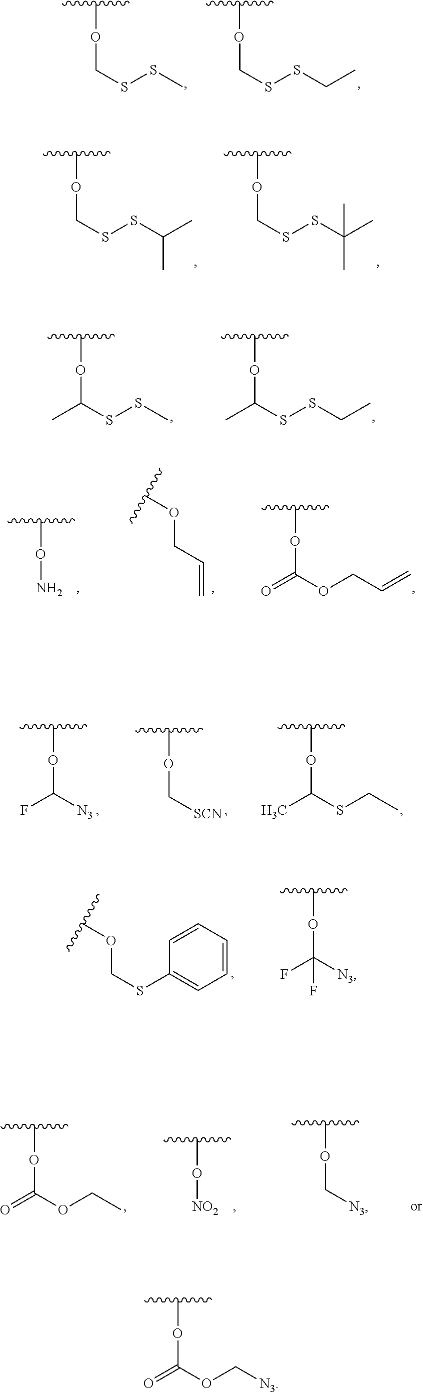

A label moiety of a modified nucleotide can be any moiety that allows the nucleotide to be detected, for example, using a spectroscopic method. Exemplary label moieties are fluorescent labels, mass labels, chemiluminescent labels, electrochemical labels, detectable labels and the like. One or more of the above moieties can be absent from a nucleotide used in the methods and compositions set forth herein. For example, a nucleotide can lack a label moiety or a blocking moiety or both. Examples of nucleotide analogues include, without limitation, 7-deaza-adenine, 7-deaza-guanine, the analogues of deoxynucleotides shown herein, analogues in which a label is attached through a cleavable linker to the 5-position of cytosine or thymine or to the 7-position of deaza-adenine or deaza-guanine, and analogues in which a small chemical moiety is used to cap the OH group at the 3′-position of deoxyribose. Nucleotide analogues and DNA polymerase-based DNA sequencing are also described in U.S. Pat. No. 6,664,079, which is incorporated herein by reference in its entirety for all purposes. Non-limiting examples of detectable labels include labels including fluorescent dyes, biotin, digoxin, haptens, and epitopes. In general, a dye is a molecule, compound, or substance that can provide an optically detectable signal, such as a colorimetric, luminescent, bioluminescent, chemiluminescent, phosphorescent, or fluorescent signal. In embodiments, the dye is a fluorescent dye. Non-limiting examples of dyes, some of which are commercially available, include CF dyes (Biotium, Inc.), Alexa Fluor dyes (Thermo Fisher), DyLight dyes (Thermo Fisher), Cy dyes (GE Healthscience), IRDyes (Li-Cor Biosciences, Inc.), and HiLyte dyes (Anaspec, Inc.). In embodiments, the label is a fluorophore.

In some embodiments, a nucleic acid includes a label. As used herein, the term “label” or “labels” is used in accordance with their plain and ordinary meanings and refer to molecules that can directly or indirectly produce or result in a detectable signal either by themselves or upon interaction with another molecule. Non-limiting examples of detectable labels include fluorescent dyes, biotin, digoxin, haptens, and epitopes. In general, a dye is a molecule, compound, or substance that can provide an optically detectable signal, such as a colorimetric, luminescent, bioluminescent, chemiluminescent, phosphorescent, or fluorescent signal. In embodiments, the label is a dye. In embodiments, the dye is a fluorescent dye. Non-limiting examples of dyes, some of which are commercially available, include CF dyes (Biotium, Inc.), Alexa Fluor dyes (Thermo Fisher), DyLight dyes (Thermo Fisher), Cy dyes (GE Healthscience), IRDyes (Li-Cor Biosciences, Inc.), and HiLyte dyes (Anaspec, Inc.). In embodiments, a particular nucleotide type is associated with a particular label, such that identifying the label identifies the nucleotide with which it is associated. In embodiments, the label is luciferin that reacts with luciferase to produce a detectable signal in response to one or more bases being incorporated into an elongated complementary strand, such as in pyrosequencing. In embodiment, a nucleotide includes a label (such as a dye). In embodiments, the label is not associated with any particular nucleotide, but detection of the label identifies whether one or more nucleotides having a known identity were added during an extension step (such as in the case of pyrosequencing). Examples of detectable agents (i.e., labels) include imaging agents, including fluorescent and luminescent substances, molecules, or compositions, including, but not limited to, a variety of organic or inorganic small molecules commonly referred to as “dyes,” “labels,” or “indicators.” Examples include fluorescein, rhodamine, acridine dyes, Alexa dyes, and cyanine dyes. In embodiments, the detectable moiety is a fluorescent molecule (e.g., acridine dye, cyanine, dye, fluorine dye, oxazine dye, phenanthridine dye, or rhodamine dye). In embodiments, the detectable moiety is a fluorescent molecule (e.g., acridine dye, cyanine, dye, fluorine dye, oxazine dye, phenanthridine dye, or rhodamine dye). The term “cyanine” or “cyanine moiety” as described herein refers to a detectable moiety containing two nitrogen groups separated by a polymethine chain. In embodiments, the cyanine moiety has 3 methine structures (i.e., cyanine 3 or Cy3). In embodiments, the cyanine moiety has 5 methine structures (i.e., cyanine 5 or Cy5). In embodiments, the cyanine moiety has 7 methine structures (i.e., cyanine 7 or Cy7).

The term “nucleoside” refers, in the usual and customary sense, to a glycosylamine including a nucleobase and a five-carbon sugar (ribose or deoxyribose). Non-limiting examples of nucleosides include cytidine, uridine, adenosine, guanosine, thymidine and inosine. Nucleosides may be modified at the base and/or the sugar. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g., polynucleotides contemplated herein include any types of RNA, e.g., mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site www.ncbi.nlm.nih.gov/BLAST/ or the like). Such sequences are then said to be “substantially identical.” This definition also refers to, or may be applied to, the complement of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

As used herein, the term “removable” group, e.g., a label or a blocking group or protecting group, is used in accordance with its plain and ordinary meaning and refers to a chemical group that can be removed from a nucleotide analogue such that a DNA polymerase can extend the nucleic acid (e.g., a primer or extension product) by the incorporation of at least one additional nucleotide. Removal may be by any suitable method, including enzymatic, chemical, or photolytic cleavage. Removal of a removable group, e.g., a blocking group, does not require that the entire removable group be removed, only that a sufficient portion of it be removed such that a DNA polymerase can extend a nucleic acid by incorporation of at least one additional nucleotide using a nucleotide or nucleotide analogue. In general, the conditions under which a removable group is removed are compatible with a process employing the removable group (e.g., an amplification process or sequencing process).

As used herein, the terms “reversible blocking groups” and “reversible terminators” are used in accordance with their plain and ordinary meanings and refer to a blocking moiety located, for example, at the 3′ position of a modified nucleotide and may be a chemically cleavable moiety such as an allyl group, an azidomethyl group or a methoxymethyl group, or may be an enzymatically cleavable group such as a phosphate ester. Non-limiting examples of nucleotide blocking moieties are described in applications WO 2004/018497, WO 96/07669, U.S. Pat. Nos. 7,057,026, 7,541,444, 5,763,594, 5,808,045, 5,872,244 and 6,232,465 the contents of which are incorporated herein by reference in their entirety. The nucleotides may be labelled or unlabeled. They may be modified with reversible terminators useful in methods provided herein and may be 3′-O-blocked reversible or 3′-unblocked reversible terminators. In nucleotides with 3′-O-blocked reversible terminators, the blocking group —OR [reversible terminating (capping) group] is linked to the oxygen atom of the 3′-OH of the pentose, while the label is linked to the base, which acts as a reporter and can be cleaved. The 3′-O-blocked reversible terminators are known in the art, and may be, for instance, a 3′-ONH₂ reversible terminator, a 3′-O-allyl reversible terminator, or a 3′-O-azidomethyl reversible terminator. In embodiments, the reversible terminator moiety is attached to the 3′-oxygen of the nucleotide, having the formula:

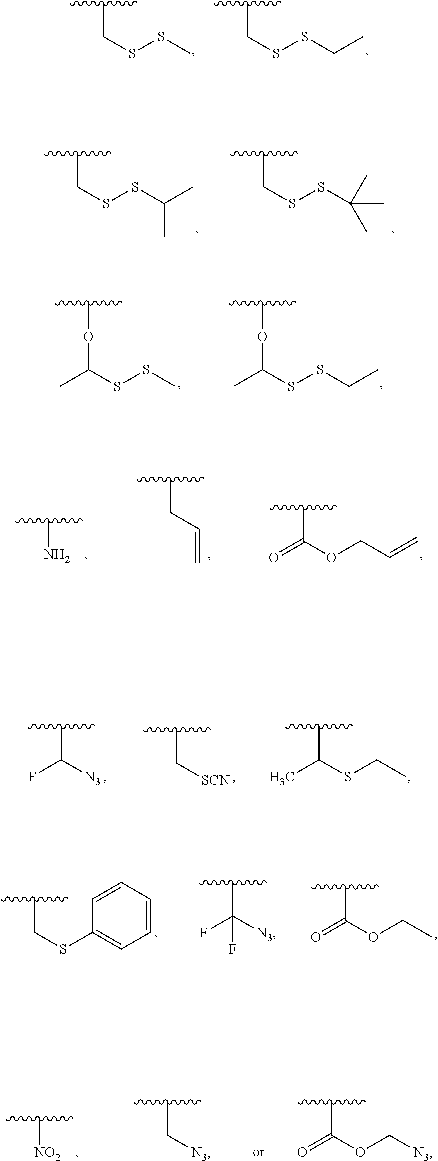

wherein the 3′ oxygen of the nucleotide is not shown in the formulae above. The term “allyl” as described herein refers to an unsubstituted methylene attached to a vinyl group (i.e., —CH═CH₂). In embodiments, the reversible terminator moiety is

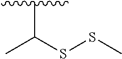 [SMILES: *C(C)SSC]

as described in U.S. Pat. No. 10,738,072, which is incorporated herein by reference for all purposes. For example, a nucleotide including a reversible terminator moiety may be represented by the formula:

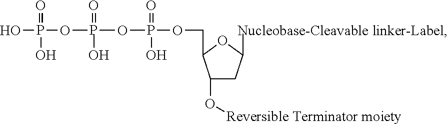

where the nucleobase is adenine or adenine analogue, thymine or thymine analogue, guanine or guanine analogue, or cytosine or cytosine analogue.

In some embodiments, a nucleic acid (e.g., a probe or a primer) includes a molecular identifier or a molecular barcode. As used herein, the term “molecular barcode” (which may be referred to as a “tag”, a “barcode”, a “molecular identifier”, an “identifier sequence” or a “unique molecular identifier” (UMI)) refers to any material (e.g., a nucleotide sequence, a nucleic acid molecule feature) that is capable of distinguishing an individual molecule in a large heterogeneous population of molecules. In embodiments, a barcode is unique in a pool of barcodes that differ from one another in sequence, or is uniquely associated with a particular sample polynucleotide in a pool of sample polynucleotides. In embodiments, every barcode in a pool of adapters is unique, such that sequencing reads including the barcode can be identified as originating from a single sample polynucleotide molecule on the basis of the barcode alone. In other embodiments, individual barcode sequences may be used more than once, but adapters including the duplicate barcodes are associated with different sequences and/or in different combinations of barcoded adaptors, such that sequence reads may still be uniquely distinguished as originating from a single sample polynucleotide molecule on the basis of a barcode and adjacent sequence information (e.g., sample polynucleotide sequence, and/or one or more adjacent barcodes). In embodiments, barcodes are about or at least about 5, 6, 7, 8, 9, 10, 15, 20, 25, 30, 40, 50, 75 or more nucleotides in length. In embodiments, barcodes are shorter than 20, 15, 10, 9, 8, 7, 6, or 5 nucleotides in length. In embodiments, barcodes are about 10 to about 50 nucleotides in length, such as about 15 to about 40 or about 20 to about 30 nucleotides in length. In a pool of different barcodes, barcodes may have the same or different lengths. In general, barcodes are of sufficient length and include sequences that are sufficiently different to allow the identification of sequencing reads that originate from the same sample polynucleotide molecule. In embodiments, each barcode in a plurality of barcodes differs from every other barcode in the plurality by at least three nucleotide positions, such as at least 3, 4, 5, 6, 7, 8, 9, 10, or more nucleotide positions. In some embodiments, substantially degenerate barcodes may be known as random. In some embodiments, a barcode may include a nucleic acid sequence from within a pool of known sequences. In some embodiments, the barcodes may be pre-defined. In embodiments, the barcodes are selected to form a known set of barcodes, e.g., the set of barcodes may be distinguished by a particular Hamming distance. In embodiments, each barcode sequence is unique within the known set of barcodes. In embodiments, each barcode sequence is associated with a particular oligonucleotide probe.

In embodiments, a nucleic acid (e.g., an adapter or primer) includes a sample barcode. In general, a “sample barcode” is a nucleotide sequence that is sufficiently different from other sample barcode to allow the identification of the sample source based on sample barcode sequence(s) with which they are associated. In embodiments, a plurality of nucleotides (e.g., all nucleotides from a particular sample source, or sub-sample thereof) are joined to a first sample barcode, while a different plurality of nucleotides (e.g., all nucleotides from a different sample source, or different subsample) are joined to a second sample barcode, thereby associating each plurality of polynucleotides with a different sample barcode indicative of sample source. In embodiments, each sample barcode in a plurality of sample barcodes differs from every other sample barcode in the plurality by at least three nucleotide positions, such as at least 3, 4, 5, 6, 7, 8, 9, 10, or more nucleotide positions. In some embodiments. substantially degenerate sample barcodes may be known as random. In some embodiments, a sample barcode may include a nucleic acid sequence from within a pool of known sequences. In some embodiments, the sample barcodes may be pre-defined. In embodiments, the sample barcode includes about 1 to about 10 nucleotides. In embodiments, the sample barcode includes about 3, 4, 5, 6, 7, 8, 9, or about 10 nucleotides. In embodiments, the sample barcode includes about 3 nucleotides. In embodiments, the sample barcode includes about 5 nucleotides. In embodiments, the sample barcode includes about 7 nucleotides. In embodiments, the sample barcode includes about 10 nucleotides. In embodiments, the sample barcode includes about 6 to about 10 nucleotides.

As used herein, the term “DNA polymerase” and “nucleic acid polymerase” are used in accordance with their plain ordinary meanings and refer to enzymes capable of synthesizing nucleic acid molecules from nucleotides (e.g., deoxyribonucleotides). Exemplary types of polymerases that may be used in the compositions and methods of the present disclosure include the nucleic acid polymerases such as DNA polymerase, DNA- or RNA-dependent RNA polymerase, and reverse transcriptase. In some cases, the DNA polymerase is 9° N polymerase or a variant thereof, E. Coli DNA polymerase I, Bacteriophage T4 DNA polymerase, Sequenase, Taq DNA polymerase, DNA polymerase from Bacillus stearothermophilus, Bst 2.0 DNA polymerase, 9° N polymerase (exo-)A485L/Y409V, Phi29 DNA Polymerase (φ29 DNA Polymerase), T7 DNA polymerase, DNA polymerase II, DNA polymerase III holoenzyme, DNA polymerase IV, DNA polymerase V, VentR DNA polymerase, Therminator™ II DNA Polymerase, Therminator™ III DNA Polymerase, or or Therminator™ IX DNA Polymerase. In embodiments, the polymerase is a protein polymerase. Typically, a DNA polymerase adds nucleotides to the 3′-end of a DNA strand, one nucleotide at a time. In embodiments, the DNA polymerase is a Pol I DNA polymerase, Pol II DNA polymerase, Pol III DNA polymerase, Pol IV DNA polymerase, Pol V DNA polymerase, Pol γ DNA polymerase, Pol μ DNA polymerase, Pol λ DNA polymerase, Pol σ DNA polymerase, Pol α DNA polymerase, Pol δ DNA polymerase, Pol ε DNA polymerase, Pol η DNA polymerase, Pol ξ DNA polymerase, Pol κ DNA polymerase, Pol ζ DNA polymerase, Pol γ DNA polymerase, Pol θ DNA polymerase, Pol ν DNA polymerase, or a thermophilic nucleic acid polymerase (e.g. Therminator γ, 9° N polymerase (exo-), Therminator II, Therminator III, or Therminator IX). In embodiments, the DNA polymerase is a modified archaeal DNA polymerase. In embodiments, the polymerase is a reverse transcriptase. In embodiments, the polymerase is a mutant P. abyssi polymerase (e.g., such as a mutant P. abyssi polymerase described in WO 2018/148723 or WO 2020/056044). In embodiments, the polymerase is an enzyme described in US 2021/0139884. For example, a polymerase catalyzes the addition of a next correct nucleotide to the 3′-OH group of the primer via a phosphodiester bond, thereby chemically incorporating the nucleotide into the primer. Optionally, the polymerase used in the provided methods is a processive polymerase. Optionally, the polymerase used in the provided methods is a distributive polymerase.

As used herein, the term “thermophilic nucleic acid polymerase” refers to a family of DNA polymerases (e.g., 9° N™) and mutants thereof derived from the DNA polymerase originally isolated from the hyperthermophilic archaea, Thermococcus sp. 9 degrees N-7, found in hydrothermal vents at that latitude (East Pacific Rise) (Southworth M W, et al. PNAS. 1996; 93(11):5281-5285). A thermophilic nucleic acid polymerase is a member of the family B DNA polymerases. Site-directed mutagenesis of the 3′-5′ exo motif I (Asp-Ile-Glu or DIE) to AIA, AIE, EIE, EID or DIA yielded polymerase with no detectable 3′ exonuclease activity. Mutation to Asp-Ile-Asp (DID) resulted in reduction of 3′-5′ exonuclease specific activity to <1% of wild type, while maintaining other properties of the polymerase including its high strand displacement activity. The sequence AIA (D141A, E143A) was chosen for reducing exonuclease. Subsequent mutagenesis of key amino acids results in an increased ability of the enzyme to incorporate dideoxynucleotides, ribonucleotides and acyclonucleotides (e.g., Therminator II enzyme from New England Biolabs with D141A/E143A/Y409V/A485L mutations); 3′-amino-dNTPs, 3′-azido-dNTPs and other 3′-modified nucleotides (e.g., NEB Therminator III DNA Polymerase with D141A/E143A/L408S/Y409A/P410V mutations, NEB Therminator IX DNA polymerase), or 7-phosphate labeled nucleotides (e.g., Therminator 7: D141A/E143A/W355A/L408 W/R460A/Q461S/K464E/D480V/R484 W/A485L). Typically, these enzymes do not have 5′-3′ exonuclease activity. Additional information about thermophilic nucleic acid polymerases may be found in (Southworth M W, et al. PNAS. 1996; 93(11):5281-5285; Bergen K, et al. ChemBioChem. 2013; 14(9):1058-1062; Kumar S, et al. Scientific Reports. 2012; 2:684; Fuller C W, et al. 2016; 113(19):5233-5238; Guo J, et al. Proceedings of the National Academy of Sciences of the United States of America. 2008; 105(27):9145-9150), which are incorporated herein in their entirety for all purposes.

As used herein, the term “exonuclease activity” is used in accordance with its ordinary meaning in the art, and refers to the removal of a nucleotide from a nucleic acid by an enzyme (e.g. DNA polymerase, a lambda exonuclease, Exo I, Exo III, T5, Exo V, Exo VII or the like). For example, during polymerization, nucleotides are added to the 3′ end of the primer strand. Occasionally a DNA polymerase incorporates an incorrect nucleotide to the 3′-OH terminus of the primer strand, wherein the incorrect nucleotide cannot form a hydrogen bond to the corresponding base in the template strand. Such a nucleotide, added in error, is removed from the primer as a result of the 3′ to 5′ exonuclease activity of the DNA polymerase. In embodiments, exonuclease activity may be referred to as “proofreading.” When referring to 3′-5′ exonuclease activity, it is understood that the DNA polymerase facilitates a hydrolyzing reaction that breaks phosphodiester bonds at the 3′ end of a polynucleotide chain to excise the nucleotide. In embodiments, 3′-5′ exonuclease activity refers to the successive removal of nucleotides in single-stranded DNA in a 3′→5′ direction, releasing deoxyribonucleoside 5′-monophosphates one after another. Methods for quantifying exonuclease activity are known in the art, see for example Southworth et al, PNAS Vol 93, 8281-8285 (1996). In embodiments, 5′-3′ exonuclease activity refers to the successive removal of nucleotides in double-stranded DNA in a 5′→3′ direction. In embodiments, the 5′-3′ exonuclease is lambda exonuclease. For example, lambda exonuclease catalyzes the removal of 5′ mononucleotides from duplex DNA, with a preference for 5′ phosphorylated double-stranded DNA. In other embodiments, the 5′-3′ exonuclease is E. coli DNA Polymerase I.

As used herein, the term “endonuclease” refers to enzymes that cleave the phosphodiester bond within a polynucleotide chain. The polynucleotide may be double-stranded DNA (dsDNA), single-stranded DNA (ssDNA), RNA, double-stranded hybrids of DNA and RNA, and synthetic DNA (for example, containing bases other than A, C, G, and T). An endonuclease may cut a polynucleotide symmetrically, leaving “blunt” ends, or in positions that are not directly opposing, creating overhangs, which may be referred to as “sticky ends.” An endonuclease may cut a double-stranded polynucleotide on a single strand. The methods and compositions described herein may be applied to cleavage sites generated by endonucleases. In some alternatives of the system, the system can further provide nucleic acids that encode an endonuclease, such as Cas9, TALEN, or MegaTAL, or a fusion protein comprising a domain of an endonuclease, for example, Cas9, TALEN, or MegaTAL, or one or more portion thereof. These examples are not meant to be limiting and other endonucleases and alternatives of the system and methods comprising other endonucleases and variants and modifications of these exemplary alternatives are possible without undue experimentation. All such variations and modifications are within the scope of the current teachings.

As used herein, the term “nicking endonuclease” refers to any enzyme, naturally occurring or engineered, that is capable of breaking a phosphodiester bond on a single DNA strand, leaving a 3′-hydroxyl at a defined sequence. Nicking endonucleases can be engineered by modifying restriction enzymes to eliminate cutting activity for one DNA strand, or produced by fusing a nicking subunit to a DNA binding domain, for example, zinc fingers and DNA recognition domains from transcription activator-like effectors.

As used herein, “nick” generally refers to enzymatic cleavage of only one strand of a double-stranded nucleic acid at a particular region, while leaving the other strand intact, regardless of whether one or more bases are removed. In some cases, one or more bases are removed while in other cases no bases are removed and only phosphodiester bonds are broken. In some instances, such cleavage events leave behind intact double-stranded regions lacking nicks that are a short distance apart from each other on the double-stranded nucleic acid, for example a distance of about or at least about 1, 2, 3, 4, 5, 6, 7, 8, 9, 10, 11, 12, 13, 14, 15 bases or more. In some cases, the distance between the intact double-stranded regions is equal to or less than 15, 14, 13, 12, 11, 10, 9, 8, 7, 6, 5, 4, 3, or 2 bases. In some instances, the distance between the intact double-stranded regions is 2 to 10 bases, 3 to 9 bases, or 4 to 8 bases. In embodiments, nicking breaks a phosphodiester bind in a nucleic acid molecule. In embodiments, nicking breaks a covalent linkage between two adjacent nucleotides in a nucleic acid molecule.

As used herein, the term “incorporating” or “chemically incorporating,” when used in reference to a primer and cognate nucleotide, refers to the process of joining the cognate nucleotide to the primer or extension product thereof by formation of a phosphodiester bond.

As used herein, the term “selective” or “selectivity” or the like of a compound refers to the compound's ability to discriminate between molecular targets. For example, a chemical reagent may selectively modify one nucleotide type in that it reacts with one nucleotide type (e.g., cytosines) and not other nucleotide types (e.g., adenine, thymine, or guanine). When used in the context of sequencing, such as in “selectively sequencing,” this term refers to sequencing one or more target polynucleotides from an original starting population of polynucleotides, and not sequencing non-target polynucleotides from the starting population. Typically, selectively sequencing one or more target polynucleotides involves differentially manipulating the target polynucleotides based on known sequence. For example, target polynucleotides may be hybridized to a probe oligonucleotide that may be labeled (such as with a member of a binding pair) or bound to a surface. In embodiments, hybridizing a target polynucleotide to a probe oligonucleotide includes the step of displacing one strand of a double-stranded nucleic acid. Probe-hybridized target polynucleotides may then be separated from non-hybridized polynucleotides, such as by removing probe-bound polynucleotides from the starting population or by washing away polynucleotides that are not bound to a probe. The result is a selected subset of the starting population of polynucleotides, which is then subjected to sequencing, thereby selectively sequencing the one or more target polynucleotides.

As used herein, the term “template polynucleotide” refers to any polynucleotide molecule that may be bound by a polymerase and utilized as a template for nucleic acid synthesis. A template polynucleotide may be a target polynucleotide. In general, the term “target polynucleotide” refers to a nucleic acid molecule or polynucleotide in a starting population of nucleic acid molecules having a target sequence whose presence, amount, and/or nucleotide sequence, or changes in one or more of these, are desired to be determined. The target sequence may be a portion of a gene, a regulatory sequence, genomic DNA, cDNA, RNA including mRNA, miRNA, rRNA, or others. The target sequence may be a target sequence from a sample or a secondary target such as a product of an amplification reaction. A target polynucleotide is not necessarily any single molecule or sequence. For example, a target polynucleotide may be any one of a plurality of target polynucleotides in a reaction, or all polynucleotides in a given reaction, depending on the reaction conditions. For example, in a nucleic acid amplification reaction with random primers, all polynucleotides in a reaction may be amplified. As a further example, a collection of targets may be simultaneously assayed using polynucleotide primers directed to a plurality of targets in a single reaction. As yet another example, all or a subset of polynucleotides in a sample may be modified by the addition of a primer-binding sequence (such as by the ligation of adapters containing the primer binding sequence), rendering each modified polynucleotide a target polynucleotide in a reaction with the corresponding primer polynucleotide(s). In embodiments, the template polynucleotide includes a target nucleic acid sequence and one or more barcode sequences. In embodiments, the template polynucleotide is a barcode sequence.

In embodiments, a target polynucleotide is a cell-free polynucleotide. In general, the terms “cell-free,” “circulating,” and “extracellular” as applied to polynucleotides (e.g. “cell-free DNA” (cfDNA) and “cell-free RNA” (cfRNA)) are used interchangeably to refer to polynucleotides present in a sample from a subject or portion thereof that can be isolated or otherwise manipulated without applying a lysis step to the sample as originally collected (e.g., as in extraction from cells or viruses). Cell-free polynucleotides are thus unencapsulated or “free” from the cells or viruses from which they originate, even before a sample of the subject is collected. Cell-free polynucleotides may be produced as a byproduct of cell death (e.g., apoptosis or necrosis) or cell shedding, releasing polynucleotides into surrounding body fluids or into circulation. Accordingly, cell-free polynucleotides may be isolated from a non-cellular fraction of blood (e.g., serum or plasma), from other bodily fluids (e.g., urine), or from non-cellular fractions of other types of samples.

As used herein, the terms “specific”, “specifically”, “specificity”, or the like of a compound refers to the compound's ability to cause a particular action, such as binding, to a particular molecular target with minimal or no action to other proteins in the cell.

The terms “attached,” “bind,” and “bound” as used herein are used in accordance with their plain and ordinary meanings and refer to an association between atoms or molecules. The association can be direct or indirect. For example, attached molecules may be directly bound to one another, e.g., by a covalent bond or non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). As a further example, two molecules may be bound indirectly to one another by way of direct binding to one or more intermediate molecules, thereby forming a complex.

“Specific binding” is where the binding is selective between two molecules. A particular example of specific binding is that which occurs between an antibody and an antigen. Typically, specific binding can be distinguished from non-specific when the dissociation constant (KD) is less than about 1×10-5 M or less than about 1×10-6 M or 1×10-7 M. Specific binding can be detected, for example, by ELISA, immunoprecipitation, coprecipitation, with or without chemical crosslinking, two-hybrid assays and the like. In embodiments, the KD (equilibrium dissociation constant) between two specific binding molecules is less than 10-6 M, less than 10-7 M, less than 10-8 M, less than 10-9 M, less than 10-9 M, less than 10-11 M, or less than about 10-12 M or less.

As used herein, the terms “sequencing”, “sequence determination”, “determining a nucleotide sequence”, and the like include determination of a partial or complete sequence information (e.g., a sequence) of a polynucleotide being sequenced, and particularly physical processes for generating such sequence information. That is, the term includes sequence comparisons, consensus sequence determination, contig assembly, fingerprinting, and like levels of information about a target polynucleotide, as well as the express identification and ordering of nucleotides in a target polynucleotide. The term also includes the determination of the identification, ordering, and locations of one, two, or three of the four types of nucleotides within a target polynucleotide. In some embodiments, a sequencing process described herein includes contacting a template and an annealed primer with a suitable polymerase under conditions suitable for polymerase extension and/or sequencing. In embodiments, sequencing includes sequencing by synthesis (i.e., iterative cycles of incorporating labeled nucleotides and identifying the labeled nucleotides). In embodiments, sequencing includes generating a sequencing read. In embodiments, sequencing includes sequencing by hybridization (i.e., iterative hybridization of fluorescently labeled probes) to generate a sequencing read.

As used herein, the term “polymer” refers to macromolecules having one or more structurally unique repeating units. The repeating units are referred to as “monomers,” which are polymerized for the polymer. Typically, a polymer is formed by monomers linked in a chain-like structure. A polymer formed entirely from a single type of monomer is referred to as a “homopolymer.” A polymer formed from two or more unique repeating structural units may be referred to as a “copolymer.” A polymer may be linear or branched, and may be random, block, polymer brush, hyperbranched polymer, bottlebrush polymer, dendritic polymer, or polymer micelles. The term “polymer” includes homopolymers, copolymers, tripolymers, tetra polymers and other polymeric molecules made from monomeric subunits. Copolymers include alternating copolymers, periodic copolymers, statistical copolymers, random copolymers, block copolymers, linear copolymers and branched copolymers. The term “polymerizable monomer” is used in accordance with its meaning in the art of polymer chemistry and refers to a compound that may covalently bind chemically to other monomer molecules (such as other polymerizable monomers that are the same or different) to form a polymer.

Polymers can be hydrophilic, hydrophobic or amphiphilic, as known in the art. Thus, “hydrophilic polymers” are substantially miscible with water and include, but are not limited to, polyethylene glycol and the like. “Hydrophobic polymers” are substantially immiscible with water and include, but are not limited to, polyethylene, polypropylene, polybutadiene, polystyrene, polymers disclosed herein, and the like. “Amphiphilic polymers” have both hydrophilic and hydrophobic properties and are typically copolymers having hydrophilic segment(s) and hydrophobic segment(s). Polymers include homopolymers, random copolymers, and block copolymers, as known in the art. The term “homopolymer” refers, in the usual and customary sense, to a polymer having a single monomeric unit. The term “copolymer” refers to a polymer derived from two or more monomeric species. The term “random copolymer” refers to a polymer derived from two or more monomeric species with no preferred ordering of the monomeric species. The term “block copolymer” refers to polymers having two or homopolymer subunits linked by covalent bond. Thus, the term “hydrophobic homopolymer” refers to a homopolymer which is hydrophobic. The term “hydrophobic block copolymer” refers to two or more homopolymer subunits linked by covalent bonds and which is hydrophobic.

As used herein, the term “hydrogel” refers to a three-dimensional polymeric structure that is substantially insoluble in water, but which is capable of absorbing and retaining large quantities of water to form a substantially stable, often soft and pliable, structure. In embodiments, water can penetrate in between polymer chains of a polymer network, subsequently causing swelling and the formation of a hydrogel. In embodiments, hydrogels are super-absorbent (e.g., containing more than about 90% water) and can be comprised of natural or synthetic polymers.

As used herein, the term “substrate” refers to a solid support material. The substrate can be non-porous or porous. The substrate can be rigid or flexible. As used herein, the terms “solid support” and “solid surface” refers to discrete solid or semi-solid surface. A solid support may encompass any type of solid, porous, or hollow sphere, ball, cylinder, or other similar configuration composed of plastic, ceramic, metal, or polymeric material (e.g., hydrogel) onto which a nucleic acid may be immobilized (e.g., covalently or non-covalently). A nonporous substrate generally provides a seal against bulk flow of liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefin copolymers, polyimides etc.), nylon, ceramics, resins, Zeonor, silica or silica-based materials including silicon and modified silicon, carbon, metals, inorganic glasses, optical fiber bundles, photopatternable dry film resists, UV-cured adhesives and polymers. Particularly useful solid supports for some embodiments have at least one surface located within a flow cell. Solid surfaces can also be varied in their shape depending on the application in a method described herein. For example, a solid surface useful herein can be planar, or contain regions which are concave or convex. In embodiments, the geometry of the concave or convex regions (e.g., wells) of the solid surface conform to the size and shape of the particle to maximize the contact between as substantially circular particle. In embodiments, the wells of an array are randomly located such that nearest neighbor features have random spacing between each other. Alternatively, in embodiments the spacing between the wells can be ordered, for example, forming a regular pattern. The term solid substrate is encompassing of a substrate (e.g., a flow cell) having a surface including a polymer coating covalently attached thereto. In embodiments, the solid substrate is a flow cell. The term “flow cell” as used herein refers to a chamber including a solid surface across which one or more fluid reagents can be flowed. Examples of flow cells and related fluidic systems and detection platforms that can be readily used in the methods of the present disclosure are described, for example, in Bentley et al., Nature 456:53-59 (2008). In certain embodiments a substrate includes a surface (e.g., a surface of a flow cell, a surface of a tube, a surface of a chip), for example a metal surface (e.g., steel, gold, silver, aluminum, silicon and copper). In embodiments a substrate (e.g., a substrate surface) is coated and/or includes functional groups and/or inert materials. In certain embodiments a substrate includes a bead, a chip, a capillary, a plate, a membrane, a wafer (e.g., silicon wafers), a comb, or a pin for example. In some embodiments a substrate includes a bead and/or a nanoparticle. A substrate can be made of a suitable material, non-limiting examples of which include a plastic or a suitable polymer (e.g., polycarbonate, poly(vinyl alcohol), poly(divinylbenzene), polystyrene, polyamide, polyester, polyvinylidene difluoride (PVDF), polyethylene, polyurethane, polypropylene, and the like), borosilicate, glass, nylon, Wang resin, Merrifield resin, metal (e.g., iron, a metal alloy, sepharose, agarose, polyacrylamide, dextran, cellulose and the like or combinations thereof. In embodiments a substrate includes a magnetic material (e.g., iron, nickel, cobalt, platinum, aluminum, and the like). In embodiments a substrate includes a magnetic bead (e.g., DYNABEADS®, hematite, AMPure XP). Magnets can be used to purify and/or capture nucleic acids bound to certain substrates (e.g., substrates including a metal or magnetic material). The flow cell is typically a glass slide containing small fluidic channels (e.g., a glass slide 75 mm×25 mm×1 mm having one or more channels), through which sequencing solutions (e.g., polymerases, nucleotides, and buffers) may traverse. Though typically glass, suitable flow cell materials may include polymeric materials, plastics, silicon, quartz (fused silica), Borofloat® glass, silica, silica-based materials, carbon, metals, an optical fiber or optical fiber bundles, sapphire, or plastic materials such as COCs and epoxies. The particular material can be selected based on properties desired for a particular use. For example, materials that are transparent to a desired wavelength of radiation are useful for analytical techniques that will utilize radiation of the desired wavelength. Conversely, it may be desirable to select a material that does not pass radiation of a certain wavelength (e.g., being opaque, absorptive, or reflective). In embodiments, the material of the flow cell is selected due to the ability to conduct thermal energy. In embodiments, a flow cell includes inlet and outlet ports and a flow channel extending there between.

The term “surface” is intended to mean an external part or external layer of a substrate. The surface can be in contact with another material such as a gas, liquid, gel, polymer, organic polymer, second surface of a similar or different material, metal, or coat. The surface, or regions thereof, can be substantially flat. The substrate and/or the surface can have surface features such as wells, pits, channels, ridges, raised regions, pegs, posts or the like.

The term “microplate”, or “multiwell container” as used herein, refers to a substrate including a surface, the surface including a plurality of reaction chambers separated from each other by interstitial regions on the surface. In embodiments, the microplate has dimensions as provided and described by American National Standards Institute (ANSI) and Society for Laboratory Automation And Screening (SLAS); for example the tolerances and dimensions set forth in ANSI SLAS 1-2004 (R2012); ANSI SLAS 2-2004 (R2012); ANSI SLAS 3-2004 (R2012); ANSI SLAS 4-2004 (R2012); and ANSI SLAS 6-2012, which are incorporated herein by reference. The dimensions of the microplate as described herein and the arrangement of the reaction chambers may be compatible with an established format for automated laboratory equipment. In embodiments, the device described herein provides methods for high-throughput screening. High-throughput screening (HTS) refers to a process that uses a combination of modern robotics, data processing and control software, liquid handling devices, and/or sensitive detectors, to efficiently process a large amount of (e.g., thousands, hundreds of thousands, or millions) samples in biochemical, genetic, or pharmacological experiments, either in parallel or in sequence, within a reasonably short period of time (e.g., days). Preferably, the process is amenable to automation, such as robotic simultaneous handling of 96 samples, 384 samples, 1536 samples or more. A typical HTS robot tests up to 100,000 to a few hundred thousand compounds per day. The samples are often in small volumes, such as no more than 1 mL, 500 μl, 200 μl, 100 μl, 50 μl or less. Through this process, one can rapidly identify active compounds, small molecules, antibodies, proteins or polynucleotides in a cell.

The reaction chambers may be provided as wells of a multiwell container (alternatively referred to as reaction chambers), for example a microplate may contain 2, 4, 6, 12, 24, 48, 96, 384, or 1536 sample wells. In embodiments, the 96 and 384 wells are arranged in a 2:3 rectangular matrix. In embodiments, the 24 wells are arranged in a 3:8 rectangular matrix. In embodiments, the 48 wells are arranged in a 3:4 rectangular matrix. In embodiments, the reaction chamber is a microscope slide (e.g., a glass slide about 75 mm by about 25 mm). In embodiments the slide is a concavity slide (e.g., the slide includes a depression). In embodiments, the slide includes a coating for enhanced cell adhesion (e.g., poly-L-lysine, silanes, carbon nanotubes, polymers, epoxy resins, or gold). In embodiments, the microplate is about 5 inches by about 3.33 inches, and includes a plurality of 5 mm diameter wells. In embodiments, the microplate is about 5 inches by about 3.33 inches, and includes a plurality of 6 mm diameter wells. In embodiments, the microplate is about 5 inches by about 3.33 inches, and includes a plurality of 7 mm diameter wells. In embodiments, the microplate is about 5 inches by about 3.33 inches, and includes a plurality of 7.5 mm diameter wells. In embodiments, the microplate is 5 inches by 3.33 inches, and includes a plurality of 7.5 mm diameter wells. In embodiments, the microplate is about 5 inches by about 3.33 inches, and includes a plurality of 8 mm diameter wells. In embodiments, the microplate is a flat glass or plastic tray in which an array of wells are formed, wherein each well can hold between from a few microliters to hundreds of microliters of fluid reagents and samples. In embodiments, the microplate has a rectangular shape that measures 127.7 mm±0.5 mm in length by 85.4 mm±0.5 mm in width, and includes 6, 12, 24, 48, or 96 wells, wherein each well has an average diameter of about 5-7 mm. In embodiments, the microplate has a rectangular shape that measures 127.7 mm±0.5 mm in length by 85.4 mm±0.5 mm in width, and includes 6, 12, 24, 48, or 96 wells, wherein each well has an average diameter of about 6 mm.

The term “well” refers to a discrete concave feature in a substrate having a surface opening that is completely surrounded by interstitial region(s) of the surface. Wells can have any of a variety of shapes at their opening in a surface including but not limited to round, elliptical, square, polygonal, or star shaped (i.e., star shaped with any number of vertices). The cross section of a well taken orthogonally with the surface may be curved, square, polygonal, hyperbolic, conical, or angular. The wells of a microplate are available in different shapes, for example F-Bottom: flat bottom; C-Bottom: bottom with minimal rounded edges; V-Bottom: V-shaped bottom; or U-Bottom: U-shaped bottom. In embodiments, the well is substantially square. In embodiments, the well is square. In embodiments, the well is F-bottom. In embodiments, the microplate includes 24 substantially round flat bottom wells. In embodiments, the microplate includes 48 substantially round flat bottom wells. In embodiments, the microplate includes 96 substantially round flat bottom wells. In embodiments, the microplate includes 384 substantially square flat bottom wells.

The discrete regions (i.e., features, wells) of the microplate may have defined locations in a regular array, which may correspond to a rectilinear pattern, circular pattern, hexagonal pattern, or the like. In embodiments, the pattern of wells includes concentric circles of regions, spiral patterns, rectilinear patterns, hexagonal patterns, and the like. In embodiments, the pattern of wells is arranged in a rectilinear or hexagonal pattern A regular array of such regions is advantageous for detection and data analysis of signals collected from the arrays during an analysis. These discrete regions are separated by interstitial regions. As used herein, the term “interstitial region” refers to an area in a substrate or on a surface that separates other areas of the substrate or surface. For example, an interstitial region can separate one concave feature of an array from another concave feature of the array. The two regions that are separated from each other can be discrete, lacking contact with each other. In another example, an interstitial region can separate a first portion of a feature from a second portion of a feature. In embodiments the interstitial region is continuous whereas the features are discrete, for example, as is the case for an array of wells in an otherwise continuous surface. The separation provided by an interstitial region can be partial or full separation. In embodiments, interstitial regions have a surface material that differs from the surface material of the wells (e.g., the interstitial region contains a photoresist and the surface of the well is glass). In embodiments, interstitial regions have a surface material that is the same as the surface material of the wells (e.g., both the surface of the interstitial region and the surface of well contain a polymer or copolymer).

As used herein, the term “sequencing reaction mixture” is used in accordance with its plain and ordinary meaning and refers to an aqueous mixture that contains the reagents necessary to allow dNTP or dNTP analogue (e.g., a modified nucleotide) to add a nucleotide to a DNA strand by a DNA polymerase. In embodiments, the sequencing reaction mixture includes a buffer. In embodiments, the buffer includes an acetate buffer, 3-(N-morpholino)propanesulfonic acid (MOPS) buffer, N-(2-Acetamido)-2-aminoethanesulfonic acid (ACES) buffer, phosphate-buffered saline (PBS) buffer, 4-(2-hydroxyethyl)-1-piperazineethanesulfonic acid (HEPES) buffer, N-(1,1-Dimethyl-2-hydroxyethyl)-3-amino-2-hydroxypropanesulfonic acid (AMPSO) buffer, borate buffer (e.g., borate buffered saline, sodium borate buffer, boric acid buffer), 2-Amino-2-methyl-1,3-propanediol (AMPD) buffer, N-cyclohexyl-2-hydroxyl-3-aminopropanesulfonic acid (CAPSO) buffer, 2-Amino-2-methyl-1-propanol (AMP) buffer, 4-(cyclohexylamino)-1-butanesulfonic acid (CABS) buffer, glycine-NaOH buffer, N-Cyclohexyl-2-aminoethanesulfonic acid (CHES) buffer, tris(hydroxymethyl)aminomethane (Tris) buffer, or a N-cyclohexyl-3-aminopropanesulfonic acid (CAPS) buffer. In embodiments, the buffer is a borate buffer. In embodiments, the buffer is a CHES buffer. In embodiments, the sequencing reaction mixture includes nucleotides, wherein the nucleotides include a reversible terminating moiety and a label covalently linked to the nucleotide via a cleavable linker. In embodiments, the sequencing reaction mixture includes a buffer, DNA polymerase, detergent (e.g., Triton X), a chelator (e.g., EDTA), and/or salts (e.g., ammonium sulfate, magnesium chloride, sodium chloride, or potassium chloride).

As used herein, the term “sequencing cycle” is used in accordance with its plain and ordinary meaning and refers to incorporating one or more nucleotides (e.g., nucleotide analogues) to the 3′ end of a polynucleotide with a polymerase, and detecting one or more labels that identify the one or more nucleotides incorporated. In embodiments, one nucleotide (e.g., a modified nucleotide) is incorporated per sequencing cycle. The sequencing may be accomplished by, for example, sequencing by synthesis, pyrosequencing, and the like. In embodiments, a sequencing cycle includes extending a complementary polynucleotide by incorporating a first nucleotide using a polymerase, wherein the polynucleotide is hybridized to a template nucleic acid, detecting the first nucleotide, and identifying the first nucleotide. In embodiments, to begin a sequencing cycle, one or more differently labeled nucleotides and a DNA polymerase can be introduced. Following nucleotide addition, signals produced (e.g., via excitation and emission of a detectable label) can be detected to determine the identity of the incorporated nucleotide (based on the labels on the nucleotides). Reagents can then be added to remove the 3′ reversible terminator and to remove labels from each incorporated base. Reagents, enzymes, and other substances can be removed between steps by washing. Cycles may include repeating these steps, and the sequence of each cluster is read over the multiple repetitions.

As used herein, the term “extension” or “elongation” is used in accordance with their plain and ordinary meanings and refer to synthesis by a polymerase of a new polynucleotide strand complementary to a template strand by adding free nucleotides (e.g., dNTPs) from a reaction mixture that are complementary to the template in the 5′-to-3′ direction. Extension includes condensing the 5′-phosphate group of the dNTPs with the 3′-hydroxy group at the end of the nascent (elongating) DNA strand.

As used herein, the term “sequencing read” is used in accordance with its plain and ordinary meaning and refers to an inferred sequence of nucleotide bases (or nucleotide base probabilities) corresponding to all or part of a single polynucleotide fragment. A sequencing read may include 10, 20, 30, 40, 50, 60, 70, 80, 90, 100, 150, 200, 250, or more nucleotide bases. In embodiments, a sequencing read includes reading a barcode sequence and a template nucleotide sequence. In embodiments, a sequencing read includes reading a template nucleotide sequence. In embodiments, a sequencing read includes reading a barcode and not a template nucleotide sequence. Reads of length 20-40 base pairs (bp) are referred to as ultra-short. Typical sequencers produce read lengths in the range of 100-500 bp. Read length is a factor which can affect the results of biological studies. For example, longer read lengths improve the resolution of de novo genome assembly and detection of structural variants. In embodiments, a sequencing read includes reading a barcode and a template nucleotide sequence. In embodiments, a sequencing read includes reading a template nucleotide sequence. In embodiments, a sequencing read includes reading a barcode and not a template nucleotide sequence. In embodiments, a sequencing read includes a computationally derived string corresponding to the detected label. In some embodiments, a sequencing read may include 300, 400, 500, 600, 700, 800, 900, 1,000, 1,100, 1,200, 1,300, 1,400, 1,500, or more nucleotide bases. In embodiments, the sequenced read of a barcode is considered a code. In embodiments, the sequenced read generates a codeword.

As used herein, the term “code,” means a system of rules to convert information, such as signals obtained from a detection apparatus, into another form or representation, such as a base call or nucleic acid sequence. For example, signals that are produced by one or more incorporated nucleotides can be encoded by a digit. The digit can have several potential values, each value encoding a different signal state. For example, a binary digit will have a first value for a first signal state and a second value for a second signal state. A digit can have a higher radix including, for example, a ternary digit having three potential values, a quaternary digit having four potential values, etc. A series of digits can form a codeword. The length of the codeword is the same as the number of sequencing steps performed. Exemplary codes include, but are not limited to, a Hamming code. A Hamming code is used in accordance with its ordinary meaning in computer science, mathematics, telecommunication sciences and refers to a code that can be used to detect and correct the errors that can occur when the data is moved or stored. The Hamming distance refers to the difference in integer number between two codewords of equal length, and may be determined using known techniques in the art such as the Hamming distance test or the Hamming distance algorithm. For example, for two codewords (i.e., two sequenced barcodes that have been converted to a string of integers), a difference of 0 indicates that the codewords (i.e., the sequences) are identical. A difference of 1 in integer value indicates a Hamming distance of 1, thus 1 base difference between the oligos. Hamming distance is the number of positions for which the corresponding bit values in the two strings are different. In other words, the test measures the minimum number of substitutions that would be necessary to change one bit string into the other.

The term “multiplexing” as used herein refers to an analytical method in which the presence and/or amount of multiple targets, e.g., multiple nucleic acid target sequences, can be assayed simultaneously by using the methods and devices as described herein, each of which has at least one different detection characteristic, e.g., fluorescence characteristic (for example excitation wavelength, emission wavelength, emission intensity, FWHM (full width at half maximum peak height), or fluorescence lifetime) or a unique nucleic acid or protein sequence characteristic. As used herein, the term “multiplex” is used to refer to an assay in which multiple (i.e. at least two) different biomolecules are assayed at the same time, and more particularly in the same aliquot of the sample, or in the same reaction mixture. In embodiments, more than two different biomolecules are assayed at the same time. In embodiments, at least 2, 4, 6, 8, 10, 20, 50, 100, 200, 300, 400, 500, 600, 700, 800, 900, 1000, 1100, 1200, 1300, 1400 or 1500 or more biomolecules are detected according to the present method.

Complementary single stranded nucleic acids and/or substantially complementary single stranded nucleic acids can hybridize to each other under hybridization conditions, thereby forming a nucleic acid that is partially or fully double stranded. All or a portion of a nucleic acid sequence may be substantially complementary to another nucleic acid sequence, in some embodiments. As referred to herein, “substantially complementary” refers to nucleotide sequences that can hybridize with each other under suitable hybridization conditions. Hybridization conditions can be altered to tolerate varying amounts of sequence mismatch within complementary nucleic acids that are substantially complementary. Substantially complementary portions of nucleic acids that can hybridize to each other can be 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more complementary to each other. In some embodiments substantially complementary portions of nucleic acids that can hybridize to each other are 100% complementary. Nucleic acids, or portions thereof, that are configured to hybridize to each other often include nucleic acid sequences that are substantially complementary to each other.

“Hybridize” shall mean the annealing of a nucleic acid sequence to another nucleic acid sequence (e.g., one single-stranded nucleic acid (such as a primer) to another nucleic acid) based on the well-understood principle of sequence complementarity. In an embodiment the other nucleic acid is a single-stranded nucleic acid. In some embodiments, one portion of a nucleic acid hybridizes to itself, such as in the formation of a hairpin structure. The propensity for hybridization between nucleic acids depends on the temperature and ionic strength of their milieu, the length of the nucleic acids and the degree of complementarity. The effect of these parameters on hybridization is described in, for example, Sambrook J., Fritsch E. F., Maniatis T., Molecular cloning: a laboratory manual, Cold Spring Harbor Laboratory Press, New York (1989). As used herein, hybridization of a primer, or of a DNA extension product, respectively, is extendable by creation of a phosphodiester bond with an available nucleotide or nucleotide analogue capable of forming a phosphodiester bond, therewith. For example, hybridization can be performed at a temperature ranging from 15° C. to 95° C. In some embodiments, the hybridization is performed at a temperature of about 20° C., about 25° C., about 30° C., about 35° C., about 40° C., about 45° C., about 50° C., about 55° C., about 60° C., about 65° C., about 70° C., about 75° C., about 80° C., about 85° C., about 90° C., or about 95° C. In other embodiments, the stringency of the hybridization can be further altered by the addition or removal of components of the buffered solution.

As used herein, “specifically hybridizes” refers to preferential hybridization under hybridization conditions where two nucleic acids, or portions thereof, that are substantially complementary, hybridize to each other and not to other nucleic acids that are not substantially complementary to either of the two nucleic acids. For example, specific hybridization includes the hybridization of a primer or capture nucleic acid to a portion of a target nucleic acid (e.g., a template, or adapter portion of a template) that is substantially complementary to the primer or capture nucleic acid. In some embodiments nucleic acids, or portions thereof, that are configured to specifically hybridize are often about 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, 99% or more or 100% complementary to each other over a contiguous portion of nucleic acid sequence. A specific hybridization discriminates over non-specific hybridization interactions (e.g., two nucleic acids that a not configured to specifically hybridize, e.g., two nucleic acids that are 80% or less, 70% or less, 60% or less or 50% or less complementary) by about 2-fold or more, often about 10-fold or more, and sometimes about 100-fold or more, 1000-fold or more, 10,000-fold or more, 100,000-fold or more, or 1,000,000-fold or more. Two nucleic acid strands that are hybridized to each other can form a duplex which includes a double stranded portion of nucleic acid.

As used herein, the term “adjacent,” refers to two nucleotide sequences in a nucleic acid, can refer to nucleotide sequences separated by 0 to about 20 nucleotides, more specifically, in a range of about 1 to about 10 nucleotides, or to sequences that directly abut one another. As those of skill in the art appreciate, two nucleotide sequences that that are to ligated together will generally directly abut one another.

A nucleic acid can be amplified by a suitable method. The term “amplification,” “amplified” or “amplifying” as used herein refers to subjecting a target nucleic acid in a sample to a process that linearly or exponentially generates amplicon nucleic acids having the same or substantially the same (e.g., substantially identical) nucleotide sequence as the target nucleic acid, or segment thereof, and/or a complement thereof (which may be referred to herein as an “amplification product” or “amplification products”). In some embodiments an amplification reaction comprises a suitable thermal stable polymerase. Thermal stable polymerases are known and are stable for prolonged periods of time, at temperature greater than 80° C. when compared to common polymerases found in most mammals. In certain embodiments the term “amplification,” “amplified” or “amplifying” refers to a method that comprises a polymerase chain reaction (PCR). Conditions conducive to amplification (i.e., amplification conditions) are known and often comprise at least a suitable polymerase, a suitable template, a suitable primer or set of primers, suitable nucleotides (e.g., dNTPs), a suitable buffer, and application of suitable annealing, hybridization and/or extension times and temperatures. In certain embodiments an amplified product (e.g., an amplicon) can contain one or more additional and/or different nucleotides than the template sequence, or portion thereof, from which the amplicon was generated (e.g., a primer can contain “extra” nucleotides (such as a 5′ portion that does not hybridize to the template), or one or more mismatched bases within a hybridizing portion of the primer).

As used herein, bridge-PCR (bPCR) amplification is a method for solid-phase amplification as exemplified by the disclosures of U.S. Pat. Nos. 5,641,658; 7,115,400; and U.S. Patent Publ. No. 2008/0009420, each of which is incorporated herein by reference in its entirety. Bridge-PCR involves repeated polymerase chain reaction cycles, cycling between denaturation, annealing, and extension conditions and enables controlled, spatially-localized, amplification, to generate amplification products (e.g., amplicons) immobilized on a solid support in order to form arrays comprised of colonies (or “clusters”) of immobilized nucleic acid molecule.

Amplification according to the present teachings encompasses any means by which at least a part of at least one target nucleic acid is reproduced, typically in a template-dependent manner, including without limitation, a broad range of techniques for amplifying nucleic acid sequences, either linearly or exponentially. Illustrative means for performing an amplifying step include ligase chain reaction (LCR), ligase detection reaction (LDR), ligation followed by Q-replicase amplification, PCR, primer extension, strand displacement amplification (SDA), hyperbranched strand displacement amplification, multiple displacement amplification (MDA), nucleic acid strand-based amplification (NASBA), two-step multiplexed amplifications, rolling circle amplification (RCA), and the like, including multiplex versions and combinations thereof, for example but not limited to, OLA (oligonucleotide ligation assay)/PCR, PCR/OLA, LDR/PCR, PCR/PCR/LDR, PCR/LDR, LCR/PCR, PCR/LCR (also known as combined chain reaction-CCR), and the like. Descriptions of such techniques can be found in, among other sources, Ausbel et al.; PCR Primer: A Laboratory Manual, Diffenbach, Ed., Cold Spring Harbor Press (1995); The Electronic Protocol Book, Chang Bioscience (2002); Msuih et al., J. Clin. Micro. 34:501-07 (1996); The Nucleic Acid Protocols Handbook, R. Rapley, ed., Humana Press, Totowa, N.J. (2002); Abramson et al., Curr Opin Biotechnol. 1993 February; 4(1):41-7, U.S. Pat. Nos. 6,027,998; 6,605,451, Barany et al., PCT Publication No. WO 97/31256; Wenz et al., PCT Publication No. WO 01/92579; Day et al., Genomics, 29(1): 152-162 (1995), Ehrlich et al., Science 252:1643-50 (1991); Innis et al., PCR Protocols: A Guide to Methods and Applications, Academic Press (1990); Favis et al., Nature Biotechnology 18:561-64 (2000); and Rabenau et al., Infection 28:97-102 (2000); Belgrader, Barany, and Lubin, Development of a Multiplex Ligation Detection Reaction DNA Typing Assay, Sixth International Symposium on Human Identification, 1995 (available on the world wide web at: promega.com/geneticidproc/ussymp6proc/blegrad.html-); LCR Kit Instruction Manual, Cat. #200520, Rev. #050002, Stratagene, 2002; Barany, Proc. Natl. Acad. Sci. USA 88:188-93 (1991); Bi and Sambrook, Nucl. Acids Res. 25:2924-2951 (1997); Zirvi et al., Nucl. Acid Res. 27:e40i-viii (1999); Dean et al., Proc Natl Acad Sci USA 99:5261-66 (2002); Barany and Gelfand, Gene 109:1-11 (1991); Walker et al., Nucl. Acid Res. 20:1691-96 (1992); Polstra et al., BMC Inf. Dis. 2:18-(2002); Lage et al., Genome Res. 2003 February; 13(2):294-307, and Landegren et al., Science 241:1077-80 (1988), Demidov, V., Expert Rev Mol Diagn. 2002 November; 2(6):542-8., Cook et al., J Microbiol Methods. 2003 May; 53(2):165-74, Schweitzer et al., Curr Opin Biotechnol. 2001 February; 12(1):21-7, U.S. Pat. Nos. 5,830,711, 6,027,889, 5,686,243, PCT Publication No. WO0056927A3, and PCT Publication No. WO9803673A1.

In some embodiments, amplification includes at least one cycle of the sequential procedures of: annealing at least one primer with complementary or substantially complementary sequences in at least one target nucleic acid; synthesizing at least one strand of nucleotides in a template-dependent manner using a polymerase; and denaturing the newly-formed nucleic acid duplex to separate the strands. The cycle may or may not be repeated. Amplification can include thermocycling or can be performed isothermally.

As used herein, the term “rolling circle amplification (RCA)” refers to a nucleic acid amplification reaction that amplifies a circular nucleic acid template (e.g., single-stranded DNA circles) via a rolling circle mechanism. Rolling circle amplification reaction is initiated by the hybridization of a primer to a circular, often single-stranded, nucleic acid template. The nucleic acid polymerase then extends the primer that is hybridized to the circular nucleic acid template by continuously progressing around the circular nucleic acid template to replicate the sequence of the nucleic acid template over and over again (rolling circle mechanism). The rolling circle amplification typically produces concatemers including tandem repeat units of the circular nucleic acid template sequence. The rolling circle amplification may be a linear RCA (LRCA), exhibiting linear amplification kinetics (e.g., RCA using a single specific primer), or may be an exponential RCA (ERCA) exhibiting exponential amplification kinetics. Rolling circle amplification may also be performed using multiple primers (multiply primed rolling circle amplification or MPRCA) leading to hyper-branched concatemers. For example, in a double-primed RCA, one primer may be complementary, as in the linear RCA, to the circular nucleic acid template, whereas the other may be complementary to the tandem repeat unit nucleic acid sequences of the RCA product. Consequently, the double-primed RCA may proceed as a chain reaction with exponential (geometric) amplification kinetics featuring a ramifying cascade of multiple-hybridization, primer-extension, and strand-displacement events involving both the primers. This often generates a discrete set of concatemeric, double-stranded nucleic acid amplification products. The rolling circle amplification may be performed in-vitro under isothermal conditions using a suitable nucleic acid polymerase such as Phi29 DNA polymerase. RCA may be performed by using any of the DNA polymerases that are known in the art (e.g., a Phi29 DNA polymerase, a Bst DNA polymerase, or SD polymerase).

A nucleic acid can be amplified by a thermocycling method or by an isothermal amplification method. In some embodiments a rolling circle amplification method is used. In some embodiments amplification takes place on a solid support (e.g., within a flow cell) where a nucleic acid, nucleic acid library or portion thereof is immobilized. In certain sequencing methods, a nucleic acid library is added to a flow cell and immobilized by hybridization to anchors under suitable conditions. This type of nucleic acid amplification is often referred to as solid phase amplification. In some embodiments of solid phase amplification, all or a portion of the amplified products are synthesized by an extension initiating from an immobilized primer. Solid phase amplification reactions are analogous to standard solution phase amplifications except that at least one of the amplification oligonucleotides (e.g., primers) is immobilized on a solid support.

In some embodiments solid phase amplification includes a nucleic acid amplification reaction including only one species of oligonucleotide primer immobilized to a surface or substrate. In certain embodiments solid phase amplification includes a plurality of different immobilized oligonucleotide primer species. In some embodiments solid phase amplification may include a nucleic acid amplification reaction including one species of oligonucleotide primer immobilized on a solid surface and a second different oligonucleotide primer species in solution. Multiple different species of immobilized or solution-based primers can be used. Non-limiting examples of solid phase nucleic acid amplification reactions include interfacial amplification, bridge PCR amplification, emulsion PCR, WildFire amplification (e.g., US patent publication US20130012399), the like or combinations thereof.

As used herein, the terms “cluster” and “colony” are used interchangeably to refer to a discrete site on a solid support that includes a plurality of immobilized polynucleotides and a plurality of immobilized complementary polynucleotides. The term “clustered array” refers to an array formed from such clusters or colonies. In this context the term “array” is not to be understood as requiring an ordered arrangement of clusters. The term “array” is used in accordance with its ordinary meaning in the art, and refers to a population of different molecules that are attached to one or more solid-phase substrates such that the different molecules can be differentiated from each other according to their relative location. An array can include different molecules that are each located at different addressable features on a solid-phase substrate. The molecules of the array can be nucleic acid primers, nucleic acid probes, nucleic acid templates or nucleic acid enzymes such as polymerases or ligases. Arrays useful in the invention can have densities that ranges from about 2 different features to many millions, billions or higher. The density of an array can be from 2 to as many as a billion or more different features per square cm. For example an array can have at least about 100 features/cm², at least about 1,000 features/cm², at least about 10,000 features/cm², at least about 100,000 features/cm², at least about 10,000,000 features/cm², at least about 100,000,000 features/cm², at least about 1,000,000,000 features/cm², at least about 2,000,000,000 features/cm² or higher. In embodiments, the arrays have features at any of a variety of densities including, for example, at least about 10 features/cm², 100 features/cm², 500 features/cm², 1,000 features/cm², 5,000 features/cm², 10,000 features/cm², 50,000 features/cm², 100,000 features/cm², 1,000,000 features/cm², 5,000,000 features/cm², or higher.

Provided herein are methods, systems, and compositions for analyzing a sample (e.g., sequencing nucleic acids within a sample) in situ. The term “in situ” is used in accordance with its ordinary meaning in the art and refers to a sample surrounded by at least a portion of its native environment, such as may preserve the relative position of two or more elements. For example, an extracted human cell obtained is considered in situ when the cell is retained in its local microenvironment so as to avoid extracting the target (e.g., nucleic acid molecules or proteins) away from their native environment. An in situ sample (e.g., a cell) can be obtained from a suitable subject. An in situ cell sample may refer to a cell and its surrounding milieu, or a tissue. A sample can be isolated or obtained directly from a subject or part thereof. In embodiments, the methods described herein (e.g., sequencing a plurality of target nucleic acids of a cell in situ) are applied to an isolated cell (i.e., a cell not surrounded by least a portion of its native environment). For the avoidance of any doubt, when the method is performed within a cell (e.g., an isolated cell) the method may be considered in situ. In some embodiments, a sample is obtained indirectly from an individual or medical professional. A sample can be any specimen that is isolated or obtained from a subject or part thereof. A sample can be any specimen that is isolated or obtained from multiple subjects. Non-limiting examples of specimens include fluid or tissue from a subject, including, without limitation, blood or a blood product (e.g., serum, plasma, platelets, buffy coats, or the like), umbilical cord blood, chorionic villi, amniotic fluid, cerebrospinal fluid, spinal fluid, lavage fluid (e.g., lung, gastric, peritoneal, ductal, ear, arthroscopic), a biopsy sample, celocentesis sample, cells (blood cells, lymphocytes, placental cells, stem cells, bone marrow derived cells, embryo or fetal cells) or parts thereof (e.g., mitochondrial, nucleus, extracts, or the like), urine, feces, sputum, saliva, nasal mucous, prostate fluid, lavage, semen, lymphatic fluid, bile, tears, sweat, breast milk, breast fluid, the like or combinations thereof. Non-limiting examples of tissues include organ tissues (e.g., liver, kidney, lung, thymus, adrenals, skin, bladder, reproductive organs, intestine, colon, spleen, brain, the like or parts thereof), epithelial tissue, hair, hair follicles, ducts, canals, bone, eye, nose, mouth, throat, ear, nails, the like, parts thereof or combinations thereof. A sample may include cells or tissues that are normal, healthy, diseased (e.g., infected), and/or cancerous (e.g., cancer cells). A sample obtained from a subject may include cells or cellular material (e.g., nucleic acids) of multiple organisms (e.g., virus nucleic acid, fetal nucleic acid, bacterial nucleic acid, parasite nucleic acid). A sample may include a cell and RNA transcripts. A sample can include nucleic acids obtained from one or more subjects. In some embodiments a sample includes nucleic acid obtained from a single subject. A subject can be any living or non-living organism, including but not limited to a human, non-human animal, plant, bacterium, fungus, virus, or protist. A subject may be any age (e.g., an embryo, a fetus, infant, child, adult). A subject can be of any sex (e.g., male, female, or combination thereof). A subject may be pregnant. In some embodiments, a subject is a mammal. In some embodiments, a subject is a plant. In some embodiments, a subject is a human subject. A subject can be a patient (e.g., a human patient). In some embodiments a subject is suspected of having a genetic variation or a disease or condition associated with a genetic variation.

As used herein, the term “disease state” is used in accordance with its plain and ordinary meaning and refers to any abnormal biological or aberrant state of a cell. The presence of a disease state may be identified by the same collection of biological constituents used to determine the cell's biological state. In general, a disease state will be detrimental to a biological system. A disease state may be a consequence of, inter alia, an environmental pathogen, for example a viral infection (e.g., HIV/AIDS, hepatitis B, hepatitis C, influenza, measles, etc.), a bacterial infection, a parasitic infection, a fungal infection, or infection by some other organism. A disease state may also be the consequence of some other environmental agent, such as a chemical toxin or a chemical carcinogen. As used herein, a disease state further includes genetic disorders wherein one or more copies of a gene is altered or disrupted, thereby affecting its biological function. Exemplary genetic diseases include, but are not limited to polycystic kidney disease, familial multiple endocrine neoplasia type I, neurofibromatoses, Tay-Sachs disease, Huntington's disease, sickle cell anemia, thalassemia, and Down's syndrome, as well as others (see, e.g., The Metabolic and Molecular Bases of Inherited Diseases, 7th ed., McGraw-Hill Inc., New York). Other exemplary diseases include, but are not limited to, cancer, hypertension, Alzheimer's disease, neurodegenerative diseases, and neuropsychiatric disorders such as bipolar affective disorders or paranoid schizophrenic disorders. Disease states are monitored to determine the level or severity (e.g., the stage or progression) of one or more disease states of a subject and, more specifically, detect changes in the biological state of a subject which are correlated to one or more disease states (see, e.g., U.S. Pat. No. 6,218,122, which is incorporated by reference herein in its entirety). In embodiments, methods provided herein are also applicable to monitoring the disease state or states of a subject undergoing one or more therapies. Thus, the present disclosure also provides, in some embodiments, methods for determining or monitoring efficacy of a therapy or therapies (i.e., determining a level of therapeutic effect) upon a subject. In embodiments, methods of the present disclosure can be used to assess therapeutic efficacy in a clinical trial, e.g., as an early surrogate marker for success or failure in such a clinical trial. Within eukaryotic cells, there are hundreds to thousands of signaling pathways that are interconnected. For this reason, perturbations in the function of proteins within a cell have numerous effects on other proteins and the transcription of other genes that are connected by primary, secondary, and sometimes tertiary pathways. This extensive interconnection between the function of various proteins means that the alteration of any one protein is likely to result in compensatory changes in a wide number of other proteins. In particular, the partial disruption of even a single protein within a cell, such as by exposure to a drug or by a disease state which modulates the gene copy number (e.g., a genetic mutation), results in characteristic compensatory changes in the transcription of enough other genes that these changes in transcripts can be used to define a “signature” of particular transcript alterations which are related to the disruption of function, e.g., a particular disease state or therapy, even at a stage where changes in protein activity are undetectable.

The terms “polypeptide,” “peptide” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymer. A protein may refer to a protein expressed in a cell.

A polypeptide, or a cell is “recombinant” when it is artificial or engineered, or derived from or contains an artificial or engineered protein or nucleic acid (e.g., non-natural or not wild type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

As used herein, a “single cell” refers to one cell. Single cells useful in the methods described herein can be obtained from a tissue of interest, or from a biopsy, blood sample, or cell culture. Additionally, cells from specific organs, tissues, tumors, neoplasms, or the like can be obtained and used in the methods described herein. In general, cells from any population can be used in the methods, such as a population of prokaryotic or eukaryotic organisms, including bacteria or yeast.

The term “cellular component” is used in accordance with its ordinary meaning in the art and refers to any organelle, nucleic acid, protein, or analyte that is found in a prokaryotic, eukaryotic, archaeal, or other organismic cell type. Examples of cellular components (e.g., a component of a cell) include RNA transcripts, proteins, membranes, lipids, and other analytes.

A “gene” refers to a polynucleotide that is capable of conferring biological function after being transcribed and/or translated.

As used herein, the terms “biomolecule” or “analyte” refer to an agent (e.g., a compound, macromolecule, or small molecule), and the like derived from a biological system (e.g., an organism, a cell, or a tissue). The biomolecule may contain multiple individual components that collectively construct the biomolecule, for example, in embodiments, the biomolecule is a polynucleotide wherein the polynucleotide is composed of nucleotide monomers. The biomolecule may be or may include DNA, RNA, organelles, carbohydrates, lipids, proteins, or any combination thereof. These components may be extracellular. In some examples, the biomolecule may be referred to as a clump or aggregate of combinations of components. In some instances, the biomolecule may include one or more constituents of a cell but may not include other constituents of the cell. In embodiments, a biomolecule is a molecule produced by a biological system (e.g., an organism). The biomolecule may be any substance (e.g. molecule) or entity that is desired to be detected by the method of the invention. The biomolecule is the “target” of the assay method of the invention. The biomolecule may accordingly be any compound that may be desired to be detected, for example a peptide or protein, or nucleic acid molecule or a small molecule, including organic and inorganic molecules. The biomolecule may be a cell or a microorganism, including a virus, or a fragment or product thereof. Biomolecules of particular interest may thus include proteinaceous molecules such as peptides, polypeptides, proteins or prions or any molecule which includes a protein or polypeptide component, etc., or fragments thereof. The biomolecule may be a single molecule or a complex that contains two or more molecular subunits, which may or may not be be covalently bound to one another, and which may be the same or different. Thus, in addition to cells or microorganisms, such a complex biomolecule may also be a protein complex. Such a complex may thus be a homo- or hetero-multimer. Aggregates of molecules e.g., proteins may also be target analytes, for example aggregates of the same protein or different proteins. The biomolecule may also be a complex between proteins or peptides and nucleic acid molecules such as DNA or RNA. Of particular interest may be the interactions between proteins and nucleic acids, e.g., regulatory factors, such as transcription factors, and interactions between DNA or RNA molecules

As used herein, “biomaterial” refers to any biological material produced by an organism. In some embodiments, biomaterial includes secretions, extracellular matrix, proteins, lipids, organelles, membranes, cells, portions thereof, and combinations thereof. In some embodiments, cellular material includes secretions, extracellular matrix, proteins, lipids, organelles, membranes, cells, portions thereof, and combinations thereof. In some embodiments, biomaterial includes viruses. In some embodiments, the biomaterial is a replicating virus and thus includes virus infected cells. In embodiments, a biological sample includes biomaterials.

In some embodiments, a sample includes one or more nucleic acids, or fragments thereof. A sample can include nucleic acids obtained from one or more subjects. In some embodiments a sample includes nucleic acid obtained from a single subject. In some embodiments, a sample includes a mixture of nucleic acids. A mixture of nucleic acids can include two or more nucleic acid species having different nucleotide sequences, different fragment lengths, different origins (e.g., genomic origins, cell or tissue origins, subject origins, the like or combinations thereof), or combinations thereof. A sample may include synthetic nucleic acid.

A subject can be any living or non-living organism, including but not limited to a human, non-human animal, plant, bacterium, fungus, virus or protist. A subject may be any age (e.g., an embryo, a fetus, infant, child, adult). A subject can be of any sex (e.g., male, female, or combination thereof). A subject may be pregnant. In some embodiments, a subject is a mammal. In some embodiments, a subject is a human subject. A subject can be a patient (e.g., a human patient). In some embodiments a subject is suspected of having a genetic variation or a disease or condition associated with a genetic variation.

The methods and kits of the present disclosure may be applied, mutatis mutandis, to the sequencing of RNA, or to determining the identity of a ribonucleotide.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., packaging, buffers, written instructions for performing a method, etc.) from one location to another. For example, kits include one or more enclosures (e.g., boxes) containing the relevant reaction reagents and/or supporting materials. As used herein, the term “fragmented kit” refers to a delivery system including two or more separate containers that each contain a subportion of the total kit components. The containers may be delivered to the intended recipient together or separately. For example, a first container may contain an enzyme for use in an assay, while a second container contains oligonucleotides. In contrast, a “combined kit” refers to a delivery system containing all of the components of a reaction assay in a single container (e.g., in a single box housing each of the desired components). The term “kit” includes both fragmented and combined kits.

As used herein the term “determine” can be used to refer to the act of ascertaining, establishing or estimating. A determination can be probabilistic. For example, a determination can have an apparent likelihood of at least 50%, 75%, 90%, 95%, 98%, 99%, 99.9% or higher. In some cases, a determination can have an apparent likelihood of 100%. An exemplary determination is a maximum likelihood analysis or report. As used herein, the term “identify,” when used in reference to a thing, can be used to refer to recognition of the thing, distinction of the thing from at least one other thing or categorization of the thing with at least one other thing. The recognition, distinction or categorization can be probabilistic. For example, a thing can be identified with an apparent likelihood of at least 50%, 75%, 90%, 95%, 98%, 99%, 99.9% or higher. A thing can be identified based on a result of a maximum likelihood analysis. In some cases, a thing can be identified with an apparent likelihood of 100%.

The terms “bioconjugate group,” “bioconjugate reactive moiety,” and “bioconjugate reactive group” refer to a chemical moiety which participates in a reaction to form a bioconjugate linker (e.g., covalent linker). Non-limiting examples of bioconjugate reactive groups and the resulting bioconjugate reactive linkers may be found in the Bioconjugate Table below:

| Bioconjugate | Bioconjugate | |
|---|---|---|
| reactive group 1 | reactive group 2 | |
| (e.g., electrophilic | (e.g., nucleophilic | |
| bioconjugate | bioconjugate | Resulting Bioconjugate |
| reactive moiety) | reactive moiety) | reactive linker |
| activated esters | amines/anilines | carboxamides |
| acrylamides | thiols | thioethers |
| acyl azides | amines/anilines | carboxamides |
| acyl halides | amines/anilines | carboxamides |
| acyl halides | alcohols/phenols | esters |
| acyl nitriles | alcohols/phenols | esters |
| acyl nitriles | amines/anilines | carboxamides |
| aldehydes | amines/anilines | imines |
| aldehydes or ketones | hydrazines | hydrazones |
| aldehydes or ketones | hydroxylamines | oximes |
| alkyl halides | amines/anilines | alkyl amines |
| alkyl halides | carboxylic acids | esters |
| alkyl halides | thiols | thioethers |
| alkyl halides | alcohols/phenols | ethers |
| alkyl sulfonates | thiols | thioethers |
| alkyl sulfonates | carboxylic acids | esters |
| alkyl sulfonates | alcohols/phenols | ethers |
| anhydrides | alcohols/phenols | esters |
| anhydrides | amines/anilines | carboxamides |
| aryl halides | thiols | thiophenols |
| aryl halides | amines | aryl amines |
| aziridines | thiols | thioethers |
| boronates | glycols | boronate esters |
| carbodiimides | carboxylic acids | N-acylureas or anhydrides |
| diazoalkanes | carboxylic acids | esters |
| epoxides | thiols | thioethers |
| haloacetamides | thiols | thioethers |
| haloplatinate | amino | platinum complex |
| haloplatinate | heterocycle | platinum complex |
| haloplatinate | thiol | platinum complex |
| halotriazines | amines/anilines | aminotriazines |
| halotriazines | alcohols/phenols | triazinyl ethers |
| halotriazines | thiols | triazinyl thioethers |
| imido esters | amines/anilines | amidines |
| isocyanates | amines/anilines | ureas |
| isocyanates | alcohols/phenols | urethanes |
| isothiocyanates | amines/anilines | thioureas |
| maleimides | thiols | thioethers |
| phosphoramidites | alcohols | phosphite esters |
| silyl halides | alcohols | silyl ethers |
| sulfonate esters | amines/anilines | alkyl amines |
| sulfonate esters | thiols | thioethers |
| sulfonate esters | carboxylic acids | esters |
| sulfonate esters | alcohols | ethers |
| sulfonyl halides | amines/anilines | sulfonamides |
| sulfonyl halides | phenols/alcohols | sulfonate esters |

As used herein, the term “bioconjugate reactive moiety” and “bioconjugate reactive group” refers to a moiety or group capable of forming a bioconjugate (e.g., covalent linker) as a result of the association between atoms or molecules of bioconjugate reactive groups. The association can be direct or indirect. For example, a conjugate between a first bioconjugate reactive group (e.g., —NH₂, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, bioconjugates or bioconjugate linkers are formed using bioconjugate chemistry (i.e., the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C. , 1982. In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N-hydroxysuccinimide moiety) is covalently attached to the second bioconjugate reactive group (e.g., an amine). In embodiments, the first bioconjugate reactive group (e.g., maleimide moiety) is covalently attached to the second bioconjugate reactive group (e.g., a sulfhydryl). In embodiments, the first bioconjugate reactive group (e.g., -sulfo-N-hydroxysuccinimide moiety) is covalently attached to the second bioconjugate reactive group (e.g., an amine).

Useful bioconjugate reactive groups used for bioconjugate chemistries herein include, for example: (a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc.; (c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido or maleimide groups; (e) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides, reacted with acyl halides, or bonded to metals such as gold, or react with maleimides; (h) amine or sulfhydryl groups (e.g., present in cysteine), which can be, for example, acylated, alkylated or oxidized; (i) alkenes, which can undergo, for example, cycloadditions, acylation, Michael addition, etc.; (j) epoxides, which can react with, for example, amines and hydroxyl compounds; (k) phosphoramidites and other standard functional groups useful in nucleic acid synthesis; (l) metal silicon oxide bonding; (m) metal bonding to reactive phosphorus groups (e.g., phosphines) to form, for example, phosphate diester bonds.; (n) azides coupled to alkynes using copper catalyzed cycloaddition click chemistry; (o) biotin conjugate can react with avidin or strepavidin to form a avidin-biotin complex or streptavidin-biotin complex.

An “antibody” (Ab) is a protein that binds specifically to a particular substance, known as an “antigen” (Ag). An “antibody” or “antigen-binding fragment” is an immunoglobulin that binds a specific “epitope.” The term encompasses polyclonal, monoclonal, and chimeric antibodies. In nature, antibodies are generally produced by lymphocytes in response to immune challenge, such as by infection or immunization. An “antigen” (Ag) is any substance that reacts specifically with antibodies or T lymphocytes (T cells). An antibody may include the entire antibody as well as any antibody fragments capable of binding the antigen or antigenic fragment of interest. Examples include complete antibody molecules, antibody fragments, such as Fab, F(ab′)2, CDRs, VL, VH, and any other portion of an antibody which is capable of specifically binding to an antigen. Antibodies used herein are immunospecific for, and therefore specifically and selectively bind to, for example, proteins either detected (e.g., biological targets of interest) or used for detection (e.g., probes containing oligonucleotide barcodes) in the methods and devices as described herein.

The term “covalent linker” is used in accordance with its ordinary meaning and refers to a divalent moiety which connects at least two moieties to form a molecule.

The term “non-covalent linker” is used in accordance with its ordinary meaning and refers to a divalent moiety which includes at least two molecules that are not covalently linked to each other but are capable of interacting with each other via a non-covalent bond (e.g., electrostatic interactions (e.g., ionic bond, hydrogen bond, halogen bond) or van der Waals interactions (e.g., dipole-dipole, dipole-induced dipole, London dispersion). In embodiments, the non-covalent linker is the result of two molecules that are not covalently linked to each other that interact with each other via a non-covalent bond.

As used herein a “genetically modifying agent” is a substance that alters the genetic sequence of a cell following exposure to the cell, resulting in an agent-mediated nucleic acid sequence. In embodiments, the genetically modifying agent is a small molecule, protein, pathogen (e.g., virus or bacterium), toxin, oligonucleotide, or antigen. In embodiments, the genetically modifying agent is a virus (e.g., influenza) and the agent-mediated nucleic acid sequence is the nucleic acid sequence that develops within a T-cell upon cellular exposure and contact with the virus. In embodiments, the genetically modifying agent modulates the expression of a nucleic acid sequence in a cell relative to a control (e.g., the absence of the genetically modifying agent).

Where a range of values is provided, it is understood that each intervening value, to the tenth of the unit of the lower limit unless the context clearly indicates otherwise, between the upper and lower limit of that range, and any other stated or unstated intervening value in, or smaller range of values within, that stated range is encompassed within the invention. The upper and lower limits of any such smaller range (within a more broadly recited range) may independently be included in the smaller ranges, or as particular values themselves, and are also encompassed within the invention, subject to any specifically excluded limit in the stated range. Where the stated range includes one or both of the limits, ranges excluding either or both of those included limits are also included in the invention.

As used herein, the term “upstream” refers to a region in the nucleic acid sequence that is towards the 5′ end of a particular reference point, and the term “downstream” refers to a region in the nucleic acid sequence that is toward the 3′ end of the reference point.

As used herein, the terms “incubate,” and “incubation refer collectively to altering the temperature of an object in a controlled manner such that conditions are sufficient for conducting the desired reaction. Thus, it is envisioned that the terms encompass heating a receptacle (e.g., a microplate) to a desired temperature and maintaining such temperature for a fixed time interval. Also included in the terms is the act of subjecting a receptacle to one or more heating and cooling cycles (i.e., “temperature cycling” or “thermal cycling”). While temperature cycling typically occurs at relatively high rates of change in temperature, the term is not limited thereto, and may encompass any rate of change in temperature.

As used herein, “biological activity” may include the in vivo activities of a compound or physiological responses that result upon in vivo administration of a compound, composition or other mixture. Biological activity, thus, may encompass therapeutic effects and pharmaceutical activity of such compounds, compositions and mixtures. Biological activities may be observed in vitro systems designed to test or use such activities.

The term “isolated” means altered or removed from the natural state. For example, a nucleic acid or a polypeptide naturally present in a living animal is not isolated, but the same nucleic acid or polypeptide partially or completely separated from the coexisting materials of its natural state is isolated. An isolated nucleic acid or protein can exist in substantially purified form, or can exist in a non-native environment such as, for example, a host cell. In embodiments, “isolated” refers to a nucleic acid, polynucleotide, polypeptide, protein, or other component that is partially or completely separated from components with which it is normally associated (other proteins, nucleic acids, cells, etc.).

The term “synthetic target” as used herein refers to a modified protein or nucleic acid such as those constructed by synthetic methods. In embodiments, a synthetic target is artificial or engineered, or derived from or contains an artificial or engineered protein or nucleic acid (e.g., non-natural or not wild type). For example, a polynucleotide that is inserted or removed such that it is not associated with nucleotide sequences that normally flank the polynucleotide as it is found in nature is a synthetic target polynucleotide.

The term “nucleic acid sequencing device” and the like means an integrated system of one or more chambers, ports, and channels that are interconnected and in fluid communication and designed for carrying out an analytical reaction or process, either alone or in cooperation with an appliance or instrument that provides support functions, such as sample introduction, fluid and/or reagent driving means, temperature control, detection systems, data collection and/or integration systems, for the purpose of determining the nucleic acid sequence of a template polynucleotide. Nucleic acid sequencing devices may further include valves, pumps, and specialized functional coatings on interior walls. Nucleic acid sequencing devices may include a receiving unit, or platen, that orients the flow cell such that a maximal surface area of the flow cell is available to be exposed to an optical lens. Other nucleic acid sequencing devices include those provided by Singular Genomics™ (e.g., the G4™ system), Illumina™ (e.g., HiSeq™, MiSeq™, NextSeq™, or NovaSeq™ systems), Life Technologies™ (e.g., ABI PRISM™, or SOLiD™ systems), Pacific Biosciences (e.g., systems using SMRT™ Technology such as the Sequel™ or RS II™ systems), or Qiagen (e.g., Genereader™ system). Nucleic acid sequencing devices may further include fluidic reservoirs (e.g., bottles), valves, pressure sources, pumps, sensors, control systems, valves, pumps, and specialized functional coatings on interior walls. In embodiments, the device includes a plurality of a sequencing reagent reservoirs and a plurality of clustering reagent reservoirs. In embodiments, the clustering reagent reservoir includes amplification reagents (e.g., an aqueous buffer containing enzymes, salts, and nucleotides, denaturants, crowding agents, etc.) In embodiments, the reservoirs include sequencing reagents (such as an aqueous buffer containing enzymes, salts, and nucleotides); a wash solution (an aqueous buffer); a cleave solution (an aqueous buffer containing a cleaving agent, such as a reducing agent); or a cleaning solution (a dilute bleach solution, dilute NaOH solution, dilute HCl solution, dilute antibacterial solution, or water). The fluid of each of the reservoirs can vary. The fluid can be, for example, an aqueous solution which may contain buffers (e.g., saline-sodium citrate (SSC), ascorbic acid, tris(hydroxymethyl)aminomethane or “Tris”), aqueous salts (e.g., KCl or (NH₄)₂SO₄)), nucleotides, polymerases, cleaving agent (e.g., tri-n-butyl-phosphine, triphenyl phosphine and its sulfonated versions (i.e., tris(3-sulfophenyl)-phosphine, TPPTS), and tri(carboxyethyl)phosphine (TCEP) and its salts, cleaving agent scavenger compounds (e.g., 2′-Dithiobisethanamine or 11-Azido-3,6,9-trioxaundecane-1-amine), chelating agents (e.g., EDTA), detergents, surfactants, crowding agents, or stabilizers (e.g., PEG, Tween, BSA). Non-limited examples of reservoirs include cartridges, pouches, vials, containers, and eppendorf tubes. In embodiments, the device is configured to perform fluorescent imaging. In embodiments, the device includes one or more light sources (e.g., one or more lasers). In embodiments, the illuminator or light source is a radiation source (i.e., an origin or generator of propagated electromagnetic energy) providing incident light to the sample. A radiation source can include an illumination source producing electromagnetic radiation in the ultraviolet (UV) range (about 200 to 390 nm), visible (VIS) range (about 390 to 770 nm), or infrared (IR) range (about 0.77 to 25 microns), or other range of the electromagnetic spectrum. In embodiments, the illuminator or light source is a lamp such as an arc lamp or quartz halogen lamp. In embodiments, the illuminator or light source is a coherent light source. In embodiments, the light source is a laser, LED (light emitting diode), a mercury or tungsten lamp, or a super-continuous diode. In embodiments, the light source provides excitation beams having a wavelength between 200 nm to 1500 nm. In embodiments, the laser provides excitation beams having a wavelength of 405 nm, 470 nm, 488 nm, 514 nm, 520 nm, 532 nm, 561 nm, 633 nm, 639 nm, 640 nm, 800 nm, 808 nm, 912 nm, 1024 nm, or 1500 nm. In embodiments, the illuminator or light source is a light-emitting diode (LED). The LED can be, for example, an Organic Light Emitting Diode (OLED), a Thin Film Electroluminescent Device (TFELD), or a Quantum dot based inorganic organic LED. The LED can include a phosphorescent OLED (PHOLED). In embodiments, the nucleic acid sequencing device includes an imaging system (e.g., an imaging system as described herein). The imaging system capable of exciting one or more of the identifiable labels (e.g., a fluorescent label) linked to a nucleotide and thereafter obtain image data for the identifiable labels. The image data (e.g., detection data) may be analyzed by another component within the device. The imaging system may include a system described herein and may include a fluorescence spectrophotometer including an objective lens and/or a solid-state imaging device. The solid-state imaging device may include a charge coupled device (CCD) and/or a complementary metal oxide semiconductor (CMOS). The system may also include circuitry and processors, including systems using microcontrollers, reduced instruction set computers (RISC), application specific integrated circuits (ASICs), field programmable gate array (FPGAs), logic circuits, and any other circuit or processor capable of executing functions described herein. The set of instructions may be in the form of a software program. As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. In embodiments, the device includes a thermal control assembly useful to control the temperature of the reagents.

The term “image” is used according to its ordinary meaning and refers to a representation of all or part of an object. The representation may be an optically detected reproduction. For example, an image can be obtained from fluorescent, luminescent, scatter, or absorption signals. The part of the object that is present in an image can be the surface or other xy plane of the object. Typically, an image is a 2 dimensional representation of a 3 dimensional object. An image may include signals at differing intensities (i.e., signal levels). An image can be provided in a computer readable format or medium. An image is derived from the collection of focus points of light rays coming from an object (e.g., the sample), which may be detected by any image sensor.

As used herein, the term “signal” is intended to include, for example, fluorescent, luminescent, scatter, or absorption impulse or electromagnetic wave transmitted or received. Signals can be detected in the ultraviolet (UV) range (about 200 to 390 nm), visible (VIS) range (about 391 to 770 nm), infrared (IR) range (about 0.771 to 25 microns), or other range of the electromagnetic spectrum. The term “signal level” refers to an amount or quantity of detected energy or coded information. For example, a signal may be quantified by its intensity, wavelength, energy, frequency, power, luminance, or a combination thereof. Other signals can be quantified according to characteristics such as voltage, current, electric field strength, magnetic field strength, frequency, power, temperature, etc. Absence of signal is understood to be a signal level of zero or a signal level that is not meaningfully distinguished from noise.

The term “xy coordinates” refers to information that specifies location, size, shape, and/or orientation in an xy plane. The information can be, for example, numerical coordinates in a Cartesian system. The coordinates can be provided relative to one or both of the x and y axes or can be provided relative to another location in the xy plane (e.g., a fiducial). The term “xy plane” refers to a 2 dimensional area defined by straight line axes x and y. When used in reference to a detecting apparatus and an object observed by the detector, the xy plane may be specified as being orthogonal to the direction of observation between the detector and object being detected.

As used herein, the term “tissue section” refers to a piece of tissue that has been obtained from a subject, optionally fixed and attached to a surface, e.g., a microscope slide.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### II. Compositions & Kits

In an aspect, provided herein are kits for use in accordance with any of the compounds, compositions, or methods disclosed herein, and including one or more elements thereof. In embodiments, a kit includes labeled nucleotides including differently labeled nucleotides, enzymes, buffers, oligonucleotides, and related solvents and solutions. In embodiments, the kit includes one or more oligonucleotide probes (e.g., an oligonucleotide probe as described herein). The kit may also include a template nucleic acid (DNA and/or RNA), one or more primer polynucleotides, nucleoside triphosphates (including, e.g., deoxyribonucleotides, dideoxynucleotides, ribonucleotides, labeled nucleotides, and/or modified nucleotides), buffers, salts, and/or labels (e.g., fluorophores). In embodiments, the kit includes components useful for circularizing template polynucleotides using a ligation enzyme (e.g., Circligase enzyme, Taq DNA Ligase, HiFi Taq DNA Ligase, T4 ligase, SplintR ligase, or Ampligase DNA Ligase). For example, such a kit further includes the following components: (a) reaction buffer for controlling pH and providing an optimized salt composition for a ligation enzyme (e.g., Circligase enzyme, Taq DNA Ligase, HiFi Taq DNA Ligase, T4 ligase, SplintR ligase, or Ampligase DNA Ligase), and (b) ligation enzyme cofactors. In embodiments, the kit further includes instructions for use thereof. In embodiments, kits described herein include a polymerase. In embodiments, the polymerase is a DNA polymerase. In embodiments, the DNA polymerase is a thermophilic nucleic acid polymerase. In embodiments, the DNA polymerase is a modified archaeal DNA polymerase. In embodiments, the kit includes a sequencing solution. In embodiments, the sequencing solution include labeled nucleotides including differently labeled nucleotides, wherein the label (or lack thereof) identifies the type of nucleotide. For example, each adenine nucleotide, or analog thereof; a thymine nucleotide; a cytosine nucleotide, or analog thereof; and a guanine nucleotide, or analog thereof may be labeled with a different fluorescent label.

In an aspect is provided a kit including a circularizable probe, a ligase, and an endonuclease, wherein the circularizable probe includes a first hybridization sequence capable of hybridizing to a first sequence of a target polynucleotide, a second hybridization sequence capable of hybridizing to a second sequence of the target polynucleotide, and a sequence recognized by the endonuclease.

Padlock probes (e.g., circularizable oligonucleotides, also referred to as circularizable probes) are specialized ligation probes, examples of which are known in the art, see for example Nilsson M, et al. Science. 1994; 265(5181):2085-2088), and has been applied to detect transcribed RNA in cells, see for example Christian A T, et al. Proc Natl Acad. Sci USA. 2001; 98(25):14238-14243, both of which are incorporated herein by reference in their entireties. In embodiments, the padlock probe is approximately 50 to 200 nucleotides. In embodiments, a padlock probe has a first domain that is capable of hybridizing to a first target sequence domain, and a second ligation domain, capable of hybridizing to an adjacent second sequence domain. The configuration of the padlock probe is such that upon ligation of the first and second ligation domains of the padlock probe, the probe forms a circular polynucleotide, and forms a complex with the sequence (i.e., the sequence it hybridized to, the target sequence) wherein the target sequence is “inserted” into the loop of the circle. Padlock probes are useful for the methods provided herein and include, for example, padlock probes for genomic analyses, as exemplified by Gore, A. et al. Nature 471, 63-67 (2011); Porreca, G. J. et al. Nat Methods 4, 931-936 (2007); Li, J. B. et al. Genome Res 19, 1606-1615 (2009), Zhang, K. et al. Nat Methods 6, 613-618 (2009); Noggle, S. et al. Nature 478, 70-75 (2011); and Li, J. B. et al. Science 324, 1210-1213 (2009), the content of each of which is incorporated by reference in its entirety.

In embodiments, the circularizable probe (e.g., the circularizable oligonucleotide) comprises a 5′ end and a 3′ end, wherein a first region at the 5′ end is complementary to a first sequence of a target polynucleotide, and wherein a second region at the 3′ end is complementary to a second sequence of the target polynucleotide. In embodiments, the first sequence and the second sequence of the target polynucleotide are adjacent to each other. In embodiments, the first sequence and the second sequence of the target polynucleotide are separated by 1 or more nucleotides. In embodiments, the first sequence and the second sequence of the target polynucleotide are separated by 1, 5, 10, 20, 30, 40, 50, 75, 100, or more nucleotides. In embodiments, the first sequence and the second sequence of the target polynucleotide flank a target sequence. In embodiments, the target sequence is a barcode sequence.

In embodiments, the circularizable oligonucleotide includes a primer binding sequence. In embodiments, the circularizable oligonucleotide includes at least one primer binding sequence. In embodiments, the circularizable oligonucleotide includes at least two primer binding sequences. In embodiments, the circularizable oligonucleotide includes a primer binding sequence from a known set of primer binding sequences. In embodiments, the circularizable oligonucleotide includes at least two primer binding sequences from a known set of primer binding sequences. In embodiments, the circularizable oligonucleotide includes up to 50 different primer binding sequences from a known set of primer binding sequences. In embodiments, the circularizable oligonucleotide includes up to 10 different primer binding sequences from a known set of primer binding sequences. In embodiments, the circularizable oligonucleotide includes up to 5 different primer binding sequences from a known set of primer binding sequences. In embodiments, the circularizable oligonucleotide includes two or more sequencing primer binding sequences from a known set of sequencing primer binding sequences. In embodiments, the circularizable oligonucleotide includes 1, 2, 3, 4, 5, 6, 7, 8, 9, or 10 primer binding sequences from a known set of primer binding sequences. In embodiments, the circularizable oligonucleotide includes two or more different primer binding sequences from a known set of primer binding sequences. In embodiments, the circularizable oligonucleotide includes 1, 2, 3, 4, 5, 6, 7, 8, 9, or 10 different primer binding sequences from a known set of primer binding sequences. In embodiments, the circularizable oligonucleotide includes 2 to 5 primer binding sequences from a known set of primer binding sequences. In embodiments, the circularizable oligonucleotide includes 2 to 5 different primer binding sequences from a known set of primer binding sequences. In embodiments, the circularizable oligonucleotide includes 2 to 5 sequencing primer binding sequences from a known set of sequencing primer binding sequences. In embodiments, the circularizable oligonucleotide includes 2 to 5 different sequencing primer binding sequences from a known set of sequencing primer binding sequences. In embodiments, the circularizable oligonucleotide includes at least two different primer binding sequences. In embodiments, the circularizable oligonucleotide includes two different sequencing primer binding sequences.

In embodiments, the circularizable oligonucleotide includes one or more ribonucleotides. In embodiments, the circularizable oligonucleotide includes at least one ribonucleotide at or near the ligation site (i.e., any of the 10 nucleotides within 5 nucleotides of the ligation site, wherein the ligation site includes the 5′ or 3′ end of the circularizable oligonucleotide). In embodiments, the circularizable oligonucleotide includes a ribonucleotide at a 3′ terminal and/or 3′ penultimate nucleotide. In embodiments, the circularizable oligonucleotide does not include a ribonucleotide at the 5′ end. In embodiments, the circularizable oligonucleotide does not include more than 4 consecutive ribonucleotides. Additional compositions and methods thereof of circularizable oligonucleotides including ribonucleotides are described in, e.g., U.S. Pat. Pub. No. US 2020/0224244, which is incorporated herein by reference in its entirety.

In embodiments, the endonuclease is a nicking endonuclease. These nicking endonucleases typically recognize non-palindromes. They can be bona fide nicking enzymes, such as frequent cutter Nt.CviPII and Nt.CviQII, or rare-cutting homing endonucleases I-BasI and I-HmuI, both of which recognize a degenerate 24-bp sequence. As well, isolated large subunits of heterodimeric Type IIS restriction endonucleases such as BtsI, BsrDI and BstNBI/BspD6I display nicking activity. Thus, properties of restriction endonucleases that make double-strand cuts may be retained by engineering variants of these enzymes such that they make single-strand breaks. In various embodiments, recognition sequence-specific nicking endonucleases are used as cleavage agents that cleave only a single-strand of double-stranded DNA at a cleavage site. Nicking endonucleases useful in various embodiments of methods and compositions described herein include Nb.BbvCI, Nb.BsmI, Nb.BsrDI, Nb.BtsI, Nt.AlwI, Nt.BbvCI, Nt.BsmAI, Nt.BspQI, Nt.BstNBI, and Nt.CviPII, used either alone or in various combinations. In various embodiments, nicking endonucleases that cleave outside of their recognition sequence, e.g. Nb.BsrDI, Nb.BtsI, Nt.AlwI, Nt.BsmAI, Nt.BspQI, Nt.BstNBI, and Nt.CviPII, are used. In some instances, nicking endonucleases that cut within their recognition sequences, e.g. Nb.BbvCI, Nb.BsmI, or Nt.BbvCI are used. Recognition sites for the various specific cleavage agents used herein, such as the nicking endonucleases, comprise a specific nucleic acid sequence.

The nickase Nb.BbvCI (New England Biolabs, Ipswich, Mass.) nicks at the following cleavage site with respect to its recognition site (with “I” specifying the nicking (cleavage) site and “N” representing any nucleoside, e.g. one of C, A, G or T): 5′-CCTCAGC-3′ (SEQ ID NO:1) and 3′-GGAGTICG-5′ (SEQ ID NO:2). The nickase Nb.BsmI (New England Biolabs, Ipswich, Mass.) nicks at the following cleavage site with respect to its recognition site: 5′-GAATGCN-3′ (SEQ ID NO:3) and 3′-CTTACIGN-5′ (SEQ ID NO:4). The nickase Nb.BsrDI (New England Biolabs, Ipswich, Mass.) nicks at the following cleavage site with respect to its recognition site: 5′-GCAATGNN-3′ (SEQ ID NO:5) and 3′-CGTTACINN-5′ (SEQ ID NO:6). The nickase Nb.BtsI (New England Biolabs, Ipswich, Mass.) nicks at the following cleavage site with respect to its recognition site: 5′-GCAGTGNN-3′ (SEQ ID NO:7) and 3′-CGTCACINN-5′ (SEQ ID NO:8). The nickase Nt.AlwI (New England Biolabs, Ipswich, Mass.) nicks at the following cleavage site with respect to its recognition site: 5′-GGATCNNNNIN-3′ (SEQ ID NO:9) and 3′-CCTAGNNNNN-5′ (SEQ ID NO:10). The nickase Nt.BbvCI (New England Biolabs, Ipswich, Mass.) nicks at the following cleavage site with respect to its recognition site: 5′-CCITCAGC-3′ (SEQ ID NO:11) and 3′-GGAGTCG-5′ (SEQ ID NO:12). The nickase Nt.BsmAI (New England Biolabs, Ipswich, Mass.) nicks at the following cleavage site with respect to its recognition site: 5′-GTCTCNIN-3′ (SEQ ID NO:13) and 3′-CAGAGNN-5′ (SEQ ID NO.: 14). The nickase Nt.BspQI (New England Biolabs, Ipswich, Mass.) nicks at the following cleavage site with respect to its recognition site: 5′-GCTCTTCNI-3′ (SEQ ID NO.: 15) and 3′-CGAGAAGN-5′ (SEQ ID NO:16). The nickase Nt.BstNBI (New England Biolabs, Ipswich, Mass.) nicks at the following cleavage site with respect to its recognition site: 5′-GAGTCNNNNIN-3′ (SEQ ID NO:17) and 3′-CTCAGNNNNN-5′ (SEQ ID NO:18). The nickase Nt.CviPII (New England Biolabs, Ipswich, Mass.) nicks at the following cleavage site with respect to its recognition site (wherein D denotes A or G or T and wherein H denotes A or C or T: 5′-|CCD-3′ (SEQ ID NO:19) and 3′-GGH-5′ (SEQ ID NO:20).

In embodiments, the endonuclease includes one or more endonucleases selected from the group consisting of Nb.BbvCI, Nb.BsmI, NbBsrDI, Nb.BtsI, Nt.AlwI, Nt.BbvCI, Nb.BssSI, Nt.BsmAI, Nt.BspQI, Nt.BstNBI, and Nt.CviPII. In embodiments, the endonuclease includes Nb.BbvCI. In embodiments, the endonuclease is Nb.BbvCI. In embodiments, the endonuclease is Nt.BsmAI.

In embodiments, the circularizable oligonucleotide includes any one of the sequences of SEQ ID NO:1 to SEQ ID NO:20. In embodiments, the circularizable oligonucleotide includes one or more different sequences of SEQ ID NO:1 to SEQ ID NO:20. In embodiments, the circularizable oligonucleotide includes two or more different sequences of SEQ ID NO:1 to SEQ ID NO:20. In embodiments the circularizable oligonucleotide includes any two different sequences of SEQ ID NO:1 to SEQ ID NO:20. In embodiments, the circularizable oligonucleotide includes any three different sequences of SEQ ID NO:1 to SEQ ID NO:20. In embodiments, the circularizable oligonucleotide includes the sequence of SEQ ID NO:3 or SEQ ID NO: 4. In embodiments, the circularizable oligonucleotide includes the sequence of SEQ ID NO:13 or SEQ ID NO:14. In embodiments, the circularizable oligonucleotide includes the complement of any one of the sequences of SEQ ID NO:1 to SEQ ID NO:20. In embodiments, the circularizable oligonucleotide includes the complement of one or more different sequences of SEQ ID NO:1 to SEQ ID NO:20. In embodiments, the circularizable oligonucleotide includes the complement of two or more different sequences of SEQ ID NO:1 to SEQ ID NO:20. In embodiments, the circularizable oligonucleotide includes the complement of the sequence of SEQ ID NO:3 or SEQ ID NO: 4. In embodiments, the circularizable oligonucleotide includes the complement of the sequence of SEQ ID NO:13 or SEQ ID NO:14.

In embodiments, the circularizable oligonucleotide includes about 50 to about 150 nucleotides. In embodiments, the circularizable oligonucleotide includes about 50 to about 300 nucleotides. In embodiments, the circularizable oligonucleotide includes about 50 to about 500 nucleotides. In embodiments, the circularizable oligonucleotide includes about or more than about 50, 75, 100, 125, 150, 175, 200, 250, 300, 350, 400, or 500 nucleotides. In embodiments, the circularizable oligonucleotide includes less than about 50, 75, 100, 125, 150, 175, 200, 250, 300, 350, 400, or 500 nucleotides.

In embodiments, the circularizable oligonucleotide includes at least one amplification primer binding sequence or at least one sequencing primer binding sequence. The amplification primer binding sequence refers to a nucleotide sequence that is complementary to a primer useful in initiating amplification (i.e., an amplification primer). Likewise, a sequencing primer binding sequence is a nucleotide sequence that is complementary to a primer useful in initiating sequencing (i.e., a sequencing primer). Primer binding sequences usually have a length in the range of between 3 to 36 nucleotides, also 5 to 24 nucleotides, also from 14 to 36 nucleotides. In embodiments, an amplification primer and a sequencing primer are complementary to the same primer binding sequence, or overlapping primer binding sequences. In embodiments, an amplification primer and a sequencing primer are complementary to different primer binding sequences.

In embodiments, the amplification primer binding sequence and/or sequencing primer binding sequence includes any one of the sequences (e.g., all or a portion thereof), or complement thereof, as described in Table 2. In embodiments, the amplification primer binding sequence includes any one of the sequences, or complement thereof, of SEQ ID NO:21 to SEQ ID NO:74. In embodiments, the sequencing primer binding sequence includes any one of the sequences, or complement thereof, of SEQ ID NO:21 to SEQ ID NO:74. In embodiments, the amplification primer binding sequence includes any one of the sequences, or complement thereof, of SEQ ID NO: 21, SEQ ID NO:22, SEQ ID NO:23, SEQ ID NO:25, SEQ ID NO:27, SEQ ID NO:48, or SEQ ID NO:53. In embodiments, the sequencing primer binding sequence includes any one of the sequences, or complement thereof, of SEQ ID NO: 21, SEQ ID NO:22, SEQ ID NO:23, SEQ ID NO:25, SEQ ID NO:27, SEQ ID NO:48, or SEQ ID NO:53. In embodiments, the amplification primer binding sequence includes any one of the sequences, or complement thereof, of SEQ ID NO: 27, SEQ ID NO:62, SEQ ID NO:37, SEQ ID NO:48, SEQ ID NO:22, SEQ ID NO:67, or SEQ ID NO:53. In embodiments, the sequencing primer binding sequence includes any one of the sequences, or complement thereof, of SEQ ID NO: 27, SEQ ID NO:62, SEQ ID NO:37, SEQ ID NO:48, SEQ ID NO:22, SEQ ID NO:67, or SEQ ID NO:53. In embodiments, the amplification primer binding sequence and/or sequencing primer binding sequence includes a portion of the sequences described in Table 2 (e.g., a sequence including 80% homology, a sequence with 1, 2, 3, 4, 5 nucleotide truncations from the sequence identified in Table 2).

In embodiments, the circularizable oligonucleotide further includes a barcode sequence. In embodiments, the barcode sequence is at least 5, 6, 7, 8, 9, 10, 11, 12, 13, 14, or 15 nucleotides in length. In embodiments, the barcode (i.e., the barcode sequence) is at least 5, 6, 7, 8, 9, 10, 11, 12, 13, 14, or 15 nucleotides in length. In embodiments, the barcode is 5, 6, 7, 8, 9, 10, 11, 12, 13, 14, or 15 nucleotides in length. In embodiments, the barcode is 10 to 15 nucleotides in length. In embodiments, the barcode is at least about 1, 2, 3, 4, 5, 6, 7, 8, 9, 10, 15, 20, 25, 30, 35, 40, 45, 50 or more nucleotides in length. In embodiments, the barcode can be at most about 300, 200, 100, 90, 80, 70, 60, 50, 40, 30, 20, 15, 12, 10, 9, 8, 7, 6, 5, 4 or fewer or more nucleotides in length. In embodiments, the barcode includes between about 5 to about 8, about 5 to about 10, about 5 to about 15, about 5 to about 20, about 10 to about 150 nucleotides. In embodiments, the barcode includes between 5 to 8, 5 to 10, 5 to 15, 5 to 20, 10 to 150 nucleotides. In embodiments, the barcode is 10 nucleotides. In embodiments, the barcode may include a unique sequence (e.g., a barcode sequence) that gives the barcode its identifying functionality. The unique sequence may be random or non-random. Attachment of the barcode sequence (via binding of a proximity probe conjugated to the barcode sequence) to a protein or nucleic acid of interest (i.e., the target) may associate the barcode sequence with the protein or nucleic acid of interest. The barcode may then be used to identify the protein or nucleic acid of interest during sequencing, even when other proteins or nucleic acids of interest (e.g., including different oligonucleotide barcodes) are present. In embodiments, the barcode consists only of a unique barcode sequence. In embodiments, the 5′ end of a barcoded oligonucleotide is phosphorylated. In embodiments, the barcode is known (i.e., the nucleic sequence is known before sequencing) and is sorted into a basis-set according to their Hamming distance. Oligonucleotide barcodes (e.g., barcode sequences included in an oligonucleotide probe) can be associated with a target of interest by knowing, a priori, the target of interest, such as a gene or protein. In embodiments, the barcodes further include one or more sequences capable of specifically binding a gene or nucleic acid sequence of interest. For example, in embodiments, the barcode includes a sequence capable of hybridizing to mRNA, e.g., one containing a poly-T sequence (e.g., having several T's in a row, e.g., 4, 5, 6, 7, 8, or more T's).

In embodiments, the barcode sequence is selected from a known set of barcode sequences. In embodiments, each barcode sequence is unique within the known set of barcodes. In embodiments, the barcodes are selected to form a known set of barcodes, e.g., the set of barcodes may be distinguished by a particular Hamming distance.

In embodiments, the barcode is included as part of an oligonucleotide of longer sequence length, such as a primer or a random sequence (e.g., a random N-mer). In embodiments, the barcode contains random sequences to increase the mass or size of the oligonucleotide tag. The random sequence can be of any suitable length, and there may be one or more than one present. As non-limiting examples, the random sequence may have a length of 10 to 40, 10 to 30, 10 to 20, 25 to 50, 15 to 40, 15 to 30, 20 to 50, 20 to 40, or 20 to 30 nucleotides. In embodiments, each barcode sequence is selected from a known set of barcode sequences. In embodiments, each of the known set of barcode sequences is associated with a targeting sequence from a known set of targeting sequences. In embodiments, a first barcode sequence is associated with a first targeting sequence, and wherein a second barcode sequence is associated with a second targeting sequence (e.g., wherein the second targeting sequence is included in an oligonucleotide probe targeting a different target nucleic acid than the first targeting sequence). In embodiments, the same barcode sequence is associated with a plurality of oligonucleotide probes targeting different sequences of the same target nucleic acid (e.g., the same target polynucleotide).

In embodiments, the barcode is taken from a “pool” or “set” or “basis-set” of potential oligonucleotide barcode sequences. The set of barcodes may be selected using any suitable technique, e.g., randomly, or such that the sequences allow for error detection and/or correction, or having a particular feature, such as by being separated by a certain distance (e.g., Hamming distance). In embodiments, the method includes selecting a basis-set of oligonucleotide barcodes having a specified Hamming distance (e.g., a Hamming distance of 10; a Hamming distance of 5). The pool may have any number of potential barcode sequences, e.g., at least 100, at least 300, at least 500, at least 1,000, at least 3,000, at least 5,000, at least 10,000, at least 30,000, at least 50,000, at least 100,000, at least 300,000, at least 500,000, or at least 1,000,000 barcode sequences. In embodiments, a barcode is a degenerate or partially-degenerate sequence, such that one or more nucleotides are selected at random from a set of two or more different nucleotides at one or more positions, with each of the different nucleotides selected at one or more positions represented in a pool of oligonucleotides including the degenerate or partially-degenerate sequence. The number of possible barcodes in a given set of barcodes will vary with the number of degenerate positions, and the number of bases permitted at each such position. For example, a barcode of five nucleotides (consecutive or non-consecutive), in which each position can be any of A, T, G, or C represents 54, or 1024 possible barcodes. In embodiments, certain barcode sequences may be excluded from a pool, such as barcodes in which every position is the same base. In embodiments, there are about, 102, 103 104, 105, 106, 107, 108, 109, or a number or a range between any two of these values, unique nucleotide barcode sequences. In embodiments, there are at least, or at most 102, 103 104, 105, 106, 107, 108, 109 unique barcode sequences. In embodiments, a barcode is about, 1, 2, 3, 4, 5, 10, 15, 20, 25, 30, 35, 40, 45, 50, or a number or a range between any two of these values, nucleotides in length. A barcode can be at least, or at most, 1, 2, 3, 4, 5, 10, 15, 20, 25, 30, 35, 40, 45, 50, 100, or 200 nucleotides in length.

In embodiments, the barcodes in the known set of barcodes have a specified Hamming distance. In embodiments, the Hamming distance is 4 to 15. In embodiments, the Hamming distance is 8 to 12. In embodiments, the Hamming distance is 10. In embodiments, the Hamming distance is 0 to 100. In embodiments, the Hamming distance is 0 to 15. In embodiments, the Hamming distance is 0 to 10. In embodiments, the Hamming distance is 1 to 10. In embodiments, the Hamming distance is 5 to 10. In embodiments, the Hamming distance is 1 to 100. In embodiments, the Hamming distance between any two barcode sequences of the set is at least 2, 3, 4, or 5. In embodiments, the Hamming distance between any two barcode sequences of the set is at least 3. In embodiments, the Hamming distance between any two barcode sequences of the set is at least 4.

In embodiments, the target polynucleotide includes a cancer-associated gene nucleic acid sequence, a viral nucleic acid sequence, a bacterial nucleic acid sequence, or a fungal nucleic acid sequence.

In embodiments, the target nucleic acid (i.e., the target polynucleotide) includes a nucleic acid sequence encoding a TCR alpha chain, a TCR beta chain, a TCR delta chain, a TCR gamma chain, or any fragment thereof (e.g., variable regions including VDJ or VJ regions, constant regions, transmembrane regions, fragments thereof, combinations thereof, and combinations of fragments thereof). In embodiments, the target nucleic acid includes a nucleic acid sequence encoding a B cell receptor heavy chain, B cell receptor light chain, or any fragment thereof (e.g., variable regions including VDJ or VJ regions, constant regions, transmembrane regions, fragments thereof, combinations thereof, and combinations of fragments thereof). In embodiments, the target nucleic acid includes a CDR3 nucleic acid sequence. In embodiments, the target nucleic acid includes a TCRA gene sequence or a TCRB gene sequence. In embodiments, the target nucleic acid includes a TCRA gene sequence and a TCRB gene sequence. In embodiments, the target nucleic acid includes sequences of various T cell receptor alpha variable genes (TRAV genes), T cell receptor alpha joining genes (TRAJ genes), T cell receptor alpha constant genes (TRAC genes), T cell receptor beta variable genes (TRBV genes), T cell receptor beta diversity genes (TRBD genes), T cell receptor beta joining genes (TRBJ genes), T cell receptor beta constant genes (TRBC genes), T cell receptor gamma variable genes (TRGV genes), T cell receptor gamma joining genes (TRGJ genes), T cell receptor gamma constant genes (TRGC genes), T cell receptor delta variable genes (TRDV genes), T cell receptor delta diversity genes (TRDD genes), T cell receptor delta joining genes (TRDJ genes), or T cell receptor delta constant genes (TRDC genes).

In embodiments, the first sequence includes a nucleic acid sequence encoding a B cell receptor V region, and wherein the second sequence includes a nucleic acid sequence encoding a B cell receptor J region.

In embodiments, the first sequence and the second sequence flank a CDR3 nucleic acid sequence.

In embodiments, the target polynucleotide includes a cancer-associated gene nucleic acid sequence, a viral nucleic acid sequence, a bacterial nucleic acid sequence, or a fungal nucleic acid sequence. In embodiments, the cancer-associated gene is a nucleic acid sequence identified within The Cancer Genome Atlas Program, accessible at www.cancer.gov/tcga.

In embodiments, the target polynucleotide includes a CD4, CD68, CD20, CD11c, CD8, HLA-DR, Ki67, CD45RO, PanCK, CD3e, CD44, CD45, HLA-A, CD14, CD56, CD57, CD19, CD2, CD1a, CD107a, CD21, Pax5, FOXP3, Granzyme B, CD38, CD39, CD79a, TIGIT, TOX, TP63, S100A4, TFAM, GP100, LaminBi, CK19, CK17, GATA3, SOX2, Bcl2, EpCAM, Caveolin, CD163, CD11b, MPO, CD141, iNOS, PD-1, PD-L1, ICOS, TIM3, LAG3, IDO1, CD40, HLA-E, IFNG, CD69, E-cadherin, CD31, Histone H3, Beta-actin, Podoplanin, SMA, Vimentin, Collagen IV, CD34, Beta-catenin, MMP-9, ZEB1, ASCT2, Na/K ATPase, HK1, LDHA, G6PD, IDH2, GLUT1, pNRF2, ATPA5, SDHA, Citrate Synthase, CPT1A, PARP, BAK, BCL-XL, BAX, BAD, Cytochrome c, LC3B, Beclin-1, H2AX, pRPS6, PCNA, Cyclin D1, HLA-DPB1, LEF1, GAL9, CD138, MC Tryptase, OX40, ZAP70, CD7, C1Qa, CCR6, CD15, AXL, and/or CD227 nucleic acid sequence.

In embodiments, the target polynucleotide can include any polynucleotide of interest. The polynucleotide can include DNA, RNA, peptide nucleic acid, morpholino nucleic acid, locked nucleic acid, glycol nucleic acid, threose nucleic acid, mixtures thereof, and hybrids thereof. In embodiments, the polynucleotide is obtained from one or more source organisms. In some embodiments, the polynucleotide can include a selected sequence or a portion of a larger sequence. In embodiments, sequencing a portion of a polynucleotide or a fragment thereof can be used to identify the source of the polynucleotide. With reference to nucleic acids, polynucleotides and/or nucleotide sequences a “portion,” “fragment” or “region” can be at least 5 consecutive nucleotides, at least 10 consecutive nucleotides, at least 15 consecutive nucleotides, at least 20 consecutive nucleotides, at least 25 consecutive nucleotides, at least 50 consecutive nucleotides, at least 100 consecutive nucleotides, or at least 150 consecutive nucleotides.

In embodiments, the entire sequence of the target polynucleotide is about 1 to 3 kb, and only a portion of that target (e.g., 50 to 100 nucleotides) is sequenced. In embodiments, the target polynucleotide is about 1 to 3 kb. In embodiments, the target polynucleotide is about 1 to 2 kb. In embodiments, the target polynucleotide is about 1 kb. In embodiments, the target polynucleotide is about 2 kb. In embodiments, the target polynucleotide is less than 1 kb. In embodiments, the target polynucleotide is about 500 nucleotides. In embodiments, the target polynucleotide is about 200 nucleotides. In embodiments, the target polynucleotide is about 100 nucleotides. In embodiments, the target polynucleotide is less than 100 nucleotides. In embodiments, the target polynucleotide is about 5 to 50 nucleotides.

In embodiments, the target polynucleotide is an RNA nucleic acid sequence or DNA nucleic acid sequence. In embodiments, the target polynucleotide is an RNA nucleic acid sequence or DNA nucleic acid sequence from the same cell. In embodiments, the target polynucleotide is an RNA nucleic acid sequence. In embodiments, the RNA nucleic acid sequence is stabilized using known techniques in the art. For example, RNA degradation by RNase should be minimized using commercially available solutions, e.g., RNA Later®, RNA Lysis Buffer, or Keratinocyte serum-free medium). In embodiments, the target polynucleotide is messenger RNA (mRNA), transfer RNA (tRNA), micro RNA (miRNA), small interfering RNA (siRNA), small nucleolar RNA (snoRNA), small nuclear RNA (snRNA), Piwi-interacting RNA (piRNA), enhancer RNA (eRNA), or ribosomal RNA (rRNA). In embodiments, the target polynucleotide is pre-mRNA. In embodiments, the target polynucleotide is heterogeneous nuclear RNA (hnRNA). In embodiments, the target polynucleotide is mRNA, tRNA (transfer RNA), rRNA (ribosomal RNA), or noncoding RNA (such as lncRNA (long noncoding RNA)). In embodiments, the target polynucleotides are on different regions of the same RNA nucleic acid sequence.

In embodiments, the target polynucleotide includes RNA nucleic acid sequences. In embodiments the target polynucleotide is an RNA transcript. In embodiments the target polynucleotide is a single stranded RNA nucleic acid sequence. In embodiments, the target polynucleotide is an RNA nucleic acid sequence or a DNA nucleic acid sequence (e.g., cDNA). In embodiments, the target polynucleotide is a cDNA target polynucleotide nucleic acid sequence and before step i), the RNA nucleic acid sequence is reverse transcribed to generate the cDNA target polynucleotide nucleic acid sequence. In embodiments, reverse transcription of the RNA nucleic acid is performed with a reverse transcriptase, for example, Tth DNA polymerase or mutants thereof. In embodiments, the target polynucleotide is genomic DNA (gDNA), mitochondrial DNA, chloroplast DNA, episomal DNA, viral DNA, or copy DNA (cDNA). In embodiments, the target polynucleotide is coding RNA such as messenger RNA (mRNA), and non-coding RNA (ncRNA) such as transfer RNA (tRNA), microRNA (miRNA), small nuclear RNA (snRNA), or ribosomal RNA (rRNA). In embodiments, the target polynucleotide is a cancer-associated gene. In embodiments, to minimize amplification errors or bias, the target polynucleotide is not reverse transcribed to generate cDNA.

In embodiments, the circularizable oligonucleotide includes locked nucleic acids (LNAs), Bis-locked nucleic acids (bisLNAs), twisted intercalating nucleic acids (TINAs), bridged nucleic acids (BNAs), 2′-O-methyl RNA:DNA chimeric nucleic acids, minor groove binder (MGB) nucleic acids, morpholino nucleic acids, C5-modified pyrimidine nucleic acids, peptide nucleic acids (PNAs), or combinations thereof. In embodiments, the circularizable oligonucleotide includes one or more LNA nucleotides. In embodiments, the sequence complementary to the first hybridization sequence and/or the second sequence complementary to the second hybridization sequence of the circularizable oligonucleotide includes one or more LNA nucleotides.

In embodiments, each of the plurality of immobilized oligonucleotides (e.g., immobilized primers) is about 5 to about 25 nucleotides in length. In embodiments, each of the plurality of immobilized oligonucleotides (e.g., immobilized primers) is about 10 to about 40 nucleotides in length. In embodiments, each of the plurality of immobilized oligonucleotides (e.g., immobilized primers) is about 5 to about 100 nucleotides in length. In embodiments, each of the plurality of immobilized oligonucleotides (e.g., immobilized primers) is about 20 to 200 nucleotides in length. In embodiments, each of the plurality of immobilized oligonucleotides (e.g., immobilized primers) about or at least about 5, 6, 7, 8, 9, 10, 12, 15, 18, 20, 25, 30, 35, 40, 50 or more nucleotides in length. In embodiments, one or more immobilized oligonucleotides include blocking groups at their 3′ ends that prevent polymerase extension. A blocking moiety prevents formation of a covalent bond between the 3′ hydroxyl moiety of the nucleotide and the 5′ phosphate of another nucleotide. In embodiments, the 3′ modification is a 3′-phosphate modification, including a 3′ phosphate moiety, which is removed by a PNK enzyme or a phosphatase enzyme. Alternatively, abasic site cleavage with certain endonucleases (e.g., Endo IV) results in a 3′-OH at the cleavable site from the 3′-diesterase activity.

In embodiments, the kit includes a first immobilized oligonucleotide (i.e., a first immobilized primer) including a sequence (e.g., all or a portion thereof), or complement thereof, as described in Table 2. In embodiments, the first immobilized primer is a sequence selected from the group consisting of SEQ ID NO:21 to SEQ NO:74, or a complement thereof. In embodiments the kit includes a second immobilized oligonucleotide (i.e., a second immobilized primer) including a sequence selected from the group consisting of SEQ ID NO:21 to SEQ NO:74, or a complement thereof. In embodiments, the kit includes a third immobilized oligonucleotide (i.e., a third immobilized primer) including a sequence selected from the group consisting of SEQ ID NO:21 to SEQ NO:74, or a complement thereof. In embodiments, the first immobilized primer and the second immobilized primer are different sequences selected from the group consisting of SEQ ID NO:21 to SEQ NO:74, or a complement thereof. In embodiments, the first immobilized primer and the third immobilized primer are the same sequence selected from the group consisting of SEQ ID NO:21 to SEQ NO:74, or a complement thereof.

In embodiments, the first immobilized primer is SEQ ID NO:25 and the second immobilized primer is SEQ ID NO:22. In embodiments, the first immobilized primer is SEQ ID NO:27 and the second immobilized primer is SEQ ID NO:48. In embodiments, the first immobilized primer is SEQ ID NO:21 and the second immobilized primer is SEQ ID NO:23. In embodiments, the first immobilized primer is SEQ ID NO:27 and the second immobilized primer is SEQ ID NO:22. In embodiments, the first immobilized primer is SEQ ID NO:27 and the second immobilized primer is SEQ ID NO:53. In embodiments, the third immobilized primer is SEQ ID NO:21. In embodiments, the third immobilized primer is SEQ ID NO:25. In embodiments, the third immobilized primer is SEQ ID NO:27. In embodiments, the first immobilized primer and the third immobilized primer are SEQ ID NO:25 and the second immobilized primer is SEQ ID NO:22. In embodiments, the first immobilized primer and the third immobilized primer are SEQ ID NO:27 and the second immobilized primer is SEQ ID NO:48. In embodiments, the first immobilized primer and the third immobilized primer are SEQ ID NO:21 and the second immobilized primer is SEQ ID NO:23. In embodiments, the first immobilized primer and the third immobilized primer are SEQ ID NO:27 and the second immobilized primer is SEQ ID NO:22. In embodiments, the first immobilized primer and the third immobilized primer are SEQ ID NO:27 and the second immobilized primer is SEQ ID NO:53.

In embodiments, the first immobilized primer is SEQ ID NO:27 and the second immobilized primer is SEQ ID NO:62. In embodiments, the first immobilized primer is SEQ ID NO:37 and the second immobilized primer is SEQ ID NO:48. In embodiments, the first immobilized primer is SEQ ID NO:37 and the second immobilized primer is SEQ ID NO:62. In embodiments, the first immobilized primer is SEQ ID NO:37 and the second immobilized primer is SEQ ID NO:22. In embodiments, the first immobilized primer is SEQ ID NO:27 and the second immobilized primer is SEQ ID NO:67. In embodiments, the first immobilized primer is SEQ ID NO:37 and the second immobilized primer is SEQ ID NO:53. In embodiments, the first immobilized primer is SEQ ID NO:37 and the second immobilized primer is SEQ ID NO:67. In embodiments, the third immobilized primer is SEQ ID NO:27. In embodiments, the third immobilized primer is SEQ ID NO:37. In embodiments, the first immobilized primer and the third immobilized primer are SEQ ID NO:27 and the second immobilized primer is SEQ ID NO:62. In embodiments, the first immobilized primer and the third immobilized primer are SEQ ID NO:37 and the second immobilized primer is SEQ ID NO:48. In embodiments, the first immobilized primer and the third immobilized primer are SEQ ID NO:37 and the second immobilized primer is SEQ ID NO:62. In embodiments, the first immobilized primer and the third immobilized primer are SEQ ID NO:37 and the second immobilized primer is SEQ ID NO:22. In embodiments, the first immobilized primer and the third immobilized primer are SEQ ID NO:27 and the second immobilized primer is SEQ ID NO:67. In embodiments, the first immobilized primer and the third immobilized primer are SEQ ID NO:37 and the second immobilized primer is SEQ ID NO:53. In embodiments, the first immobilized primer and the third immobilized primer are SEQ ID NO:37 and the second immobilized primer is SEQ ID NO:67.

In embodiments, the immobilized oligonucleotides include one or more phosphorothioate nucleotides. In embodiments, the immobilized oligonucleotides include a plurality of phosphorothioate nucleotides. In embodiments, about or at least about 20%, 30%, 40%, 50%, 60%, 70%, 80%, 90%, or about 100% of the nucleotides in the immobilized oligonucleotides are phosphorothioate nucleotides. In embodiments, most of the nucleotides in the immobilized oligonucleotides are phosphorothioate nucleotides. In embodiments, all of the nucleotides in the immobilized oligonucleotides are phosphorothioate nucleotides. In embodiments, none of the nucleotides in the immobilized oligonucleotides are phosphorothioate nucleotides. In embodiments, the 5′ end of the immobilized oligonucleotide includes one or more phosphorothioate nucleotides. In embodiments, the 5′ end of the immobilized oligonucleotide includes between one and five phosphorothioate nucleotides.

In embodiments, the kit includes a microplate, and reagents for sample preparation and purification, amplification, and/or sequencing (e.g., one or more sequencing reaction mixtures). In embodiments, the kit includes for protein detection includes a plurality of proximity probes linked to an oligonucleotide (e.g., DNA-conjugated antibodies). In embodiments, the kit includes a multiwell container. In embodiments, the kit includes a flow cell. In embodiments, the kit includes a glass slide including a polymer coating.

In embodiments, the kit includes a single restriction endonuclease. In embodiments, the restriction endonuclease may include XbaI, EcoRI-HF, NheI, BamHI, XcmI, PflMI, BstEII, NcoI, HpaI, BsgI, AfeI, StuI, BsrGI, or a CRISPR-Cas9 nuclease (e.g., to achieve an approximate 95% cleavage or digestion rate, or the cleaving activity, as described by Zhang et al (see, Zhang Y et al. PLoS ONE. 2020. 15(12): e0244464, which is incorporated herein by reference in its entirety)). In embodiments, the restriction endonuclease may include XbaI, EcoRI, BamHI, XcmI or BstEII (e.g., to achieve an approximate 98% or greater cleavage or digestion rate, or the cleaving activity, as described by Zhang et al.). In embodiments, the restriction endonuclease may include EcoRI or XbaI (e.g., to achieve an approximate 99% or greater cleavage or digestion rate, or the cleaving activity, as described by Zhang et al.).

In embodiments, the kit includes a programmable endonuclease. In embodiments, the kit further includes a guide oligonucleotide (e.g., a guide oligonucleotide that complexes with the programmable endonuclease and targets the programmable endonuclease to a target nucleic acid sequence). In embodiments, the programmable endonuclease is an argonaute enzyme. In embodiments, the argonaute enzyme is Thermus thermophilus argonaute (TtAgo), or a mutant thereof. In embodiments, the programmable endonuclease is from the haloalkaliphilic archaebacterium N. gregoryi SP2 (NgAgo), or a mutant thereof.

In embodiments, amplification reagents and other reagents may be provided in lyophilized form. In embodiments, amplification reagents and other reagents may be provided in a container that includes wells within which the lyophilized reagent may be reconstituted.

In embodiments, the kit includes components useful for circularizing template polynucleotides using a ligation enzyme (e.g., Circligase enzyme, Taq DNA Ligase, HiFi Taq DNA Ligase, T4 ligase, SplintR ligase, or Ampligase DNA Ligase). For example, such a kit further includes the following components: (a) reaction buffer for controlling pH and providing an optimized salt composition for a ligation enzyme (e.g., Circligase enzyme, Taq DNA Ligase, HiFi Taq DNA Ligase, T4 ligase, SplintR ligase, or Ampligase DNA Ligase), and (b) ligation enzyme cofactors. In embodiments, the kit further includes instructions for use thereof. In embodiments, kits described herein include a polymerase. In embodiments, the polymerase is a DNA polymerase. In embodiments, the DNA polymerase is a thermophilic nucleic acid polymerase. In embodiments, the DNA polymerase is a modified archaeal DNA polymerase. In embodiments, the kit includes a sequencing solution. In embodiments, the sequencing solution include labeled nucleotides including differently labeled nucleotides, wherein the label (or lack thereof) identifies the type of nucleotide. For example, each adenine nucleotide, or analog thereof; a thymine nucleotide; a cytosine nucleotide, or analog thereof; and a guanine nucleotide, or analog thereof may be labeled with a different fluorescent label. In embodiments, the kit includes a modified terminal deoxynucleotidyl transferase (TdT) enzyme.

In embodiments, the kit includes a sequencing polymerase, and one or more amplification polymerases. In embodiments, the sequencing polymerase is capable of incorporating modified nucleotides. In embodiments, the polymerase is a DNA polymerase. In embodiments, the DNA polymerase is a Pol I DNA polymerase, Pol II DNA polymerase, Pol III DNA polymerase, Pol IV DNA polymerase, Pol V DNA polymerase, Pol R DNA polymerase, Pol p DNA polymerase, Pol λ DNA polymerase, Pol a DNA polymerase, Pol a DNA polymerase, Pol 6 DNA polymerase, Pol F DNA polymerase, Pol 1l DNA polymerase, Pol 1 DNA polymerase, Pol K DNA polymerase, Pol (DNA polymerase, Pol 7 DNA polymerase, Pol 0 DNA polymerase, Pol v DNA polymerase, or a thermophilic nucleic acid polymerase (e.g., Therminator 7, 9° N polymerase (exo-), Therminator II, Therminator III, or Therminator IX). In embodiments, the DNA polymerase is a thermophilic nucleic acid polymerase. In embodiments, the DNA polymerase is a modified archaeal DNA polymerase. In embodiments, the polymerase is a reverse transcriptase. In embodiments, the polymerase is a mutant P. abyssi polymerase (e.g., such as a mutant P. abyssi polymerase described in WO 2018/148723 or WO 2020/056044, each of which are incorporated herein by reference for all purposes). In embodiments, the kit includes a strand-displacing polymerase. In embodiments, the kit includes a strand-displacing polymerase, such as a phi29 polymerase, phi29 mutant polymerase or a thermostable phi29 mutant polymerase.

In embodiments, the kit includes a buffered solution. Typically, the buffered solutions contemplated herein are made from a weak acid and its conjugate base or a weak base and its conjugate acid. For example, sodium acetate and acetic acid are buffer agents that can be used to form an acetate buffer. Other examples of buffer agents that can be used to make buffered solutions include, but are not limited to, Tris, bicine, tricine, HEPES, TES, MOPS, MOPSO and PIPES. Additionally, other buffer agents that can be used in enzyme reactions, hybridization reactions, and detection reactions are known in the art. In embodiments, the buffered solution can include Tris. With respect to the embodiments described herein, the pH of the buffered solution can be modulated to permit any of the described reactions. In some embodiments, the buffered solution can have a pH greater than pH 7.0, greater than pH 7.5, greater than pH 8.0, greater than pH 8.5, greater than pH 9.0, greater than pH 9.5, greater than pH 10, greater than pH 10.5, greater than pH 11.0, or greater than pH 11.5. In other embodiments, the buffered solution can have a pH ranging, for example, from about pH 6 to about pH 9, from about pH 8 to about pH 10, or from about pH 7 to about pH 9. In embodiments, the buffered solution can include one or more divalent cations. Examples of divalent cations can include, but are not limited to, Mg2+, Mn2+, Zn2+, and Ca2+. In embodiments, the buffered solution can contain one or more divalent cations at a concentration sufficient to permit hybridization of a nucleic acid. In embodiments, the buffered solution can contain one or more divalent cations at a concentration sufficient to permit hybridization of a nucleic acid. In embodiments, the buffered solution includes about 10 mM Tris, about 20 mM Tris, about 30 mM Tris, about 40 mM Tris, or about 50 mM Tris. In embodiments the buffered solution includes about 50 mM NaCl, about 75 mM NaCl, about 100 mM NaCl, about 125 mM NaCl, about 150 mM NaCl, about 200 mM NaCl, about 300 mM NaCl, about 400 mM NaCl, or about 500 mM NaCl. In embodiments, the buffered solution includes about 0.05 mM EDTA, about 0.1 mM EDTA, about 0.25 mM EDTA, about 0.5 mM EDTA, about 1.0 mM EDTA, about 1.5 mM EDTA or about 2.0 mM EDTA. In embodiments, the buffered solution includes about 0.01% Triton X-100, about 0.025% Triton X-100, about 0.05% Triton X-100, about 0.1% Triton X-100, or about 0.5% Triton X-100. In embodiments, the buffered solution includes 20 mM Tris pH 8.0, 100 mM NaCl, 0.1 mM EDTA, 0.025% Triton X-100. In embodiments, the buffered solution includes 20 mM Tris pH 8.0, 150 mM NaCl, 0.1 mM EDTA, 0.025% Triton X-100. In embodiments, the buffered solution includes 20 mM Tris pH 8.0, 300 mM NaCl, 0.1 mM EDTA, 0.025% Triton X-100. In embodiments, the buffered solution includes 20 mM Tris pH 8.0, 400 mM NaCl, 0.1 mM EDTA, 0.025% Triton X-100. In embodiments, the buffered solution includes 20 mM Tris pH 8.0, 500 mM NaCl, 0.1 mM EDTA, 0.025% Triton X-100.

In embodiments, the kit includes one or more sequencing reaction mixtures. In embodiments, the sequencing reaction mixture includes a buffer. In embodiments, the buffer includes an acetate buffer, 3-(N-morpholino)propanesulfonic acid (MOPS) buffer, N-(2-Acetamido)-2-aminoethanesulfonic acid (ACES) buffer, phosphate-buffered saline (PBS) buffer, 4-(2-hydroxyethyl)-1-piperazineethanesulfonic acid (HEPES) buffer, N-(1,1-Dimethyl-2-hydroxyethyl)-3-amino-2-hydroxypropanesulfonic acid (AMPSO) buffer, borate buffer (e.g., borate buffered saline, sodium borate buffer, boric acid buffer), 2-Amino-2-methyl-1,3-propanediol (AMPD) buffer, N-cyclohexyl-2-hydroxyl-3-aminopropanesulfonic acid (CAPSO) buffer, 2-Amino-2-methyl-1-propanol (AMP) buffer, 4-(Cyclohexylamino)-1-butanesulfonic acid (CABS) buffer, glycine-NaOH buffer, N-Cyclohexyl-2-aminoethanesulfonic acid (CHES) buffer, tris(hydroxymethyl)aminomethane (Tris) buffer, or a N-cyclohexyl-3-aminopropanesulfonic acid (CAPS) buffer. In embodiments, the buffer is a borate buffer. In embodiments, the buffer is a CHES buffer. In embodiments, the sequencing reaction mixture includes nucleotides, wherein the nucleotides include a reversible terminating moiety and a label covalently linked to the nucleotide via a cleavable linker. In embodiments, the sequencing reaction mixture includes a buffer, DNA polymerase, detergent (e.g., Triton X), a chelator (e.g., EDTA), and/or salts (e.g., ammonium sulfate, magnesium chloride, sodium chloride, or potassium chloride).

In embodiments, the kit includes, without limitation, nucleic acid primers, probes, adapters, enzymes, and the like, and are each packaged in a container, such as, without limitation, a vial, tube or bottle, in a package suitable for commercial distribution, such as, without limitation, a box, a sealed pouch, a blister pack and a carton. The package typically contains a label or packaging insert indicating the uses of the packaged materials. As used herein, “packaging materials” includes any article used in the packaging for distribution of reagents in a kit, including without limitation containers, vials, tubes, bottles, pouches, blister packaging, labels, tags, instruction sheets and package inserts.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, digital storage medium, etc., on which the information has been recorded. Yet another means that may be present is a website address which may be used via the Internet to access the information at a removed site. Any convenient means may be present in the kits.

Adapters and/or primers may be supplied in the kits ready for use, as concentrates-requiring dilution before use, or in a lyophilized or dried form requiring reconstitution prior to use. If required, the kits may further include a supply of a suitable diluent for dilution or reconstitution of the primers and/or adapters. Optionally, the kits may further include supplies of reagents, buffers, enzymes, and dNTPs for use in carrying out nucleic acid amplification and/or sequencing. Further components which may optionally be supplied in the kit include sequencing primers suitable for sequencing templates prepared using the methods described herein.

In embodiments, the kit can further include one or more biological stain(s) (e.g., any of the biological stains as described herein). For example, the kit can further include eosin and hematoxylin. In other examples, the kit can include a biological stain such as acridine orange, Bismarck brown, carmine, coomassie blue, crystal violet, DAPI, eosin, ethidium bromide, acid fuchsine, hematoxylin, Hoechst stains, iodine, methyl green, methylene blue, neutral red, Nile blue, Nile red, osmium tetroxide, propidium iodide, rhodamine, safranin, or any combination thereof.

Provided herein in another aspect is a tissue, wherein the tissue is attached to a solid support, and includes a plurality of single stranded polynucleotides immobilized to a cellular component or a matrix within the tissue. In embodiments, the polynucleotide fragments are generated according to the methods described herein.

### III. Methods

In an aspect is provided a method of profiling a sample (e.g., a cell). In embodiments, the method includes determining information (e.g., gene and protein expression) about the transcriptome of an organism thus elucidating subcellular substances and processes while gaining valuable spatial localization information within a cell. In embodiments, the method includes simultaneously sequencing a plurality of nucleic acids, such as RNA transcripts, in situ within an optically resolved volume of a sample (e.g., a voxel). RNA transcripts are responsible for the process of converting DNA into an organism's phenotype, thus by determining the types and quantity of RNA present in a sample (e.g., a cell), it is possible to assign a phenotype to the cell. RNA transcripts include coding RNA and non-coding RNA molecules, such as messenger RNA (mRNA), transfer RNA (tRNA), micro RNA (miRNA), small interfering RNA (siRNA), small nucleolar RNA (snoRNA), small nuclear RNA (snRNA), Piwi-interacting RNA (piRNA), enhancer RNA (eRNA), or ribosomal RNA (rRNA). In embodiments, the target is pre-mRNA. In embodiments, the target is heterogeneous nuclear RNA (hnRNA).

In an aspect is provided a method of forming single-stranded polynucleotides in situ, the method including: (a) extending a first primer hybridized to a circular polynucleotide with a strand-displacing polymerase to generate a first extension product including one or more complements of the circular polynucleotide within a cell or tissue; (b) contacting the first extension product with a second immobilized primer and extending the second immobilized primer with a polymerase to generate a second immobilized extension product, wherein the second primer is immobilized to a cellular component or a matrix within the cell or tissue; and (c) nicking the first extension product with an endonuclease, thereby generating one or more polynucleotide fragments, and removing the polynucleotide fragments, thereby forming single-stranded polynucleotides in situ. In embodiments, the second immobilized primer is covalently attached to a cell component (e.g., a protein, organelle, or lipid).

In an aspect is provided a method of forming single-stranded polynucleotides on a solid support, the method including: (a) extending a first primer hybridized to a circular polynucleotide with a strand-displacing polymerase to generate a first extension product including one or more complements of the circular polynucleotide; (b) contacting the first extension product with a second immobilized primer and extending the second immobilized primer with a polymerase to generate a second immobilized extension product, wherein the second primer is immobilized to a solid support; and (c) nicking the first extension product with an endonuclease (e.g., an endonuclease as described herein), thereby generating one or more polynucleotide fragments, and removing the polynucleotide fragments, thereby forming single-stranded polynucleotides on a solid support.

In embodiments, the method further includes detecting the immobilized extension products. In embodiments, the method further includes: (d) hybridizing a detection probe to the second immobilized extension product and detecting the detection probe, thereby detecting the circular polynucleotide. In embodiments, detecting includes serially contacting the amplification products with labeled probes (e.g., labeled oligonucleotides or labeled nucleotides). In embodiments, the method further includes: (d) hybridizing a primer to the second immobilized extension product and with a polymerase binding a nucleotide to the second immobilized extension product, wherein the nucleotide is associated with a detectable label (e.g., one or more fluorophores), and detecting the bound nucleotide. In embodiments, the method further includes: (d) hybridizing a primer to the second immobilized extension product and with a polymerase incorporated a labeled nucleotide to the second immobilized extension product, and detecting the labeled nucleotide.

In an aspect is provided a method of forming single-stranded polynucleotides in situ, the method including: (a) within a cell or tissue, extending a first immobilized primer hybridized to a circular polynucleotide with a strand-displacing polymerase to generate a first immobilized extension product including one or more complements of the circular polynucleotide; (b) contacting the first immobilized extension product with a second immobilized primer and extending the second immobilized primer with a polymerase to generate a second immobilized extension product, wherein the second primer is immobilized to a cellular component or a matrix within the cell or tissue; and (c) nicking the first immobilized extension product with an endonuclease, thereby generating one or more polynucleotide fragments, and removing the polynucleotide fragments, thereby forming single-stranded polynucleotides in situ.

In an aspect is provided a method of forming single-stranded polynucleotides on a solid support, the method including: (a) extending a first immobilized primer hybridized to a circular polynucleotide with a strand-displacing polymerase to generate a first immobilized extension product including one or more complements of the circular polynucleotide; (b) contacting the first immobilized extension product with a second immobilized primer and extending the second immobilized primer with a polymerase to generate a second immobilized extension product, wherein the second primer is immobilized to a solid support; and (c) nicking the first immobilized extension product with an endonuclease, thereby generating one or more polynucleotide fragments, and removing the polynucleotide fragments, thereby forming single-stranded polynucleotides on a solid support.

In embodiments, the method further includes: (d) hybridizing a detection probe to the second immobilized extension product and detecting the detection probe, thereby detecting the circular polynucleotide. In embodiments, detecting includes serially contacting the amplification products with labeled probes (e.g., labeled oligonucleotides or labeled nucleotides).

In an aspect is provided a method of forming single-stranded polynucleotides in situ, the method including: (a) within a cell or tissue, extending a first primer hybridized to a circular polynucleotide with a strand-displacing polymerase to generate a first extension product including one or more complements of the circular polynucleotide; (b) contacting the first extension product with a second immobilized primer and extending the second immobilized primer with a polymerase to generate a second immobilized extension product, wherein the second primer is immobilized to a cellular component or a matrix within the cell or tissue; and (c) nicking the second immobilized extension product with an endonuclease, thereby generating one or more polynucleotide fragments, and removing the polynucleotide fragments, thereby forming single-stranded polynucleotides in situ.

In an aspect is provided a method of forming single-stranded polynucleotides on a solid support, the method including: (a) extending a first primer hybridized to a circular polynucleotide with a strand-displacing polymerase to generate a first extension product including one or more complements of the circular polynucleotide; (b) contacting the first extension product with a second immobilized primer and extending the second immobilized primer with a polymerase to generate a second immobilized extension product, wherein the second primer is immobilized to a solid support; and (c) nicking the second extension product with an endonuclease, thereby generating one or more polynucleotide fragments, and removing the polynucleotide fragments, thereby forming single-stranded polynucleotides on a solid support. In embodiments, the solid support further includes a polymer (e.g., a polymer as described herein).

In embodiments, the method further includes: (d) hybridizing a detection probe to the first extension product and detecting the detection probe, thereby detecting the circular polynucleotide. In embodiments, detecting includes serially contacting the amplification products with labeled probes (e.g., labeled oligonucleotides or labeled nucleotides).

In embodiments, the circular polynucleotide includes a barcode sequence. In embodiments, the circular polynucleotide includes an identifying sequence (e.g., 1, 2, 3, 4, or 5 nucleotides associated with the target molecule). In embodiments, the barcode sequence is determined in situ. In embodiments, the first and/or second sequence of the circularizable oligonucleotide are determined in situ. In embodiments, the identifying sequence is a nucleotide. In embodiments, the identifying sequence is 2 to 10 nucleotides. In embodiments, the identifying sequence is a barcode sequence. In embodiments, the identifying sequence includes 6 to 12 nucleotides.

In embodiments, the detection probe includes a fluorescently labeled probe. The phrase “labeled probes” refers to mixture of nucleic acids that are detectably labeled, e.g., fluorescently labeled, such that the presence of the probe, as well as, any target sequence to which the probe is bound can be detected by assessing the presence of the label. In some embodiments, the probes are about 30-300 bases in length, 40-300 bases in length, or 70-300 bases in length. In some embodiments, the probes are relatively uniform in length (e.g., an average length +/−10 bases). The probes may be uniformly labeled based on position of label and/or number of labels within the probe. In some embodiments, the probes are single-stranded. In some embodiments, the probes are double-stranded. Additional detection probes and related properties may be found in, e.g., U.S. Pat. Pub. US 2011/0039735, which is incorporated herein by reference in its entirety.

In embodiments, the method further includes, prior to step (c), contacting the second immobilized extension product with a third immobilized primer and extending the third immobilized primer with a polymerase to generate a third immobilized extension product, wherein the third immobilized primer is immobilized to the cellular component or the matrix within the cell or tissue. In embodiments, step (c) further includes nicking the second immobilized extension product with an endonuclease, thereby generating one or more additional polynucleotide fragments. In embodiments, the method further includes, after step (c), detecting the third immobilized extension product. In embodiments, the method further includes, after step (c), sequencing the third immobilized extension product.

In embodiments, the method further includes, after step (c), detecting the second immobilized extension product. In embodiments, detecting the immobilized extension product includes hybridizing an oligonucleotide associated with a detectable label to the immobilized extension product and identifying the detectable label.

In embodiments, the method further includes, after step (c), sequencing the second immobilized extension product.

In embodiments, the method further includes, after step (c), nicking the second immobilized extension product with an endonuclease, thereby generating one or more additional polynucleotide fragments. In embodiments, the method further includes detecting the first extension product (e.g., the first immobilized extension product) and the third immobilized extension product. In embodiments, the method further includes detecting the first extension product (e.g., the first immobilized extension product) and the third immobilized extension product.

In an aspect is provided a method of sequencing a circular polynucleotide, the method including: i) amplifying the circular polynucleotide in a cell or tissue by extending a first primer hybridized to the circular polynucleotide with a strand-displacing polymerase to generate a first extension product including one or more complements of the circular polynucleotide; ii) contacting the first extension product with a second primer and extending the second primer with a polymerase to generate a second immobilized extension product, wherein the second primer is immobilized to a cellular component or a matrix within the cell or tissue; iii) nicking the first extension product with an endonuclease, thereby generating one or more polynucleotide fragments, and removing the polynucleotide fragments, thereby forming single-stranded polynucleotides on the solid support; and iv) hybridizing a sequencing primer to the single-stranded polynucleotides, and extending the sequencing primer to generate a first sequencing read, wherein the sequencing primer is immobilized to a cellular component or a matrix within the cell or tissue.

In embodiments, the method further includes, prior to step (iii), contacting the second immobilized extension product with a third primer and extending the third primer with a polymerase to generate a third immobilized extension product, wherein the third primer is immobilized to a cellular component or a matrix within the cell or tissue. In embodiments, step (iii) includes nicking the second immobilized extension product with an endonuclease, thereby generating one or more additional polynucleotide fragments, and removing the additional polynucleotide fragments.

In embodiments, the method further includes binding a specific binding reagent (e.g., an antibody, affimer, or aptamer) to a protein in the cell or tissue, wherein the specific binding reagent includes an oligonucleotide barcode, and determining the oligonucleotide barcode (e.g., sequencing the oligonucleotide barcode). In embodiments, the specific binding reagent is covalently attached to the oligonucleotide barcode. In embodiments, sequencing the oligonucleotide includes hybridizing a sequencing primer to the oligonucleotide barcode and incorporating a labeled nucleotide into the sequencing primer and detecting the incorporated nucleotide. In embodiments, additional proteins may be detected with different specific binding reagents bound to different oligonucleotide barcodes, wherein the oligonucleotide barcode is associated with the identity of the specific binding reagent, and thus the protein of interest.

In embodiments, prior to step (a), the method includes forming the circular polynucleotide. In embodiments, forming the circular polynucleotide includes hybridizing a first sequence of a circularizable oligonucleotide to a target nucleic acid molecule and a second sequence of the circularizable oligonucleotide to the target nucleic acid molecule, and ligating the first sequence and the second sequence to form the circular polynucleotide. In embodiments, the target nucleic acid molecule is an RNA molecule. In embodiments, the target nucleic acid molecule is an oligonucleotide barcode (e.g., an oligonucleotide barcode attached to a specific binding reagent described supra).

In embodiments, the first sequence and the second sequence are adjacent. For example, the first sequence and the second sequence, when bound to the target nucleic acid molecule, do not include a gap sequence between the two sequences. In alternative embodiments, the first sequence and the second sequence are separated by 1 or more nucleotides. For example, in embodiments, the first sequence and the second sequence, when bound to the target nucleic acid molecule, form a gap sequence including 1, 2, 3, 4, 5, 6, 7, 8, 9, 10, or more nucleotides. In embodiments, the gap sequence is 5, 10, 15, 20, 25, 30, 40, 50, 60, 70, 80, 90, 100, 125, 150, 175, or 200 nucleotides. In embodiments, the gap sequence is 5 to 150 nucleotides. In embodiments, the gap sequence is 1, 2, 3, 4, or 5 nucleotides.

In embodiments, the first primer includes a sequence (e.g., all or a portion thereof), or complement thereof, as described in Table 2. In embodiments, the first primer is a sequence selected from the group consisting of SEQ ID NO:21 to SEQ NO:74, or a complement thereof. In embodiments, the second primer is a sequence selected from the group consisting of SEQ ID NO:21 to SEQ NO:74, or a complement thereof. In embodiments, the third primer is a sequence selected from the group consisting of SEQ ID NO:21 to SEQ NO:74, or a complement thereof. In embodiments, the first primer and the second primer are different sequences selected from the group consisting of SEQ ID NO:21 to SEQ NO:74, or a complement thereof. In embodiments, the first primer and the third primer are the same sequence selected from the group consisting of SEQ ID NO:21 to SEQ NO:74, or a complement thereof.

In embodiments, the first primer is SEQ ID NO:25 and the second primer is SEQ ID NO:22. In embodiments, the first primer is SEQ ID NO:27 and the second primer is SEQ ID NO:48. In embodiments, the first primer is SEQ ID NO:21 and the second primer is SEQ ID NO:23. In embodiments, the first primer is SEQ ID NO:27 and the second primer is SEQ ID NO:22. In embodiments, the first primer is SEQ ID NO:27 and the second primer is SEQ ID NO:53. In embodiments, the third primer is SEQ ID NO:21. In embodiments, the third primer is SEQ ID NO:25. In embodiments, the third primer is SEQ ID NO:27. In embodiments, the first primer and the third primer are SEQ ID NO:25 and the second primer is SEQ ID NO:22. In embodiments, the first primer and the third primer are SEQ ID NO:27 and the second primer is SEQ ID NO:48. In embodiments, the first primer and the third primer are SEQ ID NO:21 and the second primer is SEQ ID NO:23. In embodiments, the first primer and the third primer are SEQ ID NO:27 and the second primer is SEQ ID NO:22. In embodiments, the first primer and the third primer are SEQ ID NO:27 and the second primer is SEQ ID NO:53.

In embodiments, the first primer is SEQ ID NO:27 and the second primer is SEQ ID NO:62. In embodiments, the first primer is SEQ ID NO:37 and the second primer is SEQ ID NO:48. In embodiments, the first primer is SEQ ID NO:37 and the second primer is SEQ ID NO:62. In embodiments, the first primer is SEQ ID NO:37 and the second primer is SEQ ID NO:22. In embodiments, the first primer is SEQ ID NO:27 and the second primer is SEQ ID NO:67. In embodiments, the first primer is SEQ ID NO:37 and the second primer is SEQ ID NO:53. In embodiments, the first primer is SEQ ID NO:37 and the second primer is SEQ ID NO:67. In embodiments, the third primer is SEQ ID NO:27. In embodiments, the third primer is SEQ ID NO:37. In embodiments, the first primer and the third primer are SEQ ID NO:27 and the second primer is SEQ ID NO:62. In embodiments, the first primer and the third primer are SEQ ID NO:37 and the second primer is SEQ ID NO:48. In embodiments, the first primer and the third primer are SEQ ID NO:37 and the second primer is SEQ ID NO:62. In embodiments, the first primer and the third primer are SEQ ID NO:37 and the second primer is SEQ ID NO:22. In embodiments, the first primer and the third primer are SEQ ID NO:27 and the second primer is SEQ ID NO:67. In embodiments, the first primer and the third primer are SEQ ID NO:37 and the second primer is SEQ ID NO:53. In embodiments, the first primer and the third primer are SEQ ID NO:37 and the second primer is SEQ ID NO:67.

In embodiments, the sequencing includes sequencing by synthesis, sequencing by hybridization, sequencing by binding, sequencing by ligation, or pyrosequencing.

In embodiments, the sequencing includes extending a sequencing primer by incorporating a labeled nucleotide or labeled nucleotide analogue, and detecting the label to generate a signal for each incorporated nucleotide or nucleotide analogue, wherein the sequencing primer is hybridized to the extension product.

In embodiments, the sequencing primer includes a reversible 3′ blocking moiety. In embodiments, the reversible blocking moiety includes a dideoxy nucleotide triphosphate. In embodiments, prior to step iv), the reversible blocking moiety is removed, thereby generating an extendible sequencing primer. In embodiments, the sequencing primer is immobilized to a matrix or a cellular component of the cell. In embodiments, the sequencing primer is immobilized to a solid support.

In embodiments, the one or more immobilized oligonucleotides (e.g., the one or more immobilized primers in a cell or on a solid support) include blocking groups at their 3′ ends that prevent polymerase extension. A blocking moiety prevents formation of a covalent bond between the 3′ hydroxyl moiety of the nucleotide and the 5′ phosphate of another nucleotide. A blocking moiety can be reversible, whereby the blocking moiety can be removed or modified to allow the 3′ hydroxyl to form a covalent bond with the 5′ phosphate of another nucleotide. A blocking moiety can be effectively irreversible under particular conditions used in a method set forth herein. Non-limiting examples of 3′ blocking groups include a 3′-ONH₂ blocking group, a 3′-O-allyl blocking group, or a 3′-O-azidomethyl blocking group. In embodiments, the 3′ blocking group is a C3, C9, C12, or C18 spacer phosphoramidite, a 3′phosphate, a C3, C6, C12 amino modifier, or a reversible blocking moiety (e.g., reversible blocking moieties are described in U.S. Pat. Nos. 7,541,444 and 7,057,026). In embodiments, the 3′ modification is a 3′-phosphate modification includes a 3′ phosphate moiety, which is removed by a PNK enzyme.

In embodiments, the method further includes, prior to step i), forming the circular polynucleotide. In embodiments, forming the circular polynucleotide is performed in a cell. In embodiments, forming the circular polynucleotide is performed in solution. In embodiments, forming the circular polynucleotide is performed on a solid support. Methods for forming a circular polynucleotide are described briefly herein, and further in, e.g., U.S. Pat. Nos. 11,492,662, 11,434,525, and 11,486,004, U.S. Pat. Pub. Nos. US 2020/0224244 and US 2022/0235410, and PCT Pub. No. WO 2022/087485, each of which is incorporated herein by reference in its entirety.

In embodiments, forming the circular polynucleotide includes a) hybridizing a circularizable oligonucleotide to a target nucleic acid (e.g., an RNA molecule), wherein the circularizable oligonucleotide includes a first region at a 3′ end that hybridizes to a first complementary region of the target nucleic acid, and a second region at a 5′ end that hybridizes to a second complementary region of the target nucleic acid, wherein the second complementary region is 5′ with respect to the first complementary region and b) circularizing the circularizable oligonucleotide to generate a circular polynucleotide, wherein circularizing includes extending the 3′ end of the circularizable oligonucleotide (e.g., extending the 3′ end using a polymerase (e.g., a Thermus thermophilus (Tth) DNA polymerase) to incorporate one or more nucleotides) along the target nucleic acid to generate a complementary sequence (e.g., complementary to the target nucleic acid, for example a target RNA sequence), and ligating the complementary sequence to the 5′ end of the oligonucleotide primer.

In embodiments, forming the circular polynucleotide includes (a) hybridizing a linear template polynucleotide to a splint primer immobilized on a surface, wherein (i) the splint primer includes, in the 5′ to 3′ direction, a first sequence and a second sequence, (ii) the first sequence is complementary to a 5′ portion of the linear template polynucleotide, and (iii) the second sequence is complementary to a 3′ portion of the linear template polynucleotide; and (b) circularizing the linear template polynucleotide to form a circular template polynucleotide including a continuous strand lacking free 5′ and 3′ ends. In embodiments, the linear template polynucleotide is generated by joining a first adapter polynucleotide to a 5′ end of a sample polynucleotide, and joining a second adapter polynucleotide to a 3′ end of the sample polynucleotide. In embodiments, the first adapter polynucleotide includes a portion that hybridizes to the first sequence of the splint primer, and the second adapter polynucleotide includes a portion that hybridizes to the second sequence of the splint primer.

In an aspect is provided a method of detecting a protein in a cell or tissue. In embodiments, the method includes contacting each of the proteins with a specific binding reagent, wherein the specific binding reagent includes an oligonucleotide barcode; hybridizing a padlock probe to two nucleic acid sequences of the barcode, wherein the padlock probe is a single-stranded polynucleotide having a 5′ and a 3′ end, wherein the padlock probe comprises a primer binding sequence from a known set of primer binding sequences; amplifying the barcode sequence according to a method described herein (e.g., in an aspect provided herein); sequencing each barcode to obtain a multiplexed signal in the cell in situ; demultiplexing the multiplexed signal by comparison with the known set of barcodes; and detecting the plurality of targets by identifying the associated barcodes detected in the cell.

In an aspect is provided a method of sequencing, the method including contacting a cell or tissue including a nucleic acid molecule with a polynucleotide probe including a first target hybridization sequence and a second target hybridization sequence; hybridizing the first target hybridization sequence to the nucleic acid molecule and hybridizing the second target hybridization sequence to the nucleic acid molecule; ligating the first target hybridization sequence to the second target hybridization sequence to form a circular polynucleotide; amplifying the circular polynucleotide to form an amplification product according to the method described herein; and hybridizing a first sequencing primer to the amplification product, and sequencing the first target hybridization sequence or the second target hybridization sequence.

In embodiments, the circular polynucleotide includes an endogenous nucleic acid sequence, or a complement thereof. In embodiments, the circular polynucleotide includes a genomic sequence, or a complement thereof. In embodiments, the circular polynucleotide includes a synthetic sequence, or a complement thereof.

In embodiments, the method includes amplifying the circular polynucleotide of the cell in situ. In embodiments, amplifying the circular polynucleotide generates an amplification product. In embodiments, the amplification product includes three or more copies of the circular polynucleotide. In embodiments, the amplification product includes at least three or more copies of the circular polynucleotide. In embodiments, the amplification product includes at least five or more copies of the circular polynucleotide. In embodiments, the amplification product includes at 5 to 10 copies of the circular polynucleotide. In embodiments, the amplification product includes 10 to 20 copies of the circular polynucleotide. In embodiments, the amplification product includes 20 to 50 copies of the circular polynucleotide.

In embodiments, amplifying the circular polynucleotide includes incubating the circular polynucleotide with the strand-displacing polymerase (a) for about 1 minute to about 2 hours, and/or (b) at a temperature of about 20° C. to about 50° C. In embodiments, amplifying the circular polynucleotide includes incubating the circular polynucleotide with the strand-displacing polymerase for about 1 minute to about 2 hours. In embodiments, amplifying the circular polynucleotide includes incubating the circular polynucleotide with the strand-displacing polymerase for about 5, about 10, about 20, about 30, about 40, about 45, about 50, about 55, or about 60 minutes. In embodiments, amplifying the circular polynucleotide includes incubating the circular polynucleotide with the strand-displacing polymerase for about 5 minutes. In embodiments, amplifying the circular polynucleotide includes incubating the circular polynucleotide with the strand-displacing polymerase for about 10 minutes. In embodiments, amplifying the circular polynucleotide includes incubating the circular polynucleotide with the strand-displacing polymerase for about 20 minutes. In embodiments, amplifying the circular polynucleotide includes incubating the circular polynucleotide with the strand-displacing polymerase for about 30 minutes. In embodiments, amplifying the circular polynucleotide includes incubating the circular polynucleotide with the strand-displacing polymerase for about 45 minutes. In embodiments, amplifying the circular polynucleotide includes incubating the circular polynucleotide with the strand-displacing polymerase for about 60 minutes.

In embodiments, amplifying the circular polynucleotide includes incubating the circular polynucleotide with the strand-displacing polymerase for about 1 hour to about 12 hours. In embodiments, amplifying includes incubation with the strand-displacing polymerase for about 60 seconds to about 60 minutes. In embodiments, amplifying includes incubation with the strand-displacing polymerase for about 10 minutes to about 60 minutes. In embodiments, amplifying includes incubation with the strand-displacing polymerase for about 10 minutes to about 30 minutes. In embodiments, amplifying the circular polynucleotide includes incubating the circular polynucleotide with the strand-displacing polymerase for about 1, about 2, about 3, about 4, about 5, about 6, about 7, about 8, about 9, about 10, about 11, or about 12 hours. In embodiments, amplifying the circular polynucleotide includes incubating the circular polynucleotide with the strand-displacing polymerase for more than 12 hours.

In embodiments, amplifying the circular polynucleotide includes incubating the circular polynucleotide with the strand-displacing polymerase at a temperature of about 20° C. to about 50° C. In embodiments, incubation with the strand-displacing polymerase is at a temperature of about 20° C., about 25° C., about 30° C., about 35° C., about 40° C., about 45° C., or about 50° C. In embodiments, incubation with the strand-displacing polymerase is at a temperature of about 35° C. to 42° C. In embodiments, incubation with the strand-displacing polymerase is at a temperature of about 35° C., about 36° C., about 37° C., about 38° C., about 39° C., about 40° C., about 41° C., or about 42° C. In embodiments, the strand-displacing polymerase is a phi29 polymerase, a SD polymerase, a Bst large fragment polymerase, phi29 mutant polymerase, a Thermus aquaticus polymerase, or a thermostable phi29 mutant polymerase.

In embodiments, amplifying includes rolling circle amplification (RCA) or rolling circle transcription (RCT) (see, e.g., Lizardi et al., Nat. Genet. 19:225-232 (1998), which is incorporated herein by reference in its entirety). Several suitable rolling circle amplification methods are known in the art. For example, RCA amplifies a circular polynucleotide (e.g., DNA) by polymerase extension of an amplification primer complementary to a portion of the template polynucleotide. This process generates copies of the circular polynucleotide template such that multiple complements of the template sequence arranged end to end in tandem are generated (i.e., a concatemer) locally preserved at the site of the circle formation. In embodiments, the amplifying occurs at isothermal conditions. In embodiments, the amplifying includes hybridization chain reaction (HCR). HCR uses a pair of complementary, kinetically trapped hairpin oligomers to propagate a chain reaction of hybridization events, as described in Dirks, R. M., & Pierce, N. A. (2004) PNAS USA, 101(43), 15275-15278, which is incorporated herein by reference for all purposes. In embodiments, the amplifying includes branched rolling circle amplification (BRCA); e.g., as described in Fan T, Mao Y, Sun Q, et al. Cancer Sci. 2018; 109:2897-2906, which is incorporated herein by reference in its entirety. In embodiments, the amplifying includes hyperbranched rolling circle amplification (HRCA). Hyperbranched RCA uses a second primer complementary to the first amplification product. This allows products to be replicated by a strand-displacement mechanism, which yields drastic amplification within an isothermal reaction (Lage et al., Genome Research 13:294-307 (2003), which is incorporated herein by reference in its entirety). In embodiments, amplifying includes polymerase extension of an amplification primer. In embodiments, the polymerase is T4, T7, Sequenase, Taq, Klenow, and Pol I DNA polymerases. SD polymerase, Bst large fragment polymerase, or a phi29 polymerase or mutant thereof. In embodiments, the strand-displacing enzyme is an SD polymerase, Bst large fragment polymerase, or a phi29 polymerase or mutant thereof. In embodiments, the strand-displacing polymerase is Bst DNA Polymerase Large Fragment, Thermus aquaticus (Taq) polymerase, or a mutant thereof. In embodiments, the strand-displacing polymerase is a phi29 polymerase, a phi29 mutant polymerase or a thermostable phi29 mutant polymerase. A “phi polymerase” (or “(D29 polymerase”) is a DNA polymerase from the (29 phage or from one of the related phages that, like (D29, contain a terminal protein used in the initiation of DNA replication. For example, phi29 polymerases include the B103, GA-1, PZA, (D15, BS32, M2Y (also known as M2), Nf, G1, Cp-1, PRD1, PZE, SFS, Cp-5, Cp-7, PR4, PR5, PR722, L17, (D21, and AV-1 DNA polymerases, as well as chimeras thereof. A phi29 mutant DNA polymerase includes one or more mutations relative to naturally-occurring wild-type phi29 DNA polymerases, for example, one or more mutations that alter interaction with and/or incorporation of nucleotide analogs, increase stability, increase read length, enhance accuracy, increase photo tolerance, and/or alter another polymerase property, and can include additional alterations or modifications over the wild-type phi29 DNA polymerase, such as one or more deletions, insertions, and/or fusions of additional peptide or protein sequences. Thermostable phi29 mutant polymerases are known in the art, see for example US 2014/0322759, which is incorporated herein by reference for all purposes. For example, a thermostable phi29 mutant polymerase refers to an isolated bacteriophage phi29 DNA polymerase including at least one mutation selected from the group consisting of M8R, V51A, M97T, L123S, G197D, K209E, E221K, E239G, Q497P, K512E, E515A, and F526 (relative to wild type phi29 polymerase). In embodiments, the polymerase is a phage or bacterial RNA polymerases (RNAPs). In embodiments, the polymerase is a T7 RNA polymerase. In embodiments, the polymerase is an RNA polymerase. Useful RNA polymerases include, but are not limited to, viral RNA polymerases such as T7 RNA polymerase, T3 polymerase, SP6 polymerase, and Kll polymerase; Eukaryotic RNA polymerases such as RNA polymerase I, RNA polymerase II, RNA polymerase III, RNA polymerase IV, and RNA polymerase V; and Archaea RNA polymerase.

In embodiments, the amplification method includes a standard dNTP mixture including dATP, dCTP, dGTP and dTTP (for DNA) or dATP, dCTP, dGTP and dUTP (for RNA). In embodiments, the amplification method includes a mixture of standard dNTPs and modified nucleotides that contain functional moieties (e.g., bioconjugate reactive groups) that serve as attachment points to the cell or the matrix in which the cell is embedded (e.g. a hydrogel). In embodiments, the amplification method includes a mixture of standard dNTPs and modified nucleotides that contain functional moieties (e.g., bioconjugate reactive groups) that participate in the formation of a bioconjugate linker. The modified nucleotides may react and link the amplification product to the surrounding cell scaffold. For example, amplifying may include an extension reaction wherein the polymerase incorporates a modified nucleotide into the amplification product, wherein the modified nucleotide includes a bioconjugate reactive moiety (e.g., an alkynyl moiety) attached to the nucleobase. The bioconjugate reactive moiety of the modified nucleotide participates in the formation of a bioconjugate linker by reacting with a complementary bioconjugate reactive moiety present in the cell (e.g., a crosslinking agent, such as NHS-PEG-azide, or an amine moiety) thereby attaching the amplification product to the internal scaffold of the cell. In embodiments, the functional moiety can be covalently cross-linked, copolymerize with or otherwise non-covalently bound to the matrix. In embodiments, the functional moiety can react with a cross-linker. In embodiments, the functional moiety can be part of a ligand-ligand binding pair. Suitable exemplary functional moieties include an amine, acrydite, alkyne, biotin, azide, and thiol. In embodiments of crosslinking, the functional moiety is cross-linked to modified dNTP or dUTP or both. In embodiments, suitable exemplary cross-linker reactive groups include imidoester (DMP), succinimide ester (NHS), maleimide (Sulfo-SMCC), carbodiimide (DCC, EDC) and phenyl azide. Cross-linkers within the scope of the present disclosure may include a spacer moiety. In embodiments, such spacer moieties may be functionalized. In embodiments, such spacer moieties may be chemically stable. In embodiments, such spacer moieties may be of sufficient length to allow amplification of the nucleic acid bound to the matrix. In embodiments, suitable exemplary spacer moieties include polyethylene glycol, carbon spacers, photo-cleavable spacers and other spacers known to those of skill in the art and the like. In embodiments, amplification reactions include standard dNTPs and a modified nucleotide (e.g., amino-allyl dUTP, 5-TCO-PEG4-dUTP, C8-Alkyne-dUTP, 5-Azidomethyl-dUTP, 5-Vinyl-dUTP, or 5-Ethynyl dLTTP). For example, during amplification a mixture of standard dNTPs and aminoallyl deoxyuridine 5′-triphosphate (dUTP) nucleotides may be incorporated into the amplicon and subsequently cross-linked to the cell protein matrix by using a cross-linking reagent (e.g., an amine-reactive crosslinking agent with PEG spacers, such as (PEGylated bis(sulfosuccinimidyl)suberate) (BS(PEG)9)).

In embodiments, the circular polynucleotide is about 100 to about 1000 nucleotides in length. In embodiments, the circular polynucleotide is about 100, about 200, about 300, about 400, about 500, about 600, about 700, about 800, about 900, or about 1000 nucleotides in length. In embodiments, the circular polynucleotide is greater than 1000 nucleotides in length. In embodiments, the circular polynucleotide is about or more than about 100, 150, 200, 250, 300, 350, 400, 500, 750, 1000, or more nucleotides in length. In embodiments, the circular polynucleotide includes a plurality of sequencing primer binding sequences. In embodiments, the circular polynucleotide includes a plurality of different sequencing primer binding sequences.

In embodiments, sequencing includes a plurality of sequencing cycles. In embodiments, sequencing includes 2, 3, 4, 5, 6, 7, 8, 9, 10, 11, 12, 13, 14, or 15 sequencing cycles. In embodiments, sequencing includes at least 10, 20, 30 40, or 50 sequencing cycles. In embodiments, sequencing includes at least 10 sequencing cycles. In embodiments, sequencing includes 10 to 20 sequencing cycles. In embodiments, sequencing includes 10, 11, 12, 13, 14, or 15 sequencing cycles. In embodiments, sequencing includes (a) extending a sequencing primer by incorporating a labeled nucleotide, or labeled nucleotide analogue and (b) detecting the label to generate a signal for each incorporated nucleotide or nucleotide analogue.

In embodiments, the circular polynucleotide includes a sequence (e.g., a cleavable site) recognized by the endonuclease. In embodiments, the sequence recognized by the endonuclease is a double-stranded recognition sequence. In embodiments, the sequence recognized by the endonuclease includes a modified nucleotide. In embodiments, the sequence recognized by the endonuclease includes a native nucleotide. In embodiments, the sequence recognized by the endonuclease includes a non-native nucleotide.

In embodiments, the endonuclease lacks double-strand cleavage activity. In embodiments, the endonuclease is a nicking endonuclease. These nicking endonucleases typically recognize non-palindromes. They can be bona fide nicking enzymes, such as frequent cutter Nt.CviPII and Nt.CviQII, or rare-cutting homing endonucleases I-BasI and I-HmuI, both of which recognize a degenerate 24-bp sequence. As well, isolated large subunits of heterodimeric Type IIS restriction endonucleases such as BtsI, BsrDI and BstNBI/BspD6I display nicking activity. Thus, properties of restriction endonucleases that make double-strand cuts may be retained by engineering variants of these enzymes such that they make single-strand breaks. In various embodiments, recognition sequence-specific nicking endonucleases are used as cleavage agents that cleave only a single-strand of double-stranded DNA at a cleavage site. Nicking endonucleases useful in various embodiments of methods and compositions described herein include Nb.BbvCI, Nb.BsmI, Nb.BsrDI, Nb.BtsI, Nt.AlwI, Nt.BbvCI, Nt.BsmAI, Nt.BspQI, Nt.BstNBI, and Nt.CviPII, used either alone or in various combinations. In various embodiments, nicking endonucleases that cleave outside of their recognition sequence, e.g. Nb.BsrDI, Nb.BtsI, Nt.AlwI, Nt.BsmAI, Nt.BspQI, Nt.BstNBI, and Nt.CviPII, are used. In some instances, nicking endonucleases that cut within their recognition sequences, e.g. Nb.BbvCI, Nb.BsmI, or Nt.BbvCI are used. Recognition sites for the various specific cleavage agents used herein, such as the nicking endonucleases, comprise a specific nucleic acid sequence.

The nickase Nb.BbvCI (New England Biolabs, Ipswich, Mass.) nicks at the following cleavage site with respect to its recognition site (with “I” specifying the nicking (cleavage) site and “N” representing any nucleoside, e.g. one of C, A, G or T): 5′-CCTCAGC-3′ (SEQ ID NO:1) and 3′-GGAGTICG-5′ (SEQ ID NO:2). The nickase Nb.BsmI (New England Biolabs, Ipswich, Mass.) nicks at the following cleavage site with respect to its recognition site: 5′-GAATGCN-3′ (SEQ ID NO:3) and 3′-CTTACIGN-5′ (SEQ ID NO:4). The nickase Nb.BsrDI (New England Biolabs, Ipswich, Mass.) nicks at the following cleavage site with respect to its recognition site: 5′-GCAATGNN-3′ (SEQ ID NO:5) and 3′-CGTTACINN-5′ (SEQ ID NO:6). The nickase Nb.BtsI (New England Biolabs, Ipswich, Mass.) nicks at the following cleavage site with respect to its recognition site: 5′-GCAGTGNN-3′ (SEQ ID NO:7) and 3′-CGTCACINN-5′ (SEQ ID NO:8). The nickase Nt.AlwI (New England Biolabs, Ipswich, Mass.) nicks at the following cleavage site with respect to its recognition site: 5′-GGATCNNNNIN-3′ (SEQ ID NO:9) and 3′-CCTAGNNNNN-5′ (SEQ ID NO:10). The nickase Nt.BbvCI (New England Biolabs, Ipswich, Mass.) nicks at the following cleavage site with respect to its recognition site: 5′-CCITCAGC-3′ (SEQ ID NO:11) and 3′-GGAGTCG-5′ (SEQ ID NO:12). The nickase Nt.BsmAI (New England Biolabs, Ipswich, Mass.) nicks at the following cleavage site with respect to its recognition site: 5′-GTCTCNIN-3′ (SEQ ID NO:13) and 3′-CAGAGNN-5′ (SEQ ID NO.: 14). The nickase Nt.BspQI (New England Biolabs, Ipswich, Mass.) nicks at the following cleavage site with respect to its recognition site: 5′-GCTCTTCNI-3′ (SEQ ID NO.: 15) and 3′-CGAGAAGN-5′ (SEQ ID NO:16). The nickase Nt.BstNBI (New England Biolabs, Ipswich, Mass.) nicks at the following cleavage site with respect to its recognition site: 5′-GAGTCNNNNIN-3′ (SEQ ID NO:17) and 3′-CTCAGNNNNN-5′ (SEQ ID NO:18). The nickase Nt.CviPII (New England Biolabs, Ipswich, Mass.) nicks at the following cleavage site with respect to its recognition site (wherein D denotes A or G or T and wherein H denotes A or C or T: 5′-|CCD-3′ (SEQ ID NO:19) and 3′-GGH-5′ (SEQ ID NO:20).

In embodiments, the double-stranded recognition sequence includes SEQ ID NO:1 and SEQ ID NO:2. In embodiments, the double-stranded recognition sequence includes SEQ ID NO:3 and SEQ ID NO:4. In embodiments, the double-stranded recognition sequence includes SEQ ID NO:5 and SEQ ID NO:6. In embodiments, the double-stranded recognition sequence includes SEQ ID NO:7 and SEQ ID NO:8. In embodiments, the double-stranded recognition sequence includes SEQ ID NO:9 and SEQ ID NO:10. In embodiments, the double-stranded recognition sequence includes SEQ ID NO:11 and SEQ ID NO:12. In embodiments, the double-stranded recognition sequence includes SEQ ID NO:13 and SEQ ID NO:14. In embodiments, the double-stranded recognition sequence includes SEQ ID NO:15 and SEQ ID NO:16. In embodiments, the double-stranded recognition sequence includes SEQ ID NO:17 and SEQ ID NO:18. In embodiments, the double-stranded recognition sequence includes SEQ ID NO:19 and SEQ ID NO:20.

In embodiments, the double-stranded recognition sequence includes SEQ ID NO:1 duplexed to SEQ ID NO:2. In embodiments, the double-stranded recognition sequence includes SEQ ID NO:3 duplexed to SEQ ID NO:4. In embodiments, the double-stranded recognition sequence includes SEQ ID NO:5 duplexed to SEQ ID NO:6. In embodiments, the double-stranded recognition sequence includes SEQ ID NO:7 duplexed to SEQ ID NO:8. In embodiments, the double-stranded recognition sequence includes SEQ ID NO:9 duplexed to SEQ ID NO:10. In embodiments, the double-stranded recognition sequence includes SEQ ID NO:11 duplexed to SEQ ID NO:12. In embodiments, the double-stranded recognition sequence includes SEQ ID NO:13 duplexed to SEQ ID NO:14. In embodiments, the double-stranded recognition sequence includes SEQ ID NO:15 duplexed to SEQ ID NO:16. In embodiments, the double-stranded recognition sequence includes SEQ ID NO:17 duplexed to SEQ ID NO:18. In embodiments, the double-stranded recognition sequence includes SEQ ID NO:19 duplexed to SEQ ID NO:20.

In embodiments, the endonuclease includes one or more endonucleases selected from the group consisting of Nb.BbvCI, Nb.BsmI, NbBsrDI, Nb.BtsI, Nt.AlwI, Nt.BbvCI, Nb.BssSI, Nt.BsmAI, Nt.BspQI, Nt.BstNBI, and Nt.CviPII. In embodiments, the endonuclease is Nb.BbvCI or Nt.BsmAI. In embodiments, the endonuclease is Nb.BbvCI. In embodiments, the endonuclease is Nt.BsmAI.

In embodiments, the circular polynucleotide includes any one of the sequences of SEQ ID NO:1 to SEQ ID NO:20. In embodiments, the circular polynucleotide includes one or more different sequences of SEQ ID NO:1 to SEQ ID NO:20. In embodiments, the circular polynucleotide includes two or more different sequences of SEQ ID NO:1 to SEQ ID NO:20. In embodiments the circular polynucleotide includes any two different sequences of SEQ ID NO:1 to SEQ ID NO:20. In embodiments, the circular polynucleotide includes any three different sequences of SEQ ID NO:1 to SEQ ID NO:20. In embodiments, the circular polynucleotide includes the sequence of SEQ ID NO:3 or SEQ ID NO: 4. In embodiments, the circular polynucleotide includes the sequence of SEQ ID NO:13 or SEQ ID NO:14.

In embodiments, cleaving (e.g., nicking) includes maintaining suitable reaction conditions to permit efficient cleavage (e.g., buffer, pH, temperature conditions). In embodiments, cleaving is performed at about 20° C. to about 60° C. In embodiments, cleavage is performed at about 20° C. to about 30° C., about 30° C. to about 40° C., about 40° C. to about 50° C., or about 50° C. to about 60° C. In embodiments, cleavage is performed at about 20° C., about 25° C., about 30° C., about 35° C., about 37° C., about 40° C., about 42° C., about 45° C., about 48° C., about 50° C., about 55° C., or about 60° C. In embodiments, cleavage is performed at less than 20° C. In embodiments, cleavage is performed at greater than 60° C.

In embodiments, cleavage (e.g., nicking) is performed for about 5 seconds (sec) to about 24 hours (hrs). In embodiments, cleavage is performed for about 5 sec to about 30 sec, about 30 sec to about 60 sec, about 1 minute (min) to about 5 min, about 5 min to about 15 min, about 15 min to about 30 min, about 30 min to about 60 min, about 1 hr to about 4 hrs, about 4 hrs to about 12 hrs, or about 12 hrs to about 24 hrs. In embodiments, cleavage is performed for about 5 sec, 15 sec, 30 sec, 45 sec, 1 min, 2 min, 3 min, 4 min, 5 min, 6 min, 7 min, 8 min, 9 min, 10 min, 11 min, 12 min, 13 min, 14 min, or about 15 min. In embodiments, cleavage is performed for about 20 min, 25 min, 30 min, 35 min, 40 min, 45 min, 50 min, 55 min, or about 1 hr. In embodiments, cleavage is performed for about 2 hrs, 3 hrs, 4 hrs, 5 hrs, 6 hrs, 7 hrs, 8 hrs, 9 hrs, 10 hrs, 11 hrs, or about 12 hrs. In embodiments, cleavage is performed for about 14 hrs, 16 hrs, 18 hrs, 20 hrs, 22 hrs, or about 24 hrs.

In embodiments, cleavage (e.g., nicking) is performed with about 1 unit (U) to about 50 U of endonuclease. The term “unit (U)” or “enzyme unit (U)” is used in accordance with its plain and ordinary meaning, and refers to the amount of the enzyme that catalyzes the conversion of one micromole of substrate per minute under the specified conditions of a given assay. In embodiments, cleavage is performed with about 1 U to about 5 U of endonuclease. In embodiments, cleavage is performed with about 5 U to about 10 U of endonuclease. In embodiments, cleavage is performed with about 10 U to about 15 U of endonuclease. In embodiments, cleavage is performed with about 15 U to about 20 U of endonuclease. In embodiments, cleavage is performed with about 20 U to about 25 U of endonuclease. In embodiments, cleavage is performed with about 25 U to about 35 U of endonuclease. In embodiments, cleavage is performed with about 35 U to about 50 U of endonuclease. In embodiments, cleavage is performed with about 1, 2, 3, 4, 5, 6, 7, 8, 9, 10, 11, 12, 13, 14, 15, 16, 17, 18, 19, 20, 21, 22, 23, 24, 25, 30, 35, 40, 45 or 50 U of endonuclease. In embodiments, cleavage is performed with less than about 1 U of endonuclease. In embodiments, cleavage is performed with greater than about 50 U of endonuclease.

In embodiments, removing the removing the nicked extension product includes denaturing the nicked extension product. Denaturation may be performed in solutions with high pH and/or organic solutions capable of denaturing DNA. In some embodiments, the nicked extension product may be removed via heat denaturation. In embodiments, removing the nicked extension product includes contacting the nicked extension product with a denaturant, wherein the denaturant is a buffered solution including betaine, dimethyl sulfoxide (DMSO), ethylene glycol, formamide, glycerol, guanidine thiocyanate, 4-methylmorpholine 4-oxide (NMO), or a mixture thereof. In some embodiments, denaturation is achieved by exposure to chemical denaturants such as urea or formamide, with concentrations suitably adjusted, or using high or low pH (e.g., pH between 4-6 or 8-9). In embodiments, the denaturant is a buffered solution including betaine, dimethyl sulfoxide (DMSO), ethylene glycol, formamide, glycerol, guanidine thiocyanate, 4-methylmorpholine 4-oxide (NMO), or a mixture thereof. In embodiments, the first denaturant is a buffered solution including about 0% to about 50% dimethyl sulfoxide (DMSO); about 0% to about 50% ethylene glycol; about 0% to about 20% formamide; or about 0 to about 3M betaine, or a mixture thereof.

In embodiments, removing the nicked first extension product includes contacting the nicked first extension product with a chemical denaturant. In embodiments, removing the nicked second extension product includes contacting the nicked second extension product with a chemical denaturant. In embodiments, the chemical denaturant includes ethylene glycol, polyethylene glycol, 1,2-propanediol, dimethyl sulfoxide (DMSO), glycerol, formamide, 7-deaza-dGTP, acetamide, betaine, or tetramethylammonium chloride (TMAC). In embodiments, the chemical denaturant includes 100% formamide.

In embodiments, the circular polynucleotide includes primer binding sequences complementary to one or more additional primers (e.g., amplification and/or sequencing primers).

In embodiments, the circularizable oligonucleotide includes about 50 to about 150 nucleotides. In embodiments, the circularizable oligonucleotide includes about 50 to about 300 nucleotides. In embodiments, the circularizable oligonucleotide includes about 50 to about 500 nucleotides. In embodiments, the circularizable oligonucleotide includes about or more than about 50, 75, 100, 125, 150, 175, 200, 250, 300, 350, 400, or 500 nucleotides. In embodiments, the circularizable oligonucleotide includes less than about 50, 75, 100, 125, 150, 175, 200, 250, 300, 350, 400, or 500 nucleotides.

In embodiments, the circularizable oligonucleotide includes at least one amplification primer binding sequence or at least one sequencing primer binding sequence. The amplification primer binding sequence refers to a nucleotide sequence that is complementary to a primer useful in initiating amplification (i.e., an amplification primer). Likewise, a sequencing primer binding sequence is a nucleotide sequence that is complementary to a primer useful in initiating sequencing (i.e., a sequencing primer). Primer binding sequences usually have a length in the range of between 3 to 36 nucleotides, also 5 to 24 nucleotides, also from 14 to 36 nucleotides. In embodiments, an amplification primer and a sequencing primer are complementary to the same primer binding sequence, or overlapping primer binding sequences. In embodiments, an amplification primer and a sequencing primer are complementary to different primer binding sequences.

In embodiments, the amplification primer binding sequence and/or sequencing primer binding sequence includes any one of the sequences (e.g., all or a portion thereof), or complement thereof, as described in Table 2. In embodiments, the amplification primer binding sequence includes any one of the sequences, or complement thereof, of SEQ ID NO:21 to SEQ ID NO:74. In embodiments, the sequencing primer binding sequence includes any one of the sequences, or complement thereof, of SEQ ID NO:21 to SEQ ID NO:74. In embodiments, the amplification primer binding sequence includes any one of the sequences, or complement thereof, of SEQ ID NO: 21, SEQ ID NO:22, SEQ ID NO:23, SEQ ID NO:25, SEQ ID NO:27, SEQ ID NO:48, or SEQ ID NO:53. In embodiments, the sequencing primer binding sequence includes any one of the sequences, or complement thereof, of SEQ ID NO: 21, SEQ ID NO:22, SEQ ID NO:23, SEQ ID NO:25, SEQ ID NO:27, SEQ ID NO:48, or SEQ ID NO:53. In embodiments, the amplification primer binding sequence includes any one of the sequences, or complement thereof, of SEQ ID NO: 27, SEQ ID NO:62, SEQ ID NO:37, SEQ ID NO:48, SEQ ID NO:22, SEQ ID NO:67, or SEQ ID NO:53. In embodiments, the sequencing primer binding sequence includes any one of the sequences, or complement thereof, of SEQ ID NO: 27, SEQ ID NO:62, SEQ ID NO:37, SEQ ID NO:48, SEQ ID NO:22, SEQ ID NO:67, or SEQ ID NO:53.

In embodiments, the circularizable oligonucleotide further includes a barcode sequence. In embodiments, the barcode sequence is at least 5, 6, 7, 8, 9, 10, 11, 12, 13, 14, or 15 nucleotides in length. In embodiments, the barcode sequence is selected from a known set of barcode sequences. In embodiments, each barcode sequence is unique within the known set of barcodes. In embodiments, the barcodes are selected to form a known set of barcodes, e.g., the set of barcodes may be distinguished by a particular Hamming distance.

In embodiments, the barcode (i.e., the barcode sequence) is at least 5, 6, 7, 8, 9, 10, 11, 12, 13, 14, or 15 nucleotides in length. In embodiments, the barcode is 5, 6, 7, 8, 9, 10, 11, 12, 13, 14, or 15 nucleotides in length. In embodiments, the barcode is 10 to 15 nucleotides in length. In embodiments, the barcode is at least about 1, 2, 3, 4, 5, 6, 7, 8, 9, 10, 15, 20, 25, 30, 35, 40, 45, 50 or more nucleotides in length. In embodiments, the barcode can be at most about 300, 200, 100, 90, 80, 70, 60, 50, 40, 30, 20, 15, 12, 10, 9, 8, 7, 6, 5, 4 or fewer or more nucleotides in length. In embodiments, the barcode includes between about 5 to about 8, about 5 to about 10, about 5 to about 15, about 5 to about 20, about 10 to about 150 nucleotides. In embodiments, the barcode includes between 5 to 8, 5 to 10, 5 to 15, 5 to 20, 10 to 150 nucleotides. In embodiments, the barcode is 10 nucleotides. In embodiments, the barcode may include a unique sequence (e.g., a barcode sequence) that gives the barcode its identifying functionality. The unique sequence may be random or non-random. Attachment of the barcode sequence (via binding of a proximity probe conjugated to the barcode sequence) to a protein or nucleic acid of interest (i.e., the target) may associate the barcode sequence with the protein or nucleic acid of interest. The barcode may then be used to identify the protein or nucleic acid of interest during sequencing, even when other proteins or nucleic acids of interest (e.g., including different oligonucleotide barcodes) are present. In embodiments, the barcode consists only of a unique barcode sequence. In embodiments, the 5′ end of a barcoded oligonucleotide is phosphorylated. In embodiments, the barcode is known (i.e., the nucleic sequence is known before sequencing) and is sorted into a basis-set according to their Hamming distance. Oligonucleotide barcodes (e.g., barcode sequences included in an oligonucleotide probe) can be associated with a target of interest by knowing, a priori, the target of interest, such as a gene or protein. In embodiments, the barcodes further include one or more sequences capable of specifically binding a gene or nucleic acid sequence of interest. For example, in embodiments, the barcode includes a sequence capable of hybridizing to mRNA, e.g., one containing a poly-T sequence (e.g., having several T's in a row, e.g., 4, 5, 6, 7, 8, or more T's).

In embodiments, the barcode is included as part of an oligonucleotide of longer sequence length, such as a primer or a random sequence (e.g., a random N-mer). In embodiments, the barcode contains random sequences to increase the mass or size of the oligonucleotide tag. The random sequence can be of any suitable length, and there may be one or more than one present. As non-limiting examples, the random sequence may have a length of 10 to 40, 10 to 30, 10 to 20, 25 to 50, 15 to 40, 15 to 30, 20 to 50, 20 to 40, or 20 to 30 nucleotides. In embodiments, each barcode sequence is selected from a known set of barcode sequences. In embodiments, each of the known set of barcode sequences is associated with a targeting sequence from a known set of targeting sequences. In embodiments, a first barcode sequence is associated with a first targeting sequence, and wherein a second barcode sequence is associated with a second targeting sequence (e.g., wherein the second targeting sequence is included in an oligonucleotide probe targeting a different target nucleic acid than the first targeting sequence). In embodiments, the same barcode sequence is associated with a plurality of oligonucleotide probes targeting different sequences of the same target nucleic acid (e.g., the same target polynucleotide).

In embodiments, the barcode is taken from a “pool” or “set” or “basis-set” of potential oligonucleotide barcode sequences. The set of barcodes may be selected using any suitable technique, e.g., randomly, or such that the sequences allow for error detection and/or correction, or having a particular feature, such as by being separated by a certain distance (e.g., Hamming distance). In embodiments, the method includes selecting a basis-set of oligonucleotide barcodes having a specified Hamming distance (e.g., a Hamming distance of 10; a Hamming distance of 5). The pool may have any number of potential barcode sequences, e.g., at least 100, at least 300, at least 500, at least 1,000, at least 3,000, at least 5,000, at least 10,000, at least 30,000, at least 50,000, at least 100,000, at least 300,000, at least 500,000, or at least 1,000,000 barcode sequences. In embodiments, a barcode is a degenerate or partially-degenerate sequence, such that one or more nucleotides are selected at random from a set of two or more different nucleotides at one or more positions, with each of the different nucleotides selected at one or more positions represented in a pool of oligonucleotides including the degenerate or partially-degenerate sequence. The number of possible barcodes in a given set of barcodes will vary with the number of degenerate positions, and the number of bases permitted at each such position. For example, a barcode of five nucleotides (consecutive or non-consecutive), in which each position can be any of A, T, G, or C represents 54, or 1024 possible barcodes. In embodiments, certain barcode sequences may be excluded from a pool, such as barcodes in which every position is the same base. In embodiments, there are about, 102, 103 104, 105, 106, 107, 108, 109, or a number or a range between any two of these values, unique nucleotide barcode sequences. In embodiments, there are at least, or at most 102, 103 104, 105, 106, 107, 108, 109 unique barcode sequences. In embodiments, a barcode is about, 1, 2, 3, 4, 5, 10, 15, 20, 25, 30, 35, 40, 45, 50, or a number or a range between any two of these values, nucleotides in length. A barcode can be at least, or at most, 1, 2, 3, 4, 5, 10, 15, 20, 25, 30, 35, 40, 45, 50, 100, or 200 nucleotides in length.

In embodiments, the barcodes in the known set of barcodes have a specified Hamming distance. In embodiments, the Hamming distance is 4 to 15. In embodiments, the Hamming distance is 8 to 12. In embodiments, the Hamming distance is 10. In embodiments, the Hamming distance is 0 to 100. In embodiments, the Hamming distance is 0 to 15. In embodiments, the Hamming distance is 0 to 10. In embodiments, the Hamming distance is 1 to 10. In embodiments, the Hamming distance is 5 to 10. In embodiments, the Hamming distance is 1 to 100. In embodiments, the Hamming distance between any two barcode sequences of the set is at least 2, 3, 4, or 5. In embodiments, the Hamming distance between any two barcode sequences of the set is at least 3. In embodiments, the Hamming distance between any two barcode sequences of the set is at least 4.

In embodiments, the target polynucleotide includes a cancer-associated gene nucleic acid sequence, a viral nucleic acid sequence, a bacterial nucleic acid sequence, or a fungal nucleic acid sequence.

In embodiments, the target nucleic acid (i.e., the target polynucleotide) includes a nucleic acid sequence encoding a TCR alpha chain, a TCR beta chain, a TCR delta chain, a TCR gamma chain, or any fragment thereof (e.g., variable regions including VDJ or VJ regions, constant regions, transmembrane regions, fragments thereof, combinations thereof, and combinations of fragments thereof). In embodiments, the target nucleic acid includes a nucleic acid sequence encoding a B cell receptor heavy chain, B cell receptor light chain, or any fragment thereof (e.g., variable regions including VDJ or VJ regions, constant regions, transmembrane regions, fragments thereof, combinations thereof, and combinations of fragments thereof). In embodiments, the target nucleic acid includes a CDR3 nucleic acid sequence. In embodiments, the target nucleic acid includes a TCRA gene sequence or a TCRB gene sequence. In embodiments, the target nucleic acid includes a TCRA gene sequence and a TCRB gene sequence. In embodiments, the target nucleic acid includes sequences of various T cell receptor alpha variable genes (TRAV genes), T cell receptor alpha joining genes (TRAJ genes), T cell receptor alpha constant genes (TRAC genes), T cell receptor beta variable genes (TRBV genes), T cell receptor beta diversity genes (TRBD genes), T cell receptor beta joining genes (TRBJ genes), T cell receptor beta constant genes (TRBC genes), T cell receptor gamma variable genes (TRGV genes), T cell receptor gamma joining genes (TRGJ genes), T cell receptor gamma constant genes (TRGC genes), T cell receptor delta variable genes (TRDV genes), T cell receptor delta diversity genes (TRDD genes), T cell receptor delta joining genes (TRDJ genes), or T cell receptor delta constant genes (TRDC genes).

In embodiments, the first sequence includes a nucleic acid sequence encoding a B cell receptor V region, and wherein the second sequence includes a nucleic acid sequence encoding a B cell receptor J region.

In embodiments, the first sequence and the second sequence flank a CDR3 nucleic acid sequence.

In embodiments, the target polynucleotide includes a cancer-associated gene nucleic acid sequence, a viral nucleic acid sequence, a bacterial nucleic acid sequence, or a fungal nucleic acid sequence. In embodiments, the cancer-associated gene is a nucleic acid sequence identified within The Cancer Genome Atlas Program, accessible at www.cancer.gov/tcga.

In embodiments, the target polynucleotide includes a CD4, CD68, CD20, CD11c, CD8, HLA-DR, Ki67, CD45RO, PanCK, CD3e, CD44, CD45, HLA-A, CD14, CD56, CD57, CD19, CD2, CD1a, CD107a, CD21, Pax5, FOXP3, Granzyme B, CD38, CD39, CD79a, TIGIT, TOX, TP63, S100A4, TFAM, GP100, LaminBi, CK19, CK17, GATA3, SOX2, Bcl2, EpCAM, Caveolin, CD163, CD11b, MPO, CD141, iNOS, PD-1, PD-L1, ICOS, TIM3, LAG3, IDO1, CD40, HLA-E, IFNG, CD69, E-cadherin, CD31, Histone H3, Beta-actin, Podoplanin, SMA, Vimentin, Collagen IV, CD34, Beta-catenin, MMP-9, ZEB1, ASCT2, Na/K ATPase, HK1, LDHA, G6PD, IDH2, GLUT1, pNRF2, ATPA5, SDHA, Citrate Synthase, CPT1A, PARP, BAK, BCL-XL, BAX, BAD, Cytochrome c, LC3B, Beclin-1, H2AX, pRPS6, PCNA, Cyclin D1, HLA-DPB1, LEF1, GAL9, CD138, MC Tryptase, OX40, ZAP70, CD7, C1Qa, CCR6, CD15, AXL, and/or CD227 nucleic acid sequence.

In embodiments, the target polynucleotide can include any polynucleotide of interest. The polynucleotide can include DNA, RNA, peptide nucleic acid, morpholino nucleic acid, locked nucleic acid, glycol nucleic acid, threose nucleic acid, mixtures thereof, and hybrids thereof. In embodiments, the polynucleotide is obtained from one or more source organisms. In some embodiments, the polynucleotide can include a selected sequence or a portion of a larger sequence. In embodiments, sequencing a portion of a polynucleotide or a fragment thereof can be used to identify the source of the polynucleotide. With reference to nucleic acids, polynucleotides and/or nucleotide sequences a “portion,” “fragment” or “region” can be at least 5 consecutive nucleotides, at least 10 consecutive nucleotides, at least 15 consecutive nucleotides, at least 20 consecutive nucleotides, at least 25 consecutive nucleotides, at least 50 consecutive nucleotides, at least 100 consecutive nucleotides, or at least 150 consecutive nucleotides.

In embodiments, the entire sequence of the target polynucleotide is about 1 to 3 kb, and only a portion of that target (e.g., 50 to 100 nucleotides) is sequenced. In embodiments, the target polynucleotide is about 1 to 3 kb. In embodiments, the target polynucleotide is about 1 to 2 kb. In embodiments, the target polynucleotide is about 1 kb. In embodiments, the target polynucleotide is about 2 kb. In embodiments, the target polynucleotide is less than 1 kb. In embodiments, the target polynucleotide is about 500 nucleotides. In embodiments, the target polynucleotide is about 200 nucleotides. In embodiments, the target polynucleotide is about 100 nucleotides. In embodiments, the target polynucleotide is less than 100 nucleotides. In embodiments, the target polynucleotide is about 5 to 50 nucleotides.

In embodiments, the target polynucleotide is an RNA nucleic acid sequence or DNA nucleic acid sequence. In embodiments, the target polynucleotide is an RNA nucleic acid sequence or DNA nucleic acid sequence from the same cell. In embodiments, the target polynucleotide is an RNA nucleic acid sequence. In embodiments, the RNA nucleic acid sequence is stabilized using known techniques in the art. For example, RNA degradation by RNase should be minimized using commercially available solutions, e.g., RNA Later@, RNA Lysis Buffer, or Keratinocyte serum-free medium). In embodiments, the target polynucleotide is messenger RNA (mRNA), transfer RNA (tRNA), micro RNA (miRNA), small interfering RNA (siRNA), small nucleolar RNA (snoRNA), small nuclear RNA (snRNA), Piwi-interacting RNA (piRNA), enhancer RNA (eRNA), or ribosomal RNA (rRNA). In embodiments, the target polynucleotide is pre-mRNA. In embodiments, the target polynucleotide is heterogeneous nuclear RNA (hnRNA). In embodiments, the target polynucleotide is mRNA, tRNA (transfer RNA), rRNA (ribosomal RNA), or noncoding RNA (such as lncRNA (long noncoding RNA)). In embodiments, the target polynucleotides are on different regions of the same RNA nucleic acid sequence.

In embodiments, the target polynucleotide includes RNA nucleic acid sequences. In embodiments the target polynucleotide is an RNA transcript. In embodiments the target polynucleotide is a single stranded RNA nucleic acid sequence. In embodiments, the target polynucleotide is an RNA nucleic acid sequence or a DNA nucleic acid sequence (e.g., cDNA).

In embodiments, the target polynucleotide is a cDNA target polynucleotide nucleic acid sequence and before step i), the RNA nucleic acid sequence is reverse transcribed to generate the cDNA target polynucleotide nucleic acid sequence. In embodiments, reverse transcription of the RNA nucleic acid is performed with a reverse transcriptase, for example, Tth DNA polymerase or mutants thereof. In embodiments, the target polynucleotide is genomic DNA (gDNA), mitochondrial DNA, chloroplast DNA, episomal DNA, viral DNA, or copy DNA (cDNA). In embodiments, the target polynucleotide is coding RNA such as messenger RNA (mRNA), and non-coding RNA (ncRNA) such as transfer RNA (tRNA), microRNA (miRNA), small nuclear RNA (snRNA), or ribosomal RNA (rRNA). In embodiments, the target polynucleotide is a cancer-associated gene. In embodiments, to minimize amplification errors or bias, the target polynucleotide is not reverse transcribed to generate cDNA.

In embodiments, the circularizable oligonucleotide includes locked nucleic acids (LNAs), Bis-locked nucleic acids (bisLNAs), twisted intercalating nucleic acids (TINAs), bridged nucleic acids (BNAs), 2′-O-methyl RNA:DNA chimeric nucleic acids, minor groove binder (MGB) nucleic acids, morpholino nucleic acids, C5-modified pyrimidine nucleic acids, peptide nucleic acids (PNAs), or combinations thereof. In embodiments, the circularizable oligonucleotide includes one or more LNA nucleotides. In embodiments, the sequence complementary to the first hybridization sequence and/or the second sequence complementary to the second hybridization sequence of the circularizable oligonucleotide includes one or more LNA nucleotides.

In embodiments, amplifying the circular polynucleotide includes rolling circle amplification (RCA), exponential rolling circle amplification (eRCA), hyperbranched rolling circle amplification (HRCA), loop-mediated isothermal amplification (LAMP), or multiple displacement amplification (MDA). In embodiments, amplifying the circular polynucleotide includes rolling circle amplification (RCA) or exponential rolling circle amplification (eRCA). Exponential RCA is similar to the linear process except that it uses a second primer (e.g., one or more immobilized oligonucleotide(s)) having a sequence that is identical to at least a portion of the circular template (Lizardi et al. Nat. Genet. 19:225 (1998)). This two-primer system achieves isothermal, exponential amplification. Exponential RCA has been applied to the amplification of non-circular DNA through the use of a linear probe that binds at both of its ends to contiguous regions of a target DNA followed by circularization using DNA ligase (Nilsson et al. Science 265(5181):208 5(1994)).

In embodiments, the circular polynucleotide is single-stranded DNA. In embodiments, the circular polynucleotide includes an adapter. The adapter may have other functional elements including tagging sequences (i.e., a barcode), attachment sequences, palindromic sequences, restriction sites, sequencing primer binding sites, functionalization sequences, and the like. Barcodes can be of any of a variety of lengths. In embodiments, the primer includes a barcode that is 10-50, 20-30, or 4-12 nucleotides in length. In embodiments, the adapter includes a primer binding sequence that is complementary to at least a portion of a primer (e.g., a sequencing primer binding sequence complementary to at least a portion of a sequencing primer). Primer binding sites can be of any suitable length. In embodiments, a primer binding site is about or at least about 10, 15, 20, 25, 30, or more nucleotides in length. In embodiments, a primer binding site is 10-50, 15-30, or 20-25 nucleotides in length. In embodiments the circular polynucleotide is cfDNA.

In embodiments, the targets are proteins or carbohydrates. In embodiments, the targets are proteins. In embodiments, the targets are carbohydrates. In embodiments when the target are proteins and/or carbohydrates, the method includes contacting the proteins with a specific binding reagent, wherein the specific binding reagent includes an oligonucleotide barcode (e.g., the target polynucleotide is attached to the specific binding reagent). In embodiments, the specific binding reagent includes an antibody, single-chain Fv fragment (scFv), antibody fragment-antigen binding (Fab), or an aptamer. In embodiments, the specific binding reagent is a peptide, a cell penetrating peptide, an aptamer, a DNA aptamer, an RNA aptamer, an antibody, an antibody fragment, a light chain antibody fragment, a single-chain variable fragment (scFv), a lipid, a lipid derivative, a phospholipid, a fatty acid, a triglyceride, a glycerolipid, a glycerophospholipid, a sphingolipid, a saccharolipid, a polyketide, a polylysine, polyethyleneimine, diethylaminoethyl (DEAE)-dextran, cholesterol, or a sterol moiety. In embodiments, the specific binding reagent interacts (e.g., contacts, or binds) with one or more specific binding reagents on the cell surface. Carbohydrate-specific antibodies are known in the art, see for example Kappler, K., Hennet, T. Genes Immun 21, 224-239 (2020) In embodiments, the target polynucleotide is polynucleotide attached to a specific binding reagent. In embodiments, the specific binding reagent is an antibody, single-chain Fv fragment (scFv), antibody fragment-antigen binding (Fab), or an aptamer.

In embodiments, the target polynucleotide is attached to a specific binding reagent (e.g., an antibody) via a linker (e.g., a bioconjugate linker). In embodiments, the target polynucleotide is attached to the specific binding reagent via a linker formed by reacting a first bioconjugate reactive moiety (e.g., the bioconjugate reactive moiety includes an amine moiety, aldehyde moiety, alkyne moiety, azide moiety, carboxylic acid moiety, dibenzocyclooctyne (DBCO) moiety, tetrazine moiety, epoxy moiety, isocyanate moiety, furan moiety, maleimide moiety, thiol moiety, or transcyclooctene (TCO) moiety) with a second bioconjugate reactive moiety). In embodiments, the target polynucleotide includes a barcode, wherein the barcode is a known sequence associated with the specific binding reagent. In embodiments, the barcode is at least 5, 6, 7, 8, 9, 10, 11, 12, 13, 14, or 15 nucleotides in length. In embodiments, the barcode is 5, 6, 7, 8, 9, 10, 11, 12, 13, 14, or 15 nucleotides in length.

Specific antibodies tagged with known oligonucleotide sequences can be synthesized by using bifunctional crosslinkers reactive towards thiol (via maleimide) and amine (via NHS) moieties. For example, a 5′-thiol-modified oligonucleotide could be conjugated to a crosslinker via maleimide chemistry and purified. The oligos with a 5′-NHS-ester would then be added to a solution of antibodies and reacted with amine residues on the antibodies surface to generate tagged antibodies capable of binding analytes with target epitopes. These tagged antibodies include oligonucleotide sequence(s). The one or more oligonucleotide sequences may include a barcode, binding sequences (e.g., primer binding sequence or sequences complementary to hybridization pads), and/or unique molecular identifier (UMI) sequences.

In embodiments, specific binding entails a binding affinity, expressed as a KD (such as a KD measured by surface plasmon resonance at an appropriate temperature, such as 37° C.). In embodiments, the KD of a specific binding interaction is less than about 100 nM, 50 nM, 10 nM, 1 nM, 0.05 nM, or lower. In embodiments, the KD of a specific binding interaction is about 0.01-100 nM, 0.1-50 nM, or 1-10 nM. In embodiments, the KD of a specific binding interaction is less than 10 nM. The binding affinity of an antibody can be readily determined by one of ordinary skill in the art (for example, by Scatchard analysis). A variety of immunoassay formats can be used to select antibodies specifically immunoreactive with a particular antigen. For example, solid-phase ELISA immunoassays are routinely used to select monoclonal antibodies specifically immunoreactive with an analyte. See Harlow and Lane, ANTIBODIES: A LABORATORY MANUAL, Cold Springs Harbor Publications, New York, (1988) for a description of immunoassay formats and conditions that can be used to determine specific immunoreactivity. Typically a specific or selective reaction will be at least twice background signal to noise and more typically more than 10 to 100 times greater than background.

In embodiments, the methods and compositions described herein are utilized to analyze the various sequences of TCRs and BCRs from immune cells, for example various clonotypes. In embodiments, the target nucleic acid includes a nucleic acid sequence encoding a TCR alpha chain, a TCR beta chain, a TCR delta chain, a TCR gamma chain, or any fragment thereof (e.g., variable regions including VDJ or VJ regions, constant regions, transmembrane regions, fragments thereof, combinations thereof, and combinations of fragments thereof). In embodiments, the target nucleic acid includes a nucleic acid sequence encoding a B cell receptor heavy chain, B cell receptor light chain, or any fragment thereof (e.g., variable regions including VDJ or VJ regions, constant regions, transmembrane regions, fragments thereof, combinations thereof, and combinations of fragments thereof). In embodiments, the target nucleic acid includes a CDR3 nucleic acid sequence. In embodiments, the target nucleic acid includes a TCRA gene sequence or a TCRB gene sequence. In embodiments, the target nucleic acid includes a TCRA gene sequence and a TCRB gene sequence. In embodiments, the target nucleic acid includes sequences of various T cell receptor alpha variable genes (TRAV genes), T cell receptor alpha joining genes (TRAJ genes), T cell receptor alpha constant genes (TRAC genes), T cell receptor beta variable genes (TRBV genes), T cell receptor beta diversity genes (TRBD genes), T cell receptor beta joining genes (TRBJ genes), T cell receptor beta constant genes (TRBC genes), T cell receptor gamma variable genes (TRGV genes), T cell receptor gamma joining genes (TRGJ genes), T cell receptor gamma constant genes (TRGC genes), T cell receptor delta variable genes (TRDV genes), T cell receptor delta diversity genes (TRDD genes), T cell receptor delta joining genes (TRDJ genes), or T cell receptor delta constant genes (TRDC genes).

RNA, including mRNA, is highly susceptible to degradation upon exposure to one or more RNAses. RNAses are present in a wide range of locations, including water, many reagents, laboratory equipment and surfaces, skin, and mucous membranes. Working with RNA often requires preparing an RNAse-free environment and materials, as well as taking precautions to avoid introducing RNAses into an RNAse-free environment. These precautions include, but are not limited to, cleaning surfaces with an RNAse cleaning product (e.g., RNASEZAP™ and other commercially available products or 0.5% sodium dodecyl sulfate [SDS] followed by 3% H2O2); using a designated workspace, materials, and equipment (e.g., pipets, pipet tips); using barrier tips; baking designated glassware (e.g., 300° C. for 2 hours) prior to use; treating enzymes, reagents, and other solutions (e.g., with diethyl pyrocarbonate [DEPC] or dimethyl pyrocarbonate [DMPC]) or using commercially available, certified RNAse-free water or solutions, or ultrafiltered water (e.g., for Tris-based solutions); including an RNAse inhibitor while avoiding temperatures or denaturing conditions that could deactivate the inhibitor); and wearing clean gloves (while avoiding contaminated surfaces) and a clean lab coat.

In embodiments, the cell forms part of a tissue in situ. In embodiments, the cell is an isolated single cell. In embodiments, the cell is a prokaryotic cell. In embodiments, the cell is a eukaryotic cell. In embodiments, the cell is a bacterial cell (e.g., a bacterial cell or bacterial spore), a fungal cell (e.g., a fungal spore), a plant cell, or a mammalian cell. In embodiments, the cell is a stem cell. In embodiments, the stem cell is an embryonic stem cell, a tissue-specific stem cell, a mesenchymal stem cell, or an induced pluripotent stem cell. In embodiments, the cell is an endothelial cell, muscle cell, myocardial, smooth muscle cell, skeletal muscle cell, mesenchymal cell, epithelial cell; hematopoietic cell, such as lymphocytes, including T cell, e.g., (Th1 T cell, Th2 T cell, ThO T cell, cytotoxic T cell); B cell, pre-B cell; monocytes; dendritic cell; neutrophils; or a macrophage. In embodiments, the cell is a stem cell, an immune cell, a cancer cell (e.g., a circulating tumor cell or cancer stem cell), a viral-host cell, or a cell that selectively binds to a desired target. In embodiments, the cell includes a T cell receptor gene sequence, a B cell receptor gene sequence, or an immunoglobulin gene sequence. In embodiments, the cell includes a Toll-like receptor (TLR) gene sequence. In embodiments, the cell includes a gene sequence corresponding to an immunoglobulin light chain polypeptide and a gene sequence corresponding to an immunoglobulin heavy chain polypeptide. In embodiments, the cell is a genetically modified cell. In embodiments, the cell is a circulating tumor cell or cancer stem cell.

In embodiments, the cell is a prokaryotic cell. In embodiments, the cell is a bacterial cell. In embodiments, the bacterial cell is a Bacteroides, Clostridium, Faecalibacterium, Eubacterium, Ruminococcus, Peptococcus, Peptostreptococcus, or Bifidobacterium cell. In embodiments, the bacterial cell is a Bacteroides fragilis, Bacteroides melaninogenicus, Bacteroides oralis, Enterococcus faecalis, Escherichia coli, Enterobacter sp., Klebsiella sp., Bifidobacterium bifidum, Staphylococcus aureus, Lactobacillus, Clostridium perfringens, Proteus mirabilis, Clostridium tetani, Clostridium septicum, Pseudomonas aeruginosa, Salmonella enterica, Faecalibacterium prausnitzii, Peptostreptococcus sp., or Peptococcus sp. cell. In embodiments, the cell is a fungal cell. In embodiments, the fungal cell is a Candida, Saccharomyces, Aspergillus, Penicillium, Rhodotorula, Trametes, Pleospora, Sclerotinia, Bullera, or a Galactomyces cell.

In embodiments, the cell is a viral-host cell. A “viral-host cell” is used in accordance with its ordinary meaning in virology and refers to a cell that is infected with a viral genome (e.g., viral DNA or viral RNA). The cell, prior to infection with a viral genome, can be any cell that is susceptible to viral entry. In embodiments, the viral-host cell is a lytic viral-host cell. In embodiments, the viral-host cell is capable of producing viral protein. In embodiments, the viral-host cell is a lysogenic viral-host cell. In embodiments, the cell is a viral-host cell including a viral nucleic acid sequence, wherein the viral nucleic acid sequence is from a Hepadnaviridae, Adenoviridae, Herpesviridae, Poxviridae, Parvoviridae, Reoviridae, Coronaviridae, Retroviridae virus.

In embodiments, the cell is an adherent cell (e.g., epithelial cell, endothelial cell, or neural cell). Adherent cells are usually derived from tissues of organs and attach to a substrate (e.g., epithelial cells adhere to an extracellular matrix coated substrate via transmembrane adhesion protein complexes). Adherent cells typically require a substrate, e.g., tissue culture plastic, which may be coated with extracellular matrix (e.g., collagen and laminin) components to increase adhesion properties and provide other signals needed for growth and differentiation. In embodiments, the cell is a neuronal cell, an endothelial cell, epithelial cell, germ cell, plasma cell, a muscle cell, peripheral blood mononuclear cell (PBMC), a myocardial cell, or a retina cell. In embodiments, the cell is a suspension cell (e.g., a cell free-floating in the culture medium, such a lymphoblast or hepatocyte). In embodiments, the cell is a glial cell (e.g., astrocyte, radial glia), pericyte, or stem cell (e.g., a neural stem cell). In embodiments, the cell is a neuronal cell. In embodiments, the cell is an endothelial cell. In embodiments, the cell is an epithelial cell. In embodiments, the cell is a germ cell. In embodiments, the cell is a plasma cell. In embodiments, the cell is a muscle cell. In embodiments, the cell is a peripheral blood mononuclear cell (PBMC). In embodiments, the cell is a myocardial cell. In embodiments, the cell is a retina cell. In embodiments, the cell is a lymphoblast. In embodiments, the cell is a hepatocyte. In embodiments, the cell is a glial cell. In embodiments, the cell is an astrocyte. In embodiments, the cell is a radial glia. In embodiments, the cell is a pericyte. In embodiments, the cell is a stem cell. In embodiments, the cell is a neural stem cell.

In embodiments, the cell is bound to a known antigen. In embodiments, the cell is a cell that selectively binds to a desired target, wherein the target is an antibody, or antigen binding fragment, an aptamer, affimer, non-immunoglobulin scaffold, small molecule, or genetic modifying agent. In embodiments, the cell is a leukocyte (i.e., a white-blood cell). In embodiments, leukocyte is a granulocyte (neutrophil, eosinophil, or basophil), monocyte, or lymphocyte (T cells and B cells). In embodiments, the cell is a lymphocyte. In embodiments, the cell is a T cell, an NK cell, or a B cell.

In embodiments, the cell is an immune cell. In embodiments, the immune cell is a granulocyte, a mast cell, a monocyte, a neutrophil, a dendritic cell, or a natural killer (NK) cell. In embodiments, the immune cell is an adaptive cell, such as a T cell, NK cell, or a B cell. In embodiments, the cell includes a T cell receptor gene sequence, a B cell receptor gene sequence, or an immunoglobulin gene sequence. In embodiments, the immune cell is a granulocyte. In embodiments, the immune cell is a mast cell. In embodiments, the immune cell is a monocyte. In embodiments, the immune cell is a neutrophil. In embodiments, the immune cell is a dendritic cell. In embodiments, the immune cell is a natural killer (NK) cell. In embodiments, the immune cell is a T cell. In embodiments, the immune cell is a B cell. In embodiments, the cell includes a T cell receptor gene sequence. In embodiments, the cell includes a B cell receptor gene sequence. In embodiments, the cell includes an immunoglobulin gene sequence. In embodiments, the plurality of target nucleic acids includes non-contiguous regions of a nucleic acid molecule. In embodiments, the non-contiguous regions include regions of a VDJ recombination of a B cell or T cell.

In embodiments, the cell is a cancer cell. In embodiments, the cancer is lung cancer, colorectal cancer, skin cancer, colon cancer, pancreatic cancer, breast cancer, cervical cancer, lymphoma, leukemia, or a cancer associated with aberrant K-Ras, aberrant APC, aberrant Smad4, aberrant p53, or aberrant TGFβ. In embodiments, the cancer cell includes a ERBB2, KRAS, TP53, PIK3CA, or FGFR2 gene. In embodiments, the cancer cell includes a HER2 gene. In embodiments, the cancer cell includes a cancer-associated gene (e.g., an oncogene associated with kinases and genes involved in DNA repair) or a cancer-associated biomarker. A “biomarker” is a substance that is associated with a particular characteristic, such as a disease or condition. A change in the levels of a biomarker may correlate with the risk or progression of a disease or with the susceptibility of the disease to a given treatment. In embodiments, the cancer is Acute Myeloid Leukemia, Adrenocortical Carcinoma, Bladder Urothelial Carcinoma, Breast Ductal Carcinoma, Breast Lobular Carcinoma, Cervical Carcinoma, Cholangiocarcinoma, Colorectal Adenocarcinoma, Esophageal Carcinoma, Gastric Adenocarcinoma, Glioblastoma Multiforme, Head and Neck Squamous Cell Carcinoma, Hepatocellular Carcinoma, Kidney Chromophobe Carcinoma, Kidney Clear Cell Carcinoma, Kidney Papillary Cell Carcinoma, Lower Grade Glioma, Lung Adenocarcinoma, Lung Squamous Cell Carcinoma, Mesothelioma, Ovarian Serous Adenocarcinoma, Pancreatic Ductal Adenocarcinoma, Paraganglioma & Pheochromocytoma, Prostate Adenocarcinoma, Sarcoma, Skin Cutaneous Melanoma, Testicular Germ Cell Cancer, Thymoma, Thyroid Papillary Carcinoma, Uterine Carcinosarcoma, Uterine Corpus Endometrioid Carcinoma, or Uveal Melanoma. In embodiments, the cancer-associated gene is a nucleic acid sequence identified within The Cancer Genome Atlas Program, accessible at www.cancer.gov/tcga.

In embodiments, the cancer-associated biomarker is MDC, NME-2, KGF, PlGF, Flt-3L, HGF, MCP1, SAT-1, MIP-1-b, GCLM, OPG, TNF RII, VEGF-D, ITAC, MMP-10, GPI, PPP2R4, AKR1B1, AmylA, MIP-1b, P-Cadherin, or EPO. In embodiments, the cancer-associated gene is a AKT1, AKT2, AKT3, ALK, AR, ARAF, ARID1A, ATM, ATR, ATRX, AXL, BAP1, BRAF, BRCA1, BRCA2, BTK, CBL, CCND1, CCND2, CCND3, CCNE1, CDK12, CDK2, CDK4, CDK6, CDKN1B, CDKN2A, CDKN2B, CHEK1, CHEK2, CREBBP, CSF1R, CTNNB1, DDR2, EGFR, ERBB2, ERBB3, ERBB4, ERCC2, ERG, ESR1, ETV1, ETV4, ETV5, EZH2, FANCA, FANCD2, FANCI, FBXW7, FGF19, FGF3, FGFR1, FGFR2, FGFR3, FGFR4, FGR, FLT3, FOXL2, GATA2, GNA11, GNAQ, GNAS, H3F3A, HIST1H3B, HNF1A, HRAS, IDH1, IDH2, IGF1R, JAK1, JAK2, JAK3, KDR, KIT, KNSTRN, KRAS, MAGOH, MAP2K1, MAP2K2, MAP2K4, MAPK1, MAX, MDM2, MDM4, MED12, MET, MLH1, MRE11A, MSH2, MSH6, MTOR, MYB, MYBL1, MYC, MYCL, MYCN, MYD88, NBN, NF1, NF2, NFE2L2, NOTCH1, NOTCH2, NOTCH3, NOTCH4, NRAS, NRG1, NTRK1, NTRK2, NTRK3, NUTM1, PALB2, PDGFRA, PDGFRB, PIK3CA, PIK3CB, PIK3R1, PMS2, POLE, PPARG, PPP2R1A, PRKACA, PRKACB, PTCH1, PTEN, PTPN11, RAC1, RAD50, RAD51, RAD51B, RAD51C, RAD51D, RAF1, RB1, RELA, RET, RHEB, RHOA, RICTOR, RNF43, ROS1, RSPO2, RSPO3, SETD2, SF3B1, SLX4, SMAD4, SMARCA4, SMARCB1, SMO, SPOP, SRC, STAT3, STK11, TERT, TOP1, TP53, TSC1, TSC2, U2AF1, or XPO1 gene. In embodiments, the cancer-associated gene is a ABL1, AKT1, ALK, APC, ATM, BRAF, CDH1, CDKN2A, CSF1R, CTNNB1, EGFR, ERBB2, ERBB4, EZH2, FBXW7, FGFR1, FGFR2, FGFR3, FLT3, GNA11, GNAQ, GNAS, HNF1A, HRAS, IDH1, IDH2, JAK2, JAK3, KDR, KIT, KRAS, MET, MLH1, MPL, NOTCH1, NPM1, NRAS, PDGFRA, PIK3CA, PTEN, PTPN11, RB1, RET, SMAD4, SMARCB1, SMO, SRC, STK11, TP53, or VHL gene. In embodiments, the cell is a cell (e.g., a T cell) within a tumor. In embodiments, the cell is a non-allogenic cell (i.e., native cell to the subject) within a tumor. In embodiments, the cell is a tumor infiltrating lymphocyte (TIL). In embodiments, the cell is an allogenic cell. In embodiments, the cell is a circulating tumor cell.

In embodiments, the cell in situ is obtained from a subject (e.g., human or animal tissue). Once obtained, the cell is placed in an artificial environment in plastic or glass containers supported with specialized medium containing essential nutrients and growth factors to support proliferation. In embodiments, the cell is permeabilized and immobilized to a solid support surface. In embodiments, the cell is permeabilized and immobilized to an array (i.e., to discrete locations arranged in an array). In embodiments, the cell is immobilized to a solid support surface. In embodiments, the surface includes a patterned surface (e.g., suitable for immobilization of a plurality of cells in an ordered pattern. The discrete regions of the ordered pattern may have defined locations in a regular array, which may correspond to a rectilinear pattern, circular pattern, hexagonal pattern, or the like. These discrete regions are separated by interstitial regions. As used herein, the term “interstitial region” refers to an area in a substrate or on a surface that separates other areas of the substrate or surface. In embodiments, a plurality of cells are immobilized on a patterned surface that have a mean or median separation from one another of about 10-20 μm. In embodiments, a plurality of cells are immobilized on a patterned surface that have a mean or median separation from one another of about 10-20; 10-50; or 100 μm. In embodiments, a plurality of cells are arrayed on a substrate. In embodiments, a plurality of cells are immobilized in a 96-well microplate having a mean or median well-to-well spacing of about 8 mm to about 12 mm (e.g., about 9 mm). In embodiments, a plurality of cells are immobilized in a 384-well microplate having a mean or median well-to-well spacing of about 3 mm to about 6 mm (e.g., about 4.5 mm).

In embodiments, the cell is attached to the substrate via a bioconjugate reactive linker. In embodiments, the cell is attached to the substrate via a specific binding reagent. In embodiments, the specific binding reagent includes an antibody, single-chain Fv fragment (scFv), antibody fragment-antigen binding (Fab), or an aptamer. In embodiments, the specific binding reagent includes an antibody, or antigen binding fragment, an aptamer, affimer, or non-immunoglobulin scaffold. In embodiments, the specific binding reagent is a peptide, a cell penetrating peptide, an aptamer, a DNA aptamer, an RNA aptamer, an antibody, an antibody fragment, a light chain antibody fragment, a single-chain variable fragment (scFv), a lipid, a lipid derivative, a phospholipid, a fatty acid, a triglyceride, a glycerolipid, a glycerophospholipid, a sphingolipid, a saccharolipid, a polyketide, a polylysine, polyethyleneimine, diethylaminoethyl (DEAE)-dextran, cholesterol, or a sterol moiety. Substrates may be prepared for selective capture of particular cells. For example, a substrate containing a plurality of bioconjugate reactive moieties or a plurality of specific binding reagents, optionally in an ordered pattern, contacts a plurality of cells. Only cells containing complementary bioconjugate reactive moieties or complementary specific binding reagents are capable of reacting, and thus adhering, to the substrate.

In embodiments, the cell is permeabilized. In embodiments, the methods are performed in situ on isolated cells or in tissue sections that have been prepared according to methodologies known in the art. Methods for permeabilization and fixation of cells and tissue samples are known in the art, as exemplified by Cremer et al., The Nucleus: Volume 1: Nuclei and Subnuclear Components, R. Hancock (ed.) 2008; and Larsson et al., Nat. Methods (2010) 7:395-397, the content of each of which is incorporated herein by reference in its entirety. In embodiments, the cell is cleared (e.g., digested) of proteins, lipids, or proteins and lipids. In embodiments, the method includes digesting the cell by contacting the cell with an endopeptidase.

In embodiments, the cell is immobilized to a substrate. The cell may have been cultured on the surface, or the cell may have been initially cultured in suspension and then fixed to the surface. Substrates can be two- or three-dimensional and can include a planar surface (e.g., a glass slide). A substrate can include glass (e.g., controlled pore glass (CPG)), quartz, plastic (such as polystyrene (low cross-linked and high cross-linked polystyrene), polycarbonate, polypropylene and poly(methymethacrylate)), acrylic copolymer, polyamide, silicon, metal (e.g., alkanethiolate-derivatized gold), cellulose, nylon, latex, dextran, gel matrix (e.g., silica gel), polyacrolein, or composites. In embodiments, the substrate includes a polymeric coating, optionally containing bioconjugate reactive moieties capable of affixing the sample. Suitable three-dimensional substrates include, for example, spheres, microparticles, beads, membranes, slides, plates, micromachined chips, tubes (e.g., capillary tubes), microwells, microfluidic devices, channels, filters, or any other structure suitable for anchoring a sample. In embodiments, the substrate is not a flow cell. In embodiments, the substrate includes a polymer matrix material (e.g., polyacrylamide, cellulose, alginate, polyamide, cross-linked agarose, cross-linked dextran or cross-linked polyethylene glycol), which may be referred to herein as a “matrix”, “synthetic matrix”, “exogenous polymer” or “exogenous hydrogel”. In embodiments, a matrix may refer to the various components and organelles of a cell, for example, the cytoskeleton (e.g., actin and tubulin), endoplasmic reticulum, Golgi apparatus, vesicles, etc. In embodiments, the matrix is endogenous to a cell. In embodiments, the matrix is exogenous to a cell. In embodiments, the matrix includes both the intracellular and extracellular components of a cell. In embodiments, polynucleotide primers may be immobilized on a matrix including the various components and organelles of a cell. Immobilization of polynucleotide primers on a matrix of cellular components and organelles of a cell is accomplished as described herein, for example, through the interaction/reaction of complementary bioconjugate reactive moieties. In embodiments, the exogenous polymer may be a matrix or a network of extracellular components that act as a point of attachment (e.g., act as an anchor) for the cell to a substrate.”

In embodiments, the cell is exposed to paraformaldehyde (i.e., by contacting the cell with paraformaldehyde). Any suitable permeabilization and fixation technologies can be used for making the cell available for the detection methods provided herein. In embodiments the method includes affixing single cells or tissues to a transparent substrate. Exemplary tissue include those from skin tissue, muscle tissue, bone tissue, organ tissue and the like. In embodiments, the method includes immobilizing the cell in situ to a substrate and permeabilized for delivering probes, enzymes, nucleotides and other components required in the reactions. In embodiments, the cell includes many cells from a tissue section in which the original spatial relationships of the cells are retained. In embodiments, the cell in situ is within a Formalin-Fixed Paraffin-Embedded (FFPE) sample. In embodiments, the cell is subjected to paraffin removal methods, such as methods involving incubation with a hydrocarbon solvent, such as xylene or hexane, followed by two or more washes with decreasing concentrations of an alcohol, such as ethanol. The cell may be rehydrated in a buffer, such as PBS, TBS or MOPs. In embodiments, the FFPE sample is incubated with xylene and washed using ethanol to remove the embedding wax, followed by treatment with Proteinase K to permeabilized the tissue. In embodiments, the cell is fixed with a chemical fixing agent. In embodiments, the chemical fixing agent is formaldehyde or glutaraldehyde. In embodiments, the chemical fixing agent includes both formaldehyde and glutaraldehyde. In embodiments, the chemical fixing agent is glyoxal or dioxolane. In embodiments, the chemical fixing agent includes one or more of ethanol, methanol, 2-propanol, acetone, and glyoxal. In embodiments, the chemical fixing agent includes formalin, Greenfix®, Greenfix® Plus, UPM, CyMol®, HOPE@, CytoSkelFix™, F-Solv®, FineFIX®, RCL2/KINFix, UMFIX, Glyo-Fixx®, Histochoice®, or PAXgene®. In embodiments, the cell is fixed within a synthetic three-dimensional matrix (e.g., polymeric material). In embodiments, the synthetic matrix includes polymeric-crosslinking material. In embodiments, the material includes polyacrylamide, poly-ethylene glycol (PEG), poly(acrylate-co-acrylic acid) (PAA), or Poly(N-isopropylacrylamide) (NIPAM).

In embodiments the cell is lysed to release nucleic acid or other materials from the cells. For example, the cells may be lysed using reagents (e.g., a surfactant such as Triton-X or SDS, an enzyme such as lysozyme, lysostaphin, zymolase, cellulase, mutanolysin, glycanases, proteases, mannase, proteinase K, etc.) or a physical lysing mechanism a physical condition (e.g., ultrasound, ultraviolet light, mechanical agitation, etc.). The cells may release, for instance, DNA, RNA, mRNA, proteins, or enzymes. The cells may arise from any suitable source. For instance, the cells may be any cells for which nucleic acid from the cells is desired to be studied or sequenced, etc., and may include one, or more than one, cell type. The cells may be for example, from a specific population of cells, such as from a certain organ or tissue (e.g., cardiac cells, immune cells, muscle cells, cancer cells, etc.), cells from a specific individual or species (e.g., human cells, mouse cells, bacteria, etc.), cells from different organisms, cells from a naturally-occurring sample (e.g., pond water, soil, etc.), or the like. In some cases, the cells may be dissociated from tissue. In embodiments, the method does not include dissociating the cell from the tissue or the cellular microenvironment. In embodiments, the method does not include lysing the cell.

In embodiments, the method further includes subjecting the cell to expansion microscopy methods and techniques. Expansion allows individual targets (e.g., mRNA or RNA transcripts) which are densely packed within a cell, to be resolved spatially in a high-throughput manner. Expansion microscopy techniques are known in the art and can be performed as described in US 2016/0116384 and Chen et al., Science, 347, 543 (2015), each of which are incorporated herein by reference in their entirety.

In embodiments, the method does not include subjecting the cell to expansion microscopy. Typically, expansion microscopy techniques utilize a swellable polymer or hydrogel (e.g., a synthetic matrix-forming material) which can significantly slow diffusion of enzymes and nucleotides. Matrix (e.g., synthetic matrix) forming materials include polyacrylamide, cellulose, alginate, polyamide, cross-linked agarose, cross-linked dextran or cross-linked polyethylene glycol. The matrix forming materials can form a matrix by polymerization and/or crosslinking of the matrix forming materials using methods specific for the matrix forming materials and methods, reagents and conditions known to those of skill in the art. Additionally, expansion microscopy techniques may render the temperature of the cell sample difficult to modulate in a uniform, controlled manner. Modulating temperature provides a useful parameter to optimize amplification and sequencing methods. In embodiments, the method does not include an exogenous matrix.

In embodiments, the circularizable oligonucleotide contains one or more functional moieties (e.g., bioconjugate reactive groups) that serve as attachment points to the cell (i.e., the internal cellular scaffold) or to the matrix in which the cell is embedded (e.g. a hydrogel). In embodiments, the bioconjugate reactive group is located at the 5′ and/or 3′ end of the oligonucleotide. In embodiments, the bioconjugate reactive group is located at an internal position of the oligonucleotide e.g., the oligonucleotide contains one or more modified nucleotides, such as aminoallyl deoxyuridine 5′-triphosphate (dUTP) nucleotide(s). In embodiments, the functional moiety can be covalently cross-linked, copolymerize with or otherwise non-covalently bound to the matrix. In embodiments, the functional moiety can react with a cross-linker. In embodiments, the functional moiety can be part of a ligand-ligand binding pair. Suitable exemplary functional moieties include an amine, acrydite, alkyne, biotin, azide, and thiol. In embodiments of crosslinking, the functional moiety is cross-linked to modified dNTP or dUTP or both. In embodiments, suitable exemplary cross-linker reactive groups include imidoester (DMP), succinimide ester (NHS), maleimide (Sulfo-SMCC), carbodiimide (DCC, EDC) and phenyl azide. Cross-linkers within the scope of the present disclosure may include a spacer moiety. In embodiments, such spacer moieties may be functionalized. In embodiments, such spacer moieties may be chemically stable. In embodiments, such spacer moieties may be of sufficient length to allow amplification of the nucleic acid bound to the matrix. In embodiments, suitable exemplary spacer moieties include polyethylene glycol, carbon spacers, photo-cleavable spacers and other spacers known to those of skill in the art and the like. In embodiments, the oligonucleotide primer contains a modified nucleotide (e.g., amino-allyl dUTP, 5-TCO-PEG4-dUTP, C8-Alkyne-dUTP, 5-Azidomethyl-dUTP, 5-Vinyl-dUTP, or 5-Ethynyl dLTTP). For example, prior to amplification, the modified nucleotide-containing primer is attached to the cell protein matrix by using a cross-linking reagent (e.g., an amine-reactive crosslinking agent with PEG spacers, such as (PEGylated bis(sulfosuccinimidyl)suberate) (BS(PEG)9)).

In embodiments, the target polynucleotide includes DNA nucleic acid sequences.

In embodiments, the target polynucleotide is a cDNA target polynucleotide and before step a), an RNA nucleic acid sequence is reverse transcribed to generate the cDNA target polynucleotide.

In embodiments, ligating includes enzymatic ligation including a ligation enzyme (e.g., Circligase enzyme, Taq DNA Ligase, HiFi Taq DNA Ligase, T4 ligase, PBCV-1 DNA Ligase (also known as SplintR ligase) or Ampligase DNA Ligase). Non-limiting examples of ligases include DNA ligases such as DNA Ligase I, DNA Ligase II, DNA Ligase III, DNA Ligase IV, T4 DNA ligase, T7 DNA ligase, T3 DNA Ligase, E. coli DNA Ligase, PBCV-1 DNA Ligase (also known as SplintR ligase) or a Taq DNA Ligase. In embodiments, the ligase enzyme includes a T4 DNA ligase, T4 RNA ligase 1, T4 RNA ligase 2, T3 DNA ligase or T7 DNA ligase. In embodiments, the enzymatic ligation is performed by a mixture of ligases. In embodiments, the ligation enzyme is selected from the group consisting of T4 DNA ligase, T4 RNA ligase 1, T4 RNA ligase 2, RtcB ligase, T3 DNA ligase, T7 DNA ligase, Taq DNA ligase, PBCV-1 DNA Ligase, a thermostable DNA ligase (e.g., 5′AppDNA/RNA ligase), an ATP dependent DNA ligase, an RNA-dependent DNA ligase (e.g., SplintR ligase), and combinations thereof. In embodiments, enzymatic ligation includes two different ligation enzymes (e.g., SplintR ligation and T4 DNA ligase, or SplintR ligase and Taq DNA ligase). In embodiments, enzymatic ligation includes more than two different ligation enzymes.

In embodiments, ligating includes chemical ligation (e.g., enzyme-free, click-mediated ligation). In embodiments, the oligonucleotides include a first bioconjugate reactive moiety capable of bonding upon contact with a second (complementary) bioconjugate reactive moiety. In embodiments, the oligonucleotides includes an alkynyl moiety at the 3′ and an azide moiety at the 5′ end that, upon hybridization to the target nucleic acid react to form a triazole linkage during suitable reaction conditions. Reaction conditions and protocols for chemical ligation techniques that are compatible with nucleic acid amplification methods are known in the art, for example El-Sagheer, A. H., & Brown, T. (2012). Accounts of chemical research, 45(8), 1258-1267; Manuguerra I. et al. Chem Commun (Camb). 2018; 54(36):4529-4532; and Odeh, F., et al. (2019). Molecules (Basel, Switzerland), 25(1), 3, each of which is incorporated herein by reference in their entirety.

In embodiments, amplifying the circular oligonucleotide includes incubation with a strand-displacing polymerase. In embodiments, amplifying includes incubation with a strand-displacing polymerase for about 10 seconds to about 60 minutes. In embodiments, amplifying includes incubation with a strand-displacing polymerase for about 60 seconds to about 60 minutes. In embodiments, amplifying includes incubation with a strand-displacing polymerase for about 10 minutes to about 60 minutes. In embodiments, amplifying includes incubation with a strand-displacing polymerase for about 10 minutes to about 30 minutes. In embodiments, amplifying includes incubation with a strand-displacing polymerase at a temperature of about 20° C. to about 50° C. In embodiments, incubation with the strand-displacing polymerase is at a temperature of about 35° C. to 42° C. In embodiments, the strand-displacing polymerase is phi29 polymerase, SD polymerase, Bst large fragment polymerase, phi29 mutant polymerase, or a thermostable phi29 mutant polymerase.

In embodiments, the method includes forming the template polynucleotide. The template polynucleotide can be a circular, dumbbell-shaped, or other closed nucleic acid molecule configuration that does not have a free 3′ or 5′ end. Typical library preparation steps may be performed on a linear template such that it is circularized (e.g., such as the protocols described in Kershaw, C. J., & O'Keefe, R. T. (2012) 941, 257-269). The initial template polynucleotide molecules can vary length, such as about 100-300 nucleotides long, about 300-500 nucleotides long, or about 500-1000 nucleotides long. In embodiments, the initial template polynucleotide molecular is about 100-1000 nucleotides, about 150-950 nucleotides, about 200-900 nucleotides, about 250-850 nucleotides, about 300-800 nucleotides, about 350-750 nucleotides, about 400-700 nucleotides, or about 450-650 nucleotides. In embodiments, the initial template polynucleotide molecule is about 150 nucleotides. In embodiments, the initial template polynucleotide is about 100-1000 nucleotides long. In embodiments, the initial template polynucleotide is about 100-300 nucleotides long. In embodiments, the initial template polynucleotide is about 300-500 nucleotides long. In embodiments, the initial template polynucleotide is about 500-1000 nucleotides long. In embodiments, the initial template polynucleotide molecule is about 100 nucleotides. In embodiments, the initial template polynucleotide molecule is about 300 nucleotides. In embodiments, the initial template polynucleotide molecule is about 500 nucleotides. In embodiments, the initial template polynucleotide molecule is about 1000 nucleotides.

In embodiments, the amplification primer is attached to the solid surface. In embodiments, the amplification primer is in solution. In embodiments, the amplification primer includes one or more phosphorothioate nucleotides. In embodiments, the amplification primer includes a plurality of phosphorothioate nucleotides. In embodiments, about or at least about 20%, 30%, 40%, 50%, 60%, 70%, 80%, 90%, or about 100% of the nucleotides in the amplification primer are phosphorothioate nucleotides. In embodiments, most of the nucleotides in the amplification primer are phosphorothioate nucleotides. In embodiments, all of the nucleotides in the amplification primer are phosphorothioate nucleotides.

Amplification primer molecules can be fixed to a surface, cellular component, or matrix by a variety of techniques, including covalent attachment and non-covalent attachment. In embodiments, the amplification primers are confined to an area of a discrete region (referred to as a cluster). The discrete regions may have defined locations in a regular array, which may correspond to a rectilinear pattern, circular pattern, hexagonal pattern, or the like. A regular array of such regions is advantageous for detection and data analysis of signals collected from the arrays during an analysis. These discrete regions are separated by interstitial regions. As used herein, the term “interstitial region” refers to an area in a substrate or on a surface that separates other areas of the substrate or surface. For example, an interstitial region can separate one concave feature of an array from another concave feature of the array. The two regions that are separated from each other can be discrete, lacking contact with each other. In another example, an interstitial region can separate a first portion of a feature from a second portion of a feature. In embodiments the interstitial region is continuous whereas the features are discrete, for example, as is the case for an array of wells in an otherwise continuous surface. The separation provided by an interstitial region can be partial or full separation. Interstitial regions will typically have a surface material that differs from the surface material of the features on the surface. For example, features of an array can have primers that exceeds the amount or concentration present at the interstitial regions. In some embodiments the primers may not be present at the interstitial regions. In embodiments, the amplification primer is attached to a solid support and a template polynucleotide is hybridized to the primer. In embodiments, at least two different primers are attached to the solid support (e.g., a forward and a reverse primer), which facilitates generating multiple amplification products from the first extension product or a complement thereof.

In embodiments, extending the amplification primer includes incubation with the strand-displacing polymerase in suitable conditions and for a suitable amount of time. In embodiments, the step of extending the amplification primer includes incubation with the strand-displacing polymerase (i) for about 10 seconds to about 30 minutes, and/or (ii) at a temperature of about 20° C. to about 50° C. In embodiments, incubation with the strand-displacing polymerase is for about 0.5 minutes to about 16 minutes. In embodiments, incubation with the strand-displacing polymerase is for about 0.5 minutes to about 10 minutes. In embodiments, incubation with the strand-displacing polymerase is for about 1 minutes to about 5 minutes. In embodiments, the method includes amplifying a template polynucleotide by extending an amplification primer with a strand-displacing polymerase for about 10 seconds to about 30 minutes. In embodiments, the method includes amplifying a template polynucleotide by extending an amplification primer with a strand-displacing polymerase for about 30 seconds to about 16 minutes. In embodiments, the method includes amplifying a template polynucleotide by extending an amplification primer with a strand-displacing polymerase for about 30 seconds to about 10 minutes. In embodiments, the method includes amplifying a template polynucleotide by extending an amplification primer with a strand-displacing polymerase for about 30 seconds to about 5 minutes. In embodiments, the method includes amplifying a template polynucleotide by extending an amplification primer with a strand-displacing polymerase for about 1 second to about 5 minutes. In embodiments, the method includes amplifying a template polynucleotide by extending an amplification primer with a strand-displacing polymerase for about 1 second to about 2 minutes.

In embodiments, incubation with the strand-displacing polymerase is at a temperature of about 35° C. to 42° C. In embodiments, incubation with the strand-displacing polymerase is at a temperature of about 37° C. to 40° C. In embodiments, incubation with the thermostable strand-displacing polymerase is at a temperature of about 40° C. to 80° C. In embodiments, the method includes amplifying a template polynucleotide by extending an amplification primer with a strand-displacing polymerase at a temperature of about 20° C. to about 50° C. In embodiments, the method includes amplifying a template polynucleotide by extending an amplification primer with a strand-displacing polymerase at a temperature of about 30° C. to about 50° C. In embodiments, the method includes amplifying a template polynucleotide by extending an amplification primer with a strand-displacing polymerase at a temperature of about 25° C. to about 45° C. In embodiments, the method includes amplifying a template polynucleotide by extending an amplification primer with a strand-displacing polymerase at a temperature of about 35° C. to about 45° C. In embodiments, the method includes amplifying a template polynucleotide by extending an amplification primer with a strand-displacing polymerase at a temperature of about 35° C. to about 42° C. In embodiments, the method includes amplifying a template polynucleotide by extending an amplification primer with a strand-displacing polymerase at a temperature of about 37° C. to about 40° C.

In embodiments, the extension product includes three or more copies of the target nucleic acid (e.g., the barcode sequence). In embodiments, the extension product includes at least three or more copies of the target nucleic acid. In embodiments, the extension product includes at least five or more copies of the target nucleic acid. In embodiments, the extension product includes at 5 to 10 copies of the target nucleic acid. In embodiments, the extension product includes 10 to 20 copies of the target nucleic acid. In embodiments, the extension product includes 20 to 50 copies of the target nucleic acid.

In embodiments, the oligonucleotide (e.g., the immobilized primer) is attached to the matrix or to a cellular component via a specific binding reagent. In embodiments, the specific binding reagent includes an antibody, single-chain Fv fragment (scFv), antibody fragment-antigen binding (Fab), or an aptamer. In embodiments, the specific binding reagent includes an antibody, or antigen binding fragment, an aptamer, affimer, or non-immunoglobulin scaffold. In embodiments, the specific binding reagent is a peptide, a cell penetrating peptide, an aptamer, a DNA aptamer, an RNA aptamer, an antibody, an antibody fragment, a light chain antibody fragment, a single-chain variable fragment (scFv), a lipid, a lipid derivative, a phospholipid, a fatty acid, a triglyceride, a glycerolipid, a glycerophospholipid, a sphingolipid, a saccharolipid, a polyketide, a polylysine, polyethyleneimine, diethylaminoethyl (DEAE)-dextran, cholesterol, or a sterol moiety. For example, the matrix or cellular component (e.g., a protein) may contain a complementary specific binding reagent to the oligonucleotide containing a specific binding reagent.

Single stranded oligonucleotide primers can be attached to cellular components through several mechanisms known in the art. For example, immobilized primers can be conjugated to proteins using techniques like chemical cross-linking or biotinylation, thereby forming a stable bond between the primer and the protein. Additionally, primers can be attached to lipids through lipophilic modifications, enabling them to insert into the lipid bilayer of cellular membranes. Another approach involves incorporating primers into specific cytoplasmic components by directly introducing them via techniques such as microinjection or electroporation. In embodiments, carrier molecules such as cell-penetrating peptides and/or nanoparticles, can facilitate the delivery of oligonucleotide primers to targeted cellular components.

Chemical cross-linking involves the formation of a covalent bond between the oligonucleotide and the biomolecule within the cell or tissue via bioconjugate reactive groups. For example, amino groups in the biomolecule can react with carboxyl groups on the oligonucleotide through carbodiimide chemistry, thereby forming an amide bond, linking the molecules together. In embodiments, covalent immobilization can occur through maleimide chemistry, where thiol groups on the oligonucleotide react with maleimide groups on the biomolecule, forming a stable thioether bond. In embodiments, the primers may be immobilized to a biomolecule within the cell or tissue via biotin. For example, the biotinylated oligonucleotide can bind to streptavidin or avidin. The association between biotin and streptavidin forms a strong, non-covalent bond that can be utilized in various biotechnological applications. Alternatively, in embodiments, oligonucleotide primers may be immobilized to endogenous nucleic acid molecules in the cell or tissue. For example, terminal deoxynucleotidyl transferase (TdT) can add nucleotides to the 3′ end of an oligonucleotide using a template-independent mechanism. By incorporating modified nucleotides coupled to reactive groups, TdT can introduce covalent bonds between the oligonucleotide primer and the biomolecule. In embodiments, the immobilized primer is covalently attached to a biomolecule in the cell or tissue via a bioconjugate linker (e.g., a linker formed via a reaction between two bioconjugate reactive moieties).

In embodiments, the method further includes detecting the amplification product(s) (e.g., the amplification product(s) of step (d)). In embodiments, detecting includes two-dimensional (2D) or three-dimensional (3D) fluorescent microscopy. Suitable imaging technologies are known in the art, as exemplified by Larsson et al., Nat. Methods (2010) 7:395-397 and associated supplemental materials, the entire content of which is incorporated by reference herein in its entirety. In embodiments of the methods provided herein, the imaging is accomplished by confocal microscopy. Confocal fluorescence microscopy involves scanning a focused laser beam across the sample, and imaging the emission from the focal point through an appropriately-sized pinhole. This suppresses the unwanted fluorescence from sections at other depths in the sample. In embodiments, the imaging is accomplished by multi-photon microscopy (e.g., two-photon excited fluorescence or two-photon-pumped microscopy). Unlike conventional single-photon emission, multi-photon microscopy can utilize much longer excitation wavelength up to the red or near-infrared spectral region. This lower energy excitation requirement enables the implementation of semiconductor diode lasers as pump sources to significantly enhance the photostability of materials. Scanning a single focal point across the field of view is likely to be too slow for many sequencing applications. To speed up the image acquisition, an array of multiple focal points can be used. The emission from each of these focal points can be imaged onto a detector, and the time information from the scanning mirrors can be translated into image coordinates. Alternatively, the multiple focal points can be used just for the purpose of confining the fluorescence to a narrow axial section, and the emission can be imaged onto an imaging detector, such as a CCD, EMCCD, or s-CMOS detector. A scientific grade CMOS detector offers an optimal combination of sensitivity, readout speed, and low cost. One configuration used for confocal microscopy is spinning disk confocal microscopy. In 2-photon microscopy, the technique of using multiple focal points simultaneously to parallelize the readout has been called Multifocal Two-Photon Microscopy (MTPM). Several techniques for MTPM are available, with applications typically involving imaging in biological tissue. In embodiments of the methods provided herein, the imaging is accomplished by light sheet fluorescence microscopy (LSFM). In embodiments, detecting includes 3D structured illumination (3DSIM). In 3DSIM, patterned light is used for excitation, and fringes in the Moiré pattern generated by interference of the illumination pattern and the sample, are used to reconstruct the source of light in three dimensions. In order to illuminate the entire field, multiple spatial patterns are used to excite the same physical area, which are then digitally processed to reconstruct the final image. See York, Andrew G., et al. “Instant super-resolution imaging in live cells and embryos via analog image processing.” Nature methods 10.11 (2013): 1122-1126 which is incorporated herein by reference. In embodiments, detecting includes selective planar illumination microscopy, light sheet microscopy, emission manipulation, pinhole confocal microscopy, aperture correlation confocal microscopy, volumetric reconstruction from slices, deconvolution microscopy, or aberration-corrected multifocus microscopy. In embodiments, detecting includes digital holographic microscopy (see for example Manoharan, V. N. Frontiers of Engineering: Reports on Leading-edge Engineering from the 2009 Symposium, 2010, 5-12, which is incorporated herein by reference). In embodiments, detecting includes confocal microscopy, light sheet microscopy, or multi-photon microscopy.

In embodiments, the method includes sequencing the extension products, which includes the target sequence. A variety of sequencing methodologies can be used such as sequencing-by synthesis (SBS), pyrosequencing, sequencing by ligation (SBL), or sequencing by hybridization (SBH). Pyrosequencing detects the release of inorganic pyrophosphate (PPi) as particular nucleotides are incorporated into a nascent nucleic acid strand (Ronaghi, et al., Analytical Biochemistry 242(1), 84-9 (1996); Ronaghi, Genome Res. 11(1), 3-11 (2001); Ronaghi et al. Science 281(5375), 363 (1998); U.S. Pat. Nos. 6,210,891; 6,258,568; and. 6,274,320, each of which is incorporated herein by reference in its entirety). In pyrosequencing, released PPi can be detected by being converted to adenosine triphosphate (ATP) by ATP sulfurylase, and the level of ATP generated can be detected via light produced by luciferase. In this manner, the sequencing reaction can be monitored via a luminescence detection system. In both SBL and SBH methods, target nucleic acids, and amplicons thereof, are subjected to repeated cycles of oligonucleotide delivery and detection. SBL methods, include those described in Shendure et al. Science 309:1728-1732 (2005); U.S. Pat. Nos. 5,599,675; and 5,750,341, each of which is incorporated herein by reference in its entirety; and the SBH methodologies are as described in Bains et al., Journal of Theoretical Biology 135(3), 303-7 (1988); Drmanac et al., Nature Biotechnology 16, 54-58 (1998); Fodor et al., Science 251(4995), 767-773 (1995); and WO 1989/10977, each of which is incorporated herein by reference in its entirety.

In SBS, extension of a nucleic acid primer along a nucleic acid template is monitored to determine the sequence of nucleotides in the template. The underlying chemical process can be catalyzed by a polymerase, wherein fluorescently labeled nucleotides are added to a primer (thereby extending the primer) in a template dependent fashion such that detection of the order and type of nucleotides added to the primer can be used to determine the sequence of the template. In embodiments, sequencing includes annealing and extending a sequencing primer to incorporate a detectable label that indicates the identity of a nucleotide in the target polynucleotide, detecting the detectable label, and repeating the extending and detecting of steps. In embodiments, the methods include sequencing one or more bases of a target nucleic acid by extending a sequencing primer hybridized to a target nucleic acid (e.g., an amplification product produced by the amplification methods described herein). In embodiments, sequencing may be accomplished by a sequencing-by-synthesis (SBS) process. In embodiments, sequencing includes a sequencing by synthesis process, where individual nucleotides are identified iteratively, as they are polymerized to form a growing complementary strand. In embodiments, nucleotides added to a growing complementary strand include both a label and a reversible chain terminator that prevents further extension, such that the nucleotide may be identified by the label before removing the terminator to add and identify a further nucleotide. Such reversible chain terminators include removable 3′ blocking groups, for example as described in U.S. Pat. Nos. 7,541,444 and 7,057,026. Once such a modified nucleotide has been incorporated into the growing polynucleotide chain complementary to the region of the template being sequenced, there is no free 3′-OH group available to direct further sequence extension and therefore the polymerase cannot add further nucleotides. Once the identity of the base incorporated into the growing chain has been determined, the 3′ reversible terminator may be removed to allow addition of the next successive nucleotide. By ordering the products derived using these modified nucleotides it is possible to deduce the DNA sequence of the oligonucleotide target nucleic acid sequence.

In embodiments, sequencing is performed according to a “sequencing-by-binding” method (see, e.g., U.S. Pat. Pubs. US2017/0022553 and US2019/0048404, each of which is incorporated herein by reference in its entirety), which refers to a sequencing technique wherein specific binding of a polymerase and cognate nucleotide to a primed template nucleic acid molecule (e.g., blocked primed template nucleic acid molecule) is used for identifying the next correct nucleotide to be incorporated into the primer strand of the primed template nucleic acid molecule. The specific binding interaction need not result in chemical incorporation of the nucleotide into the primer. In some embodiments, the specific binding interaction can precede chemical incorporation of the nucleotide into the primer strand or can precede chemical incorporation of an analogous, next correct nucleotide into the primer. Thus, detection of the next correct nucleotide can take place without incorporation of the next correct nucleotide. As used herein, the “next correct nucleotide” (sometimes referred to as the “cognate” nucleotide) is the nucleotide having a base complementary to the base of the next template nucleotide. The next correct nucleotide will hybridize at the 3′-end of a primer to complement the next template nucleotide. The next correct nucleotide can be, but need not necessarily be, capable of being incorporated at the 3′ end of the primer. For example, the next correct nucleotide can be a member of a ternary complex that will complete an incorporation reaction or, alternatively, the next correct nucleotide can be a member of a stabilized ternary complex that does not catalyze an incorporation reaction. A nucleotide having a base that is not complementary to the next template base is referred to as an “incorrect” (or “non-cognate”) nucleotide.

In embodiments, the methods of sequencing a nucleic acid include a extending a polynucleotide by using a polymerase. In embodiments, the polymerase is a DNA polymerase. In embodiments, the DNA polymerase is a Pol I DNA polymerase, Pol II DNA polymerase, Pol III DNA polymerase, Pol IV DNA polymerase, Pol V DNA polymerase, Pol β DNA polymerase, Pol μ DNA polymerase, Pol λ DNA polymerase, Pol σ DNA polymerase, Pol α DNA polymerase, Pol δ DNA polymerase, Pol ε DNA polymerase, Pol η DNA polymerase, Pol ξ DNA polymerase, Pol κ DNA polymerase, Pol ζ DNA polymerase, Pol γ DNA polymerase, Pol θ DNA polymerase, Pol ν DNA polymerase, or a thermophilic nucleic acid polymerase (e.g., Therminator γ, 9° N polymerase (exo-), Therminator II, Therminator III, or Therminator IX). In embodiments, the DNA polymerase is a thermophilic nucleic acid polymerase. In embodiments, the DNA polymerase is a modified archaeal DNA polymerase. In embodiments, the polymerase is a mutant P. abyssi polymerase (e.g., such as a mutant P. abyssi polymerase described in WO 2018/148723 or WO 2020/056044, each of which are incorporated herein by reference for all purposes). In embodiments, the polymerase is a bacterial DNA polymerase, eukaryotic DNA polymerase, archaeal DNA polymerase, viral DNA polymerase, or phage DNA polymerases. Bacterial DNA polymerases include E. coli DNA polymerases I, II and III, IV and V, the Klenow fragment of E. coli DNA polymerase, Clostridium stercorarium (Cst) DNA polymerase, Clostridium thermocellum (Cth) DNA polymerase and Sulfolobus solfataricus (Sso) DNA polymerase. Eukaryotic DNA polymerases include DNA polymerases α, β, γ, δ, €, η, λ, σ, μ, and k, as well as the Revl polymerase (terminal deoxycytidyl transferase) and terminal deoxynucleotidyl transferase (TdT). Viral DNA polymerases include T4 DNA polymerase, phi-29 DNA polymerase, GA-1, phi-29-like DNA polymerases, PZA DNA polymerase, phi-15 DNA polymerase, Cpl DNA polymerase, Cpl DNA polymerase, T7 DNA polymerase, and T4 polymerase. Other useful DNA polymerases include thermostable and/or thermophilic DNA polymerases such as Thermus aquaticus (Taq) DNA polymerase, Thermus filiformis (Tfi) DNA polymerase, Thermococcus zilligi (Tzi) DNA polymerase, Thermus thermophilus (Tth) DNA polymerase, Thermus flavusu (Tfl) DNA polymerase, Pyrococcus woesei (Pwo) DNA polymerase, Pyrococcus furiosus (Pfu) DNA polymerase and Turbo Pfu DNA polymerase, Thermococcus litoralis (Tli) DNA polymerase, Pyrococcus sp. GB-D polymerase, Thermotoga maritima (Tma) DNA polymerase, Bacillus stearothermophilus (Bst) DNA polymerase, Pyrococcus Kodakaraensis (KOD) DNA polymerase, Pfx DNA polymerase, Thermococcus sp. JDF-3 (JDF-3) DNA polymerase, Thermococcus gorgonarius (Tgo) DNA polymerase, Thermococcus acidophilium DNA polymerase; Sulfolobus acidocaldarius DNA polymerase; Thermococcus sp. go N-7 DNA polymerase; Pyrodictium occultum DNA polymerase; Methanococcus voltae DNA polymerase; Methanococcus thermoautotrophicum DNA polymerase; Methanococcus jannaschii DNA polymerase; Desulfurococcus strain TOK DNA polymerase (D. Tok Pol); Pyrococcus abyssi DNA polymerase; Pyrococcus horikoshii DNA polymerase; Pyrococcus islandicum DNA polymerase; Thermococcus fumicolans DNA polymerase; Aeropyrum pernix DNA polymerase; and the heterodimeric DNA polymerase DP1/DP2. In embodiments, the polymerase is 3PDX polymerase as disclosed in U.S. Pat. No. 8,703,461, the disclosure of which is incorporated herein by reference. In embodiments, the polymerase is a reverse transcriptase. Exemplary reverse transcriptases include, but are not limited to, HIV-1 reverse transcriptase from human immunodeficiency virus type 1 (PDB 1HMV), HIV-2 reverse transcriptase from human immunodeficiency virus type 2, M-MLV reverse transcriptase from the Moloney murine leukemia virus, AMV reverse transcriptase from the avian myeloblastosis virus, and Telomerase reverse transcriptase.

In embodiments, sequencing includes a plurality of sequencing cycles. In embodiments, sequencing includes a plurality of rounds of sequencing cycles (e.g., a first round of 10 sequencing cycles; followed by a second round of 10 sequencing cycles). In embodiments, sequencing includes 20 to 100 sequencing cycles. In embodiments, sequencing includes 50 to 100 sequencing cycles. In embodiments, sequencing includes 50 to 300 sequencing cycles. In embodiments, sequencing includes 50 to 150 sequencing cycles. In embodiments, sequencing includes at least 10, 20, 30 40, or 50 sequencing cycles. In embodiments, sequencing includes at least 10 sequencing cycles. In embodiments, sequencing includes 10 to 20 sequencing cycles. In embodiments, sequencing includes 10, 11, 12, 13, 14, or 15 sequencing cycles. In embodiments, sequencing includes (a) extending a sequencing primer by incorporating a labeled nucleotide, or labeled nucleotide analogue and (b) detecting the label to generate a signal for each incorporated nucleotide or nucleotide analogue. In embodiments, prior to initiating a next round of sequencing cycles, the first sequencing primer is terminated or removed. For example, termination may occur via incorporating a non-extendable nucleotide (e.g., a ddNTP) into the first sequencing primer.

In embodiments, sequencing includes sequentially extending a plurality of sequencing primers (e.g., sequencing a first region of a target nucleic acid followed by sequencing a second region of a target nucleic acid, followed by sequencing N regions, where N is the number of sequencing primers in the known sequencing primer set). In embodiments, sequencing includes generating a plurality of sequencing reads.

In embodiments, sequencing includes sequentially sequencing a plurality of different targets by initiating sequencing with different sequencing primers. For example, a first circularizable probe includes a first primer binding site (a nucleic acid sequence complementary to a first sequencing primer) and optionally a first barcode. In a similar manner, a second and third padlock probe include a second primer binding site (a nucleic acid sequence complementary to a second, different, sequencing primer) and a third primer binding site (a nucleic acid sequence complementary to a third, different from both Primer 1 and Primer 2, sequencing primer), respectively. During the first round of sequencing (following probe circularization and amplification according to the methods described herein), using primer 1, the probe hybridized to the first region of the nucleic acid molecule is detected. In the second round of sequencing, primer 2 can hybridize and sequence an identifying sequence of the probe (e.g., a barcode sequence) hybridized to a second nucleic acid molecule. Similarly, in the third round of sequencing, primer 3 can hybridize and sequence the probe hybridized to the third nucleic acid molecule. Optionally, the probes may all bind to the same nucleic acid molecule. In embodiments, the probes bind to different nucleic acid molecules. In embodiments, the probes bind to different nucleic acid molecules, wherein each nucleic acid molecule encodes for the same gene sequence.

In embodiments, sequencing includes extending a sequencing primer to generate a sequencing read. In embodiments, sequencing includes extending a sequencing primer by incorporating a labeled nucleotide, or labeled nucleotide analogue and detecting the label to generate a signal for each incorporated nucleotide or nucleotide analogue. In embodiments, the labeled nucleotide or labeled nucleotide analogue further includes a reversible terminator moiety.

In embodiments, the labeled nucleotide or labeled nucleotide analogue further includes a reversible terminator moiety. In embodiments, the reversible terminator moiety is attached to the 3′ oxygen of the nucleotide and is independently

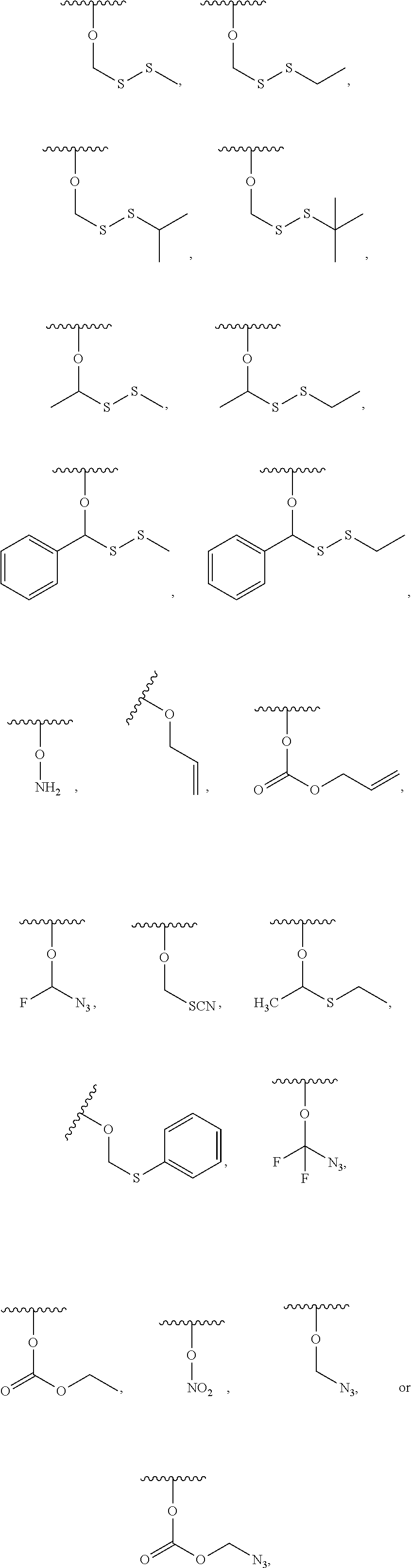

wherein the 3′ oxygen is explicitly depicted in the above formulae. Additional examples of reversible terminators may be found in U.S. Pat. No. 6,664,079, Ju J. et al. (2006) Proc Natl Acad Sci USA 103(52):19635-19640.; Ruparel H. et al. (2005) Proc Natl Acad Sci USA 102(17):5932-5937.; Wu J. et al. (2007) Proc Natl Acad Sci USA 104(104):16462-16467; Guo J. et al. (2008) Proc Natl Acad Sci USA 105(27): 9145-9150 Bentley D. R. et al. (2008) Nature 456(7218):53-59; or Hutter D. et al. (2010) Nucleosides Nucleotides & Nucleic Acids 29:879-895, which are incorporated herein by reference in their entirety for all purposes. In embodiments, a polymerase-compatible cleavable moiety includes an azido moiety or a dithiol moiety.

A variety of suitable sequencing platforms are available for implementing methods disclosed herein (e.g., for performing the sequencing reaction). Non-limiting examples include SMRT (single-molecule real-time sequencing), ion semiconductor, pyrosequencing, sequencing by synthesis, sequencing by binding, combinatorial probe anchor synthesis, SOLiD sequencing (sequencing by ligation), and nanopore sequencing. Sequencing platforms include those provided by Singular Genomics™ (e.g., the G4™ system), Illumina™, Inc. (e.g., HiSeq™ MiSeq™, NextSeq™, or NovaSeq™ systems), Life Technologies™ (e.g., ABI PRISM™, or SOLiD™ systems), Pacific Biosciences (e.g., systems using SMRT™ Technology such as the Sequel™ or RS II™ systems), or Qiagen (e.g., Genereader™ system). See, for example U.S. Pat. Nos. 7,211,390; 7,244,559; 7,264,929; 6,255,475; 6,013,445; 8,882,980; 6,664,079; and 9,416,409.

In embodiments, generating a sequencing read includes determining the identity of the nucleotides in the template polynucleotide (or complement thereof). In embodiments, a sequencing read, e.g., a first sequencing read or a second sequencing read, includes determining the identity of a portion (e.g., 1, 2, 5, 10, 20, 50 nucleotides) of the total template polynucleotide. In embodiments the first sequencing read determines the identity of 5-10 nucleotides and the second sequencing read determines the identity of more than 5-10 nucleotides (e.g., 11 to 200 nucleotides). In embodiments the first sequencing read determines the identity of more than 5-10 nucleotides (e.g., 11 to 200 nucleotides) and the second sequencing read determines the identity of 5-10 nucleotides. In embodiments, following the generation of a sequencing read, subsequent extension is performed using a plurality of standard (e.g., non-modified) dNTPs until the complementary strand is copied. In other embodiments, following the generation of a sequencing read, subsequent extension is performed using a plurality of dideoxy nucleotide triphosphates (ddNTPs) to prevent further extension of the first sequencing read product during a second sequencing read. In embodiments, following the identification of at least 5-10 (e.g., 11 to 200 nucleotides, or up to 1000 nucleotides), subsequent extension is performed using a plurality of standard (e.g., non-modified) dNTPs until the complementary strand is copied. In embodiments, following the identification of at least 5-10 (e.g., 11 to 200 nucleotides, or up to 1000 nucleotides), subsequent extension is performed using a plurality of dideoxy nucleotide triphosphates (ddNTPs) to prevent further extension of the sequencing read product.

In embodiments, the sequencing method relies on the use of modified nucleotides that can act as reversible reaction terminators. Once the modified nucleotide has been incorporated into the growing polynucleotide chain complementary to the region of the template being sequenced there is no free 3′-OH group available to direct further sequence extension and therefore the polymerase cannot add further nucleotides. Once the identity of the base incorporated into the growing chain has been determined, the 3′ reversible terminator may be removed to allow addition of the next successive nucleotide. These such reactions can be done in a single experiment if each of the modified nucleotides has attached a different label, known to correspond to the particular nucleobase, to facilitate discrimination between the bases added at each incorporation step. Alternatively, a separate reaction may be carried out containing each of the modified nucleotides separately.

The modified nucleotides may carry a label (e.g., a fluorescent label) to facilitate their detection. Each nucleotide type may carry a different fluorescent label. However, the detectable label need not be a fluorescent label. Any label can be used which allows the detection of an incorporated nucleotide. One method for detecting fluorescently labeled nucleotides includes using laser light of a wavelength specific for the labeled nucleotides, or the use of other suitable sources of illumination. The fluorescence from the label on the nucleotide may be detected (e.g., by a CCD camera or other suitable detection means).

In embodiments, the methods of sequencing a nucleic acid include extending a complementary polynucleotide (e.g., a primer) that is hybridized to the nucleic acid by incorporating a first nucleotide. In embodiments, the method includes a buffer exchange or wash step. In embodiments, the methods of sequencing a nucleic acid include a sequencing solution. The sequencing solution includes (a) an adenine nucleotide, or analog thereof; (b) (i) a thymine nucleotide, or analog thereof, or (ii) a uracil nucleotide, or analog thereof; (c) a cytosine nucleotide, or analog thereof; and (d) a guanine nucleotide, or analog thereof.

In embodiments, the method includes sequencing a plurality of target polynucleotides of a cell in situ within an optically resolved volume. In embodiments, the number of unique targets detected within an optically resolved volume of a sample is about 3, 10, 30, 50, or 100. In embodiments, the number of unique targets detected within an optically resolved volume of a sample is about 1 to 10. In embodiments, the number of unique targets detected within an optically resolved volume of a sample is about 5 to 10. In embodiments, the number of unique targets detected within an optically resolved volume of a sample is about 1 to 5. In embodiments, the number of unique targets detected within an optically resolved volume of a sample is at least 3, 10, 30, 50, or 100. In embodiments, the number of unique targets detected within an optically resolved volume of a sample is less than 3, 10, 30, 50, or 100. In embodiments, the number of unique targets detected within an optically resolved volume of a sample is about 1, 2, 3, 4, 5, 6, 7, 8, 9, 10, 20, 50, 100, 500, 1,000, 5,000, 10,000, or 200,000. In embodiments, the methods allow for detection of a single target of interest. In embodiments, the methods allow for multiplex detection of a plurality of targets of interest.

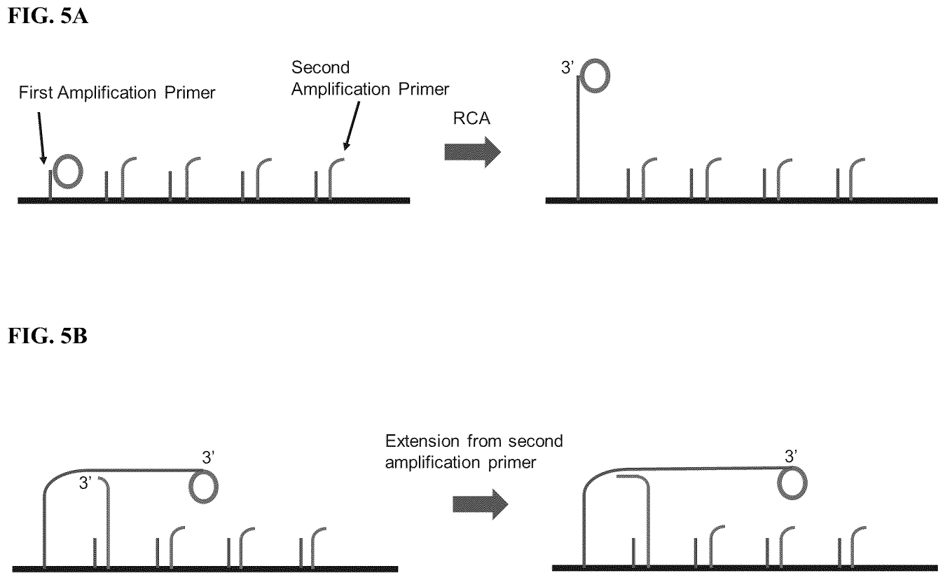

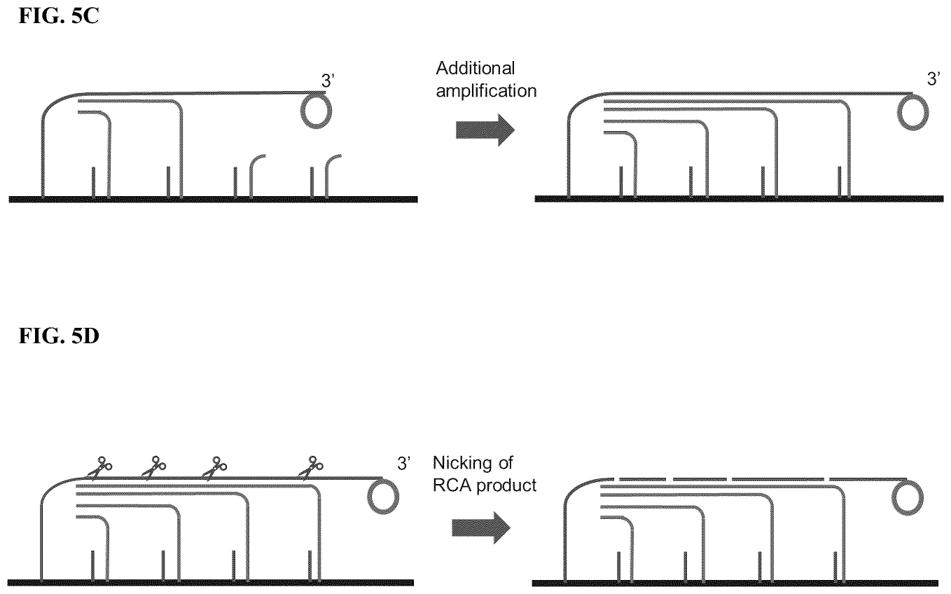

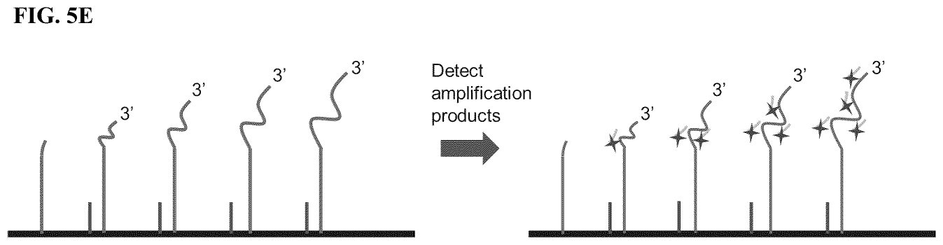

In embodiments, the optically resolved volume has an axial resolution (i.e., depth, or z) that is greater than the lateral resolution (i.e., xy plane). In embodiments, the optically resolved volume has an axial resolution that is greater than twice the lateral resolution. In embodiments, the dimensions (i.e., the x, y, and z dimensions) of the optically resolved volume are about 0.5 μm×0.5 μm×0.5 μm; 1 μm×1 μm×1 μm; 2 μm×2 μm×2 μm; 0.5 μm×0.5 μm×1 μm; 0.5 μm×0.5 μm×2 μm; 2 μm×2 μm×1 μm; or 1 μm×1 μm×2 μm. In embodiments, the dimensions (i.e., the x, y, and z dimensions) of the optically resolved volume are about 1 μm×1 μm×2 μm; 1 μm×1 μm×3 μm; 1 μm×1 μm×4 μm; or about 1 μm×1 μm×5 μm. See FIG. 5, for example. In embodiments, the dimensions (i.e., the x, y, and z dimensions) of the optically resolved volume are about 1 μm×1 μm×5 m. In embodiments, the dimensions (i.e., the x, y, and z dimensions) of the optically resolved volume are about 1 μm×1 μm×6 μm. In embodiments, the dimensions (i.e., the x, y, and z dimensions) of the optically resolved volume are about 1 μm×1 μm×7 μm. In embodiments, the optically resolved volume is a cubic micron. In embodiments, the optically resolved volume has a lateral resolution from about 100 to 200 nanometers, from 200 to 300 nanometers, from 300 to 400 nanometers, from 400 to 500 nanometers, from 500 to 600 nanometers, or from 600 to 1000 nanometers. In embodiments, the optically resolved volume has a axial resolution from about 100 to 200 nanometers, from 200 to 300 nanometers, from 300 to 400 nanometers, from 400 to 500 nanometers, from 500 to 600 nanometers, or from 600 to 1000 nanometers. In embodiments, the optically resolved volume has a axial resolution from about 1 to 2 μm, from 2 to 3 μm, from 3 to 4 μm, from 4 to 5 μm, from 5 to 6 μm, or from 6 to 10 μm.

In embodiments, the method further includes an additional imaging modality, immunofluorescence (IF), or immunohistochemistry modality (e.g., immunostaining). In embodiments, the method includes ER staining (e.g., contacting the cell with a cell-permeable dye which localizes to the endoplasmic reticula), Golgi staining (e.g., contacting the cell with a cell-permeable dye which localizes to the Golgi), F-actin staining (e.g., contacting the cell with a phalloidin-conjugated dye that binds to actin filaments), lysosomal staining (e.g., contacting the cell with a cell-permeable dye that accumulates in the lysosome via the lysosome pH gradient), mitochondrial staining (e.g., contacting the cell with a cell-permeable dye which localizes to the mitochondria), nucleolar staining, or plasma membrane staining. For example, the method includes live cell imaging (e.g., obtaining images of the cell) prior to or during fixing, immobilizing, and permeabilizing the cell. Immunohistochemistry (IHC) is a powerful technique that exploits the specific binding between an antibody and antigen to detect and localize specific antigens in cells and tissue, commonly detected and examined with the light microscope. Known IHC modalities may be used, such as the protocols described in Magaki, S., Hojat, S. A., Wei, B., So, A., & Yong, W. H. (2019). Methods in molecular biology (Clifton, N.J.), 1897, 289-298, which is incorporated herein by reference. In embodiments, the additional imaging modality includes bright field microscopy, phase contrast microscopy, Nomarski differential-interference-contrast microscopy, or dark field microscopy. In embodiments, the method further includes determining the cell morphology (e.g., the cell boundary or cell shape) using known methods in the art. For example, to determining the cell boundary includes comparing the pixel values of an image to a single intensity threshold, which may be determined quickly using histogram-based approaches as described in Carpenter, A. et al Genome Biology 7, R100 (2006) and Arce, S., Sci Rep 3, 2266 (2013)).

In embodiments, the barcodes in the known set of barcodes have a specified Hamming distance. In embodiments, the Hamming distance is 4 to 15. In embodiments, the Hamming distance is 8 to 12. In embodiments, the Hamming distance is 10. In embodiments, the Hamming distance is 0 to 100. In embodiments, the Hamming distance is 0 to 15. In embodiments, the Hamming distance is 0 to 10. In embodiments, the Hamming distance is 1 to 10. In embodiments, the Hamming distance is 5 to 10. In embodiments, the Hamming distance is 1 to 100. In embodiments, the Hamming distance between any two barcode sequences of the set is at least 2, 3, 4, or 5. In embodiments, the Hamming distance between any two barcode sequences of the set is at least 3. In embodiments, the Hamming distance between any two barcode sequences of the set is at least 4.

In embodiments, demultiplexing the multiplexed signal includes a linear decomposition of the multiplexed signal. Any of a variety of techniques may be employed for decomposition of the multiplexed signal. Examples include, but are not limited to, Zimmerman et al. Chapter 5: Clearing Up the Signal: Spectral Imaging and Linear Unmixing in Fluorescence Microscopy; Confocal Microscopy: Methods and Protocols, Methods in Molecular Biology, vol. 1075 (2014); Shirawaka H. et al.; Biophysical Journal Volume 86, Issue 3, March 2004, Pages 1739-1752; and S. Schlachter, et al, Opt. Express 17, 22747-22760 (2009); the content of each of which is incorporated herein by reference in its entirety. In embodiments, multiplexed signal includes overlap of a first signal and a second signal and is computationally resolved, for example, by imaging software.

In embodiments, the method further includes measuring an amount of one or more of the targets by counting the one or more associated sequences. In embodiments, the method further includes counting the one or more associated sequences in an optically resolved volume.

In embodiments, the number of unique targets detected within an optically resolved volume of a sample is about 3, 10, 30, 50, or 100. In embodiments, the number of unique targets detected within an optically resolved volume of a sample is about 1 to 10. In embodiments, the number of unique targets detected within an optically resolved volume of a sample is about 5 to 10. In embodiments, the number of unique targets detected within an optically resolved volume of a sample is about 1 to 5. In embodiments, the number of unique targets detected within an optically resolved volume of a sample is at least 3, 10, 30, 50, or 100. In embodiments, the number of unique targets detected within an optically resolved volume of a sample is less than 3, 10, 30, 50, or 100. In embodiments, the number of unique targets detected within an optically resolved volume of a sample is about 1, 2, 3, 4, 5, 6, 7, 8, 9, 10, 20, 50, 100, 500, 1,000, 5,000, 10,000, or 200,000. In embodiments, the methods allow for detection of a single target of interest. In embodiments, the methods allow for multiplex detection of a plurality of targets of interest. The use of oligonucleotide barcodes with unique identifier sequences as described herein allows for simultaneous detection of 2, 3, 4, 5, 6, 7, 8, 9, 10, 20, 30, 40, 50, 60, 70, 80, 90, 100, 200, 300, 400, 500, 600, 700, 800, 900, 1,000, 1,500, 2,000, 2,500, 3,000, 3,500, 4,000, 4,500, 5,000, 5,500, 6,000, 6,500, 7,000, 7,500, 8,000, 8,500, 9,000, 9,500, 10,000 or more than 10,000 unique targets within a single cell. In contrast to existing in situ detection methods, the methods presented herein have the advantage of virtually limitless numbers of individually detected molecules in parallel and in situ.

In embodiments, the total volume of the cell is about 1 to 25 μm³. In embodiments, the volume of the cell is about 5 to 10 μm³. In embodiments, the volume of the cell is about 3 to 7 μm³.

In aspects and embodiments described herein, the methods are useful in the field of predictive medicine in which diagnostic assays, prognostic assays, pharmacogenomics, and monitoring clinical trials are used for prognostic (i.e., predictive) purposes to thereby treat an individual prophylactically. Accordingly, in embodiments the methods of diagnosing and/or prognosing one or more diseases and/or disorders using one or more of expression profiling methods described herein are provided.

In an aspect is provided a method of detecting a disorder (e.g., cancer) or a disease-causing mutation or allele in a cell. In embodiments, the cell includes an oncogene (e.g., HER2, BRAF, EGFR, KRAS) and utilizing the methods described herein the oncogene is identified, thereby detecting a disorder when the presence of the oncogene is identified. In embodiments, the sample includes a nucleic acid molecule which includes a disease-causing mutation or allele. In embodiments, the method includes hybridizing an oligonucleotide primer which is correlated with the disease-causing mutation or allele. In embodiments, the method includes ligating a mutation-specific oligonucleotide only when the disease-causing mutation or allele is present in the nucleic acid target. In embodiments, the disease-causing mutation or allele is a base substitution, an insertion mutation, a deletion mutation, a gene amplification, a gene deletion, a gene fusion event, or a gene inversion event.

In embodiments, the mutation or allele is associated with an increased predisposition for one or more diseases, disorders, or other phenotypes. In embodiments, the mutation or allele is associated with a decreased predisposition for one or more diseases, disorders, or other phenotypes. For example, some mutations or alleles are associated with a cancer phenotype, such as decreased growth inhibition, evasion of immune detection, or dedifferentiation. Mutations that can be detected using the method provided herein include for example, mutations to BRAF, EGFR, Her2/ERBB2, and other somatic mutations as exemplified by Greenman et al., Nature (2007) 446:153-158, hereby incorporated by reference in its entirety.

### EXAMPLES

### Example 1. In Situ Transcriptomics

A wealth of information is reflected in the temporal and spatial variation of gene and protein expression among cells. Cellular macromolecules such as nucleic acids and proteins, occupy precise positions in cells and tissues, and a great deal of information is lost when these molecules are extracted. The methods available today for RNA sequence analysis (RNA-Seq) have the capacity to quantify the abundance of RNA molecules in a population of cells with great sensitivity. Current methods for single-cell RNA and protein analysis typically involve some method for “barcoding” the content of individual cells, followed by pooling the content and sequencing on a commercial DNA sequencing device (e.g., Singular Genomics G4™ sequencer, or Illumina NextSeq™ 500/550, MiSeq™, HiSeq™ 2500/3000/4000, or NovaSeq™). The barcoding can be done in individual wells on a microplate (e.g., a microplate with 96, 384, or 1536 wells), and more recently droplet-based methods are emerging as an essential tool for single-cell genomics research (see for example, Klein A. and Macosko E. Lab Chip. 2017; 17(15):2540-2541; and Zheng, G. X., et al. Nature communications, 2017; 8, 14049). Briefly, droplet-based methods begin with isolating a cell from a sample (e.g., a tissue) and encapsulating the cell in a droplet where unique identifying oligonucleotides (i.e., barcodes) are incorporated into the genomic sequence, often while converting RNAs to cDNAs during reverse transcription. These barcodes uniquely label the cDNA and identify the cellular origin. The cDNAs are then extracted and undergo standard library preparation for sequencing before being sequenced on a commercial sequencer. mRNA expression is then quantified by counting the number of barcodes that mapped to each cell.

These methods have found wide application dissecting transcriptomic heterogeneity and can handle upwards of 10,000 cells in an automated format, however they have several limitations and drawbacks. For example, if the cells of interest originate from a tissue sample, all information about the spatial distribution of the cells within the tissue is lost in the process of dissociating and isolating the cells prior to barcoding them. Often information about the intracellular distribution of analytes within the cellular microenvironment is also lost. This information can be vital to designing therapeutic approaches to cancers, for example, where the tumor microenvironment often creates spatial gradients of nutrients and metabolic byproducts. Droplet-based techniques are capable of barcoding and sequencing tens of thousands of cells (e.g., 10-50 thousand cells) in a single experiment but current approaches require generation of custom microfluidic devices, reagents, and sample preparation techniques (e.g., as found in the disclosures RE41,780 and US 2015/0225778). Additionally, due to the digital “counting” nature of the sequencing readout, hundreds of sequencing reads/cell are required to get information about the expression of less abundant genes. For example, if a particular abundant gene is transcribed into 500 copies of RNA, the abundant gene will dominate the sequencing run resulting in relatively inefficient use of sequencing capacity. However, cells can associate with multiple barcodes which significantly impacts single-cell analyses and rare cell events (Lareau, C. A., et al. (2020) Nature communications, 11(1), 866).

A different barcoding approach has been applied to spatial profiling of RNA & proteins in tissue. An example of this is the method developed by Spatial Transcriptomics, a Stockholm-based company purchased by 10× Genomics in 2018 and recently commercialized as “Visium Spatial” platform. This approach involves attaching a section of a frozen tissue of interest to patterned microarrays carrying spatially barcoded oligo-dT primers that capture the entire polyadenylated transcriptome contained in the tissue section. Each spot on the microarray contains a capture probe with a spatial barcode unique to that spot allowing the individual sequencing reads to be mapped to the originating spot. After cDNA synthesis on the surface via reverse transcription, the tissue is removed and the mRNA-cDNA hybrids are released from the array to be prepared for sequencing; see Vickovic, S., et al. Nat. Methods 16, 987-990 (2019) for greater detail on the approach. The current implementation of this technology includes a microarray with 100 μm spots spaced equidistant from each other, approximately 200 μm apart. The spatial resolution of this method is approximately 100 μm, which is sufficient for a coarse mapping of a pathology sample, but is insufficient to resolve individual cells, which are approximately 10-20 μm, or subcellular features (i.e., features less than 10 μm, such as the mitochondria). Wide adoption of this approach has been limited by the lack of scalability and accessible ways to automate and/or parallelize sequencing library preparation.

A number of new techniques have been described for reading out RNA transcription levels on and within tissue sections directly (i.e., in-situ), without requiring spatial barcoding, based on single molecule fluorescence in situ hybridization. These include MERFISH (Multiplexed Error-Robust Fluorescence In Situ Hybridization), STARmap (Spatially-resolved Transcript Amplicon Readout mapping), DART-FISH, seq-FISH (Sequential Fluorescence In Situ Hybridization) and others (see for example Chen, K. H., et al. (2015). Science, 348(6233), aaa6090; Wang, G., Moffitt, J. R. & Zhuang, X. Sci Rep. 2018; 8, 4847; Wang X. et al; Science, 2018; 27, Vol 361, Issue 6400, eaat5691; Cai, M. Dissertation, (2019) UC San Diego. ProQuest ID: Cai_ucsd_0033D_18822; and Sansone, A. Nat Methods 16, 458; 2019). In all of these techniques, individual RNA transcripts are individually resolved, typically with pre-amplification or requiring multiple instances of labeled probes. Some of these techniques have been combined with super-resolution microscopy, expansion microscopy, or both, to increase the resolution and allow more transcripts to be resolved and thus counted. This increases the complexity and costs of detection, and can require laborious sample preparation and time consuming wash protocols.

Described herein are methods for addressing these and other problems in the art. An aspect of the invention is to allow the readout of multiple RNA transcripts within one optically resolved volume (a voxel, see, e.g., FIG. 3). The method includes targeting specific RNA sequences, and “translating” them to an identifiable sequence (e.g., a barcode or associated sequence), with a means for local amplification. In embodiments, the method includes selecting barcodes that are widely spaced in the combinatorial space of possible barcodes (large Hamming distance). In embodiments, the method includes sequencing the barcodes. In embodiments, the method includes sequencing an identifying sequence (e.g., a sequence, or complement thereof, of the target nucleic acid molecule). The methods described herein (for example within the aspects and embodiments) reveal the distribution of specific RNA molecules in cells and tissues. In this way, patterns of differential gene expression may be observed which aids in the understanding of a particular gene's function, and ultimately the phenotype of the cell.

The method includes demultiplexing the observed signal in each voxel into the set of barcodes, or a set of identifying sequences, that includes the set of barcodes being used (e.g., by linear decomposition).

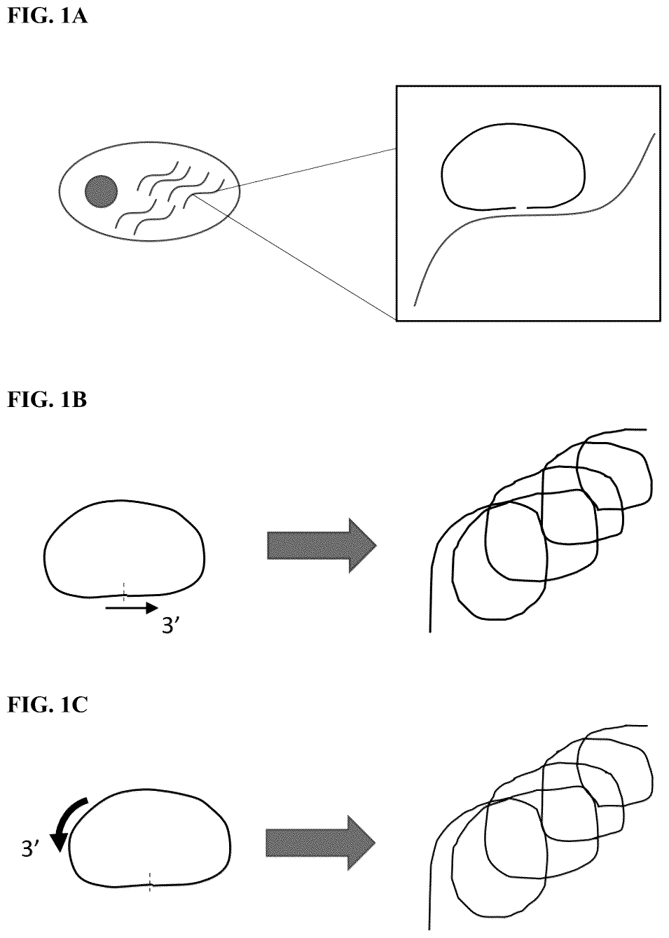

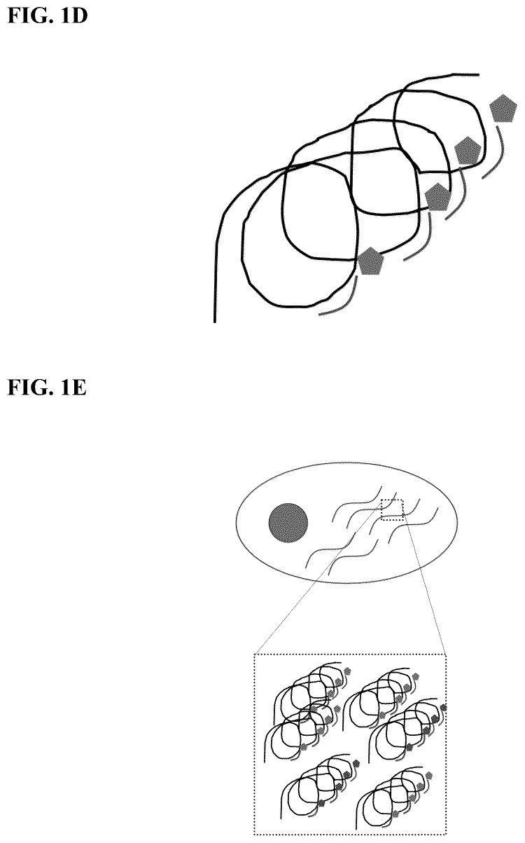

A method for “translating” or “encoding” RNA into an identifying sequence, such as a barcode, is to use one or more circularizable oligonucleotide probes (e.g., padlock probes), consisting of linear ssDNA, which are designed to have sequences complementary to 2 adjacent sequences on the target RNA. Once the padlock probes bind, the excess unbound probes are washed away, and the linear oligonucleotide is ligated to form a circle, using the RNA as a “splint”. This only occurs if the two ends of the padlock probe are adjacent to each other (see, e.g., FIG. 1A). (The 5′ end of the probe also has to be phosphorylated to enable ligation.) The padlock probe (and the resulting circle) contains several additional elements: a barcode for reading out the identity of the probe and its target; a complementary sequence for binding a sequencing primer. Optionally, the circle can contain multiple barcodes and priming sites. The circle may also contain a site for RCA priming and amplifying (Rolling Circle Amplification, see, e.g., FIGS. 1B-1C). In one embodiment, the priming site for RCA could have the same sequence as the sequencing primer, or have overlapping sequences. In embodiments, the circular template polynucleotide has both an amplification primer binding sequence and a sequencing primer binding sequence.

Optionally, the amplification (e.g., RCA) reaction can be performed with modified nucleotides that contain chemical groups that serve as attachment points to the cell or the matrix in which the cell is embedded (e.g., a crosslinked polymer or a hydrogel). The attachment of the amplified product to the matrix can help confine & fix the amplicon to a small volume. In embodiments, amplification reactions include standard dNTPs and a modified nucleotide (e.g., amino-allyl dUTP, 5-TCO-PEG4-dUTP, C8-Alkyne-dUTP, 5-Azidomethyl-dUTP, 5-Vinyl-dUTP, or 5-Ethynyl dLTTP). For example, during amplification a mixture of standard dNTPs and aminoallyl deoxyuridine 5′-triphosphate (dUTP) nucleotides may be incorporated into the amplicon and subsequently cross-linked to the cell protein matrix by using a cross-linking reagent (e.g., an amine-reactive crosslinking agent with PEG spacers, such as (PEGylated bis(sulfosuccinimidyl)suberate) (BS(PEG)₉)).

Optionally, one or more nucleotides within the amplification primer sequence, the sequencing primer sequence, and/or the immobilized oligonucleotide primer contains one or more functional moieties (e.g., bioconjugate reactive groups) that serve as attachment points to the cell, such as a cellular component or cellular biomolecule, or the matrix in which the cell is embedded (e.g., a hydrogel). In embodiments, one or more nucleotides within the amplification primer sequence, the sequencing primer sequence, and/or the immobilized oligonucleotide primer contains one or more functional moieties (e.g., bioconjugate reactive groups) that serve as attachment points to complementary bioconjugate reactive groups within the cell (e.g., a protein). In embodiments, a plurality of oligonucleotide primers are provided to the matrix in which the cell is embedded prior to amplification, wherein each of the primer oligonucleotides are attached to a specific binding reagent. In embodiments, a plurality of oligonucleotide primers are provided to the matrix in which the cell is embedded prior to amplification. In embodiments, a plurality of oligonucleotide primers is provided to the matrix in which the cell is embedded concurrently with amplification. In embodiments, the bioconjugate reactive group is located at the 5′ or 3′ end of the primer. In embodiments, the bioconjugate reactive group is located at an internal position of the primer e.g., the primer contains one or more modified nucleotides, such as aminoallyl deoxyuridine 5′-triphosphate (dUTP) nucleotide(s). In embodiments, the immobilized oligonucleotide primers may be used to aid in tethering the extension product to a confined area and may not be extended. In embodiments, the immobilized oligonucleotide primers may be used to aid in tethering the extension product to a confined area and may also be capable of being extended. For example, one or more immobilized oligonucleotides may be used to aid in tethering the extension product to a localized area and may be extended in an exponential RCA amplification reaction.

An alternate method is to start with a reverse transcription step to convert the target RNA molecule to cDNA. The cDNA would then act as the target for the circularizable padlock probe. In embodiments, the cDNA could serve as a splint for ligation. Yet another approach includes binding a relatively large probe directly to the RNA, without doing ligation or RCA. The large probe (e.g., branched DNA or a long concatemer) carries multiple sites for binding sequencing primers & reading barcodes present on the large probe. A potential drawback to this approach includes higher non-specific background and less efficient binding kinetics of a large probe. In embodiments, the method includes hybridizing the padlock probes directly to target RNA molecules.

The circularizable probes can be designed to target multiple regions on the RNA of interest. This could be done to enhance the signal, and/or to provide a level of redundancy of targeting in case of mutations in a particular region. The probes that target 2 or more regions of the same RNA transcript sequence could carry identical sequences (for redundancy), or could carry distinct identifying sequences (to independently identify the region that is being detected). In embodiments, the probes bind to the same RNA molecule. In embodiments, the probes bind to different sequences of the same gene of interest.

Imaging. To read out the probes, a sequencing primer is introduced, and the identifying sequence is read out (see, e.g., FIGS. 1D-IE) via sequencing. Preferably, the readout is done by SBS (Sequencing-By-Synthesis), with labeled and reversibly terminated nucleotides, although alternative sequencing modalities are considered. Similar to the amplification primer sequence, and the immobilized oligonucleotide primer described supra, the sequencing primer sequence may contain one or more nucleotides containing functional moieties (e.g., bioconjugate reactive groups) that serve as attachment points to the cell or the matrix in which the cell is embedded (e.g. a hydrogel) or to a cellular component, and the SBS reactions are performed with labeled and reversibly terminated nucleotides. In embodiments, the modified sequencing primer is provided to the matrix in which the cell is embedded following amplification or concurrently with the SBS mixture. The attachment of the SBS product to the matrix via the sequencing primer can help confine and fix the amplicon to a small, localized volume.

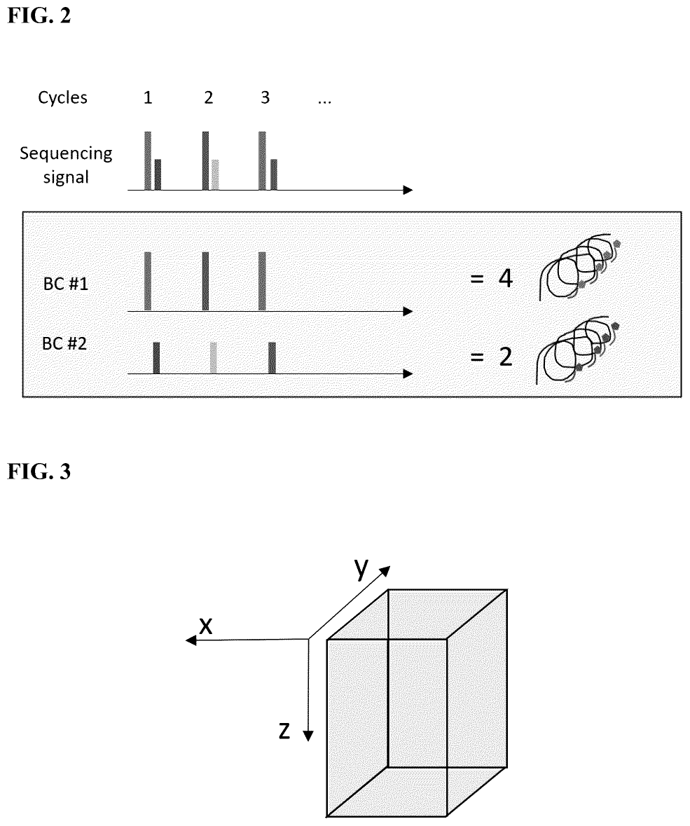

Because the identity of all the barcodes is known a priori, the resulting signal can be deconstructed (demultiplexed) into the constituent components (see, e.g., FIG. 2). Each sequencing cycle produces information about the magnitude of the signal in all 4 channels (a subset of 3 or 2 could also be used). The magnitude of the signal in all 4 channels can, for example, be represented by a signal matrix as:
Cycle 1: C₁, T₁, G₁, A₁
Cycle 2: C₂, T₂, G₂, A₂
Cycle 3: C₃, T₃, G₃, A₃
Cycle 4: C₄, T₄, G₄, A₄
Cycle 5: C₅, T₅, G₅, A₅
Cycle 6: C₆, T₆, G₆, A₆

Each barcode can also be represented by a similar matrix. For example, a 6-base barcode such as GTCATA could be represented as:
0, 0, 1, 0
0, 1, 0, 0
1, 0, 0, 0
0, 0, 0, 1
0, 1, 0, 0
0, 0, 0, 1

The signal is then fit to a linear combination of component barcodes. For example, in embodiments when using 4-color detection (i.e., one color per nucleotides) a set of 10 sequencing cycles provides information in 40 dimensions (4 channels per cycle x 10 cycles). Any of the up to 4¹⁰ possible barcodes would point to a unique position in this 40-dimensional space. Linear combinations of barcodes are thus easily resolvable, limited only by the accuracy of the sequencing signals. A typical example might be a set of 1,000-10,000 RNA targets, each encoded by a barcode selected from 4^{N} combinations, where N is the number of sequencing cycles or “digits” in the barcode. With 10 cycles, up to 4¹⁰, or approximately one million barcodes are available. This allows for the ability to select barcodes that are as far apart as possible in the available space (maximizing the Hamming distance), for more robust demultiplexing.

As illustrated in FIG. 2, each barcode can also be assigned a color (e.g., colors that are visually distinguishable) for the purpose of visualizing the spatial location of the barcode in a cell. Each sequencing cycle produces varying signal intensities in a voxel for each of the 4 channels. The brightness of the representative pixel corresponds to the local concentration of the barcode and is proportional to the product of the signal and barcode matrices (e.g., the product of the signal and barcode matrices results in a value of 4 for barcode #1 in FIG. 2, which can also be assigned a color such as green). Subsequent barcodes would also be assigned colors with pixel intensities proportional to the sequencing signal (e.g., the product of the signal and barcode matrices results in a value of 2 for barcode #2 in FIG. 2, which can also be assigned a color such as red).

Practical limitations, such as noise in the sequencing signal, will limit the total number of RNA transcripts that can be accurately detected in a single resolved volume (voxel). Reasonable upper limits might be 3, 10, 30, or >100 targets per voxel, depending on the performance of the sequencing system.

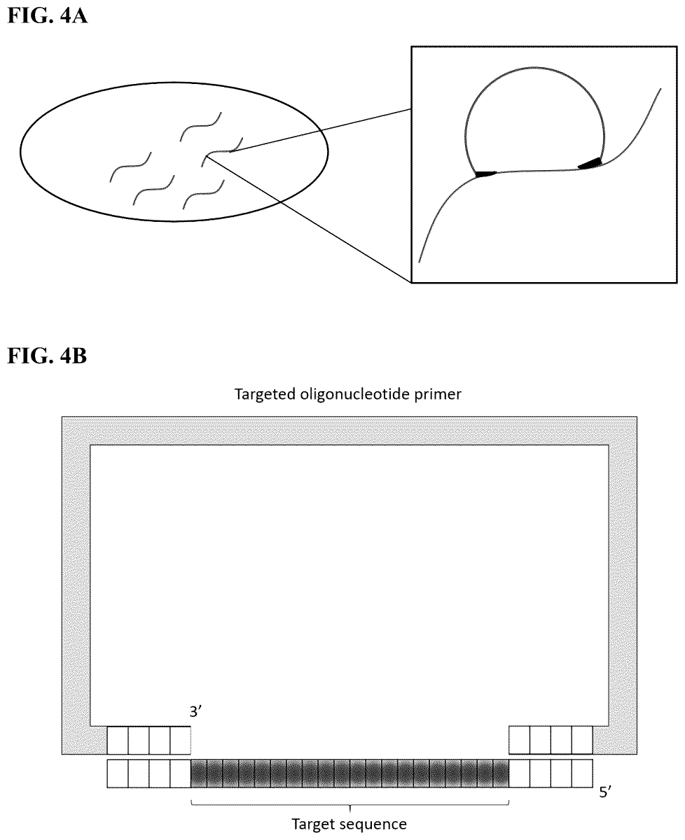

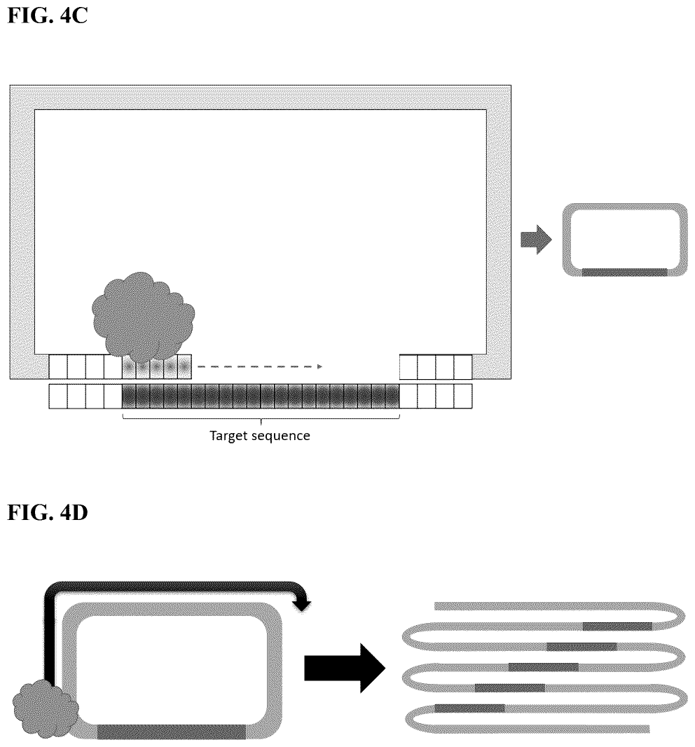

Alternatively, in situ sequencing may be performed, wherein targeted oligonucleotide probes are annealed to complementary regions which flank the nucleic acid of interest or a portion thereof (e.g., gap-fill padlock probes). As shown in FIGS. 4A-4B, the oligonucleotide probe hybridizes to regions which flank the target nucleic acid sequence or a portion thereof, referred to as the first and the second complementary regions. In the presence of a polymerase (e.g., a non-strand displacing polymerase), the complement to the target sequence is generated by extending from the first complementary region and is ligated (not shown) to the second complementary region to form a circularized oligonucleotide, as found in FIG. 4C. The resulting circularized oligonucleotide is primed with an amplification primer and extended with a strand-displacing polymerase to generate a concatemer containing multiple copies of the target nucleic acid sequence, as shown in FIG. 4D. This extension product is then primed with a sequencing primer and subjected to sequencing processes as described herein. The amplification products may also be sequenced according to an aspect of the invention as described in Example 3.

Imaging. Either 2D or 3D fluorescent imaging modalities can be used. An advantage of 3D imaging is that a larger number of individual volumes can be resolved. 3D fluorescent imaging methods include confocal microscopy, light sheet microscopy, and multi-photon microscopy. For example, if the imaging system has a lateral resolution of 0.5 um, and a depth resolution of 1.0 um, a 10×10×10 um volume would contain 20×20×10=4,000 voxels. If each voxel can resolve 10 barcodes, then this would correspond to a capacity of 40,000 reads in a 10-um cube without pushing the limits of optical resolution.

Further information can be gained by including expansion microscopy if subcellular resolution is required beyond the limits of diffraction, or if an even larger number of reads is desired.

The described methods can be applied to single cells affixed to a transparent substrate, as well as to sections of tissue on a similar substrate. In both cases (individual cells or cells in tissue), the cells are fixed and permeabilized for delivering probes, enzymes, nucleotides and other components required in the reactions.

### Example 2. In Situ Proteomics

The human genome contains on the order of 25,000 genes which work in concert to produce on the order of 1,000,000 distinct proteins. A single mass spectrometry experiment can identify about 2,000 proteins or 0.2% of the total (Mirza, S. P., & Olivier, M. (2008). Physiological genomics, 33(1), 3-11), highlighting the need for novel approaches to identify more proteins. Certainly, when one considers the levels of mRNA are not proportional to the expression level of the proteins they code for, it is beneficial to determine the proteome of a sample (e.g., a cell).

The methods described in Example 1, for spatial RNA transcriptomics can also be applied to spatial proteomics. For example, the proteins of interest are targeted by specific binding reagents, such as antibodies, fragments thereof (e.g., Fabs), aptamers, and the like, which carry a barcoded nucleic acid strand. That barcode can be used as splint for a padlock probe, as described above. The padlock probe, for example, may be amplified and sequenced by an aspect of the invention described herein and in Example 3.

If higher specificity is required, RCA-PLA (proximity ligation) methods can be used; see for example the methods, complexes, and kits described in US 2002/0064779, US 2005/0287526, and US 2014/0170654, each of which are incorporated herein by reference. With these methods, an amplified product is produced only if two specific antibodies bind to the same protein (or within˜5 nm distance). One antibody provides the DNA oligo that acts as a splint for a padlock probe, while the other antibody carries the primer for RCA. Thus, RCA reaction only occurs if both antibodies bind to their respective epitopes on the target protein (or protein complex).

### Example 3. Controlled Rolling Circle Amplification

Typically, low abundance template polynucleotides are amplified prior to performing a detection step, for example, prior to sequencing the template polynucleotides. In the context of cells and/or tissue sections, template polynucleotide are frequently circularized prior to amplification by rolling circle amplification (RCA), as processive strand-displacing polymerases (e.g., a phi29 polymerase, or variant thereof) are able to generate concatemeric amplification products including many copies of the target polynucleotide. Variations of rolling circle amplification have been developed, for example exponential RCA (eRCA) and/or hyperbranched RCA (HRCA), which include primer oligonucleotides that hybridize to the RCA product and can initiate additional amplification events, resulting in denser amplification products (e.g., larger amplicon clusters).

While having large amplification clusters provides with significantly more area for detection probes and primers to hybridize enabling greater detection efficiency and identification, for example, they may also introduce steric hindrance issues that can impair the accessibility of detection reagents to reach portions of the amplicons. For example, eRCA products may include dense double-stranded DNA portions that may not allow for efficient hybridization of detection oligonucleotides (e.g., a labeled oligonucleotide probe, and/or a sequencing primer).

Rolling circle amplification is typically performed on a circular template polynucleotide, as described herein. The circular template polynucleotide may be a circularized endogenous nucleic acid, or a circularizable probe (e.g., a padlock probe, wherein the padlock probe may include the complement of a target endogenous sequence, for example a target mRNA sequence). An amplification primer (e.g., RCA primer) and a strand-displacing polymerase (e.g., phi29 polymerase, or a variant thereof) then initiate RCA of the circular template polynucleotide. Additional RCA primers may be included to perform exponential amplification of the RCA product (e.g., eRCA). The additional RCA primers may be, for example, present in solution or immobilized to a matrix or cellular component. As the RCA product is generated, the additional RCA primers can hybridize to complementary sequences in the RCA product and be extended by the strand-displacing polymerase, forming eRCA products. As the eRCA products will be complementary to the RCA products, a cluster of double-stranded amplification products will be formed. Due to the double-stranded nature of the amplification products, hybridizing a fluorescent probe or a sequencing primer is challenging, and limits detection. Increasing the accessibility of this cluster to, for example, a detection oligonucleotide, would lead to improved detection signal.

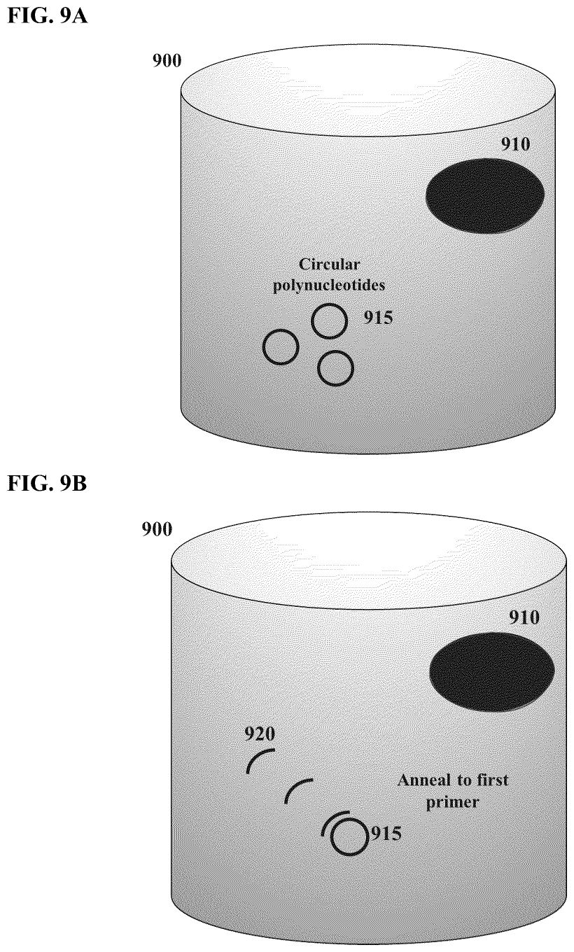

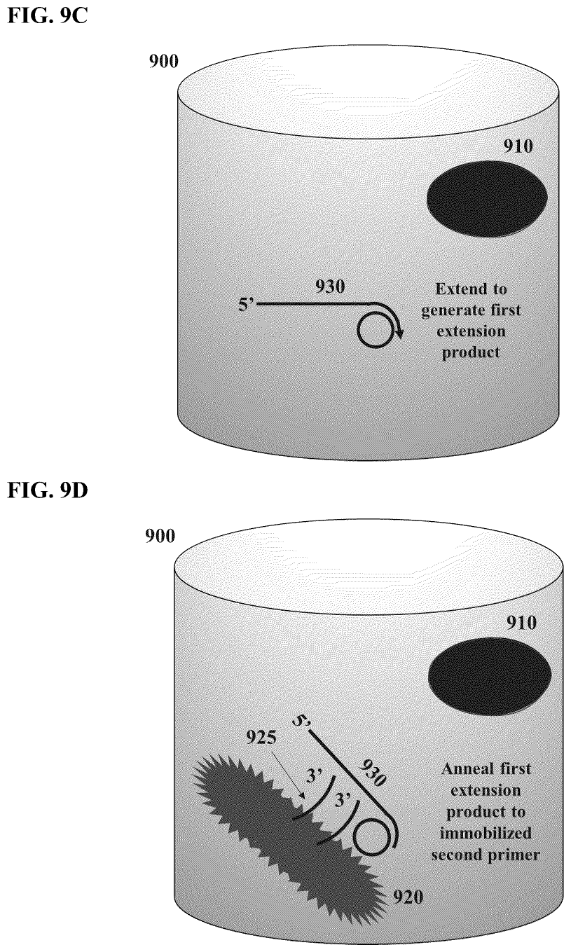

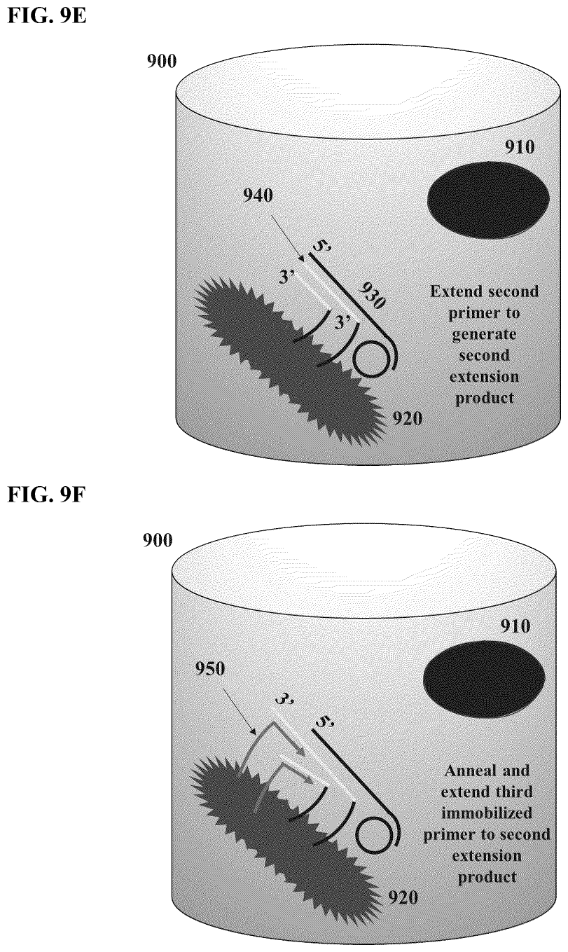

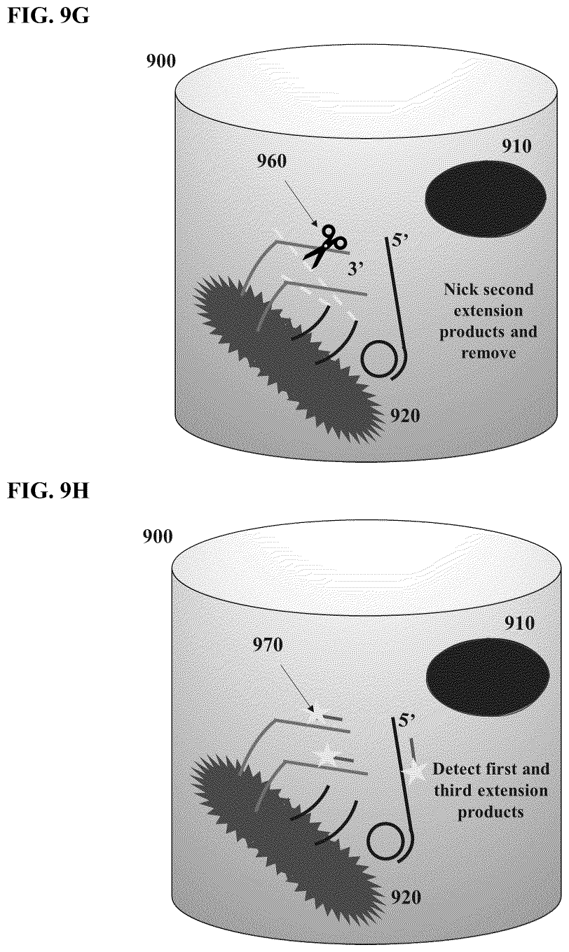

Described herein are methods for increasing the accessibility of RCA products generated using an exponential approach (e.g., eRCA or HRCA). FIGS. 9A-9H show a cartoon illustration of one embodiment of the methods. FIG. 9A depicts a fixed cellular matrix 900 including a nucleus 910 and circular polynucleotides 915. FIG. 9B depicts annealing of a first primer (e.g., a first amplification primer) 920 to one of the circular polynucleotides 915 from FIG. 9A. FIG. 9C depicts extension of primer 920 from FIG. 4B to generate a first extension product 930. FIG. 9D depicts annealing of the first extension product 930 to immobilized second primers 925 attached to a cellular component (or a matrix) 920. FIG. 9E depicts a step of extending the immobilized second primers 925 hybridized to the first extension product 930 and generating an immobilized second extension product 940. A third immobilized primer (e.g., an immobilized first primer) 950 is then annealed to the immobilized second extension product and extended to generate an immobilized third extension product, as shown in FIG. 9F. FIG. 9G depicts cleaving the immobilized second extension products with a nicking endonuclease 960, and removing the nicked fragments. In some embodiments, the first and third extension products are nicked and removed. FIG. 9H illustrates detection of the first and third extension products, for example, with a labeled probe oligonucleotide 970. In some embodiments (e.g., when the first and third extension products are nicked and removed), the second extension products are detected.

Briefly, following the eRCA reaction, a nicking endonuclease is introduced (e.g., a Nb.BbvCI nicking endonuclease) which generates nicks on one strand of the double-stranded eRCA product. Following the nicking reaction, the nicked strand is removed (e.g., by washing the eRCA product under denaturing conditions), thereby leaving behind a predominantly single-stranded amplification product. This results in realization of the benefits of eRCA (e.g., the increased speed of cluster formation), while significantly improving the accessibility of detection oligonucleotides to the nicked eRCA product. FIG. 8 illustrates a portion of a circularizable probe, wherein the circularizable probe includes a first target hybridization sequence (e.g., Arm1), a first primer binding sequence (PBS1), a nicking site complement, a second primer binding sequence (PBS2′), and a second target hybridization sequence (e.g., Arm2). Following circularization and rolling circle amplification (e.g., RCA or eRCA), an RCA amplicon strand and an RCA strand complement are generated, including complementary nicking sites.

Certain sequence-specific DNA nicking enzymes have been found to occur naturally. Nt.CviQXI and Nt.CviPII were originally found in the lysates of Chlorella viruses (Xia, Y. et al. Nucl Acids Res. 16:9477-9487, (1988); Zhang Y. N. M. et al. Virology 240:366-375 (1998). The nicking enzymes N.BstSEI and N.BstNBI were identified in bacterial sources (Abdurashitov M. A. et al. Mol Biol. (Mosk) 30:1261-1267, (1996); Morgan R. D. et al. Biol. Chem. 381:1123-1125 (2000)). Bacteriophages also encode nicking enzymes such as the gene II protein of bacteriophage f1 that is essential for viral DNA replication (Geider K. et al. Advan. Expt. Med. Biol. 179:45-54 (1984)). Sequence-specific DNA nicking enzymes have also been created by mutating naturally occurring dimeric Type IIA, Type IIS (Xu Y. et al. Proc. Natl. Acad. Sci. USA 98:12990-12995 (2001); Besnier C. E. et al. EMBL Rep. 2:782-786 (2001); Zhu Z. et al. J. Mol. Biol. 337:573-583 (2004)) or Type IIT restriction endonucleases using a variety of approaches. Additional information on nicking endonucleases and associated methods may be found in, e.g., U.S. Pat. Pub. Nos. US 2010/0330556, US 2011/0039716, US 2003/008259, US 2016/0264958, US 2011/0076720, and US 2013/0210019, each of which is incorporated herein by reference in its entirety.

FIGS. 5A-5E illustrate an embodiment of the invention described herein for amplifying (e.g., by exponential rolling circle amplification (eRCA)) a circular template polynucleotide (e.g., a circularized probe or circularized polynucleotide including a nicking site, or complement thereof). FIG. 5A depicts annealing of the circular template polynucleotide to a first immobilized amplification oligonucleotide (e.g., an oligonucleotide or primer immobilized at a 5′ end of the primer to a solid support, or immobilized at a 5′ end of the primer to a cellular component or matrix in situ), and subsequent extension (e.g., extension with a strand-displacing polymerase) of the first immobilized oligonucleotide to generate an immobilized amplicon (e.g., an immobilized concatemer including a plurality of complements of the circular template polynucleotide). While only a single circular template polynucleotide is illustrated, it will be apparent to one of skill in the art that a plurality of circular template polynucleotides may be annealed and amplified across a plurality of first immobilized oligonucleotides (e.g., a first plurality of immobilized primers) using the methods described herein. FIG. 5B depicts hybridization of a second immobilized amplification oligonucleotide (e.g., a second amplification primer) to the immobilized RCA product of FIG. 5A, followed by extension of the second amplification primer to generate an extension product complementary to a portion of the immobilized RCA product. Additional second amplification primers may anneal to the immobilized RCA product and be extended with a strand-displacing polymerase, as illustrated in FIG. 5C, generating a plurality of immobilized complements of the RCA product, wherein each immobilized complement includes the complement of a portion of the RCA product, and wherein generating each additional immobilized complement displaces the previously generated immobilized complement. While not shown for clarity, it is to be understood that as additional immobilized complements are generated and displaced, one or more of the plurality of first amplification primers are then able to hybridize to the displaced immobilized complements and be extended (e.g., in an exponential RCA reaction).

A nicking reaction is then performed (e.g., nicking by a nicking endonuclease that nicks one strand of a double-stranded nucleic acid substrate) as illustrated in FIG. 5D, generating nicks across the immobilized RCA product strand, for example. After washing under denaturing conditions, for example, the nicked portions of the RCA product are removed, leaving behind a plurality of immobilized complements of the RCA product as illustrated in FIG. 5E. While the immobilized RCA product is shown as being nicked in these illustrations, in some embodiments, the immobilized complements are nicked, for example, by changing the orientation of the nicking strand (e.g., by including a complementary nicking site sequence in the circular template polynucleotide). The immobilized complements may then be detected using, for example, labeled probes or subjected to a sequencing process as described herein. As illustrated, immobilized complements with longer portions of sequence may be bound by an increased number of labeled probes, resulting in greater signal intensity. In some embodiments, the immobilized complements are nicked and removed, and the immobilized RCA product(s) is detected.

Methods: In many cases, targeting low-abundance biomolecules as part of an in situ transcriptomics or proteomics study requires amplification of one or more probe oligonucleotides (e.g., amplifying a circular template polynucleotide including a barcode that corresponds to the target biomolecule) prior to, for example, performing in situ sequencing to detect the probe oligonucleotide. To show that the optimized eRCA approach described herein has improved and/or comparable amplification performance to standard eRCA and RCA protocols, we selected a target gene (COL5A2) in the U-138MG glioblastoma cell line (ATCC HTB-16™) for targeting with padlock probes, and subsequent amplification and detection of the probes.

Plating and Fixation: The following steps were performed in 96-well plate format. Cells were seeded at a density of 2,000 live cells/well and cultured for 3 days. Cells were washed with 1×PBS, then fixed with 4% formaldehyde in 1×PBS for 20 min at room temperature (RT) and washed 3 times with 1×PBS to remove the formaldehyde. Cells were then permeabilized with 0.5% Triton X-100 in 1×PBS for 20 min at RT, then washed 1× with 1×PBS and 2× with hybridization buffer (20% formamide and 2×SSC in water).

Hybridization and Probe Ligation: Padlock probes (PLPs) were added at a final concentration of 100 nM each with 10 mM ribonucleoside vanadyl complex (RVC) in hybridization buffer with 0.2 U/μL SUPERase-In™ RNase inhibitor (Thermo Fisher Catalog #AM2694). PLPs were then allowed to hybridize overnight at 37° C. The cells were then washed 2× with hybridization buffer for 5 min at 37° C. and 1× with 1×PBS for 5 min each at 37° C. Following the washes, SplintR® ligase (New England Biolabs Catalog #M0375S) was added at a final concentration of 2.5 U/μL with 0.2 U/μL SUPERase-In™ RNase inhibitor (Thermo Fisher Catalog #AM2694) in 1× SplintR ligase buffer and incubated for 60 min at 37° C. Cells were then washed 2× with 1×PBS and 1× with hybridization buffer.

Primer Immobilization: Cells were treated with a THPP-containing buffer (20 mM CAPS pH10.0, 1M NaCl, 3 mM THPP) for 15 min at 60° C. Amplification primers (including a PEG8 linker and a bioconjugate reactive moiety (e.g., an DBCO or maleimide moiety)) were added to the cells at a final concentration of 2 μM and immobilized via a bioconjugation reaction for 2 hr at 37° C. In some embodiments, amplification primers including a PEG4 spacer linker are added to the cells. Cells were then washed 2× with hybridization buffer and 1× with PBS.

Rolling Circle Amplification: A mutant version of phi29 DNA polymerase was then added at a final concentration of 1.8 μM and incubated in the absence of co-factors or dNTPs for 15 minutes at 37° C. Amplification was then initiated with the mutant version of phi29 DNA polymerase at a final concentration of 1.8 μM in 1× phi29 buffer, dNTPs (0.5 mM each), 12 mM MgCl₂, and 0.2 U/μL SUPERase-InT RNase inhibitor in DEPC-treated water and incubated for 30 min at 37° C. Cells were then washed 3× with 1×PBS.

Nicking and denaturation: Nb.BbvCI nicking endonuclease (Cat. #R0631S, NEB) was added at a final concentration of 0.3 U/μL in 1× rCutSmart™ buffer (NEB) and incubated for 1 hr at 37° C. Cells were then washed with a 100% formamide solution to remove the nicked strand portions.

Detection: TetraSpeck™ microspheres were added to crosslinked cells at a final concentration of 0.1 fM in 1×PBS and allowed to settle for at least 30 min at RT, or centrifuged for 3 min at 2,000 RPM. A Cy5-labeled probe and a dT30-FAM probed were then added to the cells in hybridization buffer, followed by washes with hybridization buffer and imaging. Sequencing-by-synthesis was additionally performed with a sample using the nicking and denaturantion method following a 30 min eRCA incubation, and compared to a 16 hr RCA-only condition, in situ in cells, for a total of 30 SBS cycles using labeled nucleotides.

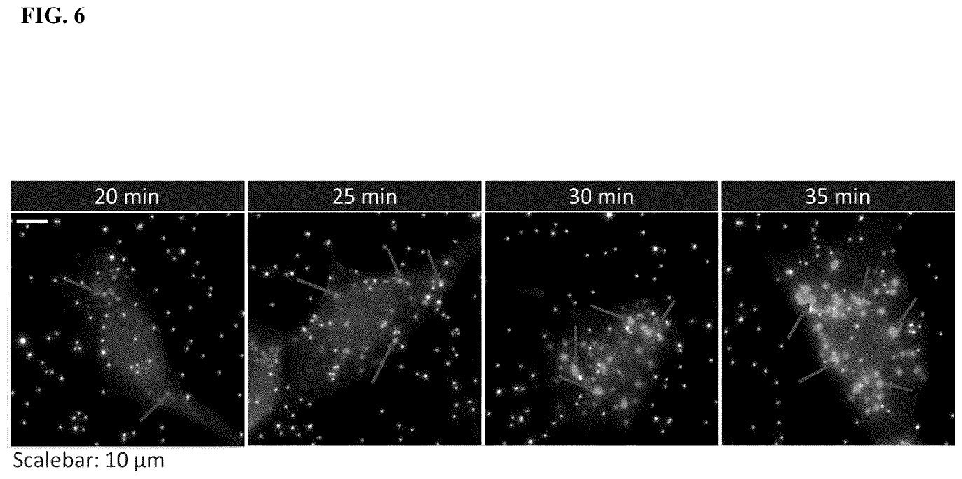

Using the methods described herein and in Example 1, we observed in situ significant eRCA product formation when the nicking and denaturation steps were included following eRCA. For context, non-exponential RCA requires overnight incubation for 12+ hours to achieve similar quantities of amplification products. FIG. 6 illustrates a series of fluorescence microscopy images of U-138MG cells targeted with circularizable probes specific for a target gene and subjected to the amplification and nicking process described herein and illustrated in FIGS. 5A-5E. Amplification (e.g., eRCA) of the circularized probes was performed for 20, 25, 30, or 35 minutes, as indicated in each panel. Following the nicking reaction, labeled probes specific for the immobilized amplification products were hybridized, and imaging was performed to detect the labeled probes. Arrows indicate the detected amplicon-specific probes. A poly-T-specific probe was also used to detect total mRNA in the cell (detected as an outline of the cell body). The intensity and number of clusters increases throughout each time point. Quantification of the amplicon cluster intensities with nicking (i.e., (+) nicking endonuclease) and without nicking ((−) nicking endonuclease) is provided in Table 1. The scattered puncta in FIG. 6 are detectable focusing beads added to the sample prior to imaging and a few of the amplification products are indicated with an arrow. We observed rapid and significantly increasing intensity of detectable amplicons in situ, especially after a 25-minute exponential amplification reaction. These results indicate that the nicking and denaturation steps remove significant portions of one strand of the double-stranded eRCA product, improving accessibility by the detection probes. We additionally performed in situ sequencing-by-synthesis and detected labeled nucleotide incorporation in 30 cycles following a 30 min eRCA incubation that led to comparable, if not improved, detectable signal to a 16 hr RCA-only incubation, indicating that the nicking and denaturation method also yields improved accessibility to sequencing reagents and improved detectability.

TABLE 1
Amplicon cluster signal intensities (relative fluorescent units,
RFUs) in U-138MG cells with nicking. The median intensity is
normalized relative to 16 hours of RCA (i.e., an overnight (o/n)
RCA reaction). The median full-width half-max (FWHM) provides
a measurement of the spatial distribution of the amplification cluster.

| | (+) nicking endonuclease | | (−) nicking endonuclease | |
|---|---|---|---|---|
| Time | Median | Median | Median | Median |
| (min) | Intensity | FWHM (μm) | Intensity | FWHM (μm) |
| 20 | 2.2 | 0.92 | 2.2 | 0.95 |
| 25 | 4.2 | 1.16 | 3.4 | 1.55 |
| 30 | 8.1 | 1.49 | 5.5 | 1.88 |
| 35 | 9.9 | 1.67 | 7.8 | 2.11 |

We found that even after 20 minutes of eRCA with the nicking/denaturation protocol described herein, the median intensity of the detectable clusters was greater than 2-fold that of an overnight RCA incubation. As described in Table 1, significant increases in median cluster intensity were seen over all amplification times (from 20 minutes to 35 minutes), indicating the superior amplification intensity and detection efficiency achieved with the methods described herein. Comparing each timepoint, it is seen that the addition of the nicking and removal steps lead to higher median cluster intensity over time, while concentrating the signal intensity into a smaller optically resolved area (as quantified by the median full-width half-max (FWHM) value). The nicking and denaturation steps described herein also advantageously keep the amplicon clusters from become too wide (i.e., having a smaller FWHM compared to only RCA or only eRCA), such that there is minimal signal overlap between individual amplicon clusters in the sample, while increasing the intensity of the detectable signal.

### Example 4. Localized Clusters and Sequencing

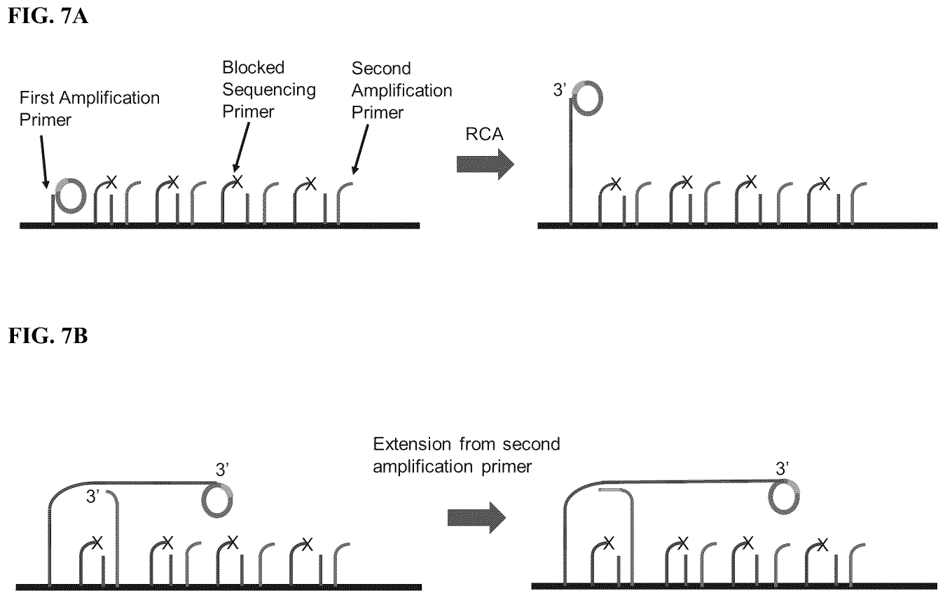

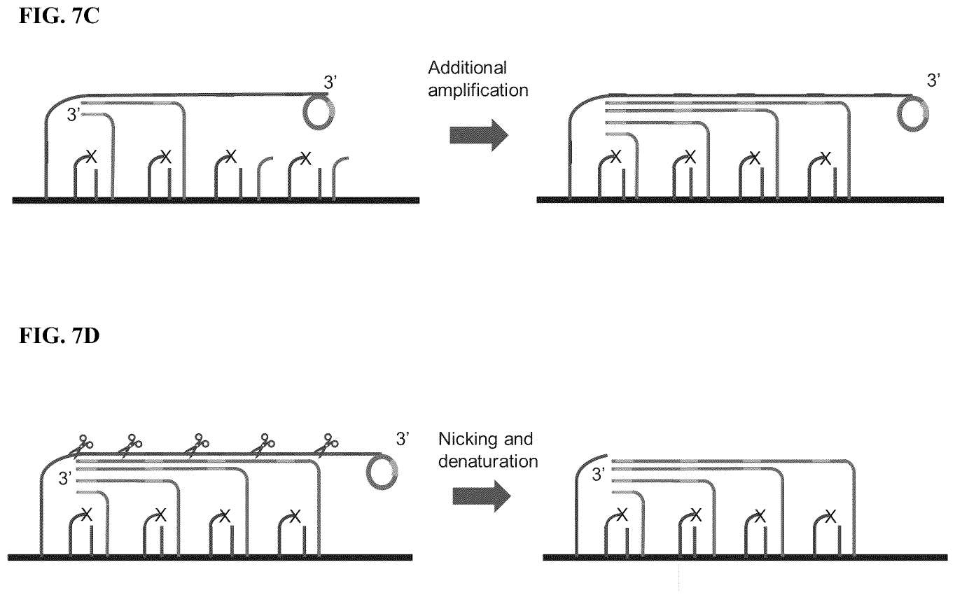

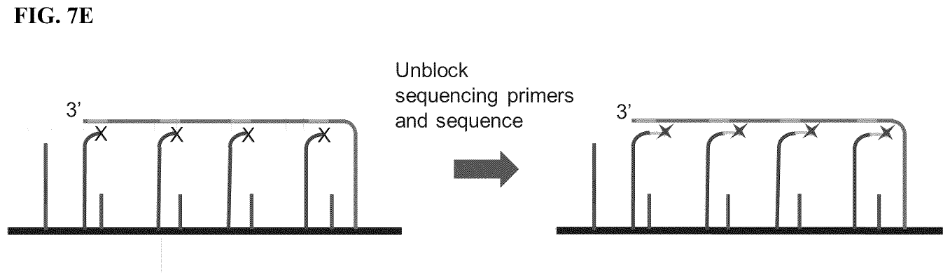

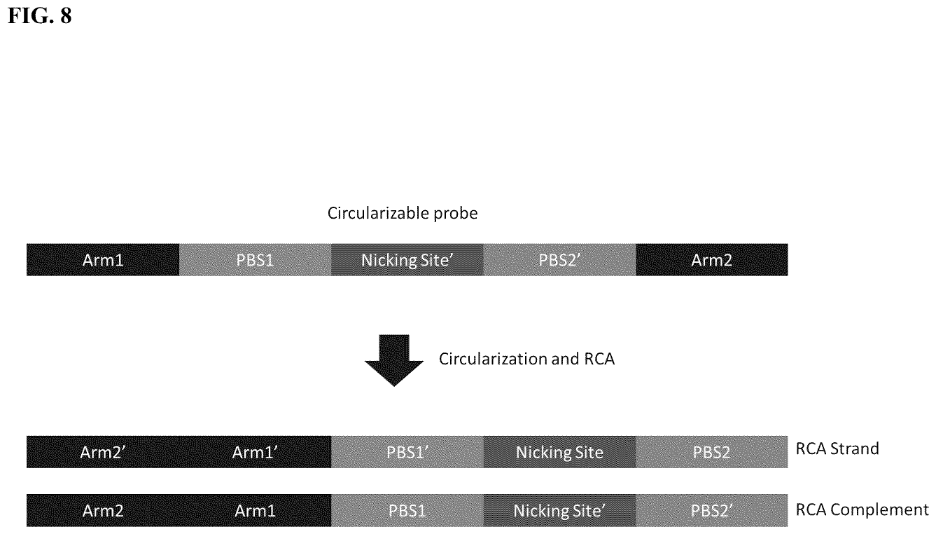

Alternatively sequencing primers (i.e., oligonucleotide sequences used for initiating sequencing reactions) may be immobilized to the solid support, or to cellular components in situ and used for sequencing following the amplification and nicking protocol described herein. FIGS. 7A-7E illustrate an embodiment of the invention described herein for amplifying (e.g., by exponential rolling circle amplification (eRCA)) a circular template polynucleotide (e.g., a circularized probe or circularized polynucleotide including a nicking site, or complement thereof) followed by sequencing with immobilized sequencing primers. FIG. 7A depicts annealing of the circular template polynucleotide to a first immobilized amplification oligonucleotide (e.g., an oligonucleotide or primer immobilized at a 5′ end of the primer to a solid support, or immobilized at a 5′ end of the primer to a cellular component or matrix in situ), and subsequent extension (e.g., extension with a strand-displacing polymerase) of the first immobilized oligonucleotide to generate an immobilized amplicon (e.g., an immobilized concatemer including a plurality of complements of the circular template polynucleotide). While only a single circular template polynucleotide is illustrated, it will be apparent to one of skill in the art that a plurality of circular template polynucleotides may be annealed and amplified across a plurality of first immobilized oligonucleotides (e.g., a first plurality of immobilized primers) using the methods described herein.

As highlighted in FIG. 7A, the immobilized amplicon includes a plurality of sequencing primer binding sequences that are complementary to the immobilized sequencing primer(s). The immobilized sequencing primer is blocked (denoted by the “X”) to prevent extension from the 3′ end until the blocking moiety (e.g., a reversible terminator moiety) is removed. FIG. 7B depicts hybridization of a second immobilized amplification oligonucleotide (e.g., a second amplification primer) to the immobilized RCA product of FIG. 7A, followed by extension of the second amplification primer to generate an extension product complementary to a portion of the immobilized RCA product. Additional second amplification primers may anneal to the immobilized RCA product and be extended with a strand-displacing polymerase, as illustrated in FIG. 7C, generating a plurality of immobilized complements of the RCA product, wherein each immobilized complement includes the complement of a portion of the RCA product, and wherein generating each additional immobilized complement displaces the previously generated immobilized complement. While not shown for clarity, it is to be understood that as additional immobilized complements are generated and displaced, one or more of the plurality of first amplification primers are then able to hybridize to the displaced immobilized complements and be extended (e.g., in an exponential RCA reaction).

A nicking reaction is then performed (e.g., nicking by a nicking endonuclease that nicks one strand of a double-stranded nucleic acid substrate) as illustrated in FIG. 7D, generating nicks across the immobilized RCA product strand, for example. After washing under denaturing conditions, for example, the nicked portions of the RCA product are removed, leaving behind a plurality of immobilized complements of the RCA product. Subsequently, the immobilized complements may hybridize to the immobilized sequencing primers, as shown in FIG. 7E. While the immobilized RCA product is shown as being nicked in these illustrations, in some embodiments, the immobilized complements are nicked, for example, by changing the orientation of the nicking strand (e.g., by including a complementary nicking site sequence in the circular template polynucleotide). For clarity, only a single immobilized amplicon is illustrated, but it will be understood that the plurality of immobilized complements of the RCA product, for example as depicted in FIG. 7D, are present and also able to hybridize to the immobilized sequencing primer. The blocking moiety is removed from the sequencing primers, and a sequencing reaction (using, for example, reversibly-terminated labeled nucleotide incorporation and detection) is performed to determine the sequence of the amplification products. In some embodiments, the immobilized complements are nicked and removed, and the immobilized RCA product(s) is detected.

### Example 5. Effective Amplification Primer Sequences

As described supra, e.g., in Example 3, rolling circle amplification is typically performed on a circular template polynucleotide, as described herein. The circular template polynucleotide may be a circularized endogenous nucleic acid, or a circularizable probe (e.g., a padlock probe, wherein the padlock probe may include the complement of a target endogenous sequence, for example a target mRNA sequence). An amplification primer (e.g., RCA primer) and a strand-displacing polymerase (e.g., phi29 polymerase, or a variant thereof) then initiate RCA of the circular template polynucleotide. Additional RCA primers may be included to perform exponential amplification of the RCA product (e.g., eRCA). The additional RCA primers may be, for example, present in solution or immobilized to a matrix or cellular component. As the RCA product is generated, the additional RCA primers can hybridize to complementary sequences in the RCA product and be extended by the strand-displacing polymerase, forming eRCA products. As the eRCA products will be complementary to the RCA products, a cluster of double-stranded amplification products will be formed. Due to the double-stranded nature of the amplification products, hybridizing a fluorescent probe or a sequencing primer is challenging, and limits detection. Increasing the accessibility of this cluster to, for example, a detection oligonucleotide would lead to improved detection signal.

We performed additional optimization of the primer sequences used for RCA and eRCA, and identified additional primer sequences that are suitable for use with the methods described herein. Briefly, following the eRCA reaction, a nicking endonuclease is introduced (e.g., a Nb.BbvCI nicking endonuclease) which generates nicks on one strand of the double-stranded eRCA product. Following the nicking reaction, the nicked strand is removed (e.g., by washing the eRCA product under denaturing conditions), thereby leaving behind a predominantly single-stranded amplification product. This results in realization of the benefits of eRCA (e.g., the increased speed of cluster formation), while significantly improving the accessibility of detection oligonucleotides to the nicked eRCA product. FIG. 8 illustrates a portion of a circularizable probe, wherein the circularizable probe includes a first target hybridization sequence (e.g., Arm1), a first primer binding sequence (PBS1), a nicking site complement, a second primer binding sequence (PBS2′), and a second target hybridization sequence (e.g., Arm2). Following circularization and rolling circle amplification (e.g., RCA or eRCA), an RCA amplicon strand and an RCA strand complement are generated, including complementary nicking sites.

FIGS. 5A-5E illustrate an embodiment of the invention described herein for amplifying (e.g., by exponential rolling circle amplification (eRCA)) a circular template polynucleotide (e.g., a circularized probe or circularized polynucleotide including a nicking site, or complement thereof). FIG. 5A depicts annealing of the circular template polynucleotide to a first immobilized amplification oligonucleotide (e.g., an oligonucleotide or primer immobilized at a 5′ end of the primer to a solid support, or immobilized at a 5′ end of the primer to a cellular component or matrix in situ), and subsequent extension (e.g., extension with a strand-displacing polymerase) of the first immobilized oligonucleotide to generate an immobilized amplicon (e.g., an immobilized concatemer including a plurality of complements of the circular template polynucleotide). While only a single circular template polynucleotide is illustrated, it will be apparent to one of skill in the art that a plurality of circular template polynucleotides may be annealed and amplified across a plurality of first immobilized oligonucleotides (e.g., a first plurality of immobilized primers) using the methods described herein. FIG. 5B depicts hybridization of a second immobilized amplification oligonucleotide (e.g., a second amplification primer) to the immobilized RCA product of FIG. 5A, followed by extension of the second amplification primer to generate an extension product complementary to a portion of the immobilized RCA product. Additional second amplification primers may anneal to the immobilized RCA product and be extended with a strand-displacing polymerase, as illustrated in FIG. 5C, generating a plurality of immobilized complements of the RCA product, wherein each immobilized complement includes the complement of a portion of the RCA product, and wherein generating each additional immobilized complement displaces the previously generated immobilized complement. While not shown for clarity, it is to be understood that as additional immobilized complements are generated and displaced, one or more of the plurality of first amplification primers are then able to hybridize to the displaced immobilized complements and be extended (e.g., in an exponential RCA reaction).

A nicking reaction is then performed (e.g., nicking by a nicking endonuclease that nicks one strand of a double-stranded nucleic acid substrate) as illustrated in FIG. 5D, generating nicks across the immobilized RCA product strand, for example. After washing under denaturing conditions, for example, the nicked portions of the RCA product are removed, leaving behind a plurality of immobilized complements of the RCA product as illustrated in FIG. 5E. While the immobilized RCA product is shown as being nicked in these illustrations, in some embodiments, the immobilized complements are nicked, for example, by changing the orientation of the nicking strand (e.g., by including a complementary nicking site sequence in the circular template polynucleotide). The immobilized complements may then be detected using, for example, labeled probes or subjected to a sequencing process as described herein. As illustrated, immobilized complements with longer portions of sequence may be bound by an increased number of labeled probes, resulting in greater signal intensity. In some embodiments, the immobilized complements are nicked and removed, and the immobilized RCA product(s) is detected.

Methods: In many cases, targeting low-abundance biomolecules as part of an in situ transcriptomics or proteomics study requires amplification of one or more probe oligonucleotides (e.g., amplifying a circular template polynucleotide including a barcode that corresponds to the target biomolecule) prior to, for example, performing in situ sequencing to detect the probe oligonucleotide. To show that the additional amplification primer sequence combinations described herein have improved and/or comparable amplification performance to the optimized eRCA approach described in Example 3, we selected a target gene (COL5A2) in the U-138MG glioblastoma cell line (ATCC HTB-16™) for targeting with padlock probes, and subsequent amplification and detection of the probes.

Plating and Fixation: The following were performed in 96-well plate format. Cells were seeded at a density of 2,000 live cells/well and cultured for 3 days. Cells were washed with 1×PBS, then fixed with 4% formaldehyde in 1×PBS for 20 min at room temperature (RT) and washed 3 times with 1×PBS to remove the formaldehyde. Cells were then permeabilized with 0.5% Triton X-100 in 1×PBS for 20 min at RT, then washed 1× with 1×PBS and 2× with hybridization buffer (20% formamide and 2×SSC in water).

Hybridization and Probe Ligation: Padlock probes (PLPs) were added at a final concentration of 100 nM each with 10 mM ribonucleoside vanadyl complex (RVC) in hybridization buffer with 0.2 U/μL SUPERase-In™ RNase inhibitor (Thermo Fisher Catalog #AM2694). PLPs were then allowed to hybridize overnight at 37° C. The cells were then washed 2× with hybridization buffer for 5 min at 37° C. and 1× with 1×PBS for 5 min each at 37° C. Following the washes, SplintR® ligase (New England Biolabs Catalog #M0375S) was added at a final concentration of 2.5 U/μL with 0.2 U/μL SUPERase-In™ RNase inhibitor (Thermo Fisher Catalog #AM2694) in 1× SplintR ligase buffer and incubated for 60 min at 37° C. Cells were then washed 2× with 1×PBS and 1× with hybridization buffer.

Primer Immobilization: Cells were treated with a THPP-containing buffer (20 mM CAPS pH10.0, 1M NaCl, 3 mM THPP) for 15 min at 60° C. Amplification primers (including a PEG8 linker and a bioconjugate reactive moiety (e.g., an DBCO or maleimide moiety)) were added to the cells at a final concentration of 2 μM and immobilized via a bioconjugation reaction for 2 hr at 37° C. Exemplary amplification primers are listed in Table 2. Primer pairs tested for eRCA include P5 (SEQ ID NO:25) and SP1 (SEQ ID NO:22), MIA (SEQ ID NO:27) and M7A (SEQ ID NO:48), S1 (SEQ ID NO:21) and S2 (SEQ ID NO:23), MIA (SEQ ID NO:27) and SP1 (SEQ ID NO:22), and MIA (SEQ ID NO:27) and M12A (SEQ ID NO:53). In some embodiments, amplification primers including a PEG4 linker are added to the cells. Cells were then washed 2× with hybridization buffer and 1× with PBS.

Rolling Circle Amplification: A mutant version of phi29 DNA polymerase was then added at a final concentration of 1.8 μM and incubated in the absence of co-factors or dNTPs for 15 minutes at 37° C. Amplification was then initiated with the mutant version of phi29 DNA polymerase at a final concentration of 1.8 μM in 1× phi29 buffer, dNTPs (0.5 mM each), 12 mM MgCl₂, and 0.2 U/μL SUPERase-InT RNase inhibitor in DEPC-treated water and incubated for 30 min at 37° C. Cells were then washed 3× with 1×PBS.

Nicking and denaturation: Nt.BsmAI nicking endonuclease (Cat. #R0121S, NEB) was added at a final concentration of 0.05 U/μL in 1× rCutSmart™ buffer (NEB) and incubated for 15 min at 37° C. Cells were then washed with a 100% formamide solution to remove the nicked strand portions.

Detection: TetraSpeck™ microspheres were added to crosslinked cells at a final concentration of 0.1 fM in 1×PBS and allowed to settle for at least 30 min at RT, or centrifuged for 3 min at 2,000 RPM. A Cy5-labeled probe and a dT30-FAM probed were then added to the cells in hybridization buffer, followed by washes with hybridization buffer and imaging.

Using the methods described herein, we observed in situ significant eRCA product formation when the nicking and denaturation steps were included following eRCA, similar to Example 3. Further, we found that each of the primer pair combinations listed supra generated comparable median intensity of the detectable clusters, indicating significant flexibility when choosing amplification primer combinations for use with the methods described herein. In some embodiments, the amplification primers include modified nucleotides (e.g., to increase the thermal stability of duplexed amplification primers). In embodiments, the

TABLE 2
Effective primer sequences. It is understood that white space, line
breaks, and text formatting are not indicative of separate sequences or structural implications.
The target polynucleotides may be amplified using primers with the sequences identified in this
table. In embodiments, one or more of the nucleotides are LNA nucleotides, e.g., nucleotides at
the 5′ end, to modulate the melting temperature.

| Primer | | SEQ ID |
|---|---|---|
| Name | Sequence (5′→3′) | Num. |
| S1 | ACAAAGGCAGCCACG CACTCCTTCCCTGT | SEQ ID |
| | | NO: 21 |
| SP1 | ACACTCTTTCCCTACA C GACGCTCTTCCGATCT | SEQ ID |
| | | NO: 22 |
| S2 | CTCCAGCGAGATGACC CTCACCAACCACT | SEQ ID |
| | | NO: 23 |
| SP2 | GTGACTGGAGTTCAGA CGTGTGCTCTTCCGATCT | SEQ ID |
| | | NO: 24 |
| P5 | AATGATACGGCGACCACCG | SEQ ID |
| | | NO: 25 |
| P7 | CAAGCAGAAGACGGCATACGAGAT | SEQ ID |
| | | NO: 26 |
| M1A | AACGCCAAACCTACGGCTTTACTTCCTGTGGC | SEQ ID |
| | | NO: 27 |
| M2A | TCTTGAGTCATTCGCAGGGCATGTGCCAGACC | SEQ ID |
| | | NO: 28 |
| M3A | TCGGCGTTGTCTGCTATCGTTCTTGGCACTCC | SEQ ID |
| | | NO: 29 |
| M4A | GGAGCAATAACCATAAGGCCGTTGACAAGCCC | SEQ ID |
| | | NO: 30 |
| M5A | GGCGTATTGCCTTGGTTCTGGCAGCCTCATTG | SEQ ID |
| | | NO: 31 |
| M1B | CAGCAGAGGGAACGATTTCAACTTCCTGTGGC | SEQ ID |
| | | NO: 32 |
| M2B | CTACTGCAAGGGTGTCTAGAATGTGCCAGACC | SEQ ID |
| | | NO: 33 |
| M3B | GACCGACTCGTGAAACGTAATCTTGGCACTCC | SEQ ID |
| | | NO: 34 |
| M4B | ACACATTCTTTGCGCCCAGAGTTGACAAGCCC | SEQ ID |
| | | NO: 35 |
| M5B | ATTTCATTCGACACCCGGTCGCAGCCTCATTG | SEQ ID |
| | | NO: 36 |
| M1A_RC | AGCCACAGGAAGTAAAGCCGTAGGTTTGGCGT | SEQ ID |
| | | NO: 37 |
| M2A_RC | AGGTCTGGCACATGCCCTGCGAATGACTCAAGA | SEQ ID |
| | | NO: 38 |
| M3A_RC | AGGAGTGCCAAGAACGATAGCAGACAACGCCGA | SEQ ID |
| | | NO: 39 |
| M4A_RC | AGGGCTTGTCAACGGCCTTATGGTTATTGCTCC | SEQ ID |
| | | NO: 40 |
| M5A_RC | ACAATGAGGCTGCCAGAACCAAGGCAATACGCC | SEQ ID |
| | | NO: 41 |
| M1B_RC | AGCCACAGGAAGTTGAAATCGTTCCCTCTGCTG | SEQ ID |
| | | NO: 42 |
| M2B_RC | AGGTCTGGCACATTCTAGACACCCTTGCAGTAG | SEQ ID |
| | | NO: 43 |
| M3B_RC | AGGAGTGCCAAGATTACGTTTCACGAGTCGGTC | SEQ ID |
| | | NO: 44 |
| M4B_RC | AGGGCTTGTCAACTCTGGGCGCAAAGAATGTGT | SEQ ID |
| | | NO: 45 |
| M5B_RC | ACAATGAGGCTGCGACCGGGTGTCGAATGAAAT | SEQ ID |
| | | NO: 46 |
| M6A | TGTTGCATCTCCACCCGGATTGAGCCTTCAGC | SEQ ID |
| | | NO: 47 |
| M7A | CACAACGGGAGCTGTGGAATTGGTTCACCTGG | SEQ ID |
| | | NO: 48 |
| M8A | TGGACTAAGACTCGTCCTCCAGCGGACCTAAG | SEQ ID |
| | | NO: 49 |
| M9A | GTATGATGGTGTTGCGGCTTCTCGCTTAACGC | SEQ ID |
| | | NO: 50 |
| M10A | TCTGAGTGCCAGTGACTTCACGCATTCGCTTG | SEQ ID |
| | | NO: 51 |
| M11A | TACGACACACTCGGGCTCTATGGGCTTCATGG | SEQ ID |
| | | NO: 52 |
| M12A | GTTTGAGTGAAGGCGGTCCAACCCTTAGTGCG | SEQ ID |
| | | NO: 53 |
| M6B | CTATAAGTTTGTCGTGCCCGTGAGCCTTCAGC | SEQ ID |
| | | NO: 54 |
| M7B | GGAGTGACACTGACTACGTTTGGTTCACCTGG | SEQ ID |
| | | NO: 55 |
| M8B | GTCAACGCCCTAGCAGACATAGCGGACCTAAG | SEQ ID |
| | | NO: 56 |
| M9B | CCAGAACCTATTGAGCCTGACTCGCTTAACGC | SEQ ID |
| | | NO: 57 |
| M10B | AGGTGTTCGTACAATGAGGCCGCATTCGCTTG | SEQ ID |
| | | NO: 58 |
| M11B | TGGTCAAGGGCAACTAATCCTGGGCTTCATGG | SEQ ID |
| | | NO: 59 |
| M12B | ACAATTACCCGTTTACCGGCACCCTTAGTGCG | SEQ ID |
| | | NO: 60 |
| M6A_RC | AGCTGAAGGCTCAATCCGGGTGGAGATGCAACA | SEQ ID NO: |
| | | 61 |
| M7A_RC | ACCAGGTGAACCAATTCCACAGCTCCCGTTGTG | SEQ ID NO: |
| | | 62 |
| M8A_RC | ACTTAGGTCCGCTGGAGGACGAGTCTTAGTCCA | SEQ ID NO: |
| | | 63 |
| M9A_RC | AGCGTTAAGCGAGAAGCCGCAACACCATCATAC | SEQ ID NO: |
| | | 64 |
| M10A_RC | ACAAGCGAATGCGTGAAGTCACTGGCACTCAGA | SEQ ID NO: |
| | | 65 |
| M11A_RC | ACCATGAAGCCCATAGAGCCCGAGTGTGTCGTA | SEQ ID NO: |
| | | 66 |
| M12A_RC | ACGCACTAAGGGTTGGACCGCCTTCACTCAAAC | SEQ ID NO: |
| | | 67 |
| M6B_RC | AGCTGAAGGCTCACGGGCACGACAAACTTATAG | SEQ ID NO: |
| | | 68 |
| M7B_RC | ACCAGGTGAACCAAACGTAGTCAGTGTCACTCC | SEQ ID NO: |
| | | 69 |
| M8B_RC | ACTTAGGTCCGCTATGTCTGCTAGGGCGTTGAC | SEQ ID NO: |
| | | 70 |
| M9B_RC | AGCGTTAAGCGAGTCAGGCTCAATAGGTTCTGG | SEQ ID NO: |
| | | 71 |
| M10B_RC | ACAAGCGAATGCGGCCTCATTGTACGAACACCT | SEQ ID NO: |
| | | 72 |
| M11B_RC | ACCATGAAGCCCAGGATTAGTTGCCCTTGACCA | SEQ ID NO: |
| | | 73 |
| M12B_RC | ACGCACTAAGGGTGCCGGTAAACGGGTAATTGT | SEQ ID NO: |
| | | 74 |

### P-EMBODIMENTS

The present disclosure provides the following illustrative embodiments.
Embodiment P1. A method of forming single-stranded polynucleotides in situ, the method comprising: (a) within a cell or tissue, extending a first primer hybridized to a circular polynucleotide with a strand-displacing polymerase to generate a first extension product comprising one or more complements of the circular polynucleotide; (b) contacting the first extension product with a second immobilized primer and extending the second immobilized primer with a polymerase to generate a second immobilized extension product, wherein the second primer is immobilized to a cellular component or a matrix within the cell or tissue; and (c) nicking the first extension product with an endonuclease, thereby generating one or more polynucleotide fragments, and removing said polynucleotide fragments, thereby forming single-stranded polynucleotides in situ.
Embodiment P2. The method of Embodiment P1, further comprising: (d) hybridizing a detection probe to the second immobilized extension product and detecting said detection probe, thereby detecting the circular polynucleotide.
Embodiment P3. The method of Embodiment P1, further comprising, prior to step (c), contacting the second immobilized extension product with a third immobilized primer and extending the third immobilized primer with a polymerase to generate a third immobilized extension product, wherein the third immobilized primer is immobilized to the cellular component or the matrix within the cell or tissue.
Embodiment P4. The method of Embodiment P3, wherein step (c) further comprises nicking the second immobilized extension product with an endonuclease, thereby generating one or more additional polynucleotide fragments.
Embodiment P5. The method of Embodiment P1, further comprising, after step (c), detecting the second immobilized extension product.
Embodiment P6. The method of Embodiment P4, further comprising, after step (c), detecting the third immobilized extension product.
Embodiment P7. The method of Embodiment P1, further comprising, after step (c), sequencing the second immobilized extension product.
Embodiment P8. The method of Embodiment P4, further comprising, after step (c), sequencing the third immobilized extension product.
Embodiment P9. The method of Embodiment P7 or Embodiment P8, wherein the sequencing comprises sequencing by synthesis, sequencing by hybridization, sequencing by binding, sequencing by ligation, or pyrosequencing.
Embodiment P10. The method of Embodiment P7 or Embodiment P8, wherein the sequencing comprises extending a sequencing primer by incorporating a labeled nucleotide or labeled nucleotide analogue, and detecting the label to generate a signal for each incorporated nucleotide or nucleotide analogue, wherein the sequencing primer is hybridized to the extension product.
Embodiment P11. A method of forming single-stranded polynucleotides on a solid support, the method comprising: (a) extending a first primer hybridized to a circular polynucleotide with a strand-displacing polymerase to generate a first extension product comprising one or more complements of the circular polynucleotide; (b) contacting the first extension product with a second immobilized primer and extending the second immobilized primer with a polymerase to generate a second immobilized extension product, wherein the second primer is immobilized to a solid support; and (c) nicking the first extension product with an endonuclease, thereby generating one or more polynucleotide fragments, and removing said polynucleotide fragments, thereby forming single-stranded polynucleotides on a solid support.
Embodiment P12. The method of claim 11, further comprising: (d) hybridizing a detection probe to the second immobilized extension product and detecting said detection probe, thereby detecting the circular polynucleotide.
Embodiment P13. A method of sequencing a circular polynucleotide, the method comprising: i) amplifying the circular polynucleotide in a cell or tissue by extending a first primer hybridized to said circular polynucleotide with a strand-displacing polymerase to generate a first extension product comprising one or more complements of the circular polynucleotide; ii) contacting the first extension product with a second primer and extending the second primer with a polymerase to generate a second immobilized extension product, wherein the second primer is immobilized to a cellular component or a matrix within the cell or tissue; iii) nicking the first extension product with an endonuclease, thereby generating one or more polynucleotide fragments, and removing said polynucleotide fragments, thereby forming single-stranded polynucleotides on said solid support; and iv) hybridizing a sequencing primer to the single-stranded polynucleotides, and extending the sequencing primer to generate a first sequencing read, wherein said sequencing primer is immobilized to a cellular component or a matrix within the cell or tissue.
Embodiment P14. The method of Embodiment P13, further comprising, prior to step (iii), contacting the second immobilized extension product with a third primer and extending the third primer with a polymerase to generate a third immobilized extension product, wherein the third primer is immobilized to a cellular component or a matrix within the cell or tissue.
Embodiment P15. The method of Embodiment P14, wherein step (iii) comprises nicking the second immobilized extension product with an endonuclease, thereby generating one or more additional polynucleotide fragments, and removing said additional polynucleotide fragments.
Embodiment P16. The method of any one of Embodiment P13 to Embodiment P15, wherein the sequencing primer comprises a reversible 3′ blocking moiety.
Embodiment P17. The method of Embodiment P16, wherein the reversible blocking moiety comprises a dideoxy nucleotide triphosphate.
Embodiment P18. The method of Embodiment P16 or Embodiment P17, wherein prior to step iv), the reversible blocking moiety is removed, thereby generating an extendible sequencing primer.
Embodiment P19. The method of any one of Embodiment P2 to Embodiment P18, wherein the method comprises amplifying the circular polynucleotide of the cell in situ.
Embodiment P20. The method of any one of Embodiment P1 to Embodiment P19, wherein the endonuclease lacks double-strand cleavage activity.
Embodiment P21. The method of any one of Embodiment P1 to Embodiment P20, wherein the endonuclease comprises one or more endonucleases selected from the group consisting of Nb.BbvCI, Nb.BsmI, NbBsrDI, Nb.BtsI, Nt.AlwI, Nt.BbvCI, Nb.BssSI, Nt.BsmAI, Nt.BspQI, Nt.BstNBI, and Nt.CviPII.
Embodiment P22. The method of Embodiment P21, wherein the endonuclease is Nb.BbvCI.
Embodiment P23. The method of Embodiment P1 or Embodiment P13, wherein removing said nicked first extension product comprises contacting said nicked first extension product with a chemical denaturant.
Embodiment P24. The method of Embodiment P4 or Embodiment P15, wherein removing said nicked second extension product comprises contacting said nicked second extension product with a chemical denaturant.
Embodiment P25. The method of Embodiment P23 or Embodiment P24, wherein the chemical denaturant comprises ethylene glycol, polyethylene glycol, 1,2-propanediol, dimethyl sulfoxide (DMSO), glycerol, formamide, 7-deaza-dGTP, acetamide, betaine, or tetramethylammonium chloride (TMAC).
Embodiment P26. The method of Embodiment P25, wherein the chemical denaturant comprises 100% formamide.
Embodiment P27. The method of any one of Embodiment P1 to Embodiment P26, wherein the circular polynucleotide comprises primer binding sequences complementary to one or more additional primers.
Embodiment P28. The method of any one of Embodiment P1 to Embodiment P27, wherein the circular polynucleotide comprises a sequence, or a complement thereof, recognized by the endonuclease.
Embodiment P29. The method of any one of Embodiment P1 to Embodiment P28, wherein amplifying the circular polynucleotide comprises rolling circle amplification (RCA), exponential rolling circle amplification (eRCA), hyperbranched rolling circle amplification (HRCA), loop-mediated isothermal amplification (LAMP), or multiple displacement amplification (MDA).
Embodiment P30. The method of Embodiment P29, wherein amplifying the circular polynucleotide comprises rolling circle amplification (RCA) or exponential rolling circle amplification (eRCA).
Embodiment P31. The method of any one of Embodiment P1 to Embodiment P30, wherein the circular polynucleotide is single-stranded DNA.
Embodiment P32. A kit comprising a circularizable probe, a ligase, and an endonuclease, wherein said circularizable probe comprises a first hybridization sequence capable of hybridizing to a first sequence of a target polynucleotide, a second hybridization sequence capable of hybridizing to a second sequence of said target polynucleotide, and a sequence recognized by said endonuclease.
Embodiment P33. The kit of Embodiment P32, wherein the endonuclease comprises one or more endonucleases selected from the group consisting of Nb.BbvCI, Nb.BsmI, NbBsrDI, Nb.BtsI, Nt.AlwI, Nt.BbvCI, Nb.BssSI, Nt.BsmAI, Nt.BspQI, Nt.BstNBI, and Nt.CviPII.
Embodiment P34. The kit of Embodiment P33, wherein the endonuclease is Nb.BbvCI.
Embodiment P35. The kit of any one of Embodiment P32 to Embodiment P34, wherein the circularizable probe further comprises a barcode sequence.
Embodiment P36. The kit of Embodiment P35, wherein the barcode sequence is at least 5, 6, 7, 8, 9, 10, 11, 12, 13, 14, or 15 nucleotides in length.
Embodiment P37. The kit of Embodiment P35 or Embodiment P36, wherein the barcode sequence is selected from a known set of barcode sequences.
Embodiment P38. The kit of any one of Embodiment P32 to Embodiment P37, wherein the target polynucleotide comprises a cancer-associated gene nucleic acid sequence, a viral nucleic acid sequence, a bacterial nucleic acid sequence, or a fungal nucleic acid sequence.
Embodiment P39. The kit of any one of Embodiment P32 to Embodiment P38, wherein the target polynucleotide is an RNA nucleic acid sequence or DNA nucleic acid sequence.
Embodiment P40. The kit of any one of Embodiment P32 to Embodiment P39, wherein the first hybridization sequence and the second hybridization sequence are each about 5 to about 35 nucleotides in length.
Embodiment P41. The kit of any one of Embodiment P32 to Embodiment P40, wherein the circularizable probe comprises one or more primer binding sequences.
Embodiment P42. The kit of any one of Embodiment P32 to Embodiment P41, wherein the circularizable probe comprises at least one amplification primer binding sequence or at least one sequencing primer binding sequence.
Embodiment P43. The kit of any one of Embodiment P32 to Embodiment P42, wherein the circularizable probe comprises a barcode sequence.
Embodiment P44. The kit of Embodiment P43, wherein the barcode sequence is at least 5, 6, 7, 8, 9, 10, 11, 12, 13, 14, or 15 nucleotides in length.
Embodiment P45. The kit of Embodiment P43 or Embodiment P44, wherein the barcode sequence is selected from a known set of barcode sequences.
Embodiment P46. The kit of any one of Embodiment P32 to Embodiment P45, wherein the first sequence comprises a nucleic acid sequence encoding a B cell receptor V region, and wherein the second sequence comprises a nucleic acid sequence encoding a B cell receptor J region.
Embodiment P47. The kit of any one of Embodiment P32 to Embodiment P46, wherein the first sequence and the second sequence flank a CDR3 nucleic acid sequence.
Embodiment P48. The kit of any one of Embodiment P32 to Embodiment P47, wherein said target polynucleotide comprises a cancer-associated gene nucleic acid sequence, a viral nucleic acid sequence, a bacterial nucleic acid sequence, or a fungal nucleic acid sequence.

What is claimed is:

1. A method of forming single-stranded polynucleotides in situ, the method comprising:
(a) extending a first primer hybridized to a circular polynucleotide with a strand-displacing polymerase to generate a first extension product comprising one or more complements of the circular polynucleotide within a cell or tissue;
(b) contacting the first extension product with a second immobilized primer and extending the second immobilized primer with a polymerase to generate a second immobilized extension product, wherein the second primer is immobilized to a cellular component or a matrix within the cell or tissue; and
(c) nicking the first extension product with an endonuclease, thereby generating one or more polynucleotide fragments, and removing said polynucleotide fragments, thereby forming single-stranded polynucleotides in situ.

2. The method of claim 1, further comprising:
(d) hybridizing a detection probe to the second immobilized extension product and detecting said detection probe, thereby detecting the circular polynucleotide.

3. The method of claim 1, further comprising, prior to step (c), contacting the second immobilized extension product with a third immobilized primer and extending the third immobilized primer with a polymerase to generate a third immobilized extension product, wherein the third immobilized primer is covalently immobilized to the cellular component or the matrix within the cell or tissue.

4. The method of claim 3, wherein step (c) further comprises nicking the second immobilized extension product with an endonuclease, thereby generating one or more additional polynucleotide fragments.

5. The method of claim 3, further comprising, after step (c), detecting the second immobilized extension product; followed by detecting the third immobilized extension product.

6. The method of claim 1, further comprising, after step (c), sequencing the second immobilized extension product.

7. The method of claim 4, further comprising, after step (c), sequencing the third immobilized extension product.

8. The method of claim 6, wherein the sequencing comprises sequencing by synthesis, sequencing by hybridization, sequencing by binding, sequencing by ligation, or pyrosequencing.

9. The method of claim 1, further comprising binding a specific binding reagent to a protein in the cell or tissue, wherein the specific binding reagent includes an oligonucleotide barcode, and sequencing the oligonucleotide barcode.

10. The method of claim 1, prior to step (a), forming the circular polynucleotide by hybridizing a first sequence of a circularizable oligonucleotide to a target nucleic acid molecule and a second sequence of the circularizable oligonucleotide to the target nucleic acid molecule, and ligating the first sequence and the second sequence to form the circular polynucleotide.

11. The method of claim 10, wherein the first sequence and the second sequence are adjacent.

12. The method of claim 10, wherein the first sequence and the second sequence are separated by 1 or more nucleotides.

13. A method of sequencing a circular polynucleotide, the method comprising:
i) amplifying the circular polynucleotide in a cell or tissue by extending a first primer hybridized to said circular polynucleotide with a strand-displacing polymerase to generate a first extension product comprising one or more complements of the circular polynucleotide;
ii) contacting the first extension product with a second primer and extending the second primer with a polymerase to generate a second immobilized extension product, wherein the second primer is immobilized to a cellular component or a matrix within the cell or tissue;
iii) nicking the first extension product with an endonuclease, thereby generating one or more polynucleotide fragments, and removing said polynucleotide fragments, thereby forming single-stranded polynucleotides on said solid support; and
iv) hybridizing a sequencing primer to the single-stranded polynucleotides, and extending the sequencing primer to generate a first sequencing read, wherein said sequencing primer is immobilized to a cellular component or a matrix within the cell or tissue.

14. The method of claim 13, further comprising, prior to step (iii), contacting the second immobilized extension product with a third primer and extending the third primer with a polymerase to generate a third immobilized extension product, wherein the third primer is immobilized to a cellular component or a matrix within the cell or tissue.

15. The method of claim 14, wherein step (iii) comprises nicking the second immobilized extension product with an endonuclease, thereby generating one or more additional polynucleotide fragments, and removing said additional polynucleotide fragments.

16. The method of claim 12, wherein the endonuclease comprises one or more endonucleases selected from the group consisting of Nb.BbvCI, Nb.BsmI, NbBsrDI, Nb.BtsI, Nt.AlwI, Nt.BbvCI, Nb.BssSI, Nt.BsmAI, Nt.BspQI, Nt.BstNBI, and Nt.CviPII.

17. The method of claim 1, wherein the circular polynucleotide comprises any one of SEQ ID NO:1 to SEQ ID NO:20, or a complement thereof.

18. A kit comprising a circularizable probe, a ligase, and an endonuclease, wherein said circularizable probe comprises a first hybridization sequence capable of hybridizing to a first sequence of a target polynucleotide, a second hybridization sequence capable of hybridizing to a second sequence of said target polynucleotide, and a sequence recognized by said endonuclease.

19. The kit of claim 18, wherein the first sequence comprises a nucleic acid sequence encoding a B cell receptor V region, and wherein the second sequence comprises a nucleic acid sequence encoding a B cell receptor J region.

20. The kit of claim 18, wherein the circular polynucleotide comprises any one of SEQ ID NO:1 to SEQ ID NO:20, or a complement thereof.
